(19) United States
(12) Patent Application Publication
(10) Pub. No.: US 2015/0340626 A1
(43) Pub. Date: Nov. 26, 2015

(54) ORGANIC COMPOUND, COMPOSITION, ORGANIC OPTOELECTRIC DEVICE, AND DISPLAY DEVICE

(71) Applicant: SAMSUNG SDI CO., LTD., Yongin-si (KR)

(72) Inventors: Han-Ill LEE, Suwon-si (KR); Chang-Ju SHIN, Suwon-si (KR); Su-Jin HAN, Suwon-si (KR); Young-Kwon KIM, Suwon-si (KR); Soo-Hyun MIN, Suwon-si (KR); Eun-Sun YU, Suwon-si (KR); Ho-Kuk JUNG, Suwon-si (KR)

(21) Appl. No.: 14/614,951

(22) Filed: Feb. 5, 2015

(30) Foreign Application Priority Data
May 20, 2014 (KR) 10-2014-0060527

(51) Int. Cl. H01L 51/00; C09K 11/02; C09K 11/06; C07D 239/70

(52) U.S. Cl. CPC H01L 51/0072; C07D 239/70; C09K 11/025; C09K 11/06; H01L 51/5012

(57) ABSTRACT

An organic compound, a composition for an organic optoelectric device, and a display device, the compound being represented by the following Chemical Formula 1:

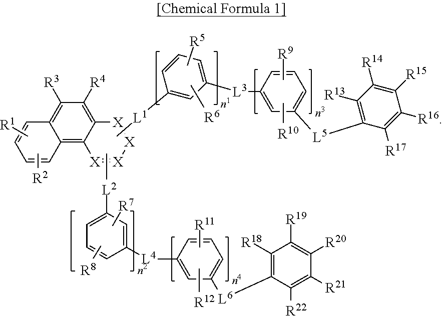

### CROSS-REFERENCE TO RELATED APPLICATION

Korean Patent Application No. 10-2014-0060527, filed on May 20, 2014, in the Korean Intellectual Property Office, and entitled: “Organic Compound and Composition and Organic Optoelectric Device and Display Device,” is incorporated by reference herein in its entirety.

### BACKGROUND

1. Field

Embodiments relate to an organic compound, a composition, an organic optoelectric device, and a display device.

2. Description of the Related Art

An organic optoelectric device is a device that converts electrical energy into photoenergy, and vice versa.

An organic optoelectric device may be classified as follows in accordance with its driving principles. One type may include an electronic device in which excitons generated by photoenergy are separated into electrons and holes and the electrons and holes are transferred to different electrodes respectively and electrical energy is generated. Another type may include a light emitting device to generate photoenergy from electrical energy by supplying a voltage or a current to electrodes.

Examples of the organic optoelectric device may include an organic photoelectric device, an organic light emitting diode, an organic solar cell, and an organic photo-conductor drum, and the like.

The organic light emitting diode (OLED) has recently drawn attention due to an increase in demand for flat panel displays. The organic light emitting diode converts electrical energy into light by applying current to an organic light emitting material, and may have a structure in which an organic is interposed between an anode and a cathode.

### SUMMARY

Embodiments are directed to an organic compound, a composition, an organic optoelectric device, and a display device.

The embodiments may be realized by providing an organic compound represented by the following Chemical Formula 1:

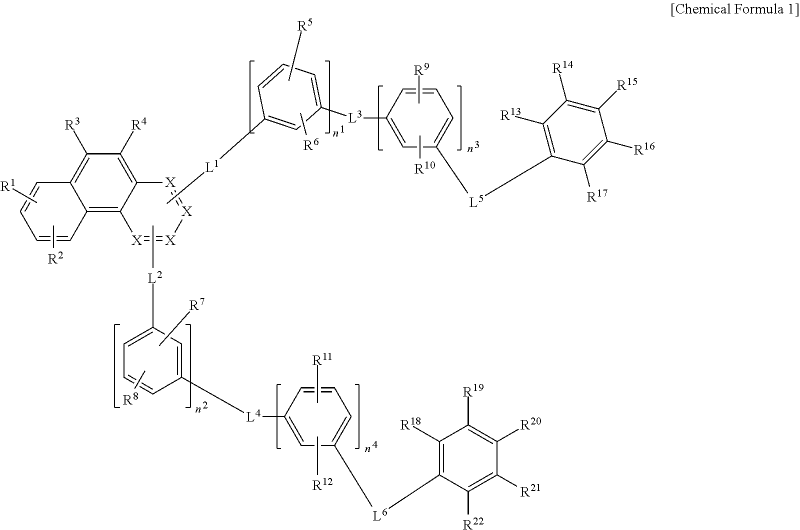

wherein, in Chemical Formula 1, two of X are nitrogen and two of X are carbon, R¹ to R⁴ are each independently hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof, R⁵ to R¹² are each independently hydrogen, deuterium, a substituted or unsubstituted C1 to C10 alkyl group, a substituted or unsubstituted C6 to C12 aryl group, or a combination thereof, R¹³ to R²² are each independently hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof, or two adjacent ones thereof are linked to each other to form a fused ring, L¹ to L⁶ are each independently a single bond, a substituted or unsubstituted phenylene group, a substituted or unsubstituted biphenylene group, a substituted or unsubstituted terphenylene group, or a substituted or unsubstituted quaterphenylene group, n¹ to n⁴ are each independently an integer of 0 to 5, and a sum of n¹ to n⁴ is an integer of 2 or more.

The compound represented by Chemical Formula 1 may be represented by one of the following Chemical Formula 2 or Chemical Formula 3:

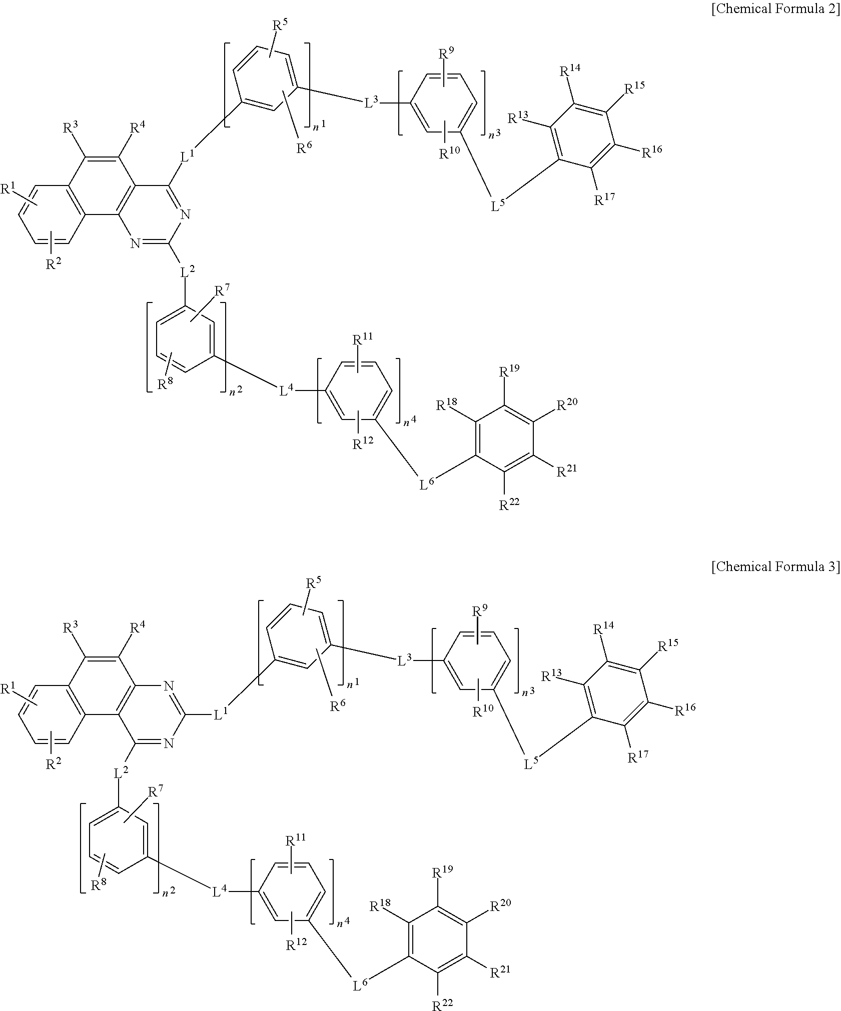

wherein, in Chemical Formulae 2 and 3, R¹ to R⁴ are each independently hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof, R⁵ to R¹² are each independently hydrogen, deuterium, a substituted or unsubstituted C1 to C10 alkyl group, a substituted or unsubstituted C6 to C12 aryl group, or a combination thereof, R¹³ to R²² are each independently hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof, or two adjacent ones thereof are linked to each other to form a fused ring, L¹ to L⁶ are each independently a single bond, a substituted or unsubstituted phenylene group, a substituted or unsubstituted biphenylene group, a substituted or unsubstituted terphenylene group, or a substituted or unsubstituted quaterphenylene group, n¹ to n⁴ are each independently an integer of 0 to 5, and a sum of n¹ to n⁴ is an integer of 2 or more, preferable 6 or less.

The compound represented by Chemical Formula 1 may be represented by one of the following Chemical Formula 2A or Chemical Formula 3A:

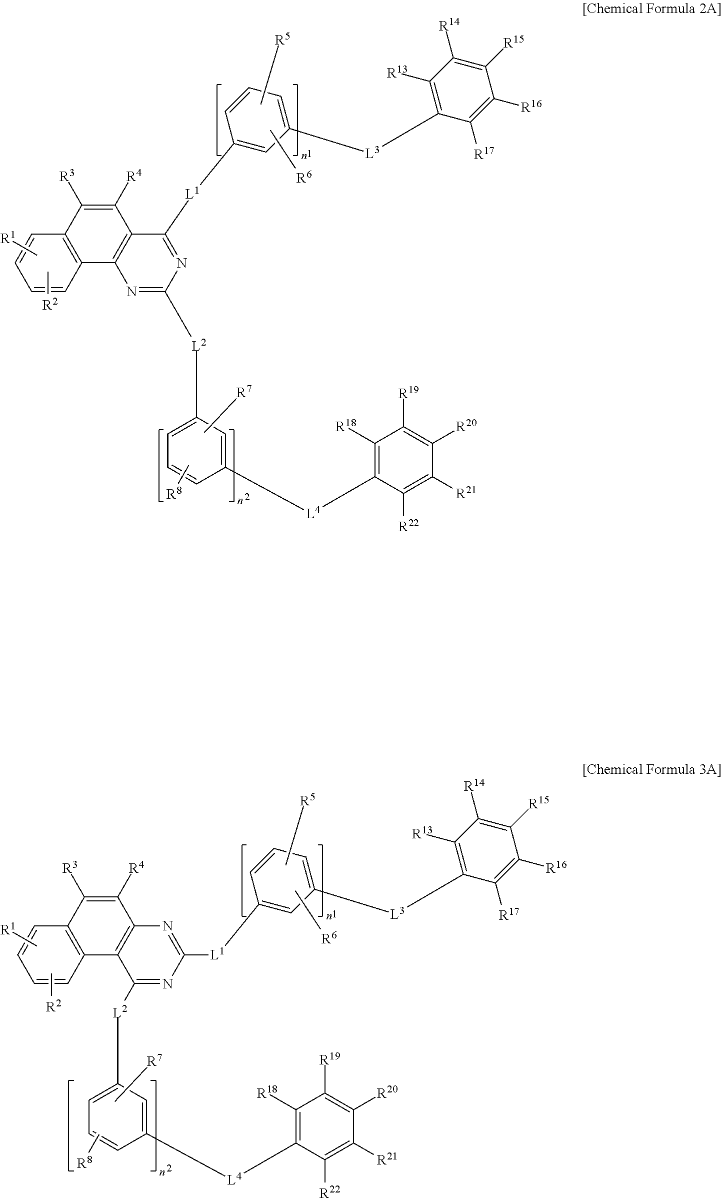

wherein, in Chemical Formulae 2A and 3A, R¹ to R⁴ are each independently hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof, R⁵ to R⁸ are each independently hydrogen, deuterium, a substituted or unsubstituted C1 to C10 alkyl group, a substituted or unsubstituted C6 to C12 aryl group, or a combination thereof, R¹³ to R²² are each independently hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof, or two adjacent ones thereof are linked to each other to form a fused ring, L¹ to L⁴ are each independently a single bond, a substituted or unsubstituted phenylene group, a substituted or unsubstituted biphenylene group, a substituted or unsubstituted terphenylene group, or a substituted or unsubstituted quaterphenylene group, n¹ and n² are each independently an integer of 0 to 5, and a sum of n¹ and n² is an integer of 2 or more.

n¹ and n² of Chemical Formula 1 may each independently be an integer of 1 to 5.

R³ and R⁴ of Chemical Formula 1 may be hydrogen.

L¹ to L⁶ of Chemical Formula 1 may each independently be a single bond or a group of the following Group 1:

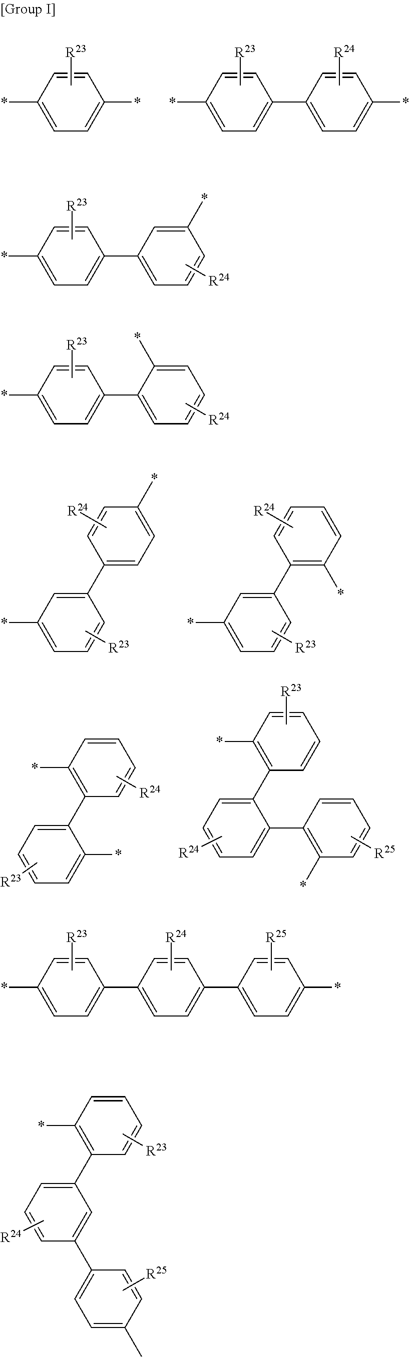

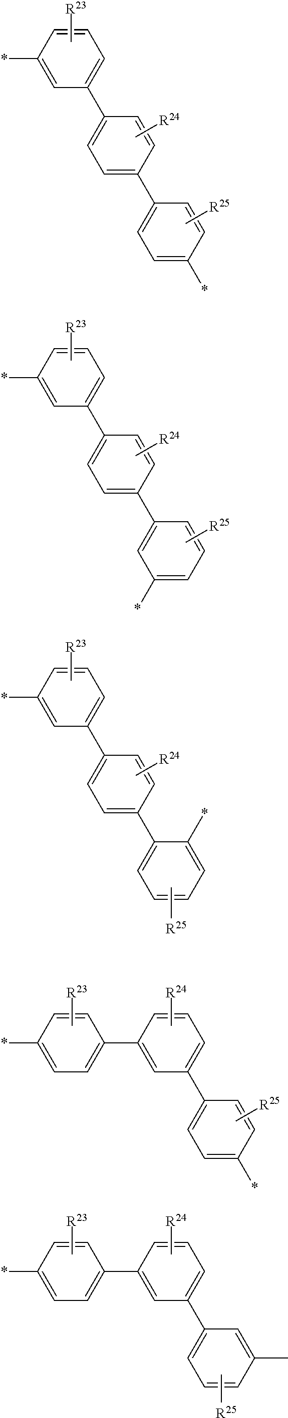

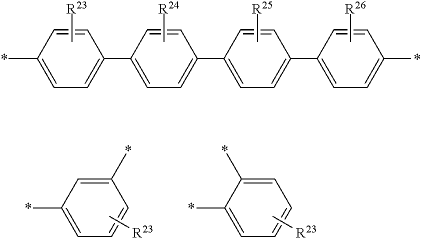

wherein, in Group 1, R²³ to R²⁶ may each independently be hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof, and * is a linking point to a neighboring atom.

R²³ to R²⁶ may be hydrogen.

The embodiments may be realized by providing a composition for an organic optoelectric device, the composition including the organic compound according to an embodiment, and a second organic compound that includes a carbazole moiety.

The second organic compound may include at least one of a compound represented by the following Chemical Formula 4 or a compound consisting of a combination of a moiety represented by the following Chemical Formula 5 and a moiety represented by the following Chemical Formula 6:

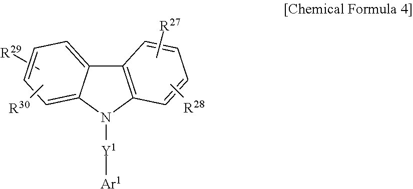

wherein, in Chemical Formula 4, Y¹ may be a single bond, a substituted or unsubstituted C1 to C20 alkylene group, a substituted or unsubstituted C2 to C20 alkenylene group, a substituted or unsubstituted C6 to C30 arylene group, a substituted or unsubstituted C2 to C30 heterocyclic group, or a combination thereof, Ar¹ may be a substituted or unsubstituted C6 to C30 aryl group, a substituted or unsubstituted C2 to C30 heterocyclic group, or a combination thereof, R²⁷ to R³⁰ may each independently be hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof, and at least one of R²⁷ to R³⁰ and Ar¹ may include a substituted or unsubstituted triphenylene group or a substituted or unsubstituted carbazole group,

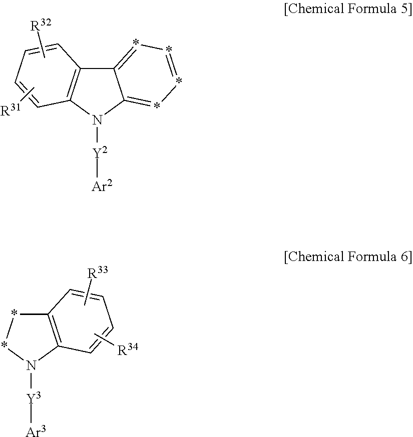

wherein, in Chemical Formulae 5 and 6, Y² and Y³ may each independently be a single bond, a substituted or unsubstituted C1 to C20 alkylene group, a substituted or unsubstituted C2 to C20 alkenylene group, a substituted or unsubstituted C6 to C30 arylene group, a substituted or unsubstituted C2 to C30 heterocyclic group, or a combination thereof, Ar^{e} and Ar^{a} may each independently be substituted or unsubstituted C6 to C30 aryl group, a substituted or unsubstituted C2 to C30 heterocyclic group, or a combination thereof, R³¹ to R³⁴ may each independently be hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof, adjacent two *s of Chemical Formula 5 may be combined with two *s of Chemical Formula 6 to form a fused ring, and *s of Chemical Formula 5 that do not form a fused ring are each independently CR^{a}, and R^{a} may be hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof.

The compound represented by Chemical Formula 4 may be represented by one of the following Chemical Formulae 4-I to 4-III:

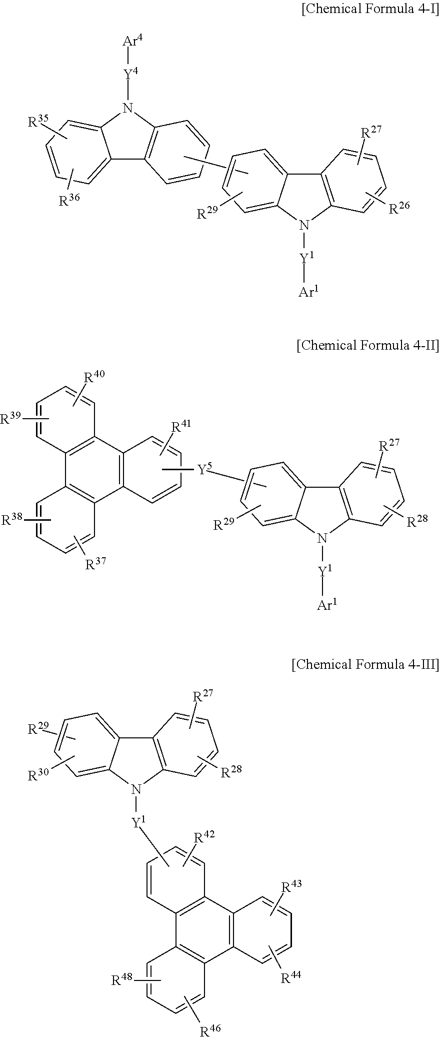

wherein, in Chemical Formulae 4-I to 4-III, Y¹, Y⁴, and Y⁵ may each independently be a single bond, a substituted or unsubstituted C1 to C20 alkylene group, a substituted or unsubstituted C2 to C20 alkenylene group, a substituted or unsubstituted C6 to C30 arylene group, a substituted or unsubstituted C2 to C30 heterocyclic group, or a combination thereof, Ar¹ and Ar⁴ may each independently be a substituted or unsubstituted C6 to C30 aryl group, a substituted or unsubstituted C2 to C30 heterocyclic group, or a combination thereof, and R²⁷ to R³⁰ and R³⁵ to R⁴⁶ may each independently be hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof.

The first organic compound and the second organic compound may be included in the composition in a weight ratio of about 1:10 to about 10:1.

The composition may further include a phosphorescent dopant.

The embodiments may be realized by providing an organic optoelectric device including an anode and a cathode facing each other, and at least one organic layer between the anode and the cathode, wherein the organic layer includes the organic compound according to an embodiment.

The organic layer may include an emission layer, and the emission layer may include the organic compound.

The organic compound may be a host in the emission layer.

The embodiments may be realized by providing a display device including the organic optoelectric device according to an embodiment.

The embodiments may be realized by providing an organic optoelectric device including an anode and a cathode facing each other, and at least one organic layer between the anode and the cathode, wherein the organic layer includes the composition according to an embodiment.

The organic layer may include an emission layer, and the emission layer may include the composition.

The composition may be a host in the emission layer.

The embodiments may be realized by providing a display device including the organic optoelectric device according to an embodiment.

### BRIEF DESCRIPTION OF THE DRAWINGS

Features will be apparent to those of skill in the art by describing in detail exemplary embodiments with reference to the attached drawings in which:

FIGS. 1 and 2 illustrate cross-sectional views of organic light emitting diodes according to the embodiments.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. Like reference numerals refer to like elements throughout.

As used herein, when a definition is not otherwise provided, the term “substituted” refers to one substituted with a deuterium, a halogen, a hydroxy group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C3 to C30 cycloalkyl group, a C3 to C30 heterocycloalkyl group, a C6 to C30 aryl group, a C6 to C30 heterocycle, a C1 to C20 alkoxy group, a fluoro group, a C1 to C10 trifluoroalkyl group such as a trifluoromethyl group, and the like, or a cyano group, instead of at least one hydrogen of a substituent or a compound.

In addition, two adjacent substituents of the substituted halogen, hydroxy group, amino group, substituted or unsubstituted C1 to C30 amine group, nitro group, substituted or unsubstituted C1 to C40 silyl group, C1 to C30 alkyl group, C1 to C10 alkylsilyl group, C3 to C30 cycloalkyl group, C3 to C30 heterocycloalkyl group, C6 to C30 aryl group, C6 to C30 heterocyclic, C1 to C20 alkoxy group, fluoro group, C1 to C10 trifluoroalkyl group such as a trifluoromethyl group, and the like, or cyano group may be fused with each other to provide a ring. For example, the substituted C6 to C30 aryl group may be fused with another adjacent substituted C6 to C30 aryl group to form a substituted or unsubstituted fluorene ring.

In the present specification, when specific definition is not otherwise provided, the term “hetero” refers to one including 1 to 3 hetero atoms selected from N, O, S, P, and Si, and remaining carbons in one functional group.

As used herein, the term “aryl group” refers to a substituent including all element of the cycle having p-orbitals which form conjugation, and may be monocyclic, polycyclic or fused ring polycyclic (i.e., rings sharing adjacent pairs of carbon atoms) functional group.

As used herein, the term “heterocycle” or “heterocyclic group” may refer to a cyclic compound such as an aryl group or a cycloalkyl group including 1 to 3 hetero atoms selected from N, O, S, P, and Si and remaining carbons in one functional group. When the heterocycle is a fused ring, the entire ring or each ring of the heterocycle may include one or more hetero atom.

For example, the substituted or unsubstituted C6 to C30 aryl group and/or the substituted or unsubstituted C2 to C30 heterocyclic group may be a substituted or unsubstituted phenyl group, a substituted or unsubstituted naphthyl group, a substituted or unsubstituted anthracenyl group, a substituted or unsubstituted phenanthryl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted chrysenyl group, a substituted or unsubstituted triphenylenyl group, a substituted or unsubstituted perylenyl group, a substituted or unsubstituted indenyl group, a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted fluorenyl group, a substituted or unsubstituted dibenzofuranyl group, a substituted or unsubstituted dibenzothiophenyl group, a substituted or unsubstituted carbazole group, a combination thereof or a fused combination thereof, but is not limited thereto.

In the specification, hole characteristics refer to characteristics capable of donating an electron to form a hole when electric field is applied, and characteristics that hole formed in the anode is easily injected into the emission layer and transported in the emission layer due to conductive characteristics according to HOMO level.

In addition, electron characteristics refer to characteristics capable of accepting an electron to form a hole when electric field is applied, and characteristics that electron formed in the cathode is easily injected into the emission layer and transported in the emission layer due to conductive characteristics according to LUMO level.

Hereinafter, an organic compound according to an embodiment is described.

The organic compound according to an embodiment may be represented by the following Chemical Formula 1.

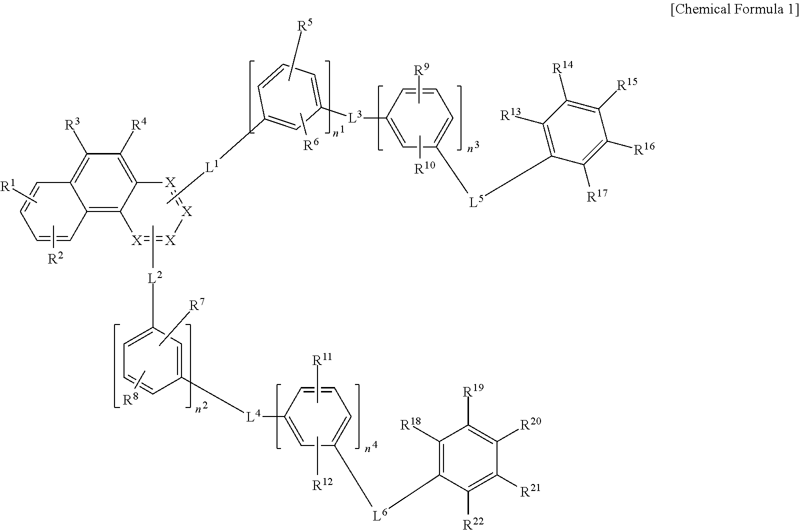

In Chemical Formula 1,

two of X may be nitrogen N and two of X may be carbon C.

R¹ to R⁴ may each independently be or include, e.g., hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof.

R⁵ to R¹² may each independently be or include, e.g., hydrogen, deuterium, a substituted or unsubstituted C1 to C10 alkyl group, a substituted or unsubstituted C6 to C12 aryl group, or a combination thereof.

R¹³ to R²² may each independently be or include, e.g., hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof.

In an implementation, R¹³ to R²² may be separate, or two adjacent ones thereof (e.g., adjacent two thereof) may be linked to each other to form a fused ring.

L¹ to L⁶ may each independently be or include, e.g., a single bond, a substituted or unsubstituted phenylene group, a substituted or unsubstituted biphenylene group, a substituted or unsubstituted terphenylene group or a substituted or unsubstituted quaterphenylene group,

n¹ to n⁴ may each independently be an integer of, e.g., 0 to 5. In an implementation, a sum of n¹ to n⁴ may be an integer of 2 or more, preferable 6 or less.

For example, the organic compound represented by the Chemical Formula 1 may include a substituted or unsubstituted benzoquinazoline moiety including two nitrogen atoms and at least two meta-bonded substituted or unsubstituted phenylene groups.

The nitrogen-containing moiety of the substituted or unsubstituted benzoquinazoline may have polarity and thus, may interact with an electrode. Accordingly, a charge may be easily injected and charge mobility may be increased by three fused rings.

The substituted or unsubstituted benzoquinazoline may have a relatively low LUMO energy level and thus, may easily inject electrons and may help improve thermal stability and electrical stability. The substituted or unsubstituted benzoquinazoline may have, e.g., a LUMO energy level ranging from about 1.7 to about 2.1 eV.

The at least two meta-bonded substituted or unsubstituted phenylene groups may help control a charge stream flowing toward the benzoquinazoline and thus, may help increase stability of the benzoquinazoline. For example, the at least two meta-bonded substituted or unsubstituted phenylene groups may help decrease oxidation of cyclic carbons neighboring a nitrogen-containing moiety (ring) of the benzoquinazoline (e.g., carbons bonded with R³ or R⁴) and thus, may help increase stability of the organic compound. Accordingly, life-span of the organic compound may be improved.

In an implementation, R³ or R⁴ may be hydrogen. In an implementation, R³ and R⁴ may both be hydrogen.

The at least two meta-bonded substituted or unsubstituted phenylene groups may be positioned at one side or both sides of the benzoquinazoline. In an implementation, the at least two meta-bonded substituted or unsubstituted phenylene groups may be positioned at both sides of the benzoquinazoline, e.g., n¹ and n² of the above Chemical Formula 1 may each independently be (an integer of) 1 to 5.

In an implementation, the organic compound may structurally have steric hindrance characteristics and may be suppressed from interaction with a neighboring molecule. Thus, crystallization may decrease and efficiency and life-span characteristics may be improved.

The organic compound may have, e.g., a molecular weight of greater than or equal to about 500. For example, the organic compound may show an increased glass transition temperature (Tg) and thus, increased stability. Degradation during a manufacturing process when applied to a device may also be suppressed.

The glass transition temperature (Tg) may be related to thermal stability of the organic compound and a device manufactured by applying the same. For example, when an organic compound having a high glass transition temperature (Tg) is applied as a thin film to an organic light emitting diode, the organic compound and the organic light emitting diode using the compound may be prevented from degrading by a (e.g., high) temperature in a subsequent process, e.g., during an encapsulation process after deposition of the organic compound, helping to secure life-span characteristics of the organic compound and the organic light emitting diode.

The organic compound may have a glass transition temperature (Tg) of, e.g., greater than or equal to about 70° C. In an implementation, the organic compound may have a glass transition temperature (Tg) of, e.g., greater than or equal to about 90° C. The glass transition temperature (Tg) may be, e.g., about 70° C. to about 150° C. or about 90° C. to about 130° C.

In an implementation, the organic compound may be represented by one of the following Chemical Formula 2 or 3, e.g., according to a position of nitrogen (N).

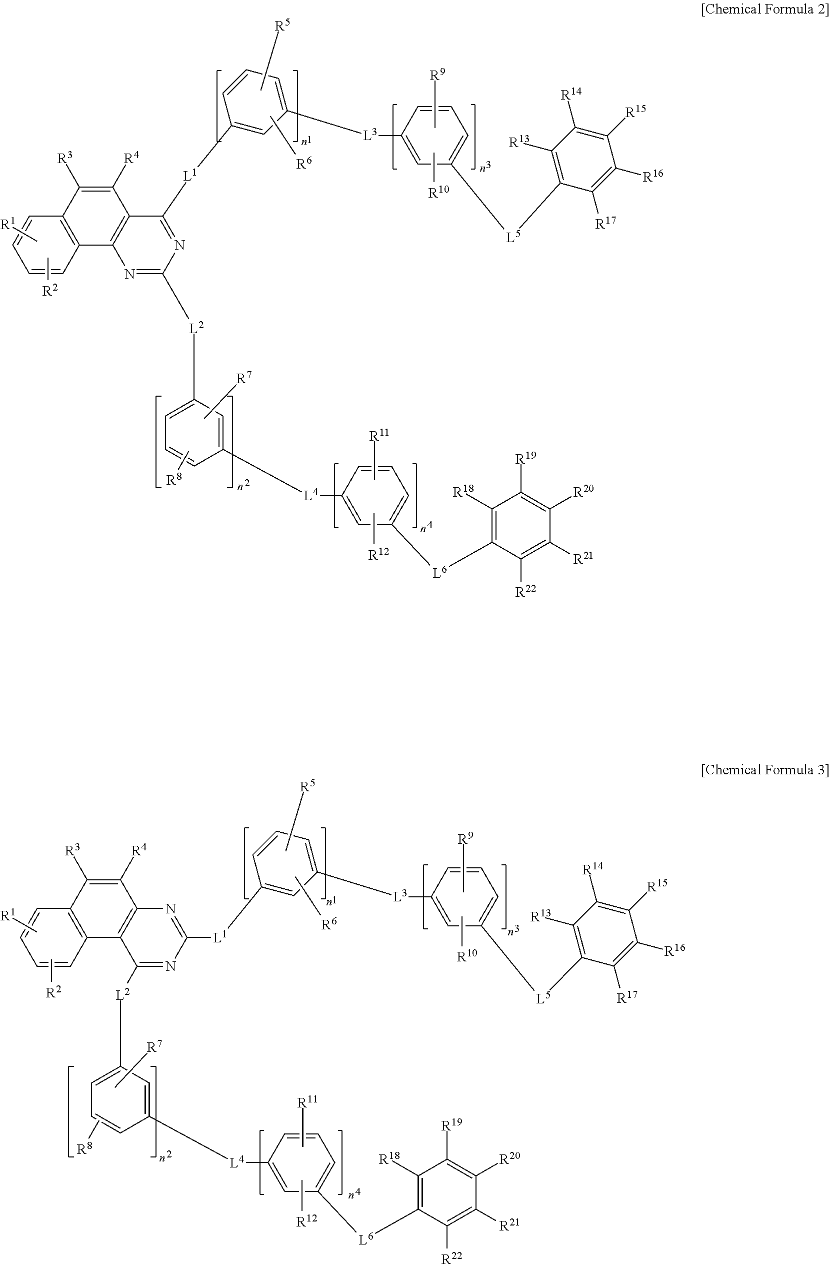

In Chemical Formulae 2 and 3, R′ to R²², L′ to L⁶, and n′ to n⁴ may be the same as described above with respect to Chemical Formula 1.

In an implementation, the compound represented by Chemical Formula 2 may include a compound represented by the following Chemical Formula 2A.

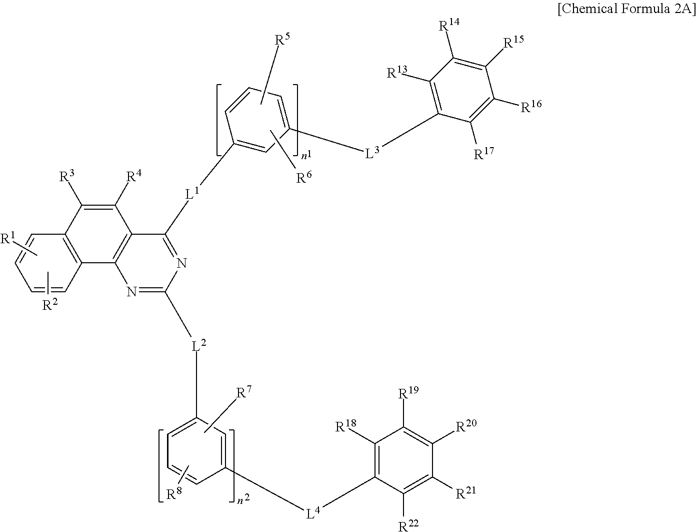

In Chemical Formula 2A, R¹ to R⁸, R¹³ to R²², L¹ to L⁴, n¹, and n² may each be the same as described above with respect to Chemical Formula 1.

In an implementation, the compound represented by Chemical Formula 2 may include a compound represented by the following Chemical Formula 3A.

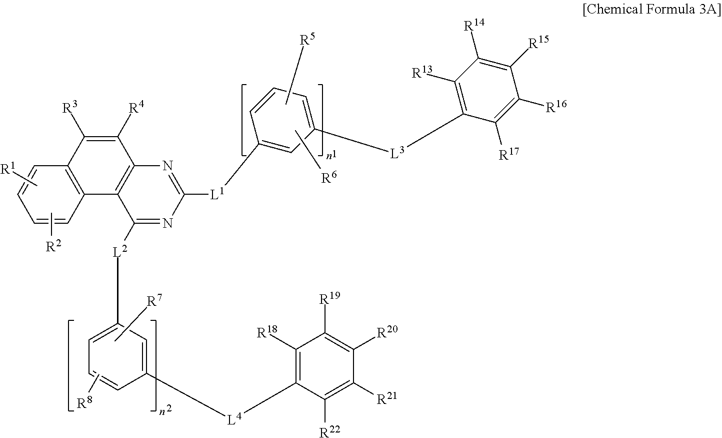

In Chemical Formula 3A, R¹ to R⁸, R¹³ to R²², L¹ to L⁴, n¹, and n² may be the same as described above with respect to Chemical Formula 1.

In an implementation, in Chemical Formulae 1 to 3, 2A and 3A, L¹ to L⁶ may each independently be or include, e.g., a single bond a of the following Group 1.

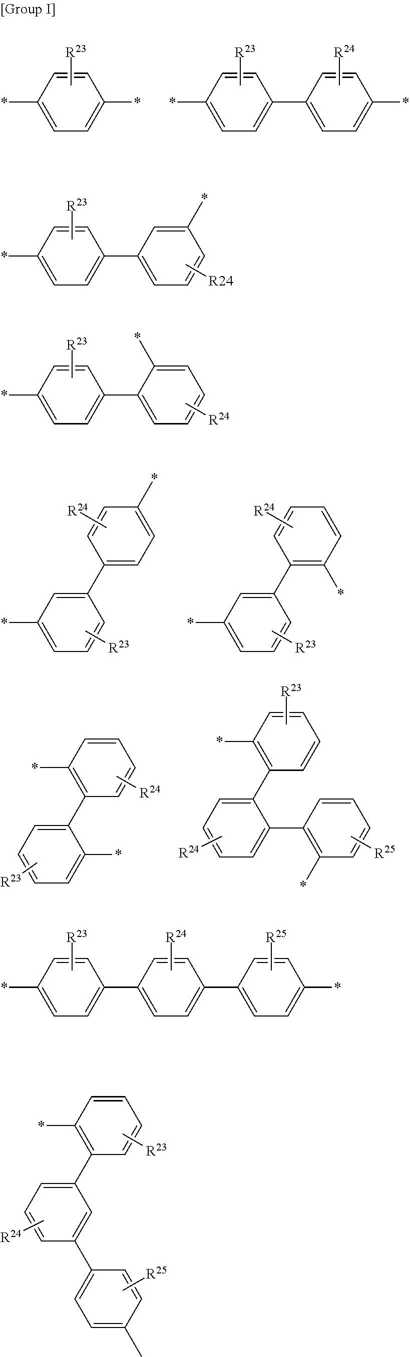

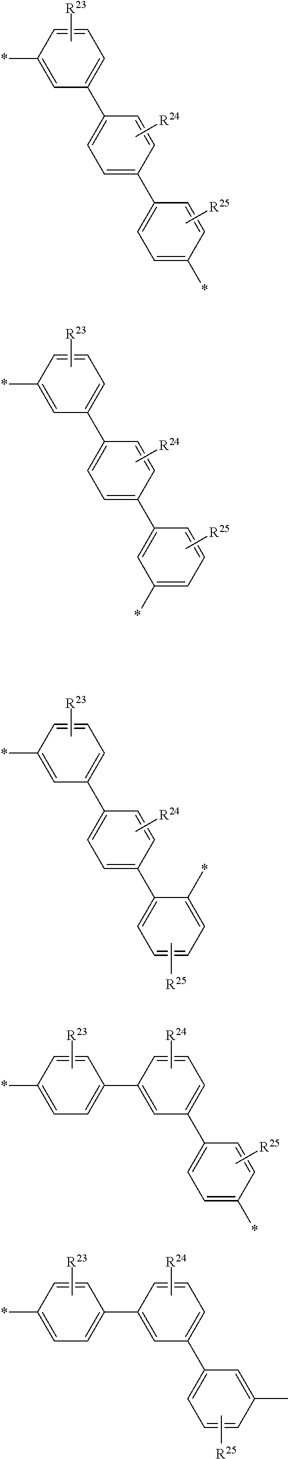

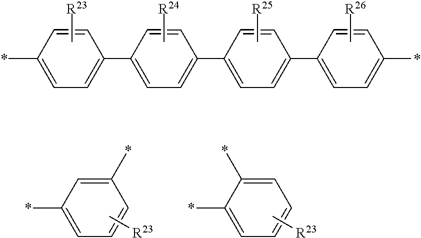

In Group 1, R²³ to R²⁶ may each independently be or include, e.g., hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof, and * is a linking point to a neighboring atom.

For example, in Group 1, R²³ to R²⁶ may each be hydrogen.

In an implementation, the organic compound may be, e.g., a compound of the following Group 2.

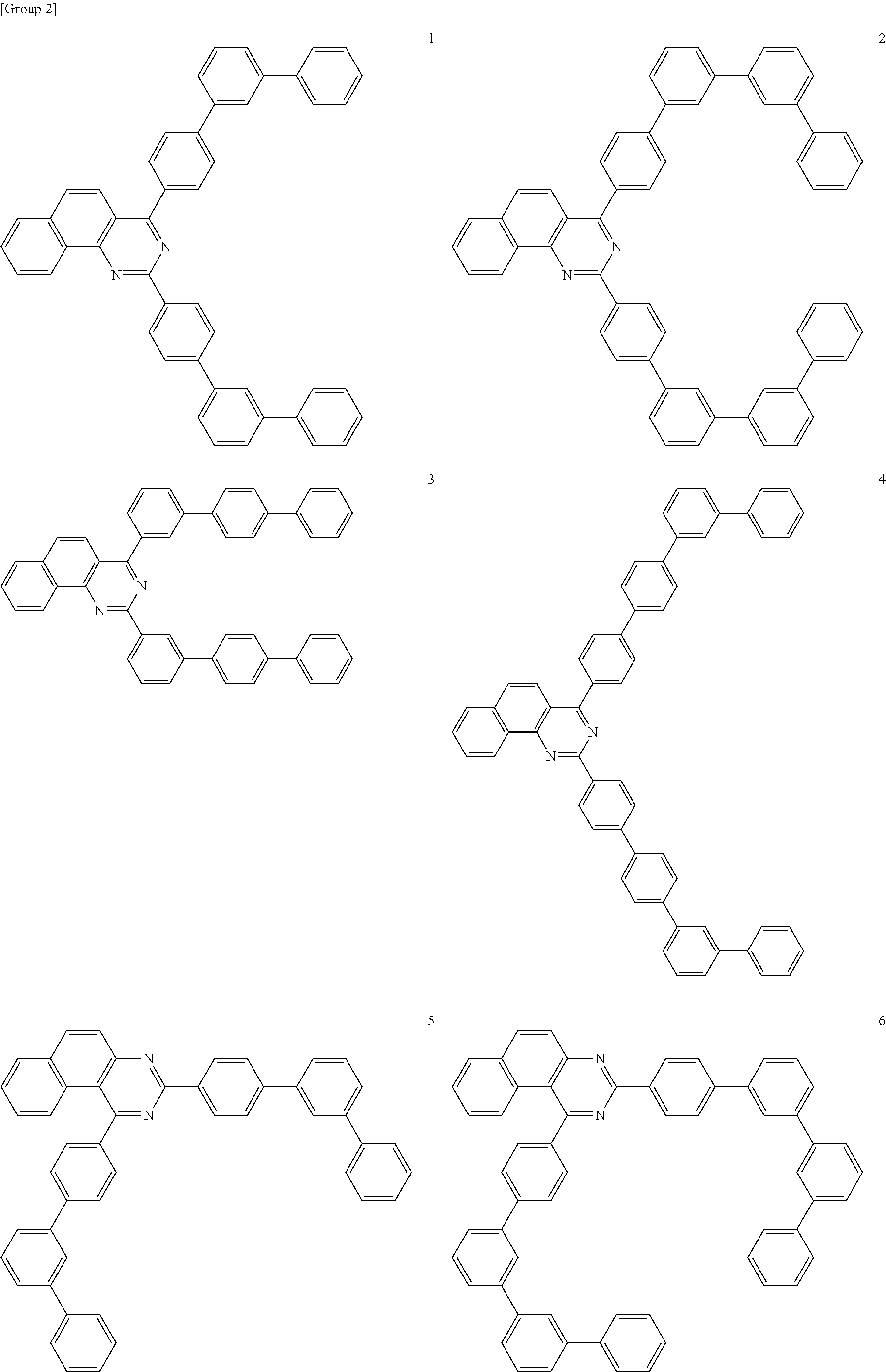

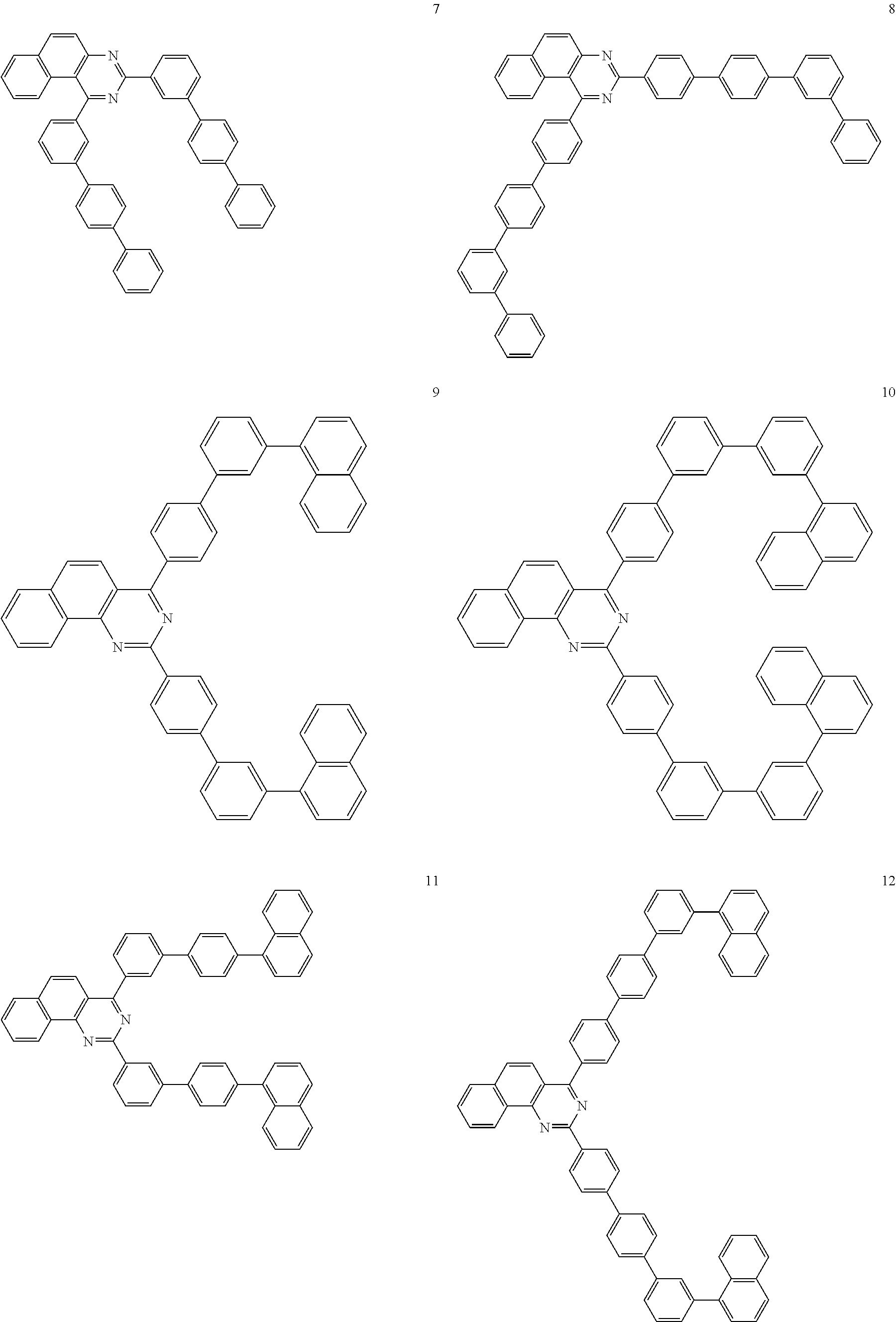

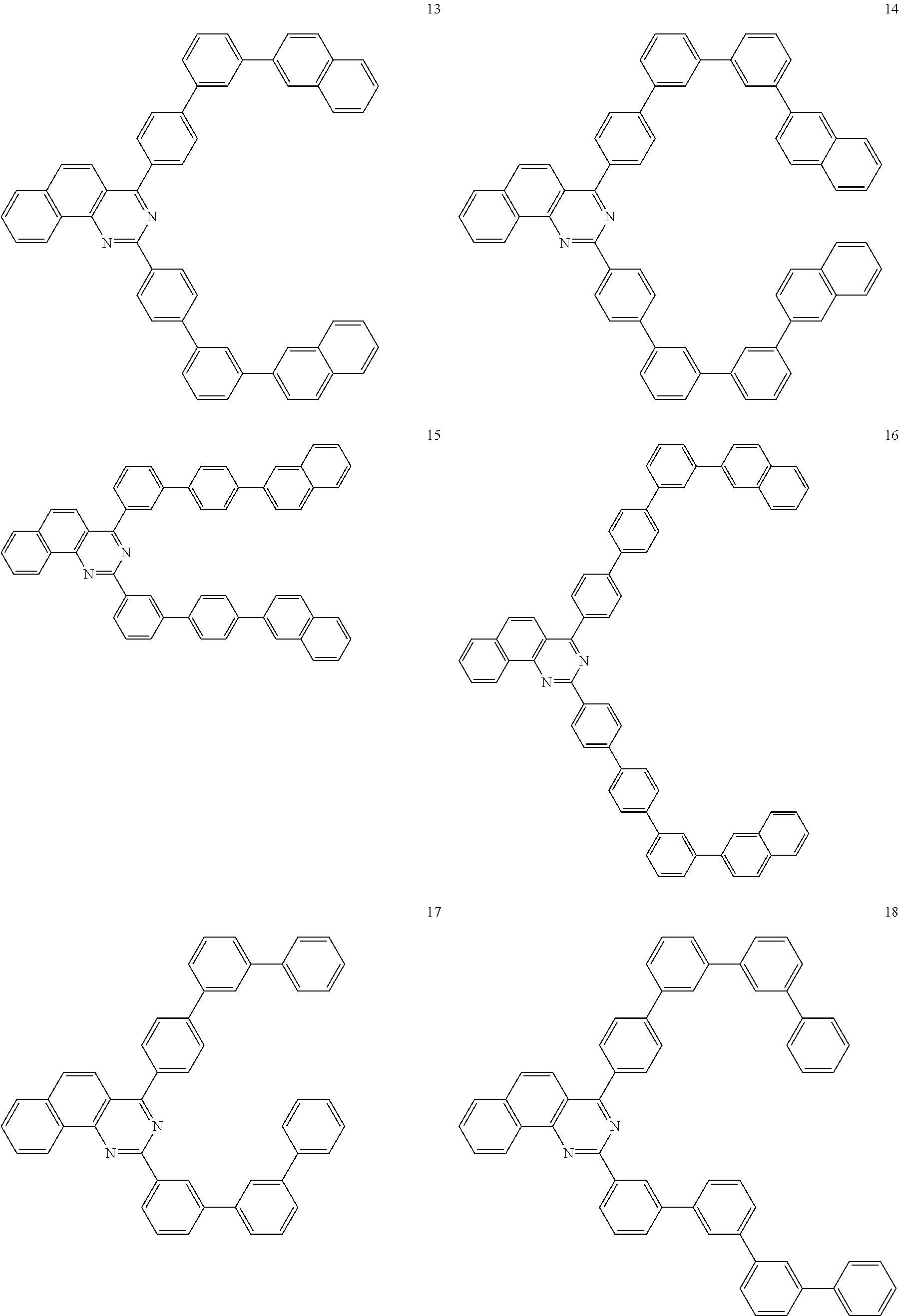

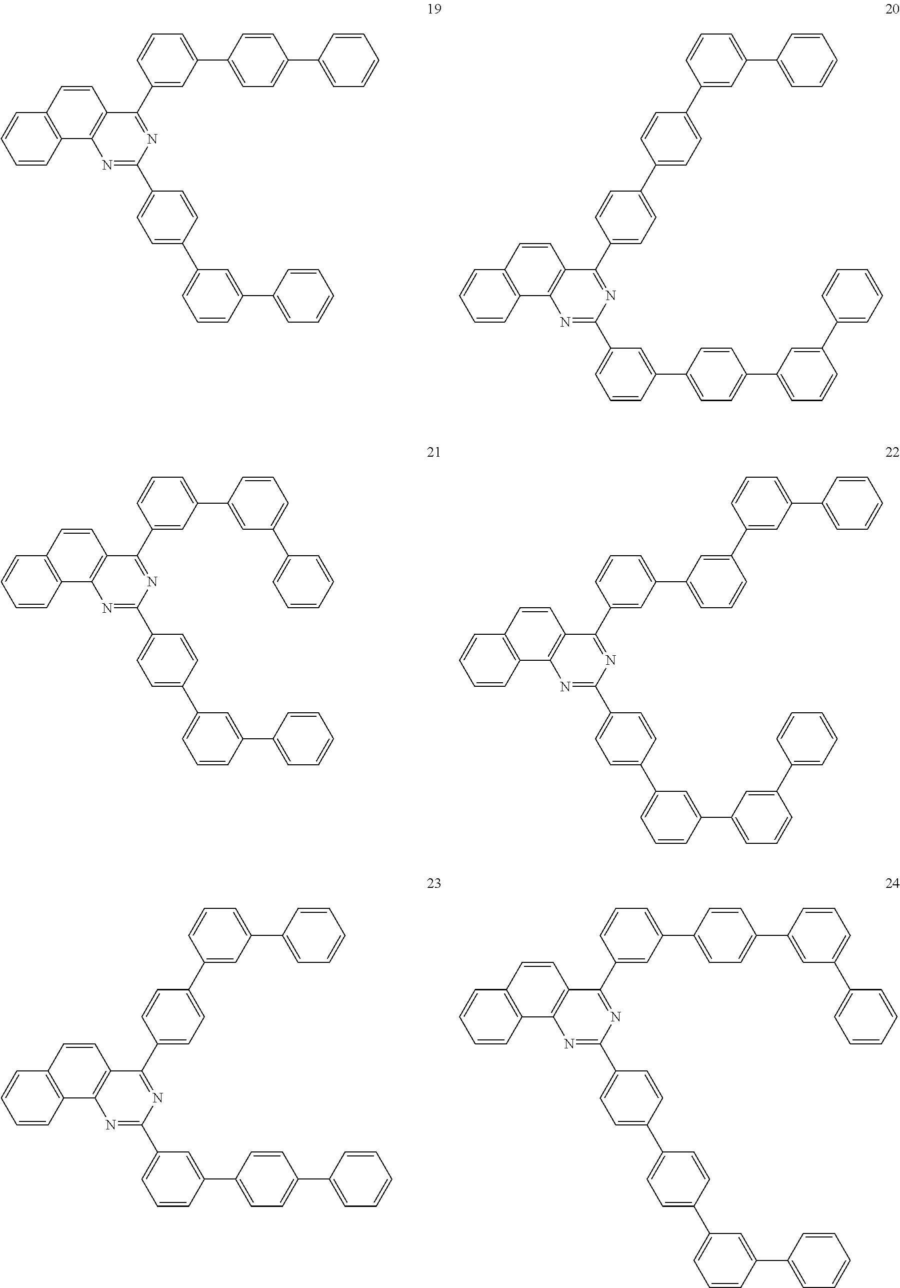

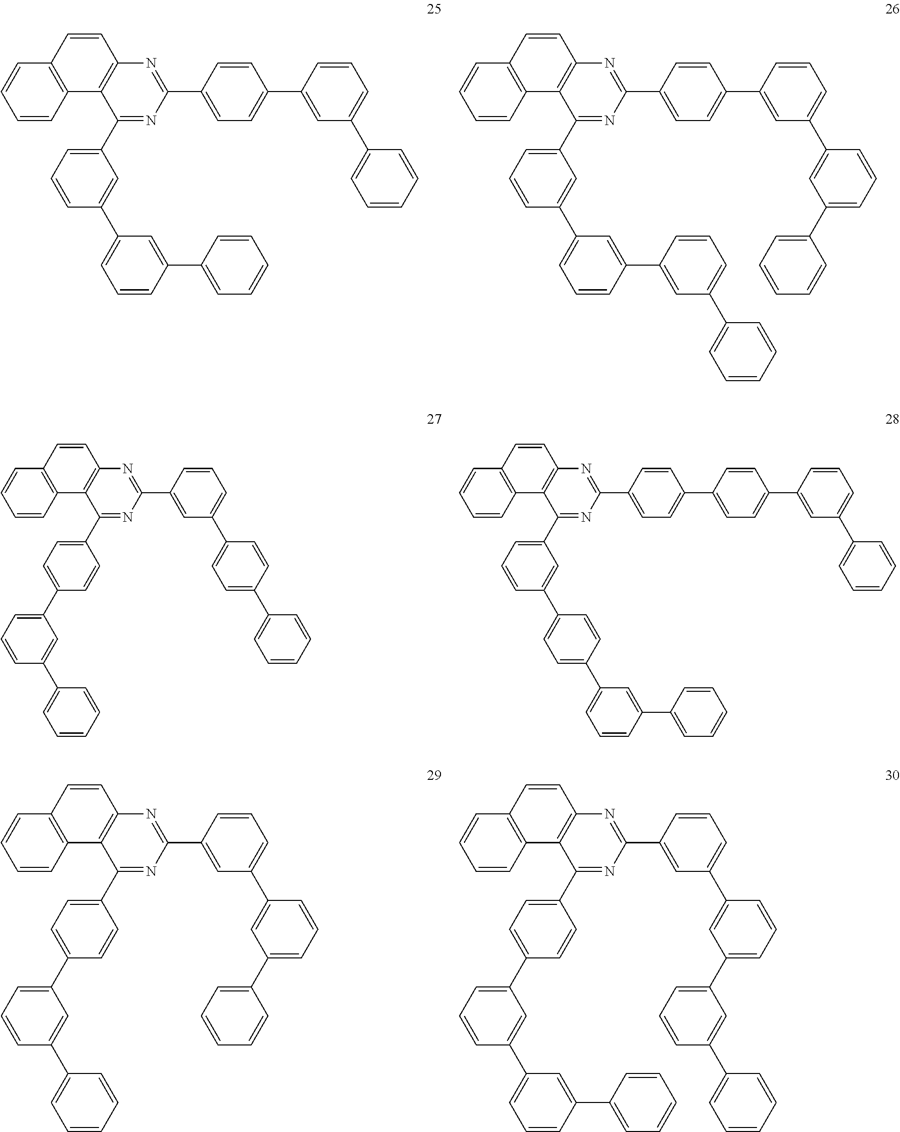

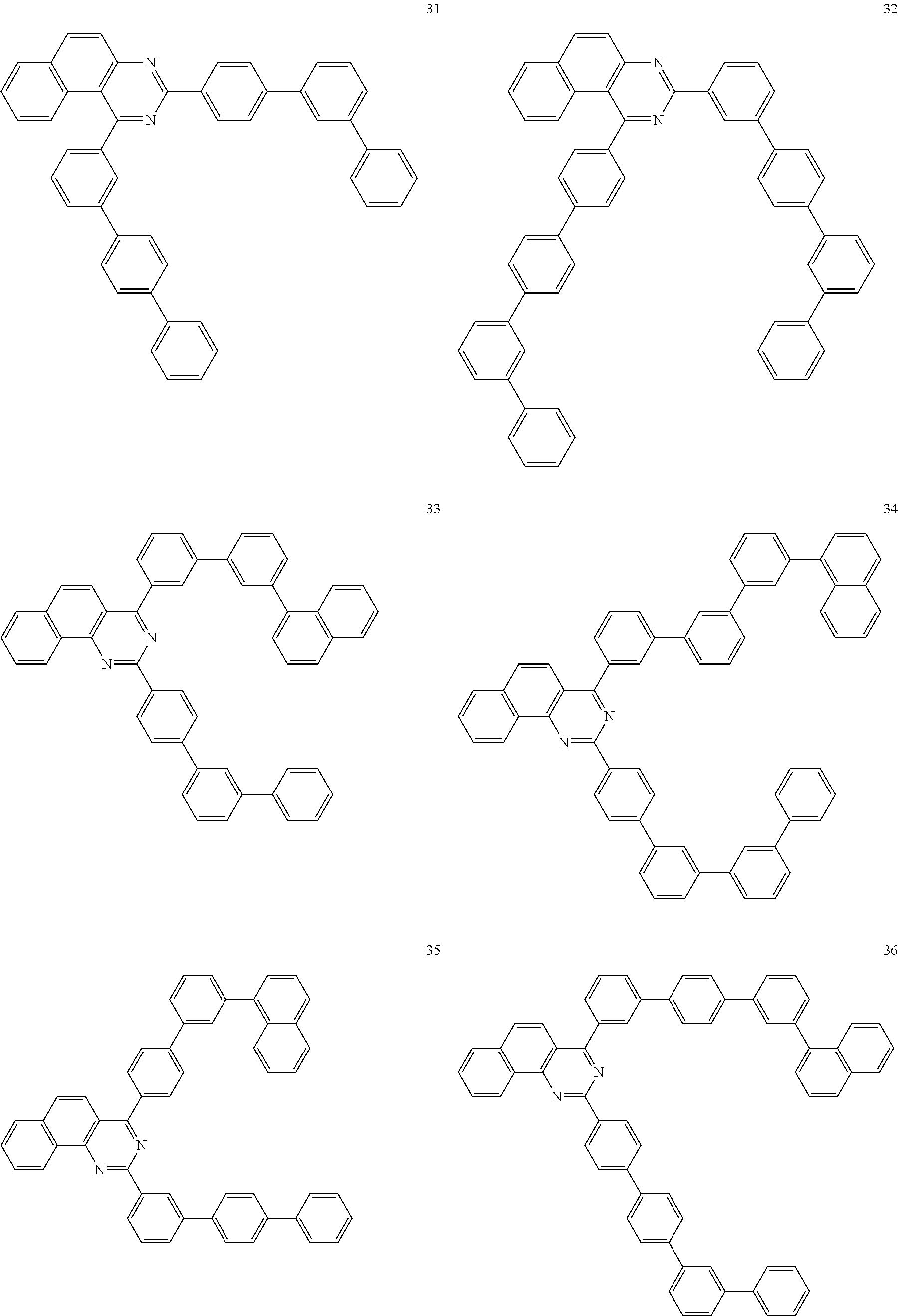

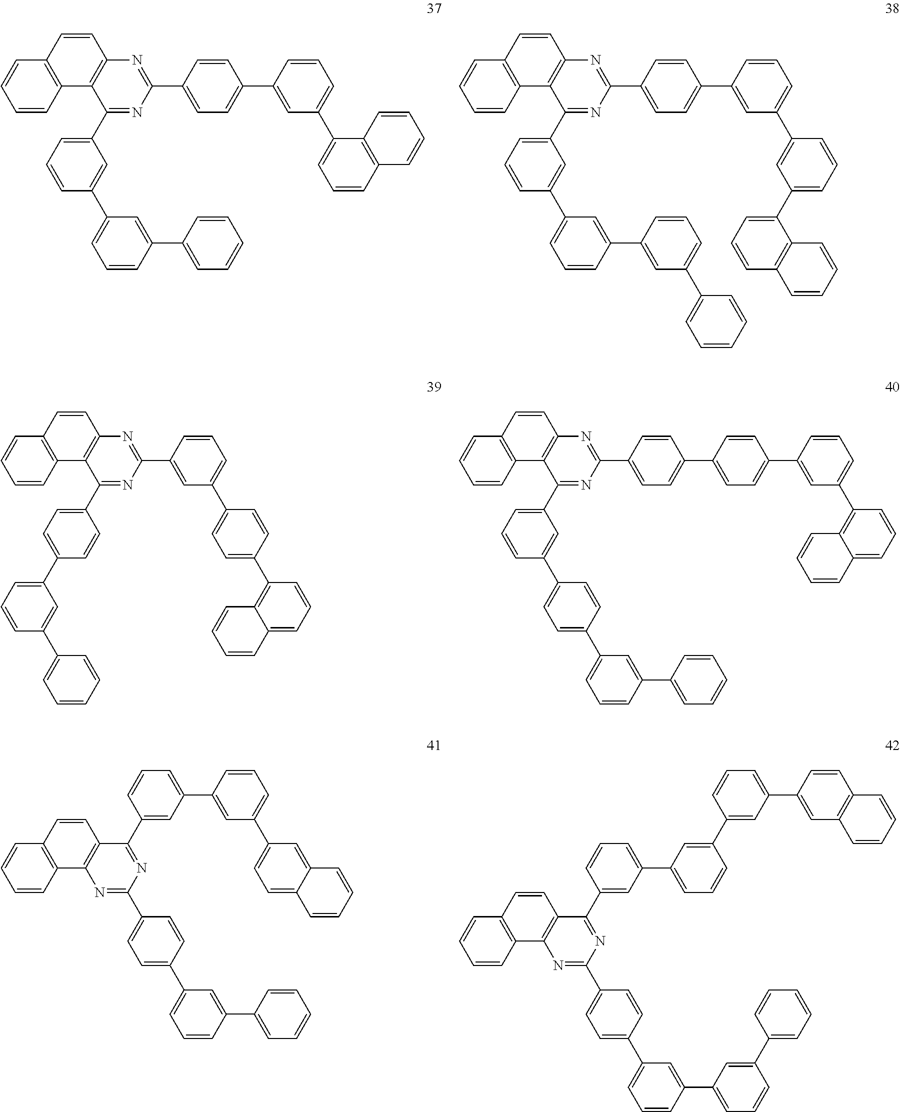

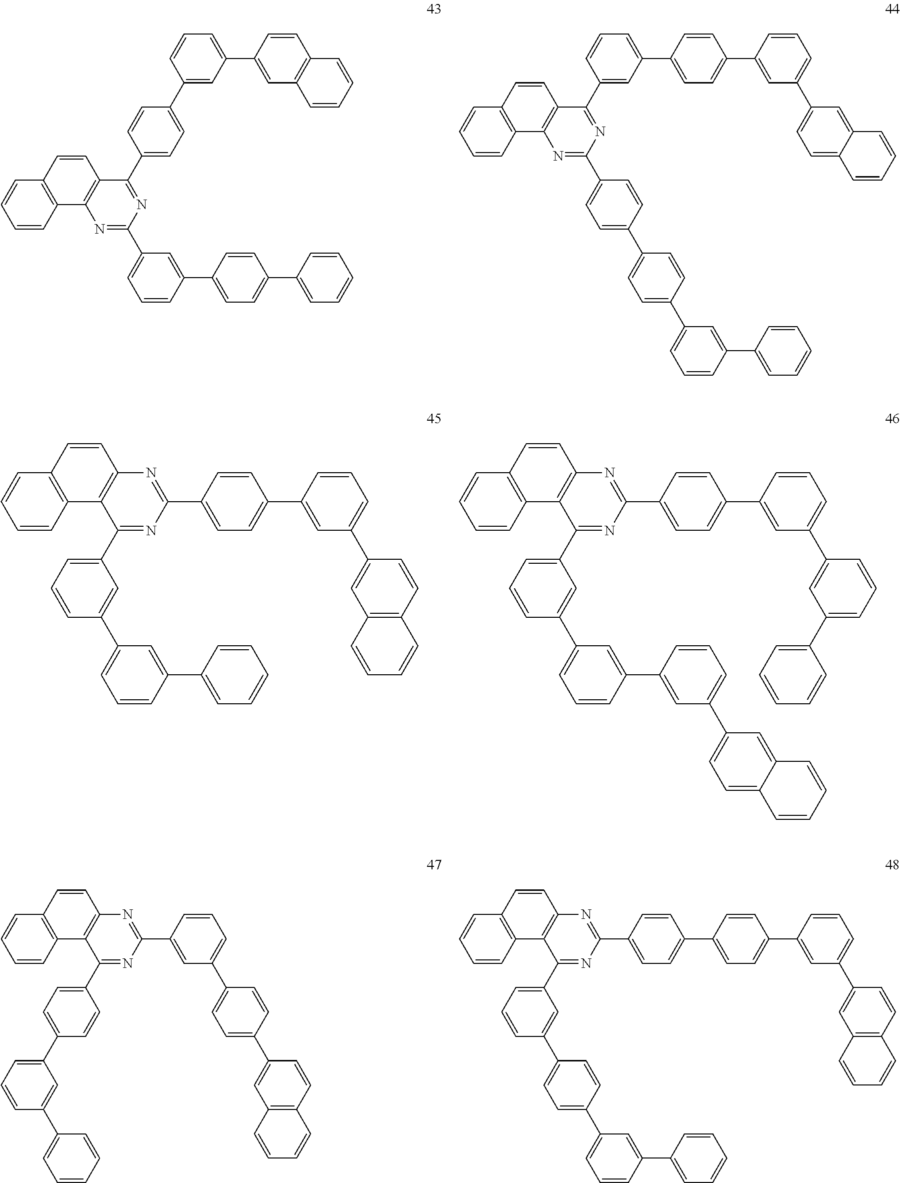

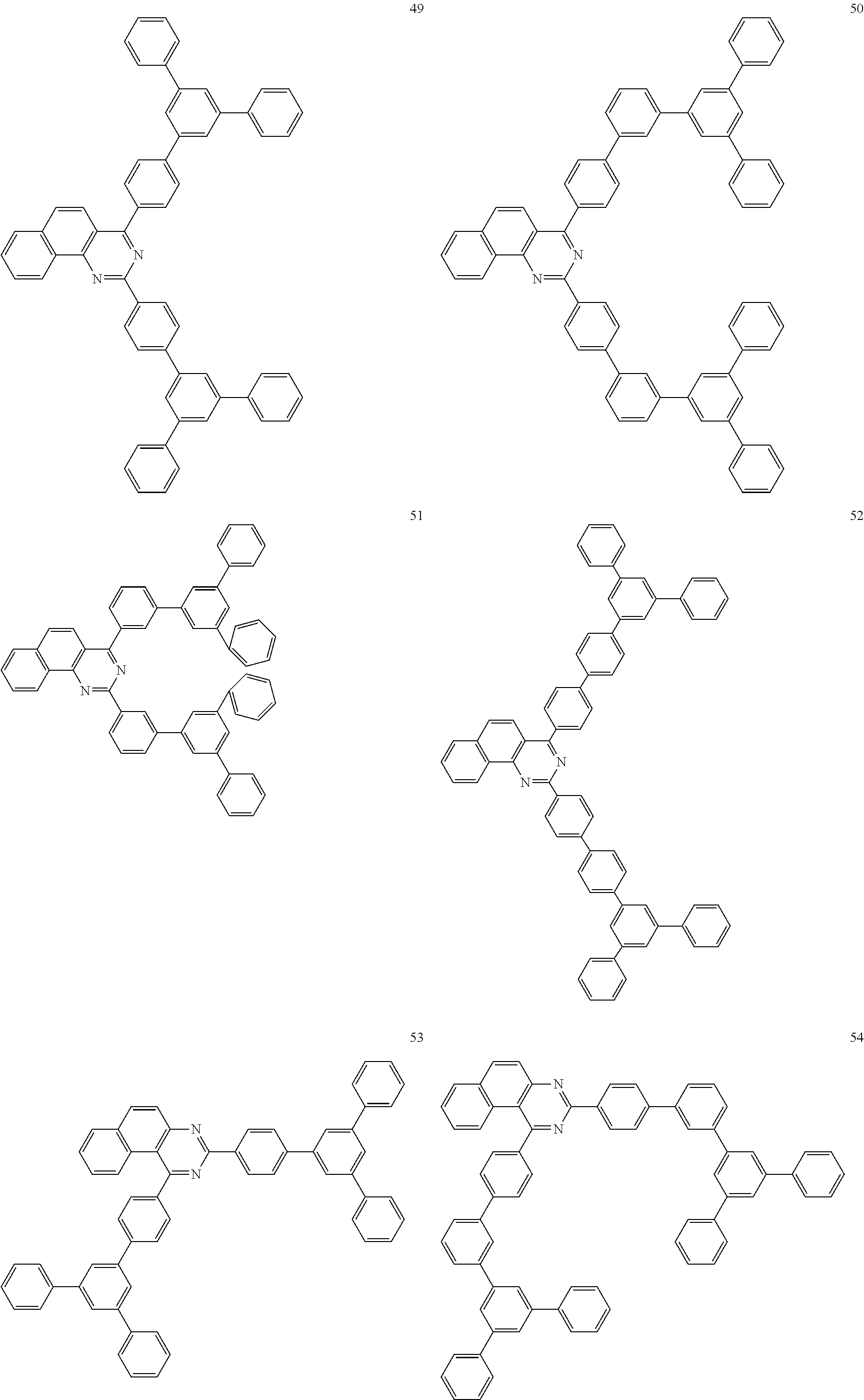

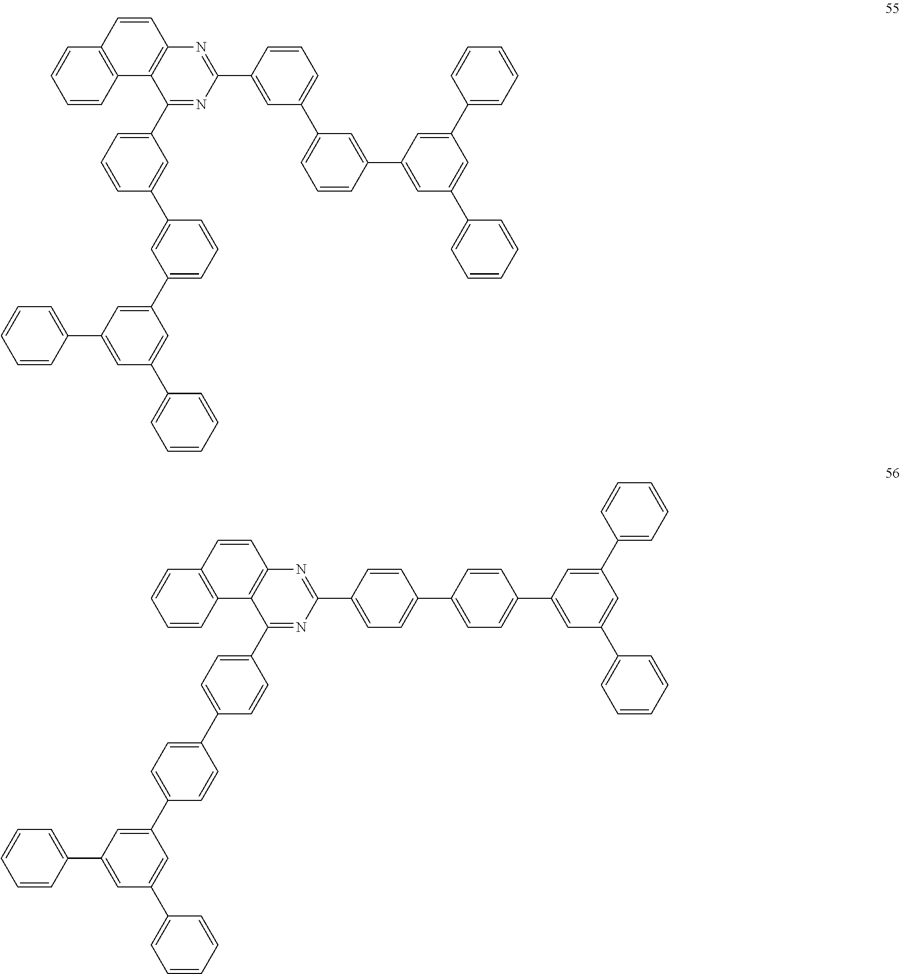 [SMILES: c1ccc(-c2cc(-c3ccccc3)cc(-c3cccc(-c4cccc(-c5nc(-c6cccc(-c7cccc(-c8cc(-c9ccccc9)cc(-c9ccccc9)c8)c7)c6)c6c(ccc7ccccc76)n5)c4)c3)c2)cc1 ; c1ccc(-c2cc(-c3ccccc3)cc(-c3ccc(-c4ccc(-c5nc(-c6ccc(-c7ccc(-c8cc(-c9ccccc9)cc(-c9ccccc9)c8)cc7)cc6)c6c(ccc7ccccc76)n5)cc4)cc3)c2)cc1]

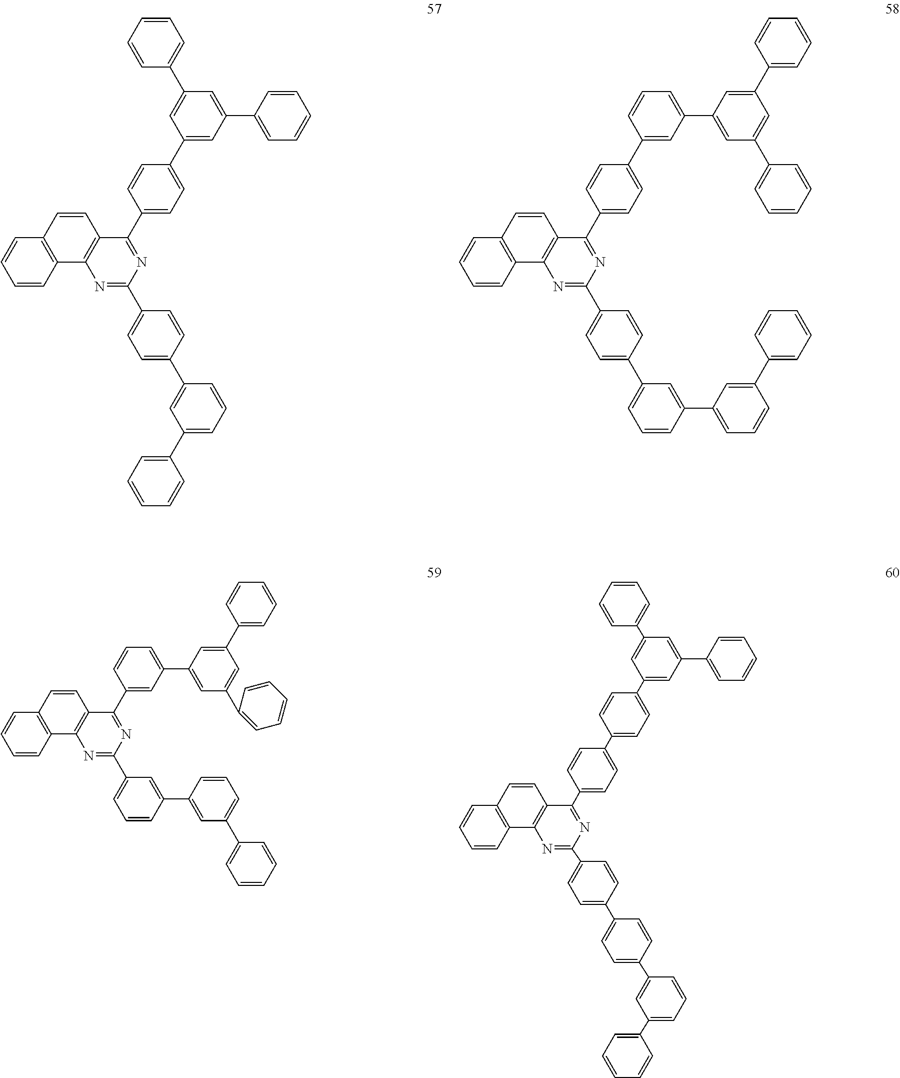 [SMILES: c1ccc(-c2cccc(-c3ccc(-c4nc(-c5ccc(-c6cc(-c7ccccc7)cc(-c7ccccc7)c6)cc5)c5ccc6ccccc6c5n4)cc3)c2)cc1 ; c1ccc(-c2cccc(-c3cccc(-c4ccc(-c5nc(-c6ccc(-c7cccc(-c8cc(-c9ccccc9)cc(-c9ccccc9)c8)c7)cc6)c6ccc7ccccc7c6n5)cc4)c3)c2)cc1 ; c1ccc(-c2cccc(-c3cccc(-c4nc(-c5cccc(-c6cc(-c7ccccc7)cc(-c7ccccc7)c6)c5)c5ccc6ccccc6c5n4)c3)c2)cc1 ; c1ccc(-c2cccc(-c3ccc(-c4ccc(-c5nc(-c6ccc(-c7ccc(-c8cc(-c9ccccc9)cc(-c9ccccc9)c8)cc7)cc6)c6ccc7ccccc7c6n5)cc4)cc3)c2)cc1]

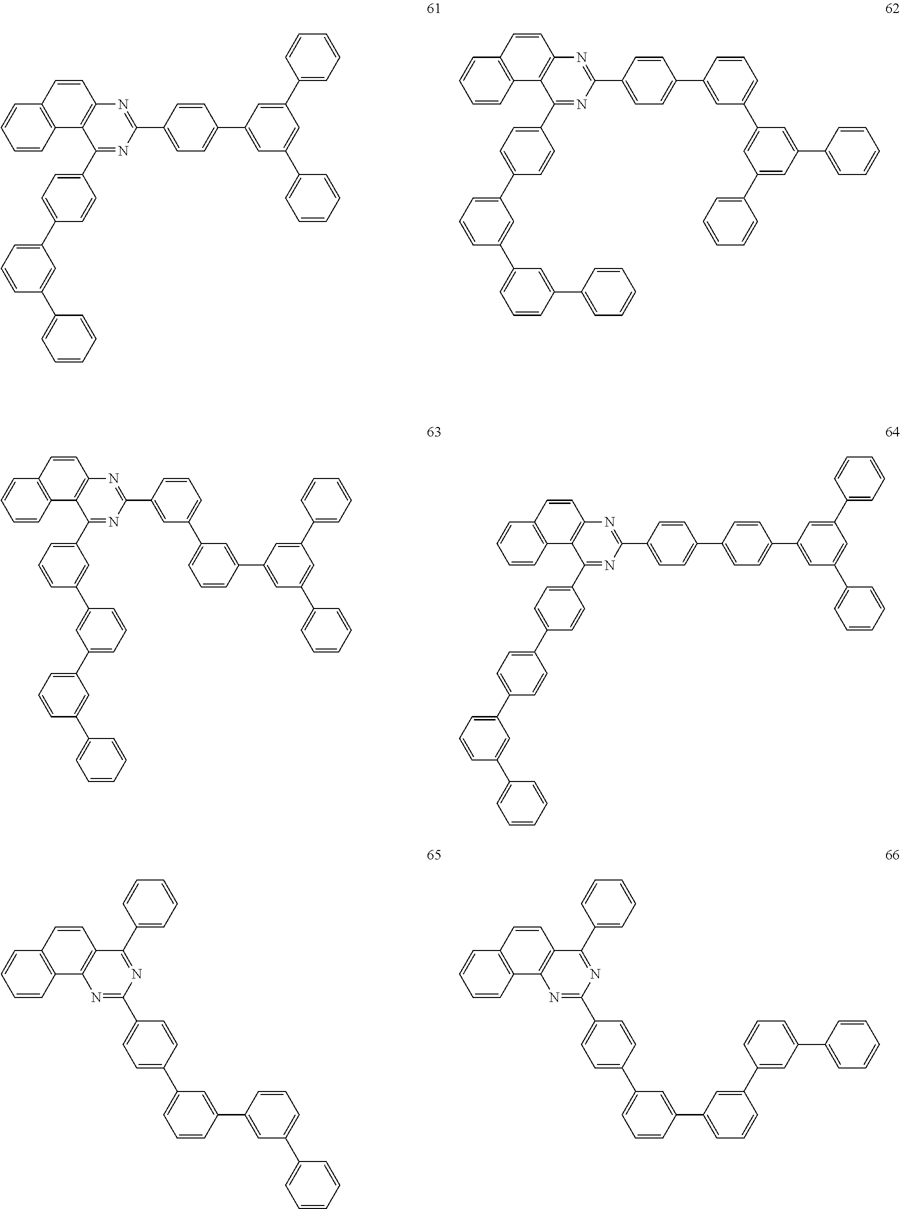

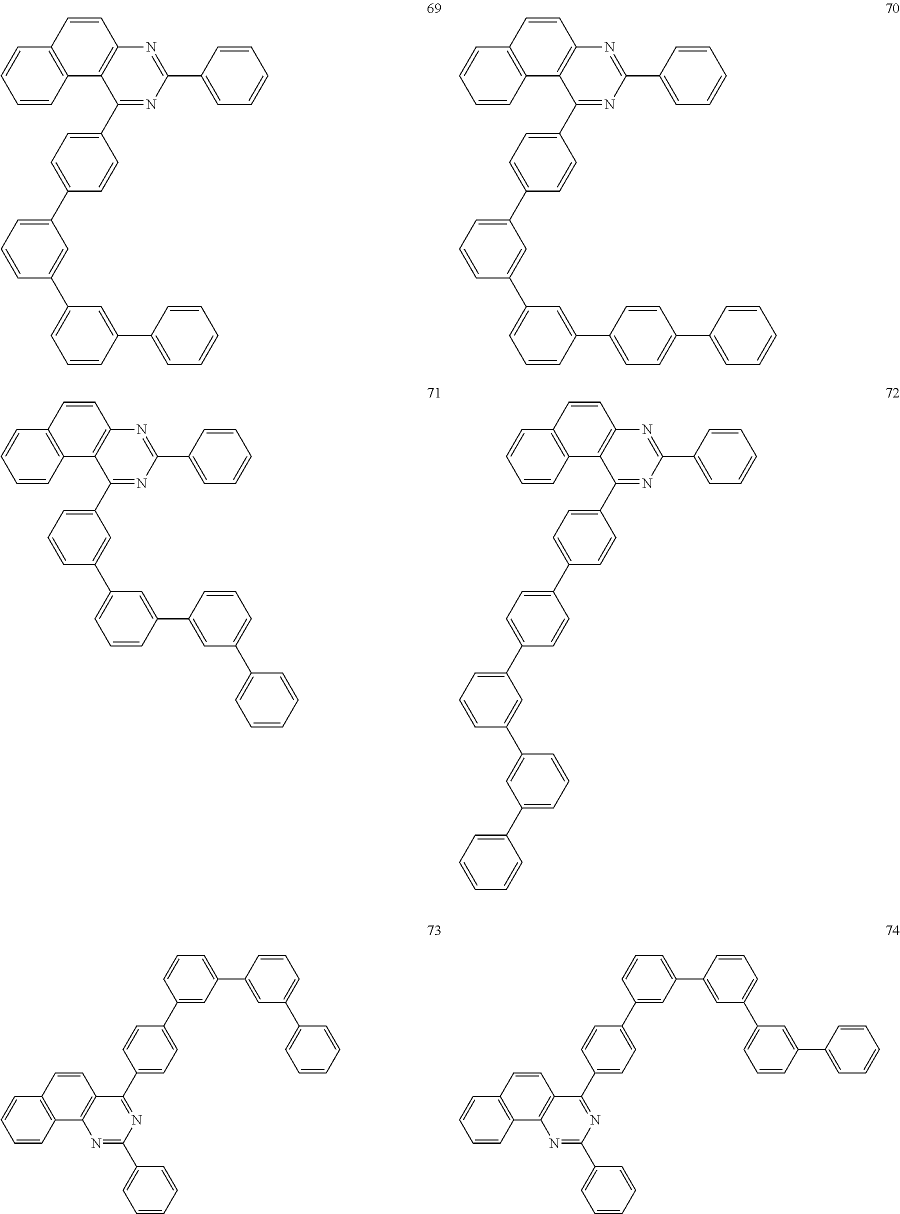

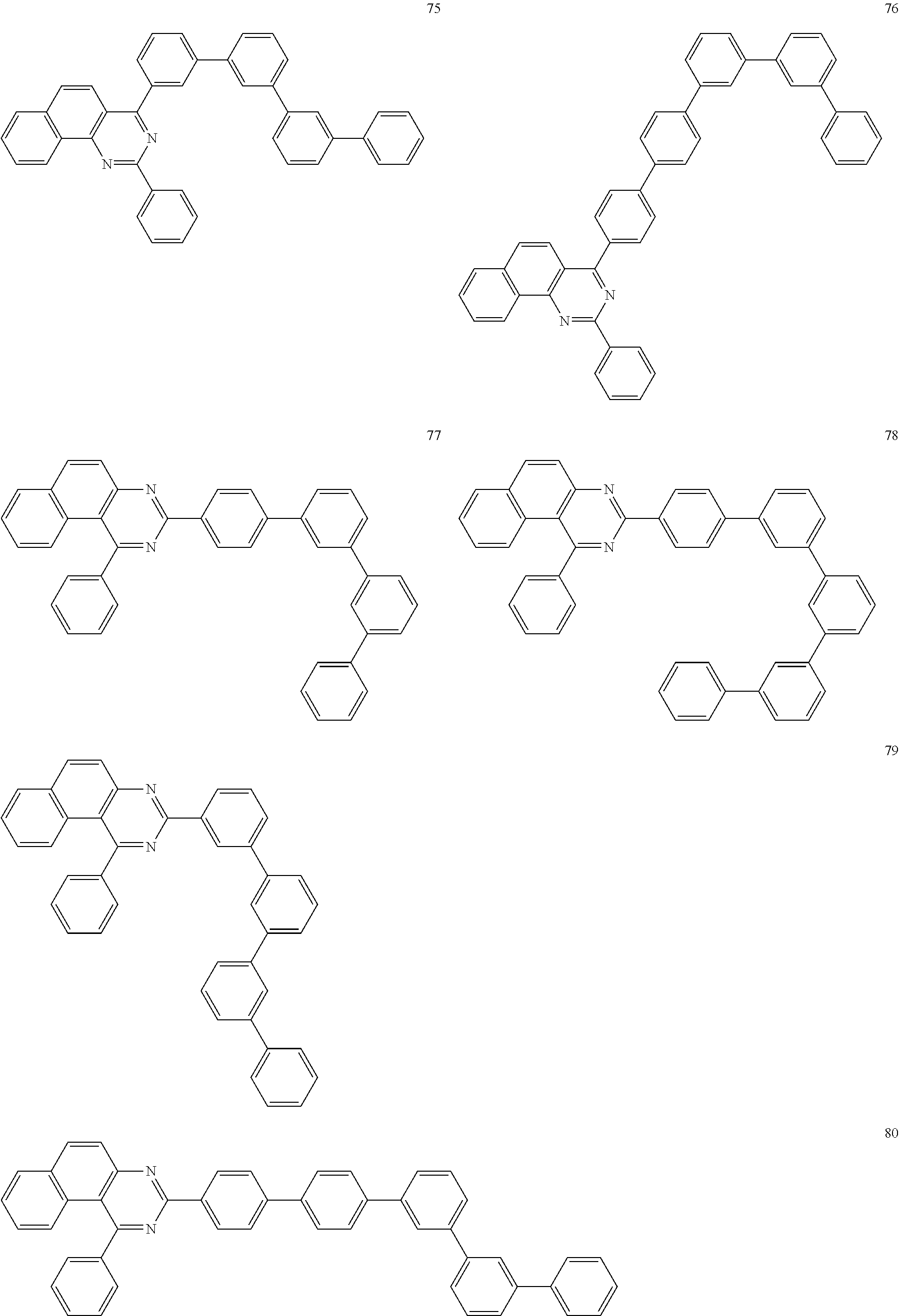

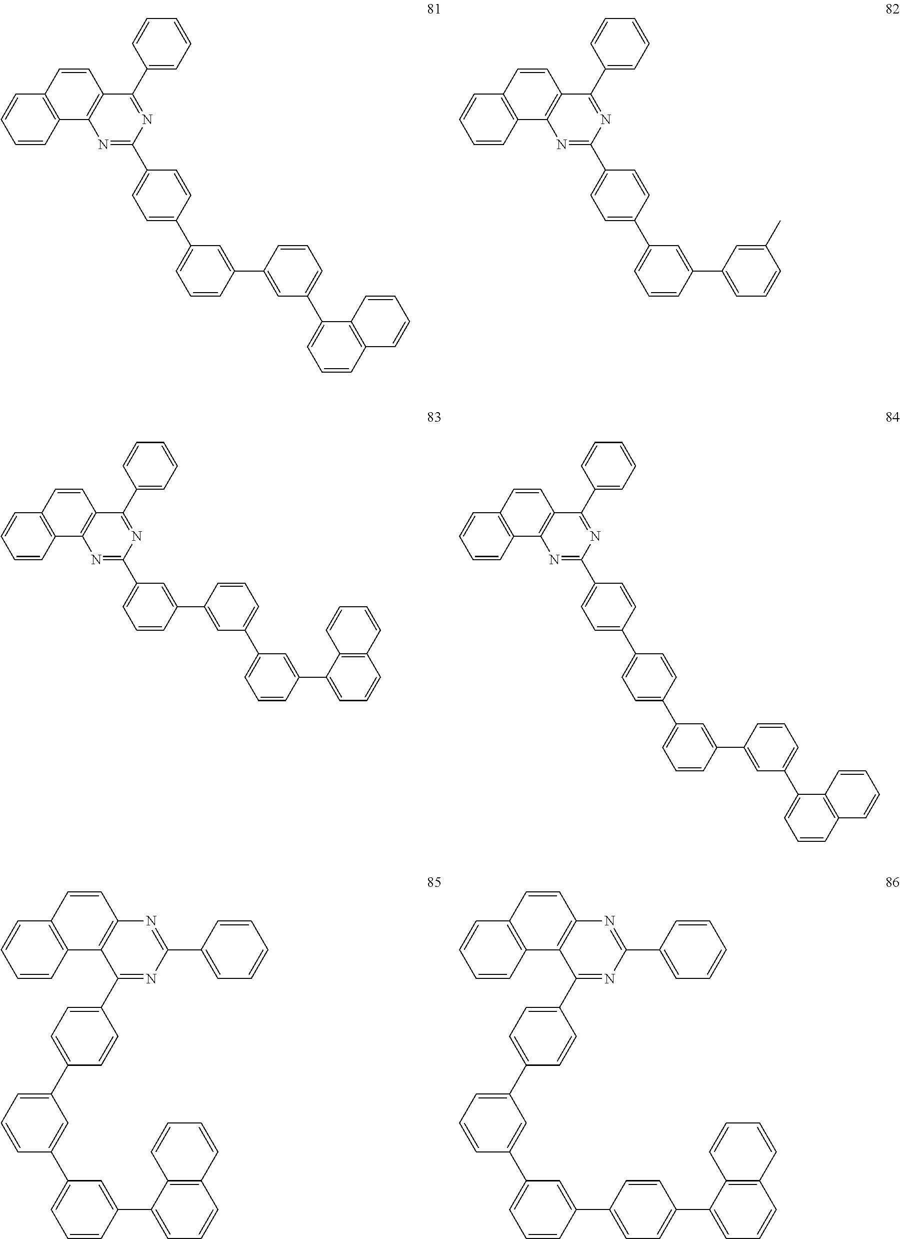

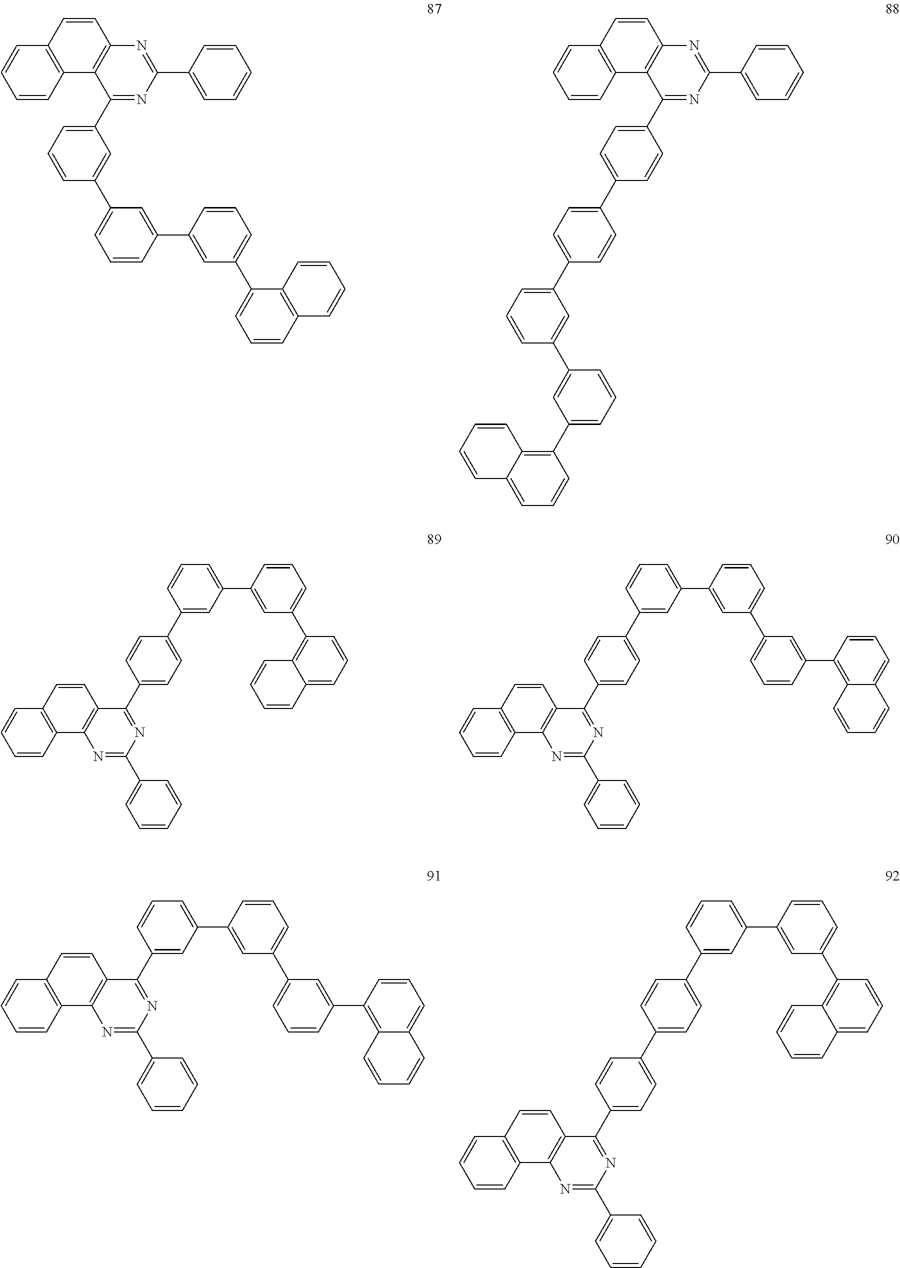

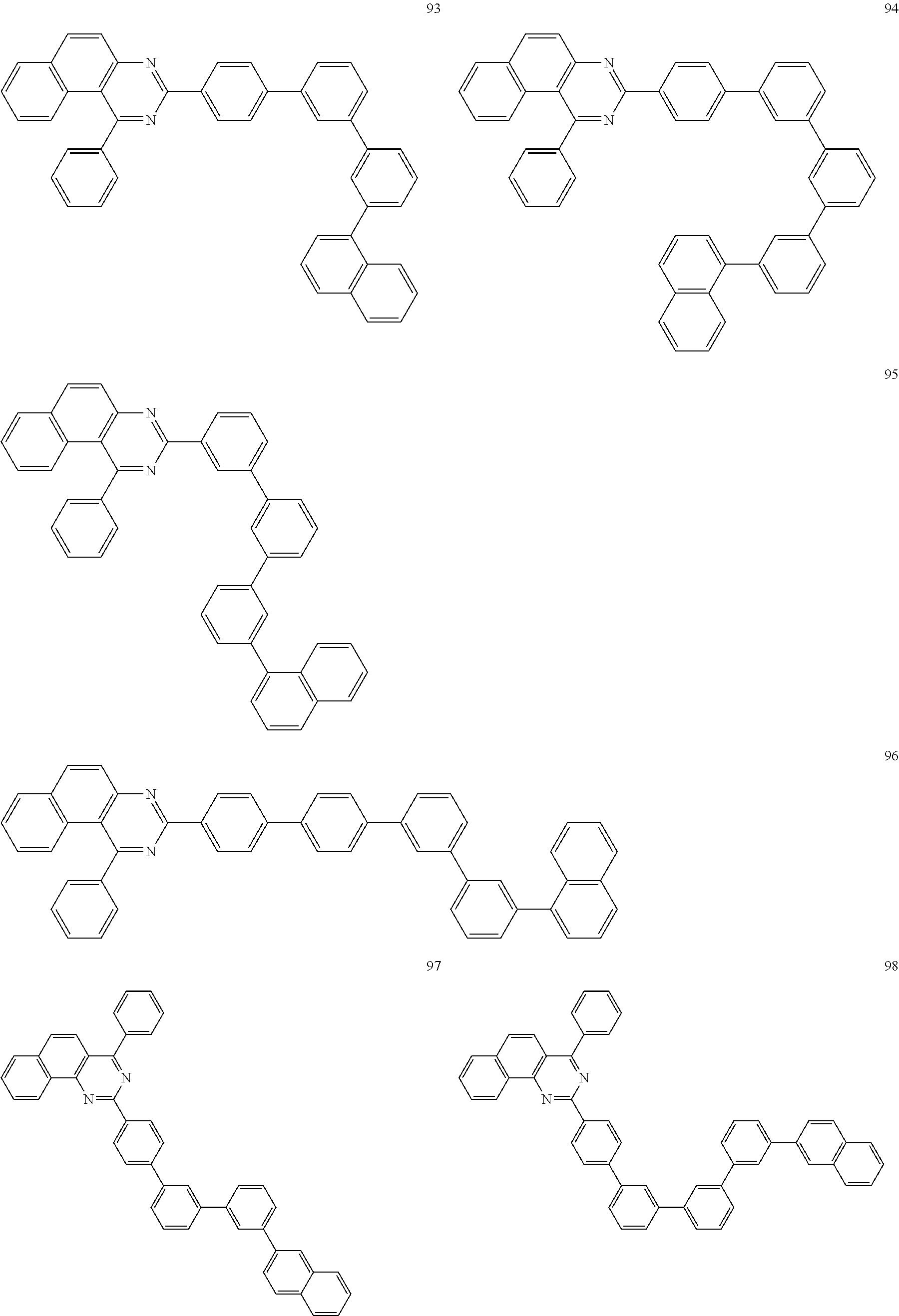

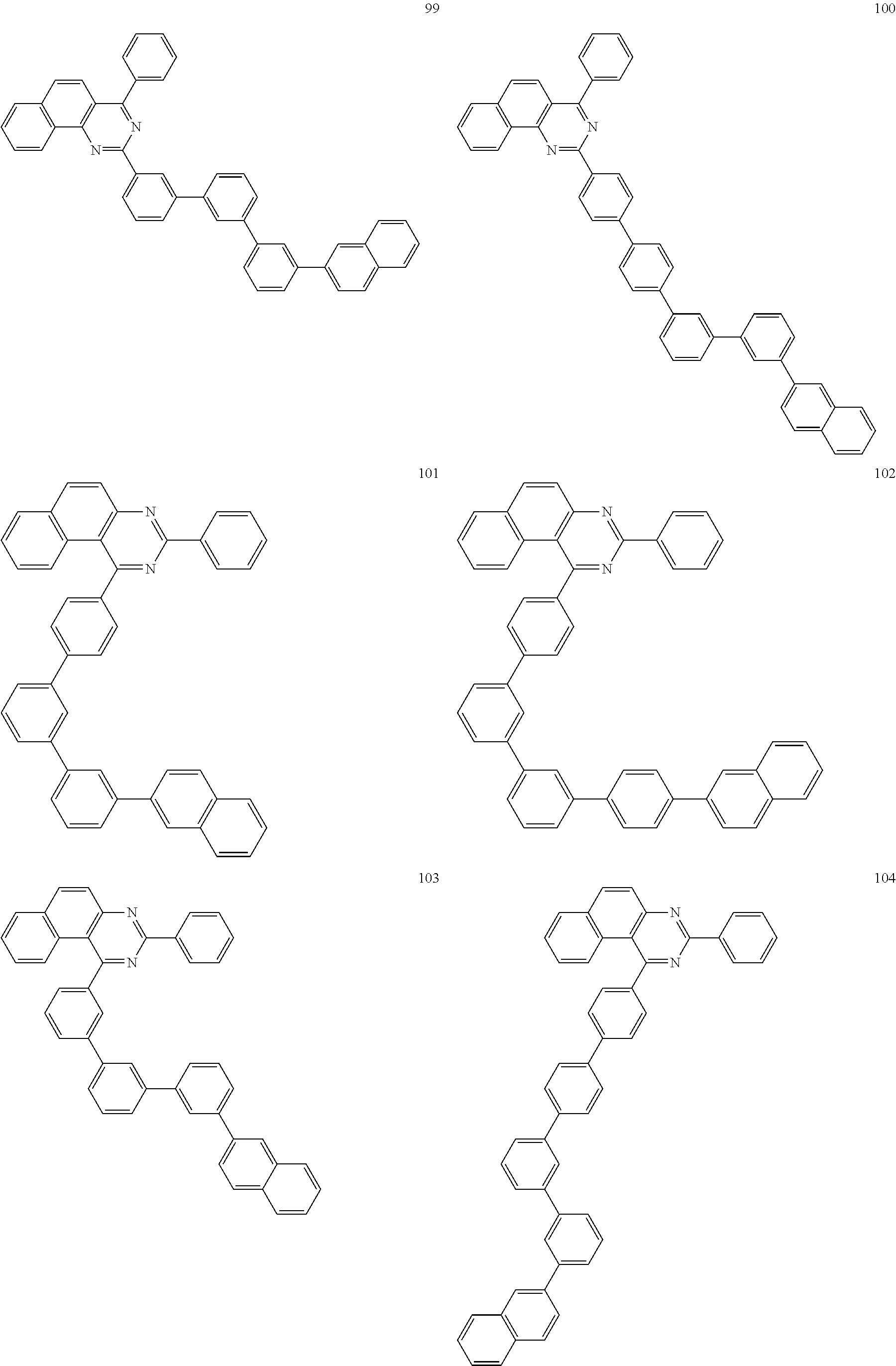

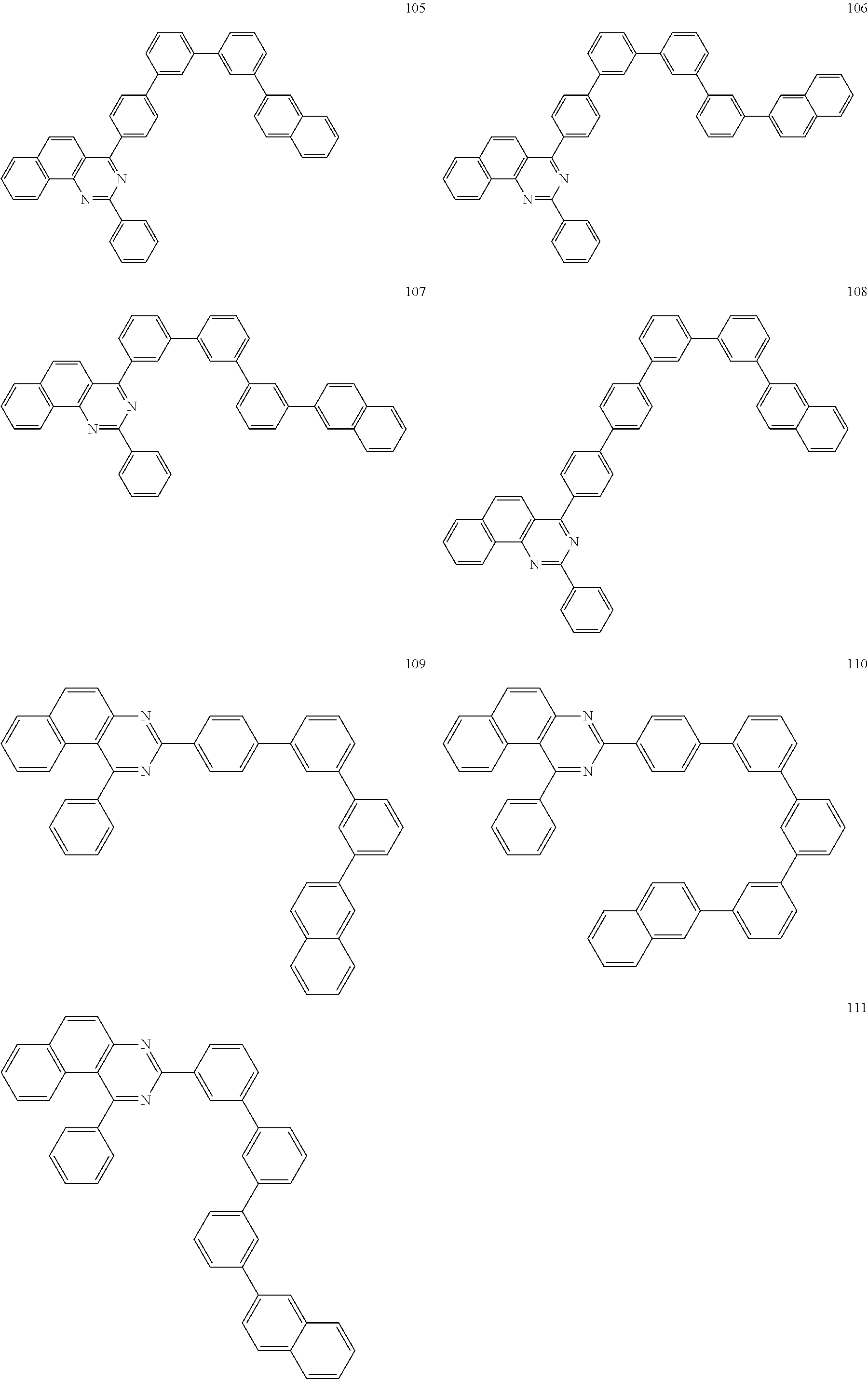

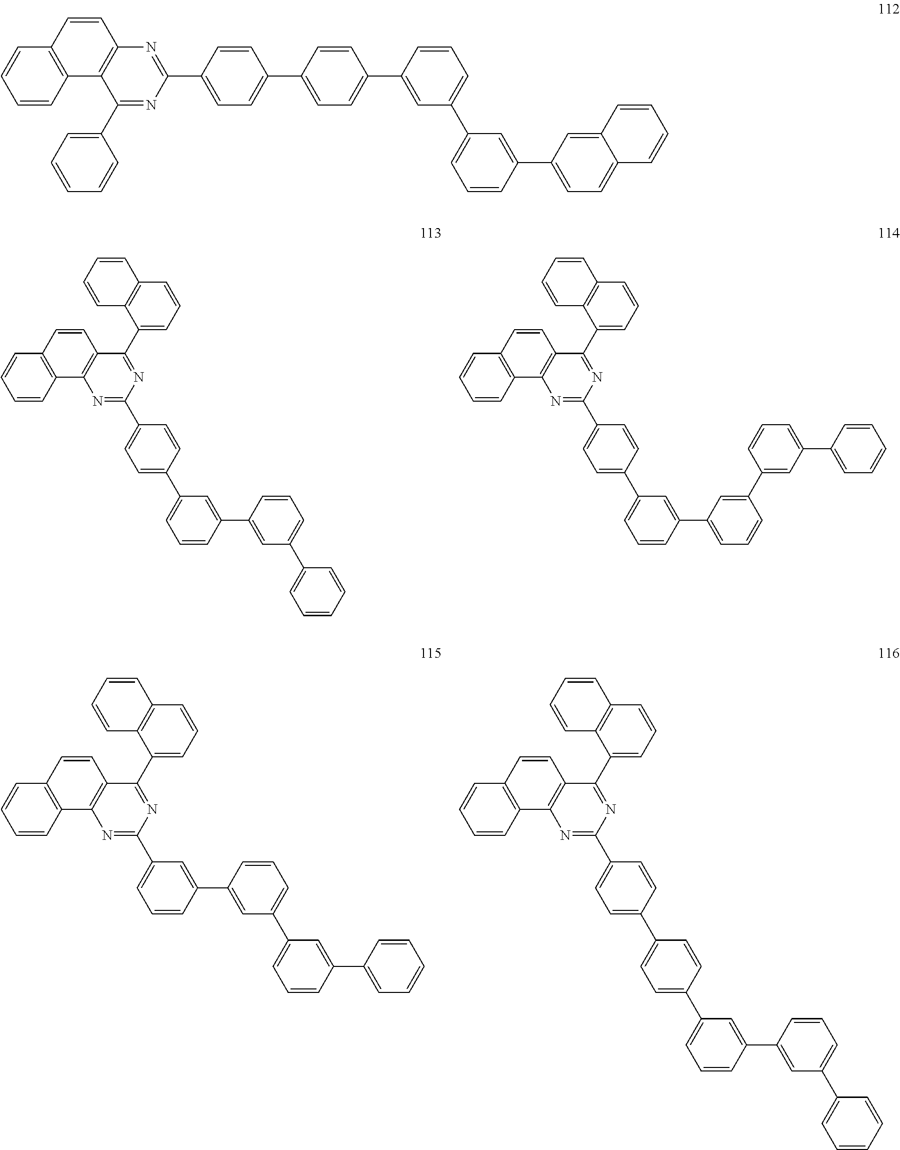 [SMILES: c1ccc(-c2nc(-c3ccc(-c4ccc(-c5cccc(-c6cccc(-c7ccc8ccccc8c7)c6)c5)cc4)cc3)nc3ccc4ccccc4c23)cc1 ; c1ccc(-c2cccc(-c3cccc(-c4ccc(-c5nc(-c6cccc7ccccc67)c6ccc7ccccc7c6n5)cc4)c3)c2)cc1 ; c1ccc(-c2cccc(-c3cccc(-c4cccc(-c5ccc(-c6nc(-c7cccc8ccccc78)c7ccc8ccccc8c7n6)cc5)c4)c3)c2)cc1 ; c1ccc(-c2cccc(-c3cccc(-c4cccc(-c5nc(-c6cccc7ccccc67)c6ccc7ccccc7c6n5)c4)c3)c2)cc1 ; c1ccc(-c2cccc(-c3cccc(-c4ccc(-c5ccc(-c6nc(-c7cccc8ccccc78)c7ccc8ccccc8c7n6)cc5)cc4)c3)c2)cc1]

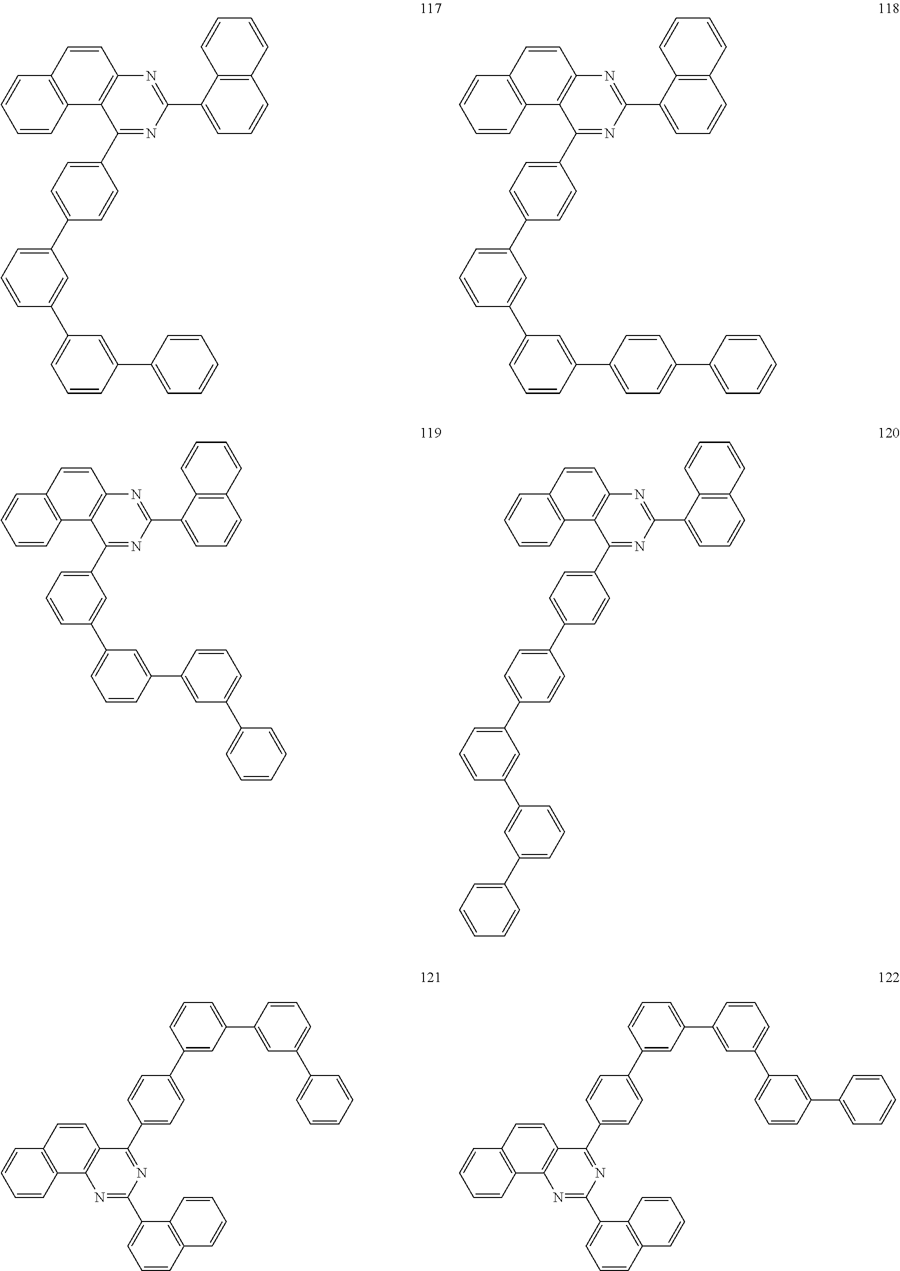

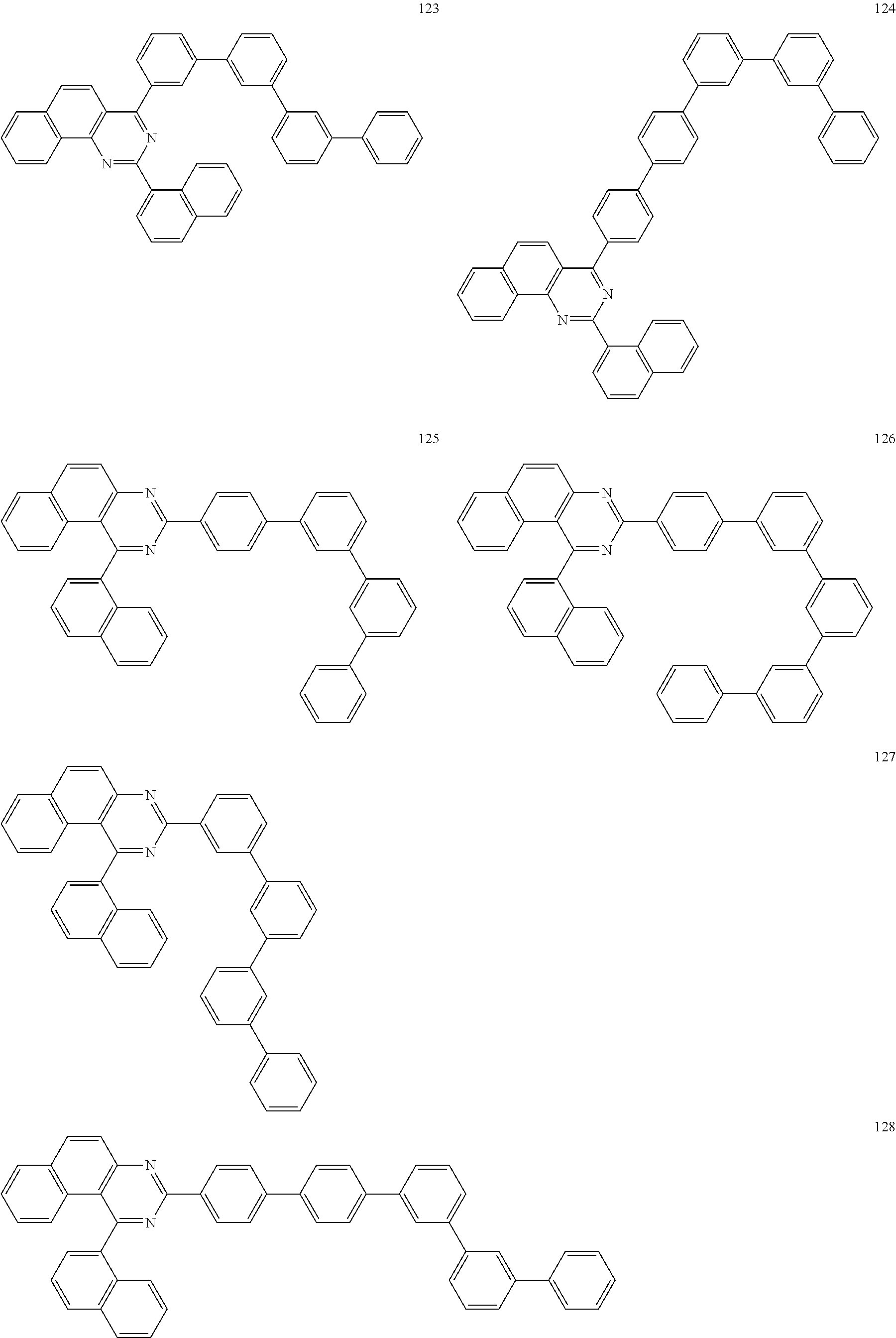

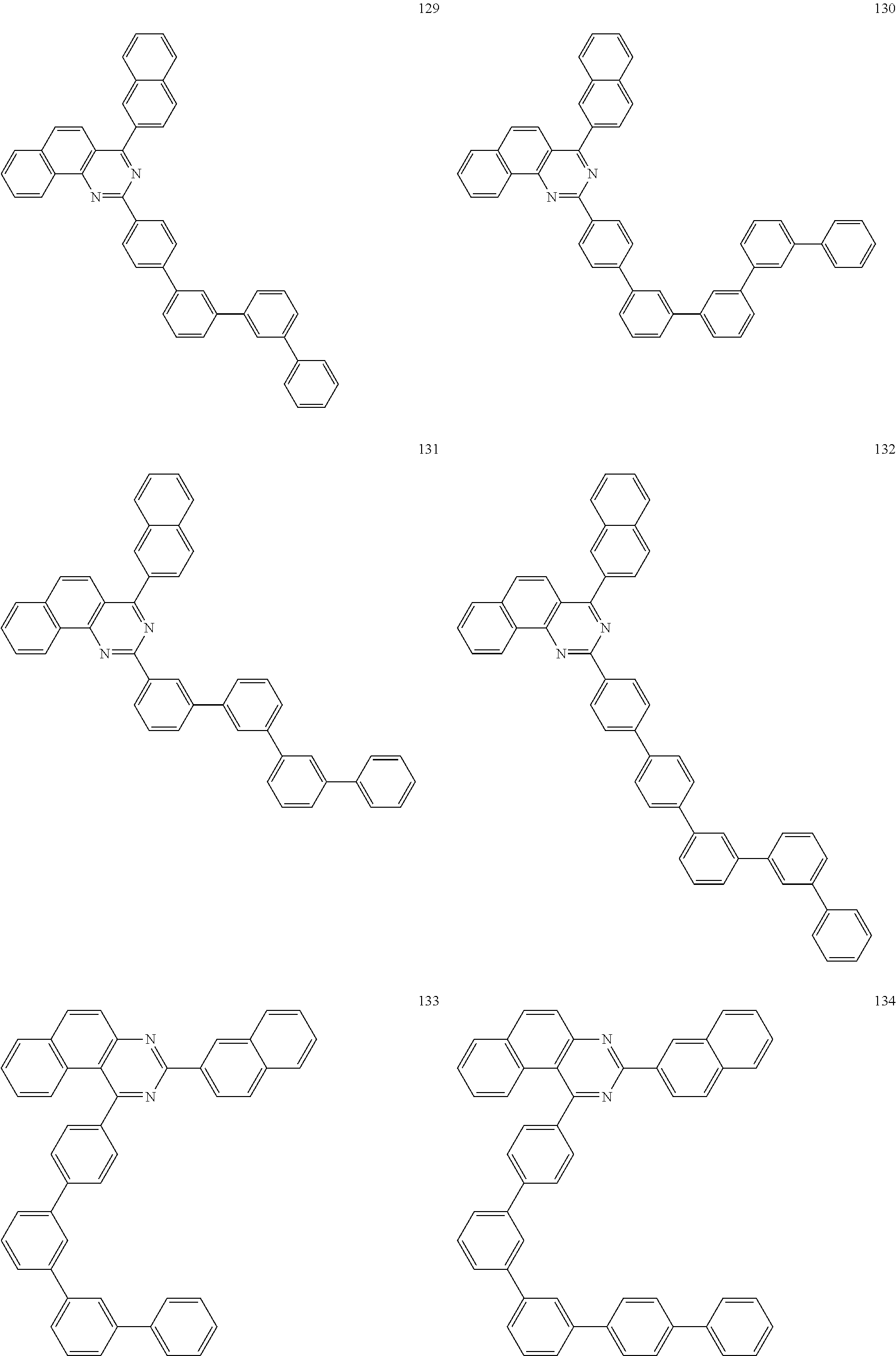

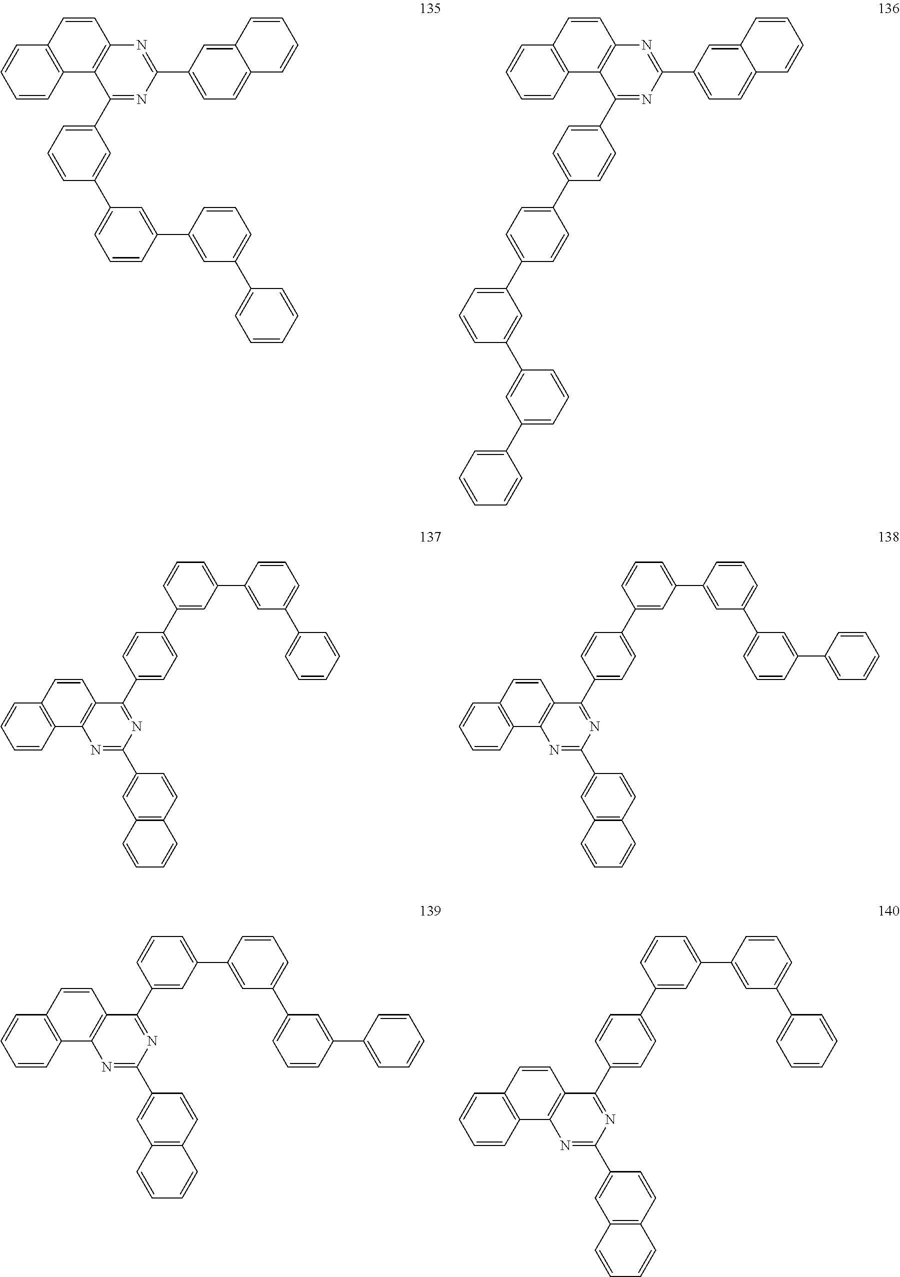

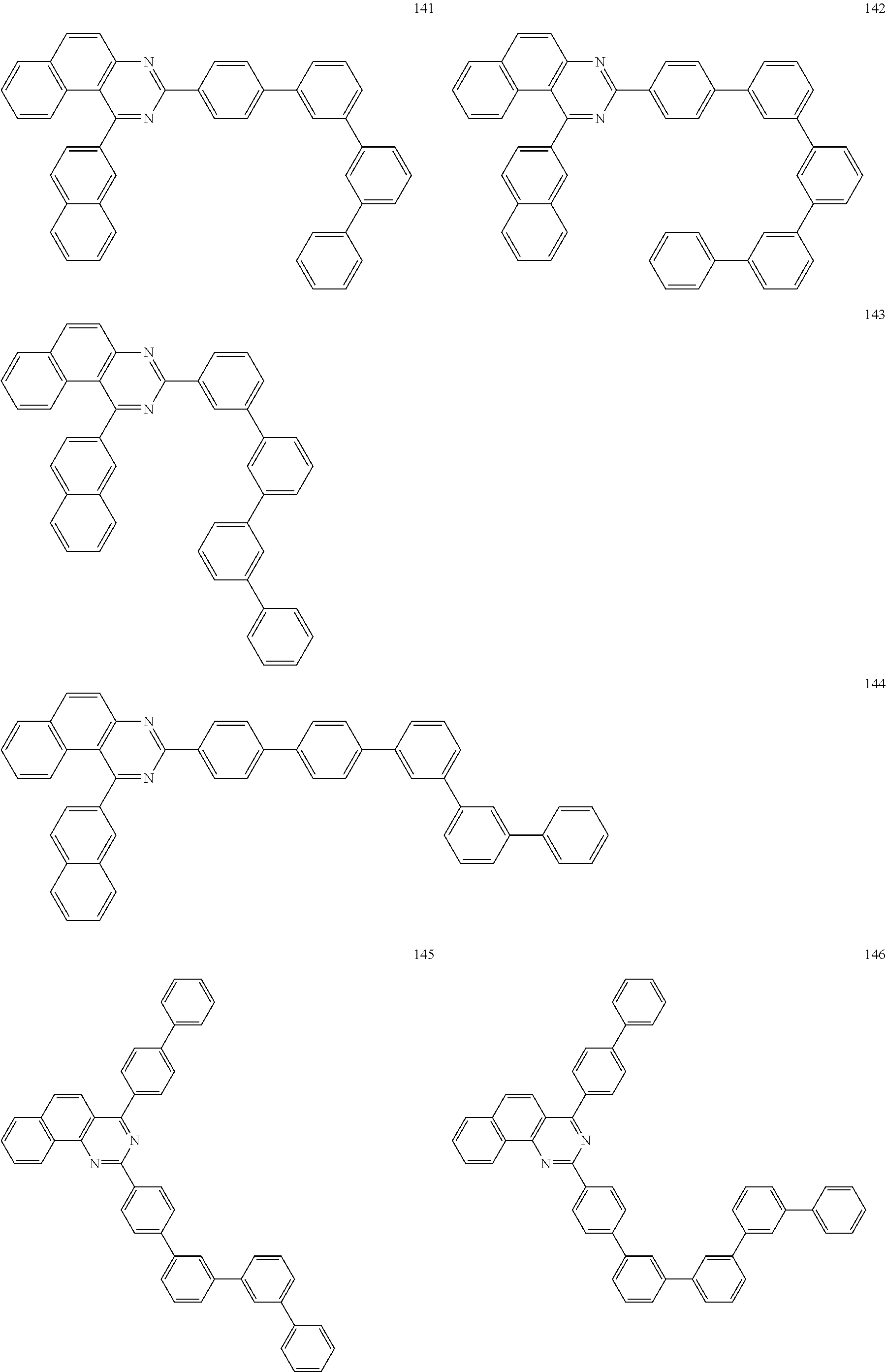

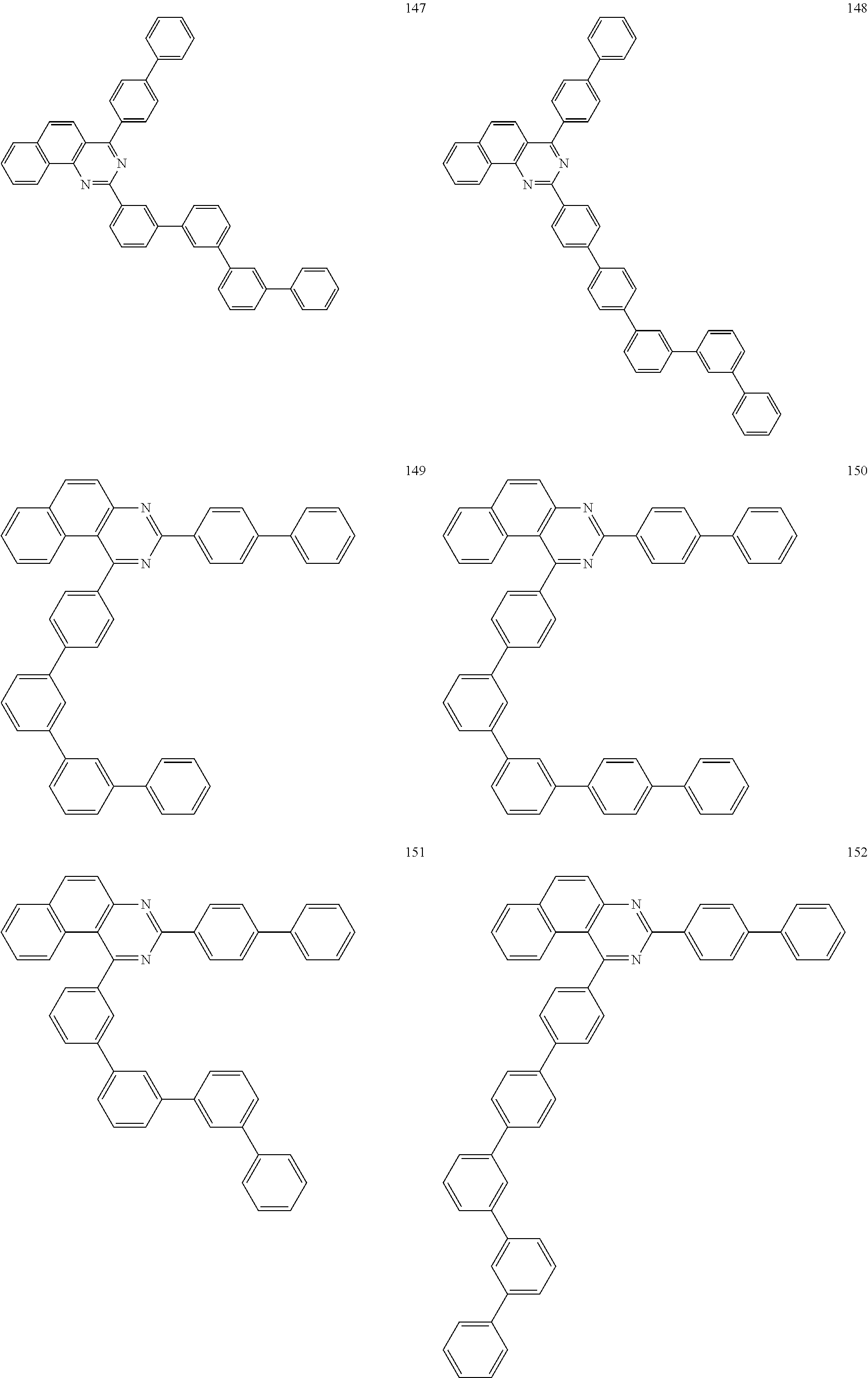

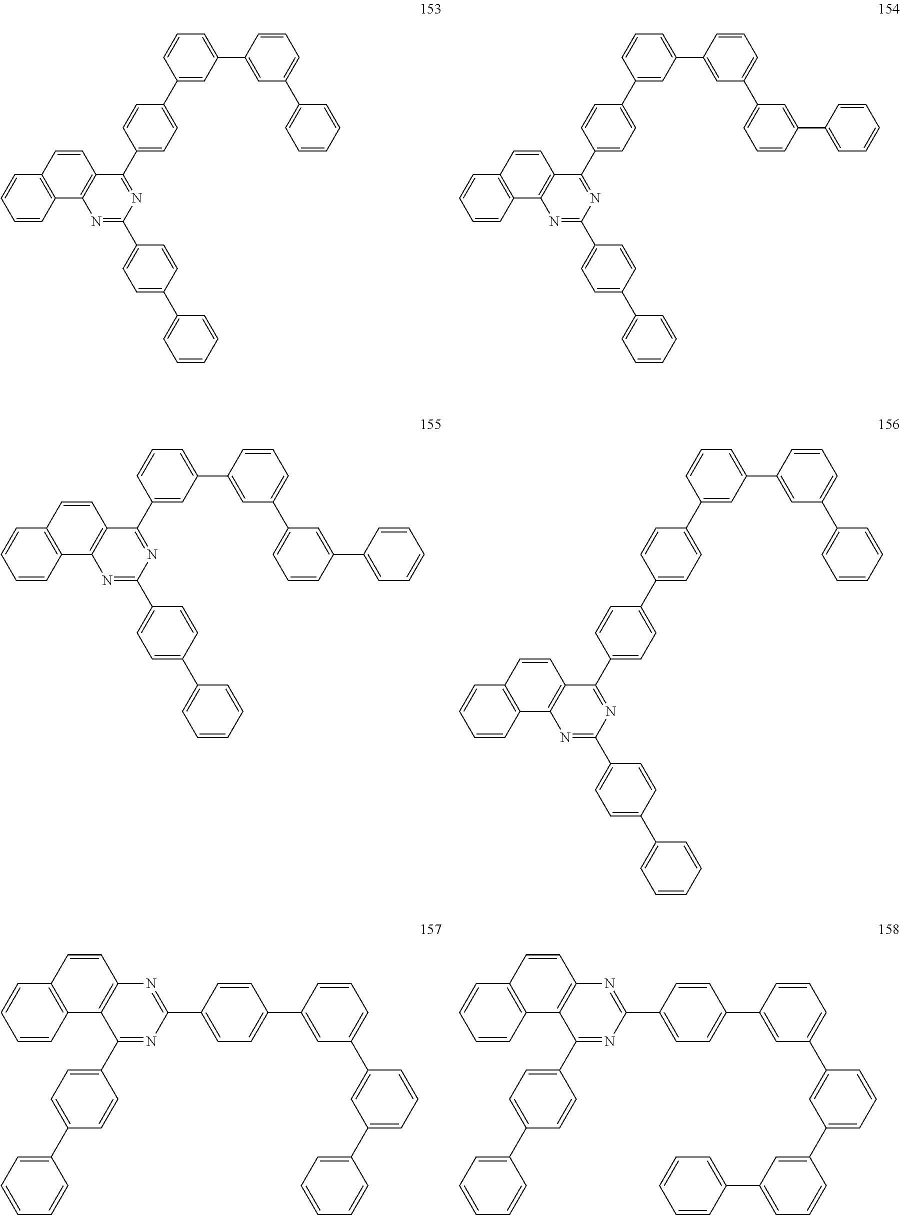

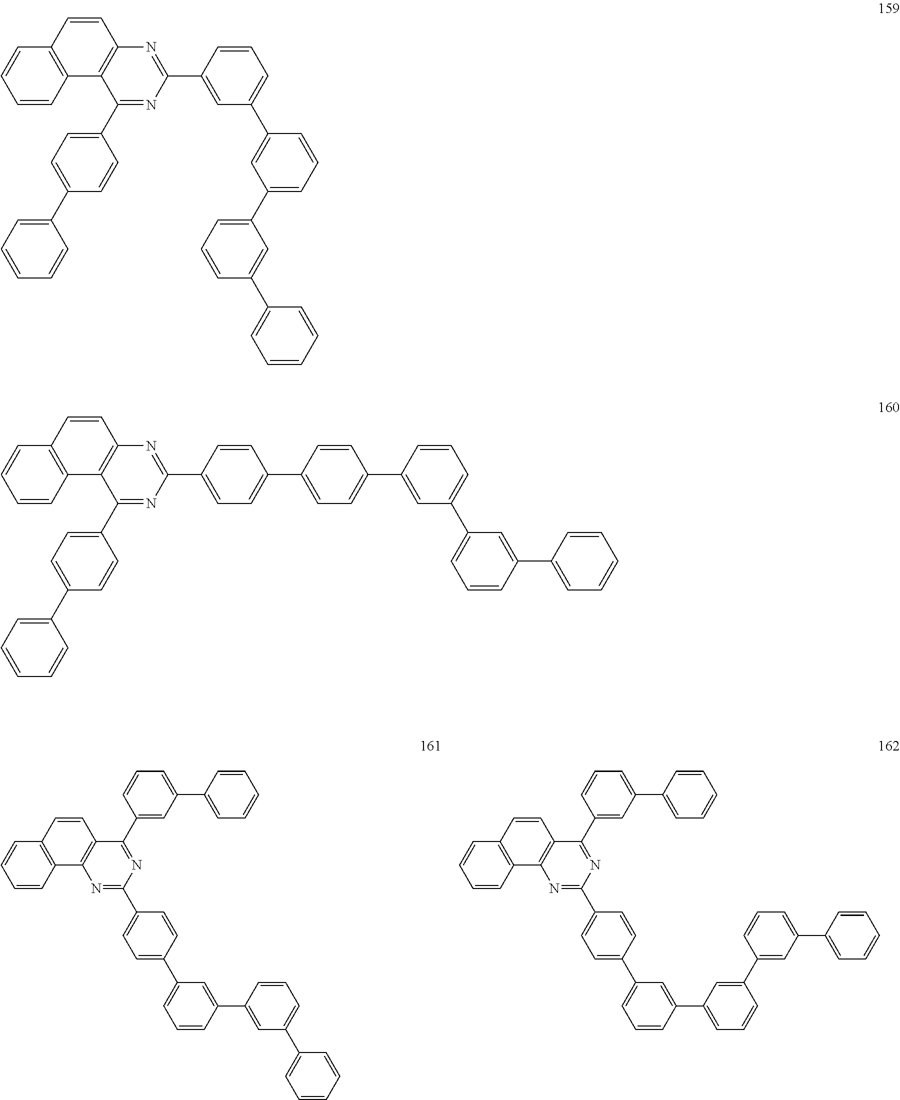 [SMILES: c1ccc(-c2ccc(-c3nc(-c4cccc(-c5cccc(-c6cccc(-c7ccccc7)c6)c5)c4)nc4ccc5ccccc5c34)cc2)cc1 ; c1ccc(-c2ccc(-c3nc(-c4ccc(-c5ccc(-c6cccc(-c7cccc(-c8ccccc8)c7)c6)cc5)cc4)nc4ccc5ccccc5c34)cc2)cc1 ; c1ccc(-c2cccc(-c3cccc(-c4ccc(-c5nc(-c6cccc(-c7ccccc7)c6)c6ccc7ccccc7c6n5)cc4)c3)c2)cc1 ; c1ccc(-c2cccc(-c3cccc(-c4cccc(-c5ccc(-c6nc(-c7cccc(-c8ccccc8)c7)c7ccc8ccccc8c7n6)cc5)c4)c3)c2)cc1]

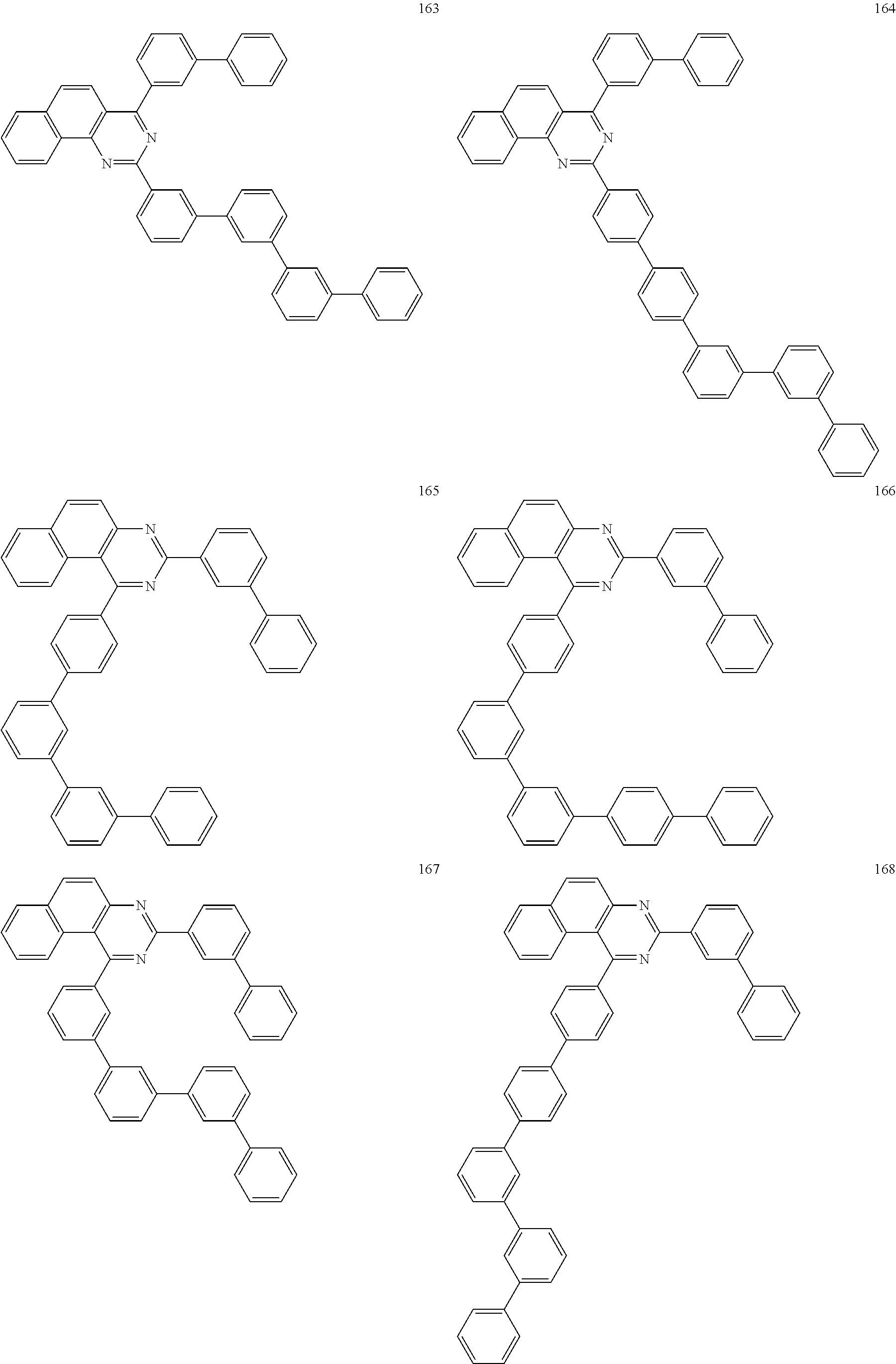

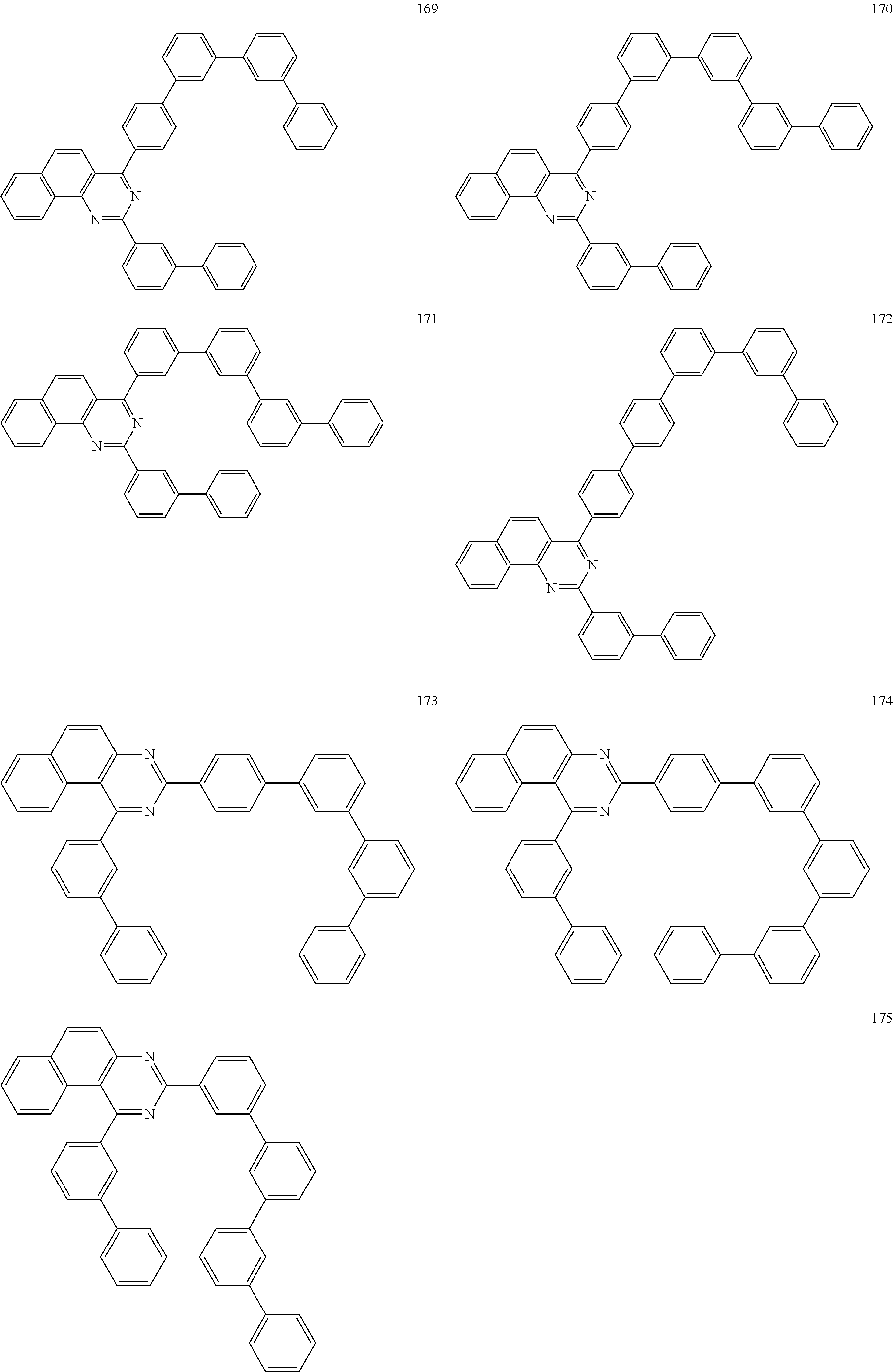

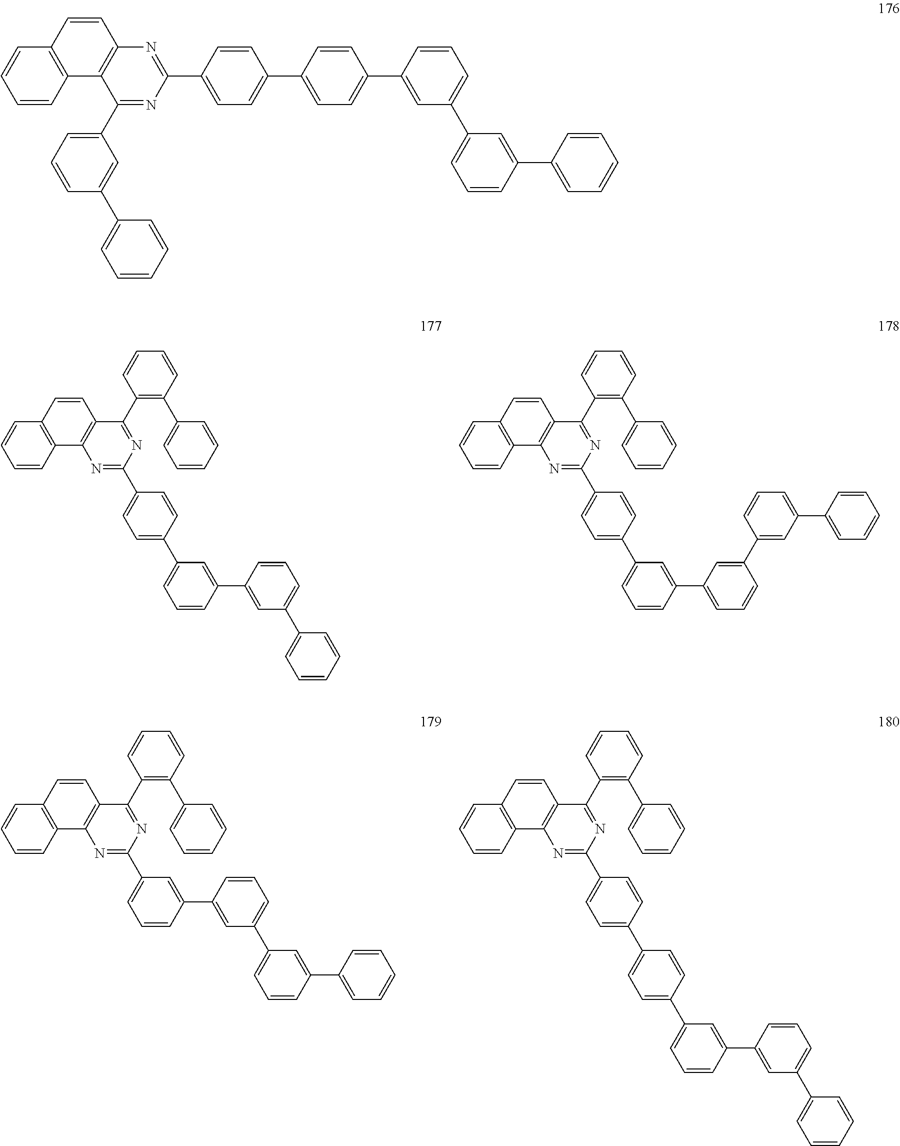 [SMILES: c1ccc(-c2cccc(-c3cccc(-c4ccc(-c5ccc(-c6nc(-c7cccc(-c8ccccc8)c7)c7c(ccc8ccccc87)n6)cc5)cc4)c3)c2)cc1 ; c1ccc(-c2cccc(-c3cccc(-c4ccc(-c5nc(-c6ccccc6-c6ccccc6)c6ccc7ccccc7c6n5)cc4)c3)c2)cc1 ; c1ccc(-c2cccc(-c3cccc(-c4cccc(-c5ccc(-c6nc(-c7ccccc7-c7ccccc7)c7ccc8ccccc8c7n6)cc5)c4)c3)c2)cc1 ; c1ccc(-c2cccc(-c3cccc(-c4cccc(-c5nc(-c6ccccc6-c6ccccc6)c6ccc7ccccc7c6n5)c4)c3)c2)cc1 ; c1ccc(-c2cccc(-c3cccc(-c4ccc(-c5ccc(-c6nc(-c7ccccc7-c7ccccc7)c7ccc8ccccc8c7n6)cc5)cc4)c3)c2)cc1]

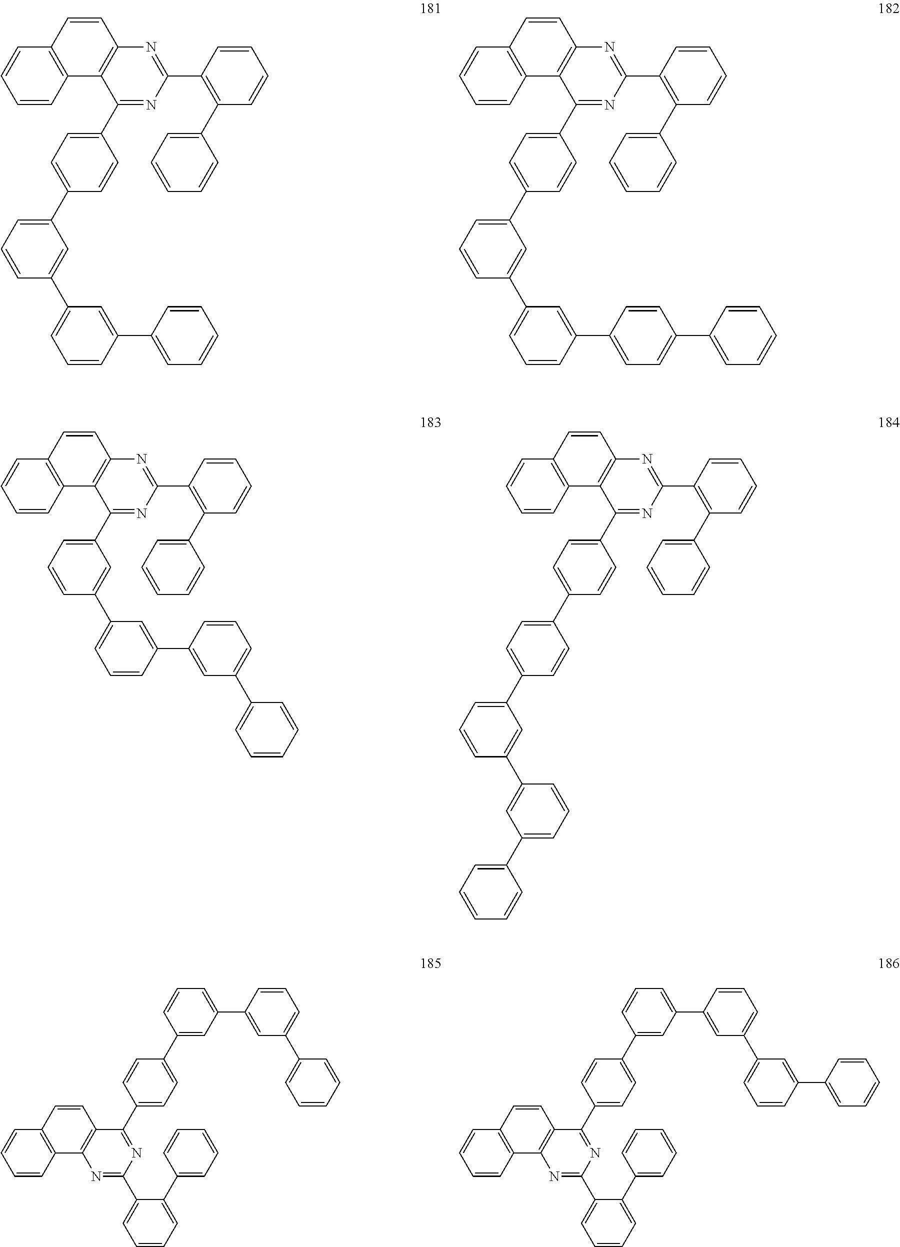

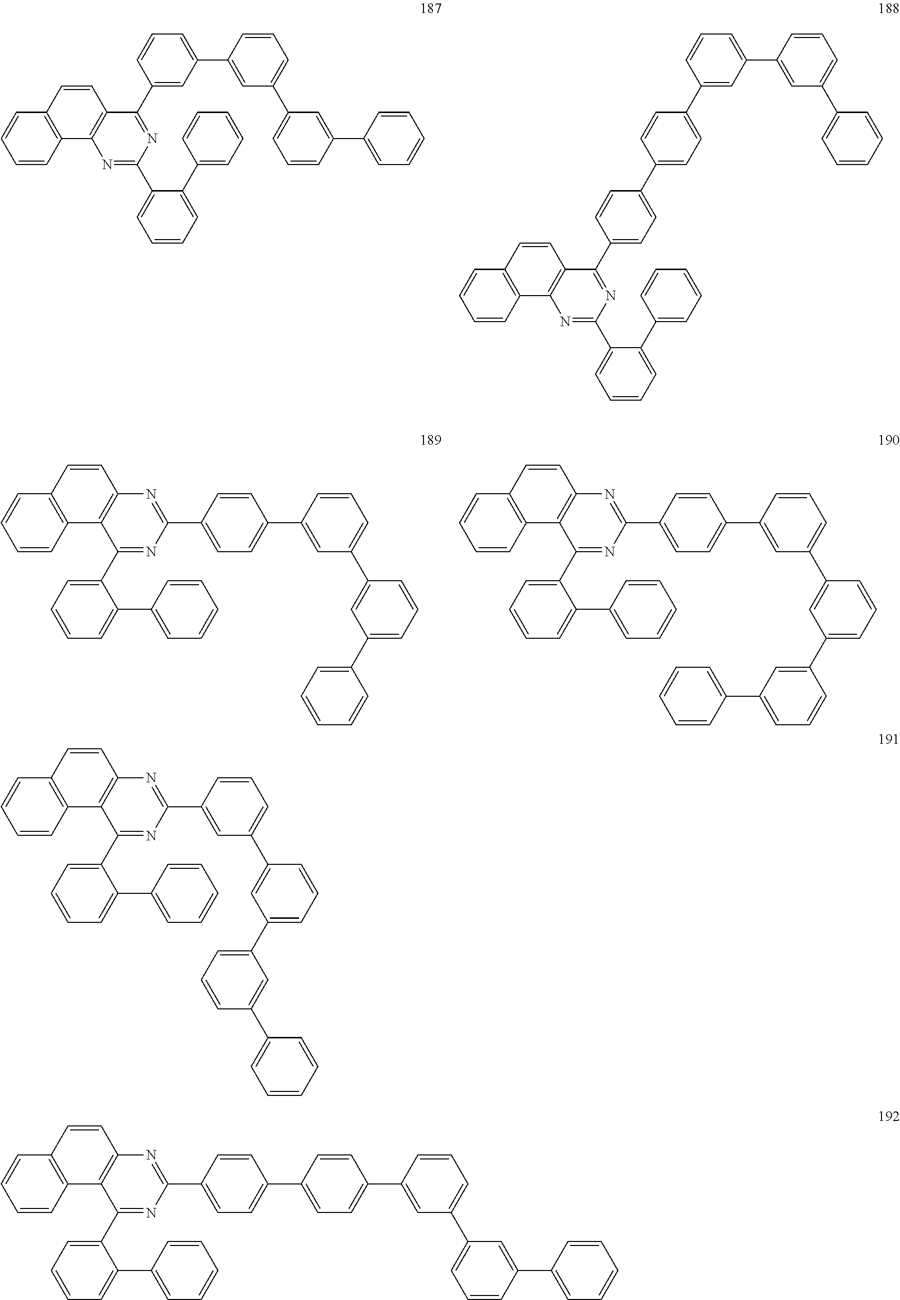

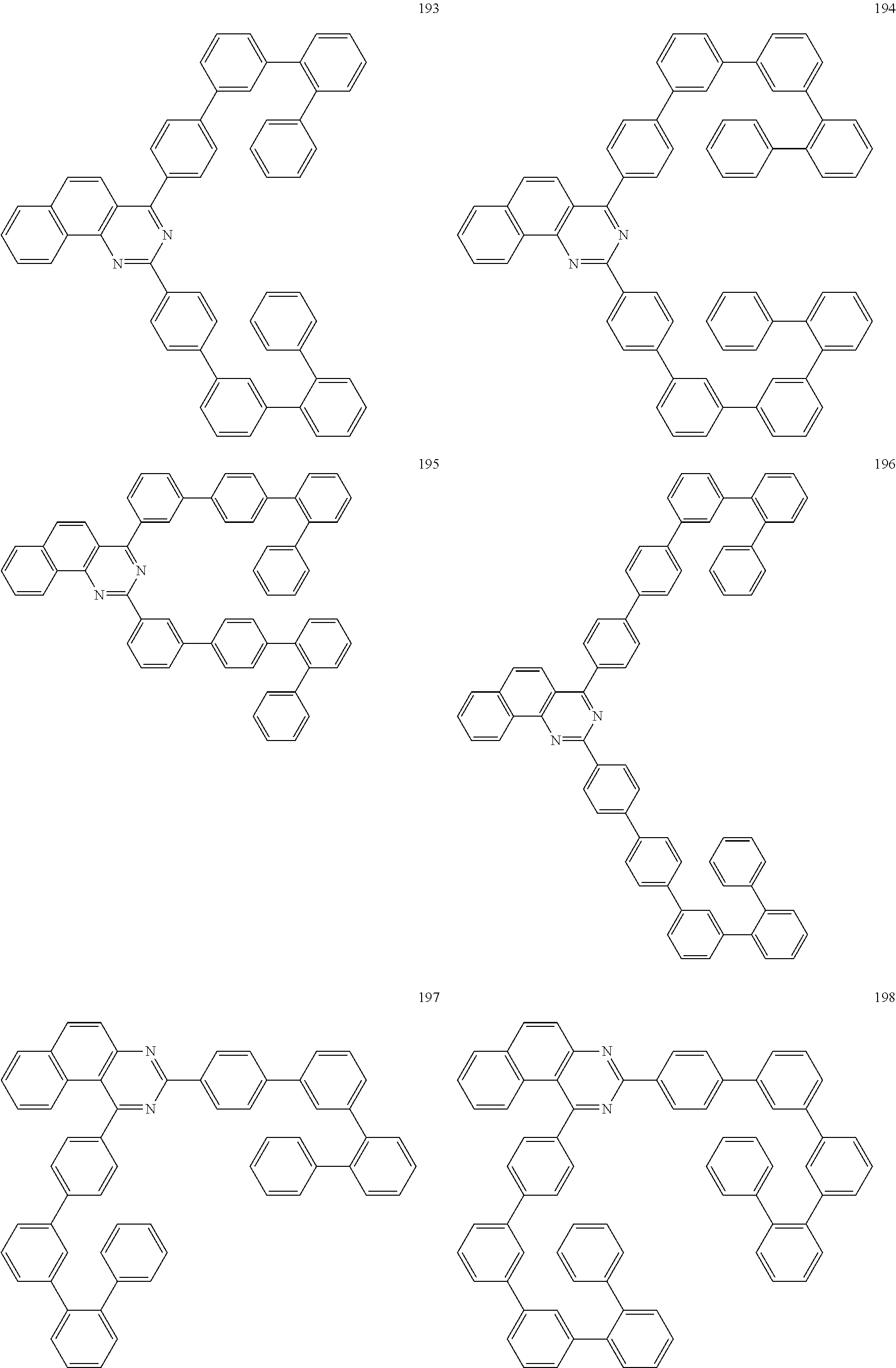

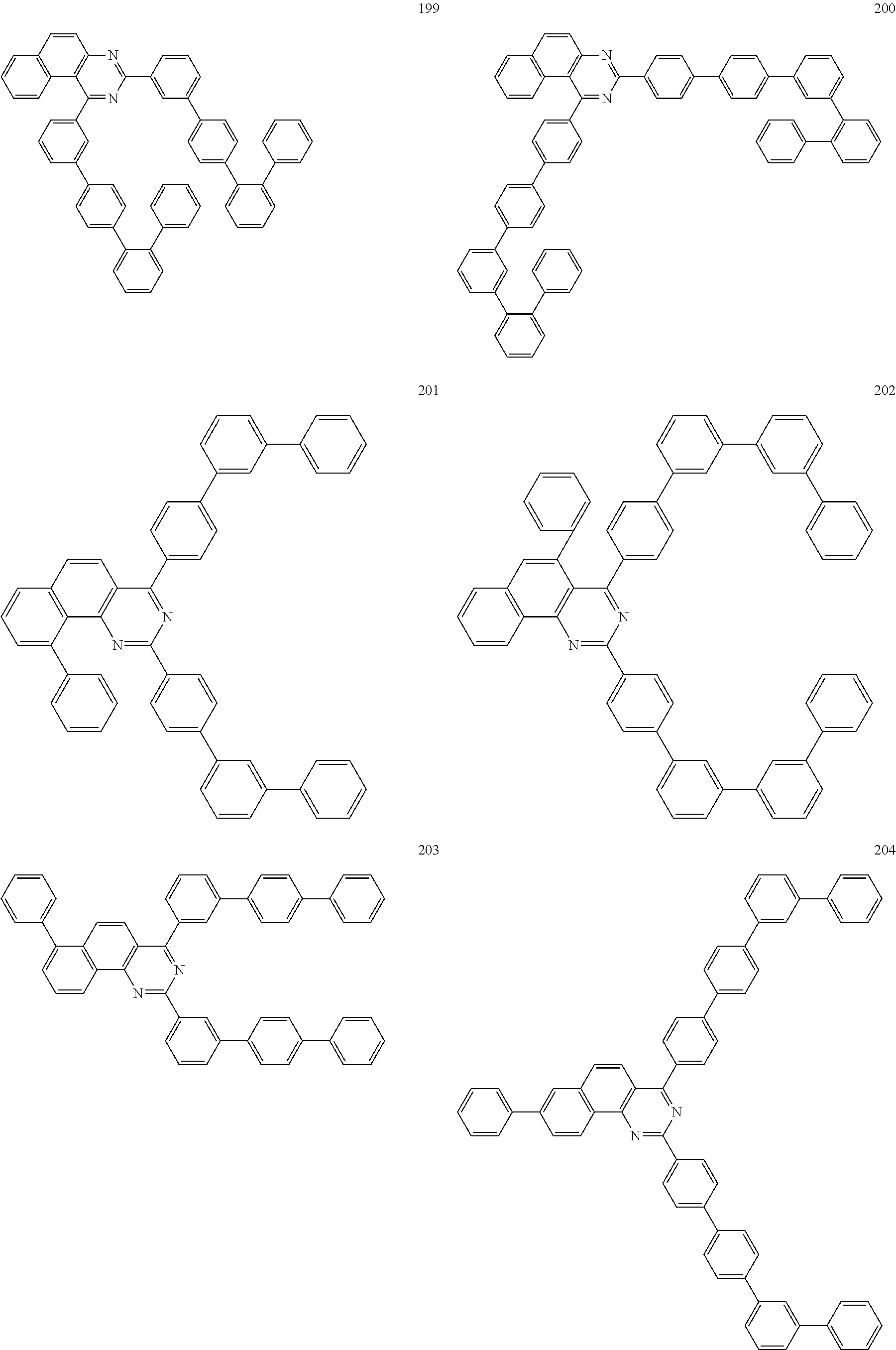

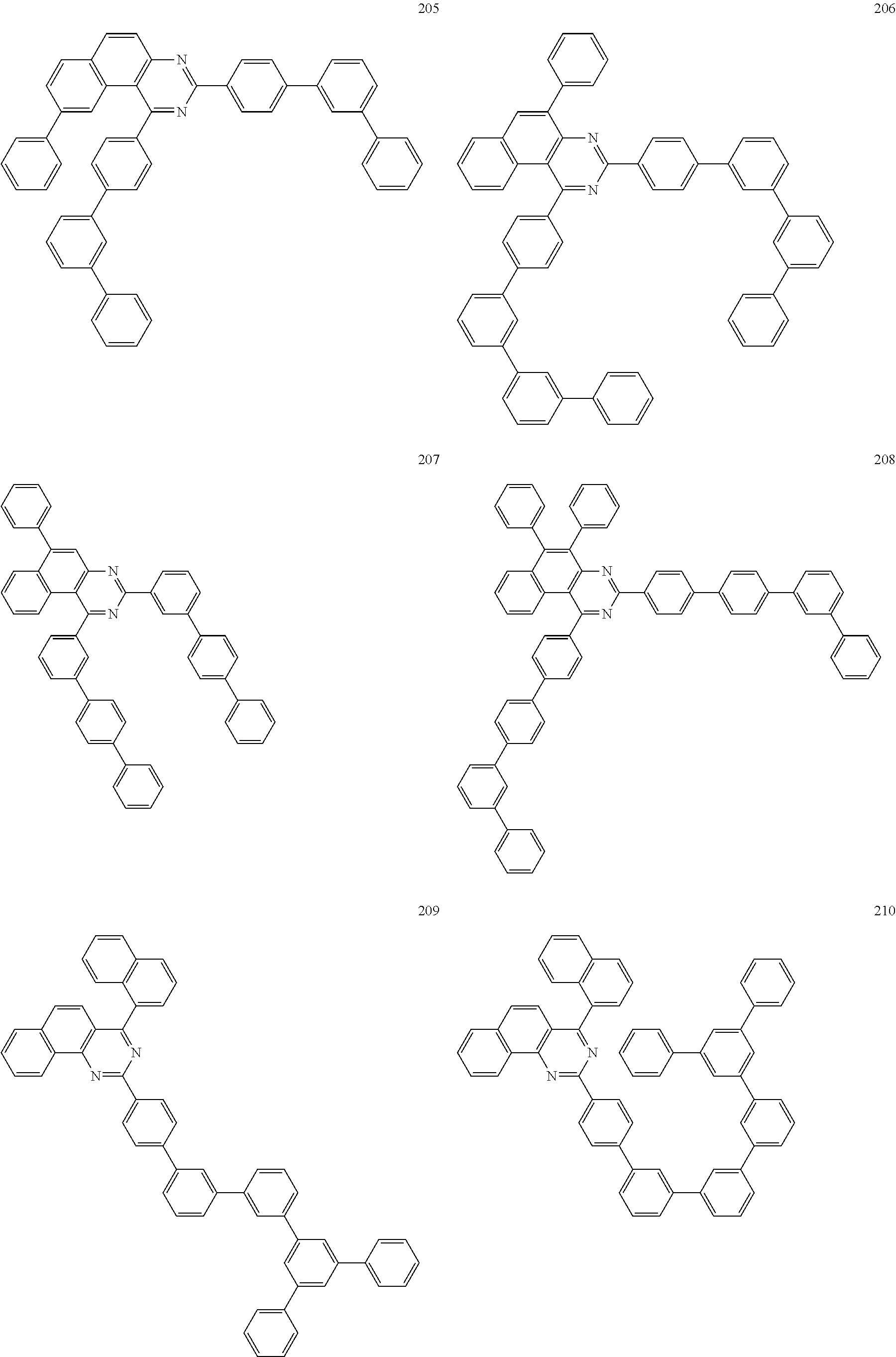

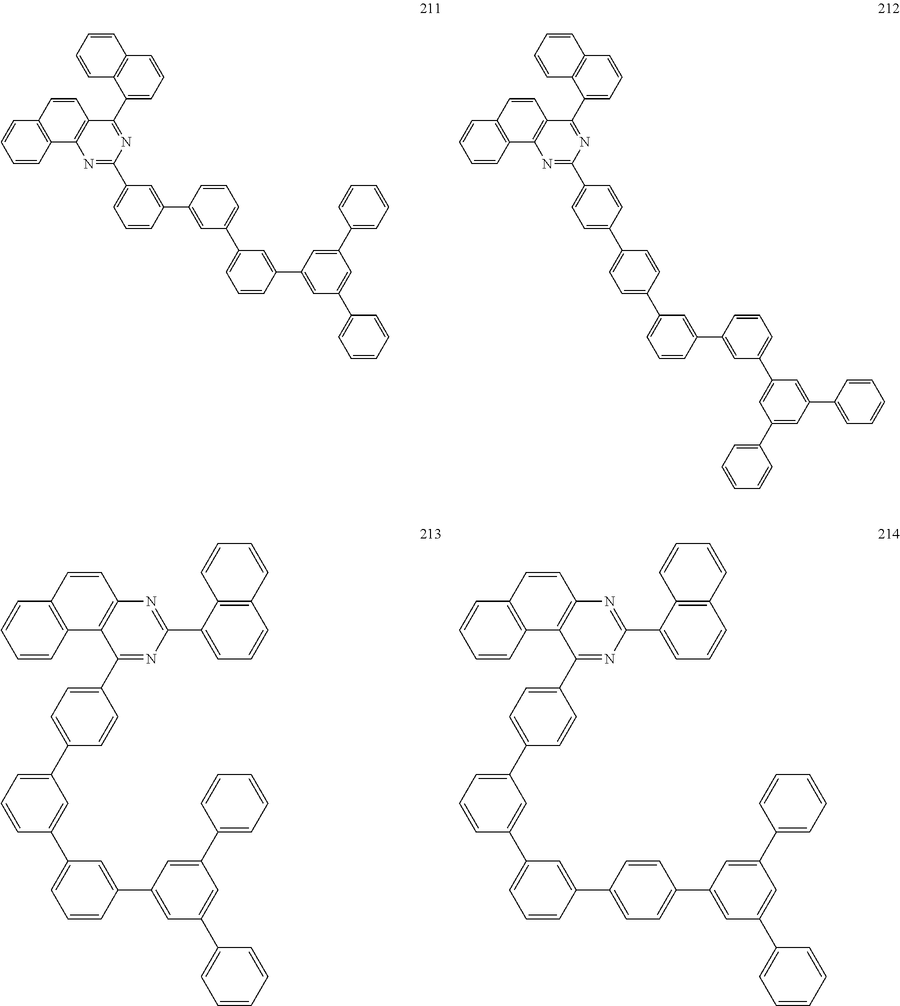 [SMILES: c1ccc(-c2cc(-c3ccccc3)cc(-c3cccc(-c4cccc(-c5cccc(-c6nc(-c7cccc8ccccc78)c7ccc8ccccc8c7n6)c5)c4)c3)c2)cc1 ; c1ccc(-c2cc(-c3ccccc3)cc(-c3cccc(-c4cccc(-c5ccc(-c6ccc(-c7nc(-c8cccc9ccccc89)c8ccc9ccccc9c8n7)cc6)cc5)c4)c3)c2)cc1 ; c1ccc(-c2cc(-c3ccccc3)cc(-c3cccc(-c4cccc(-c5ccc(-c6nc(-c7cccc8ccccc78)nc7ccc8ccccc8c67)cc5)c4)c3)c2)cc1 ; c1ccc(-c2cc(-c3ccccc3)cc(-c3ccc(-c4cccc(-c5cccc(-c6ccc(-c7nc(-c8cccc9ccccc89)nc8ccc9ccccc9c78)cc6)c5)c4)cc3)c2)cc1]

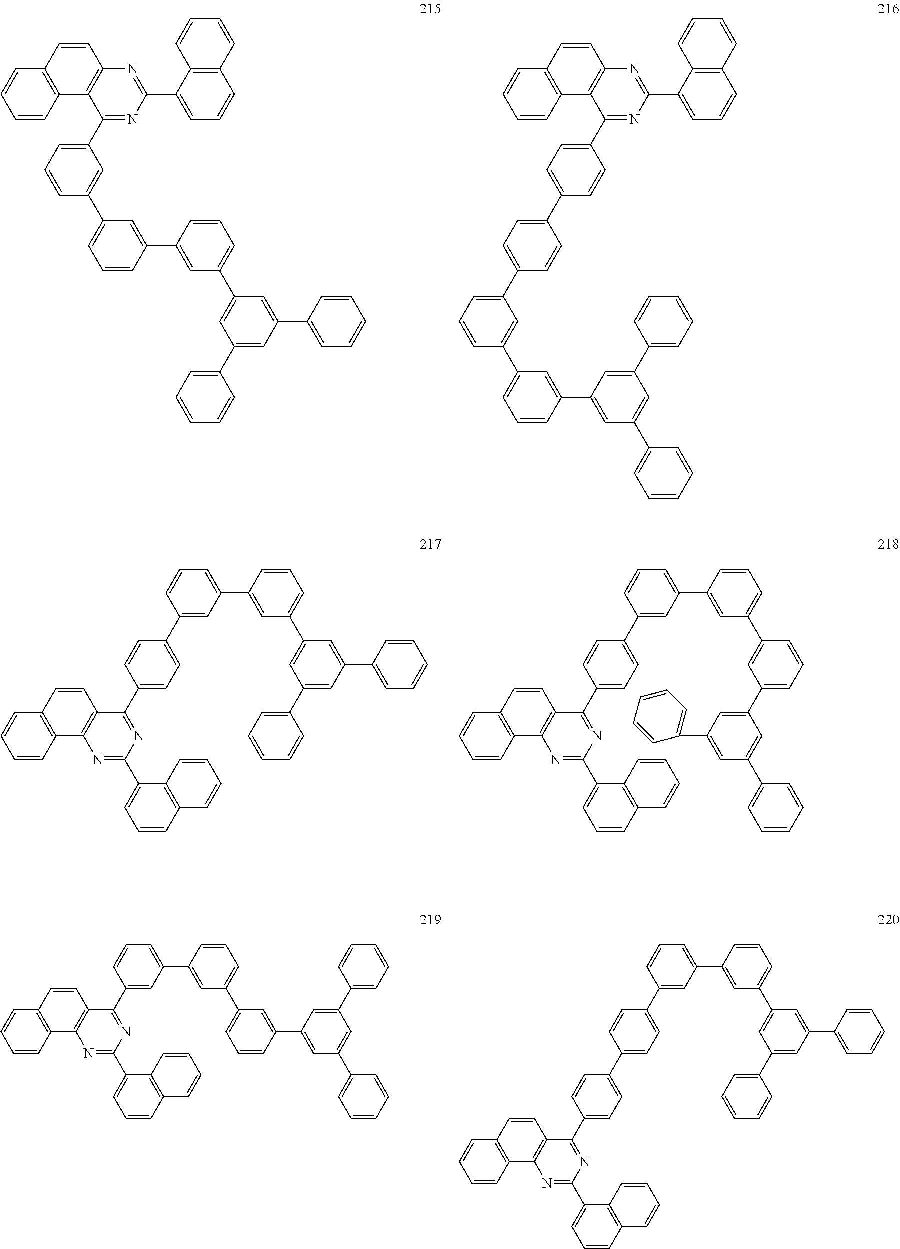

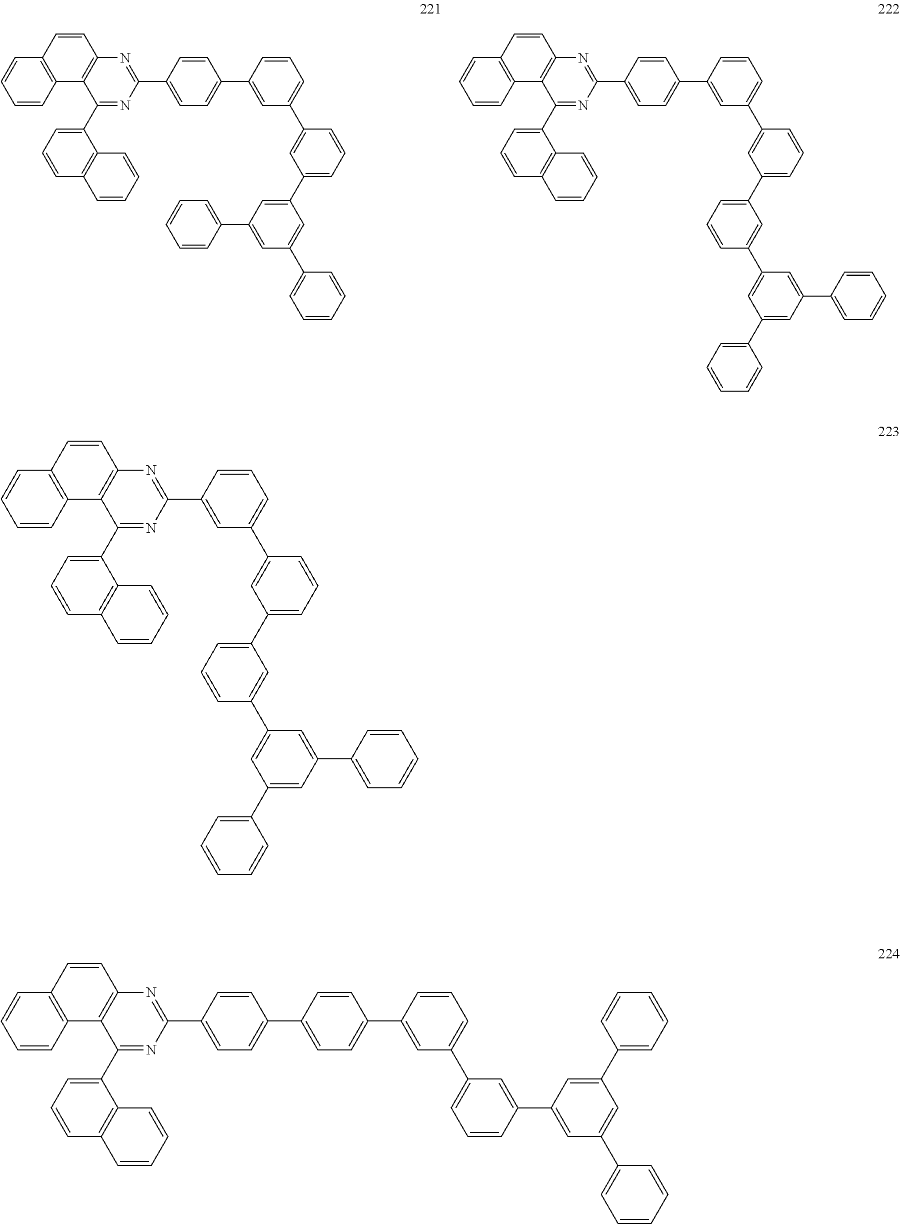 [SMILES: c1ccc(-c2cc(-c3ccccc3)cc(-c3cccc(-c4cccc(-c5ccc(-c6nc(-c7cccc8ccccc78)c7c(ccc8ccccc87)n6)cc5)c4)c3)c2)cc1 ; c1ccc(-c2cc(-c3ccccc3)cc(-c3cccc(-c4cccc(-c5cccc(-c6ccc(-c7nc(-c8cccc9ccccc89)c8c(ccc9ccccc98)n7)cc6)c5)c4)c3)c2)cc1 ; c1ccc(-c2cc(-c3ccccc3)cc(-c3cccc(-c4cccc(-c5cccc(-c6nc(-c7cccc8ccccc78)c7c(ccc8ccccc87)n6)c5)c4)c3)c2)cc1 ; c1ccc(-c2cc(-c3ccccc3)cc(-c3cccc(-c4cccc(-c5ccc(-c6ccc(-c7nc(-c8cccc9ccccc89)c8c(ccc9ccccc98)n7)cc6)cc5)c4)c3)c2)cc1]

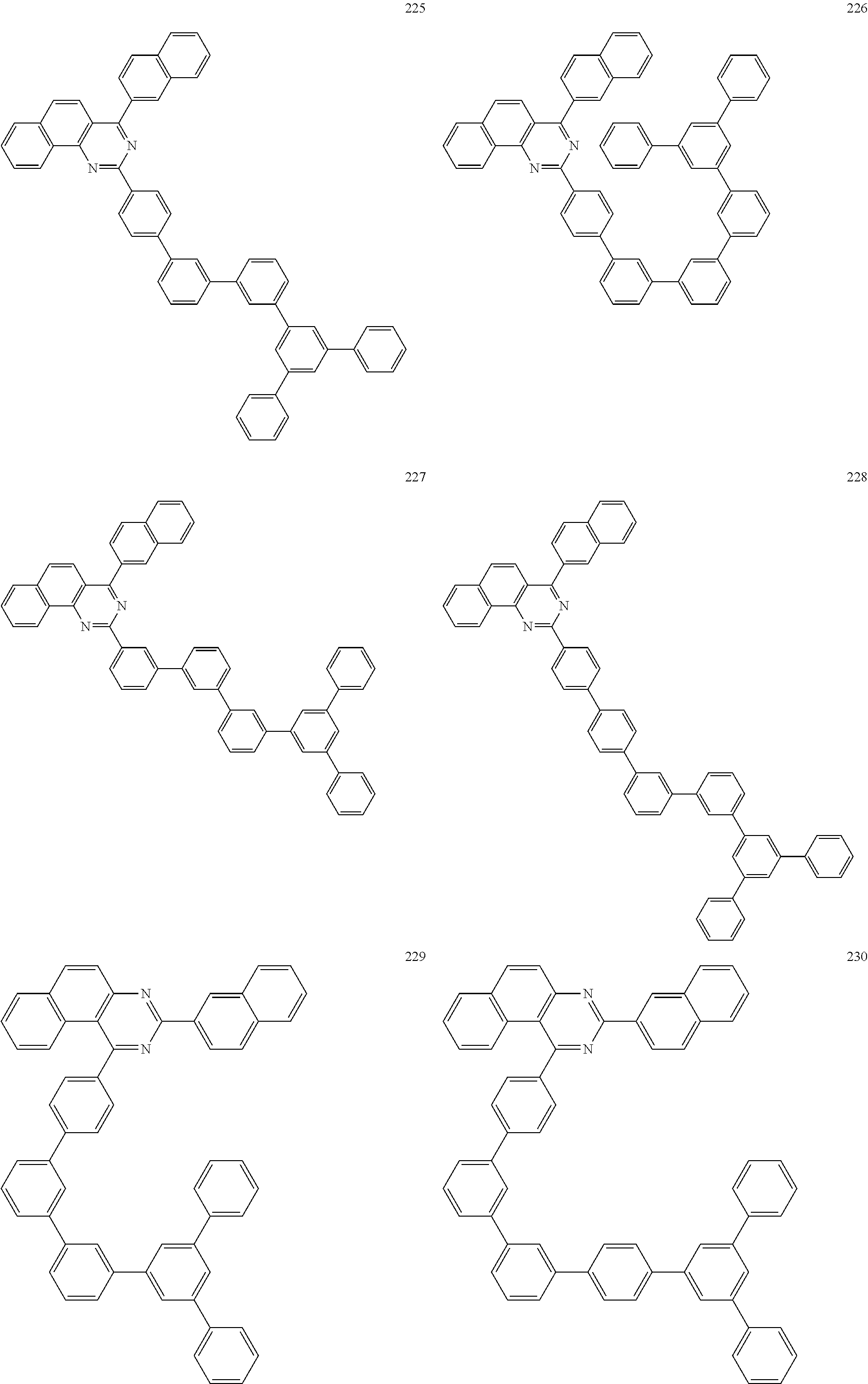

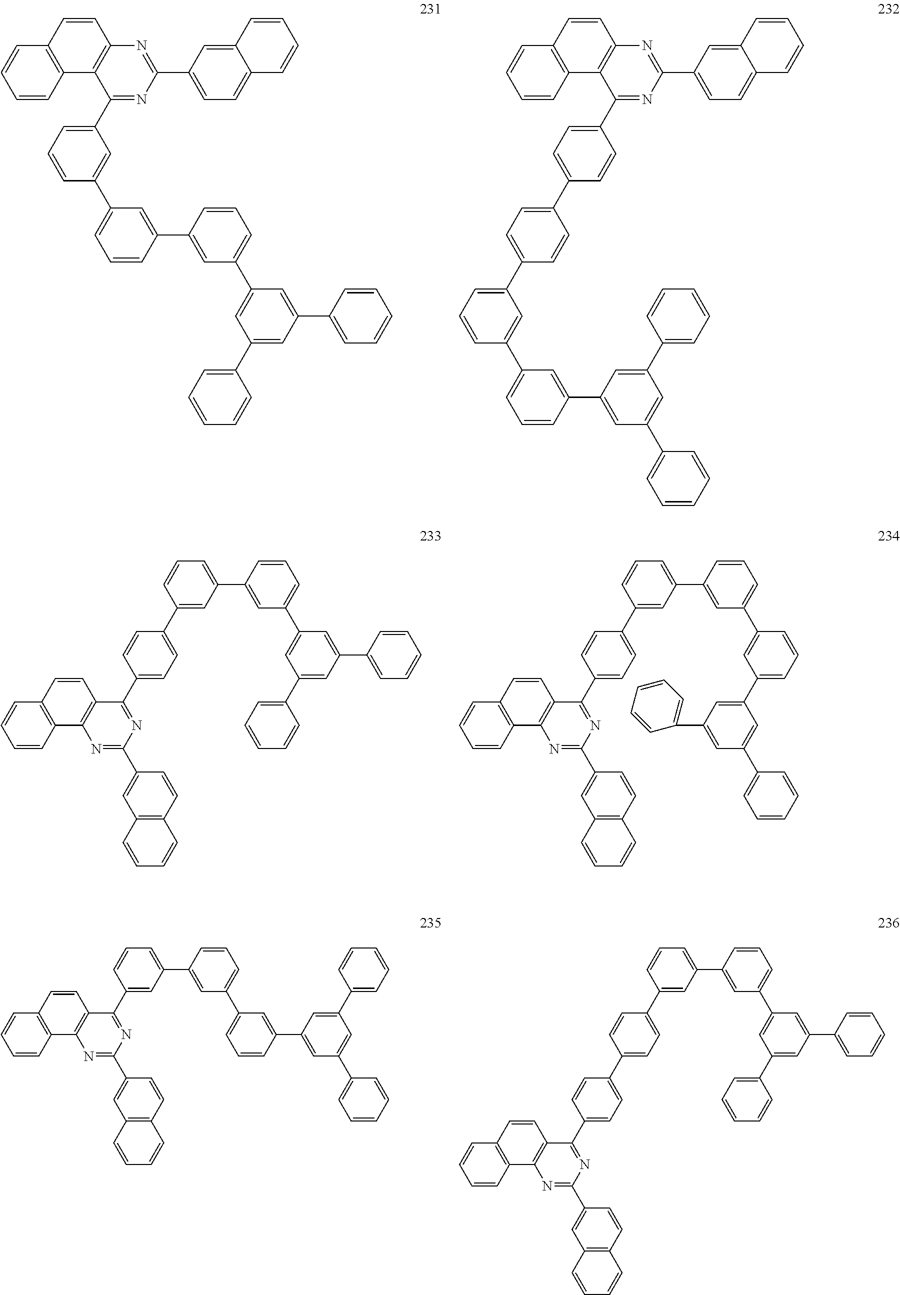

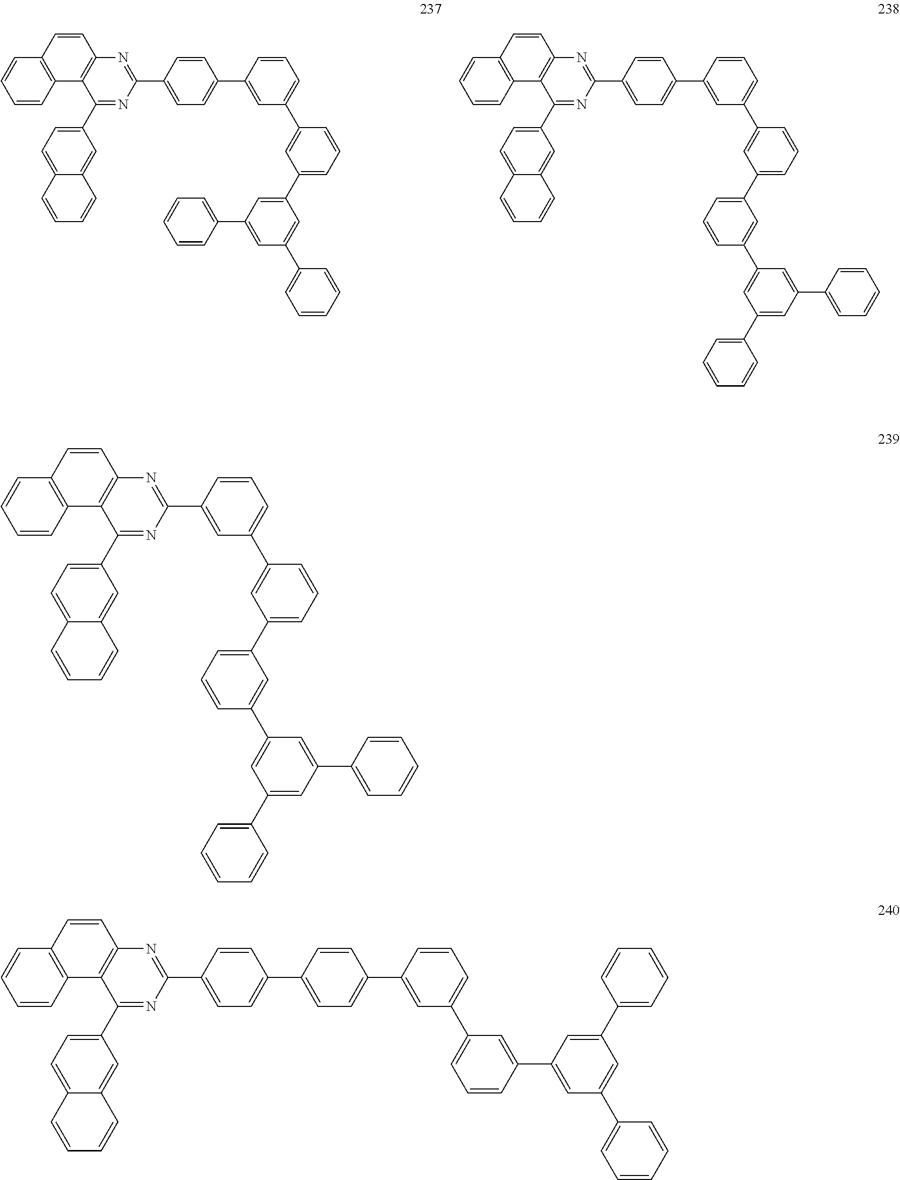 [SMILES: c1ccc(-c2cc(-c3ccccc3)cc(-c3cccc(-c4cccc(-c5ccc(-c6nc(-c7ccc8ccccc8c7)c7c(ccc8ccccc87)n6)cc5)c4)c3)c2)cc1 ; c1ccc(-c2cc(-c3ccccc3)cc(-c3cccc(-c4cccc(-c5cccc(-c6ccc(-c7nc(-c8ccc9ccccc9c8)c8c(ccc9ccccc98)n7)cc6)c5)c4)c3)c2)cc1 ; c1ccc(-c2cc(-c3ccccc3)cc(-c3cccc(-c4cccc(-c5cccc(-c6nc(-c7ccc8ccccc8c7)c7c(ccc8ccccc87)n6)c5)c4)c3)c2)cc1 ; c1ccc(-c2cc(-c3ccccc3)cc(-c3cccc(-c4cccc(-c5ccc(-c6ccc(-c7nc(-c8ccc9ccccc9c8)c8c(ccc9ccccc98)n7)cc6)cc5)c4)c3)c2)cc1]

In an implementation, the organic compound may be applied to an organic optoelectric device.

The organic compound may be applied to an organic optoelectric device at alone or with another organic compound. The organic compound may be applied as a composition with another organic compound.

Hereinafter, examples of a composition for an organic optoelectric device including the organic compound are described.

The composition for an organic optoelectric device may be, e.g., a composition including the organic compound described above (e.g., represented by Chemical Formula 1) and at least one organic compound having a carbazole moiety. Hereinafter, the organic compound represented by Chemical Formula 1 is referred to as ‘a first organic compound’, and the organic compound having a carbazole moiety is referred to as ‘a second organic compound’.

The second organic compound may be, e.g., a compound represented by the following Chemical Formula 4.

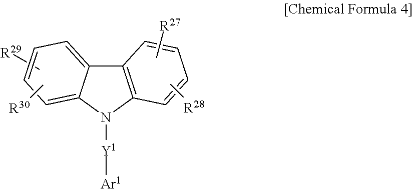

In Chemical Formula 4,

Y¹ may be or include, e.g., a single bond, a substituted or unsubstituted C1 to C20 alkylene group, a substituted or unsubstituted C2 to C20 alkenylene group, a substituted or unsubstituted C6 to C30 arylene group, a substituted or unsubstituted C2 to C30 heterocyclic group, or a combination thereof.

Ar¹ may be or include, e.g., a substituted or unsubstituted C6 to C30 aryl group, a substituted or unsubstituted C2 to C30 heterocyclic group, or a combination thereof.

R²⁷ to R³⁰ may each independently be or include, e.g., hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof. In an implementation, at least one of R²⁷ to R³⁰ and Ar¹ may include a substituted or unsubstituted triphenylene group or a substituted or unsubstituted carbazole group.

The second organic compound represented by the Chemical Formula 4 may be represented by, e.g. one of the following Chemical Formulae 4-I to 4-III.

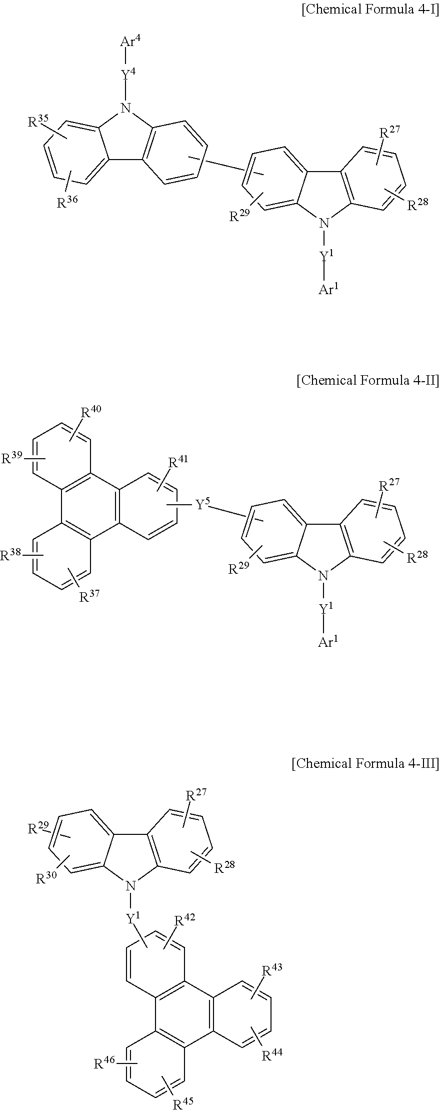

In Chemical Formulae 4-I to 4-III,

Y¹, Y⁴, and Y⁵ may each independently be or include, e.g., a single bond, a substituted or unsubstituted C1 to C20 alkylene group, a substituted or unsubstituted C2 to C20 alkenylene group, a substituted or unsubstituted C6 to C30 arylene group, a substituted or unsubstituted C2 to C30 heterocyclic group, or a combination thereof.

Ar¹ and Ar⁴ may each independently be or include, e.g., a substituted or unsubstituted C6 to C30 aryl group, a substituted or unsubstituted C2 to C30 heterocyclic group, or a combination thereof.

R²⁷ to R³⁰ and R³⁵ to R⁴⁶ may each independently be or include, e.g., hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof.

The second organic compound represented by the Chemical Formula 4 may be, e.g., one of compounds of the following Group 3.

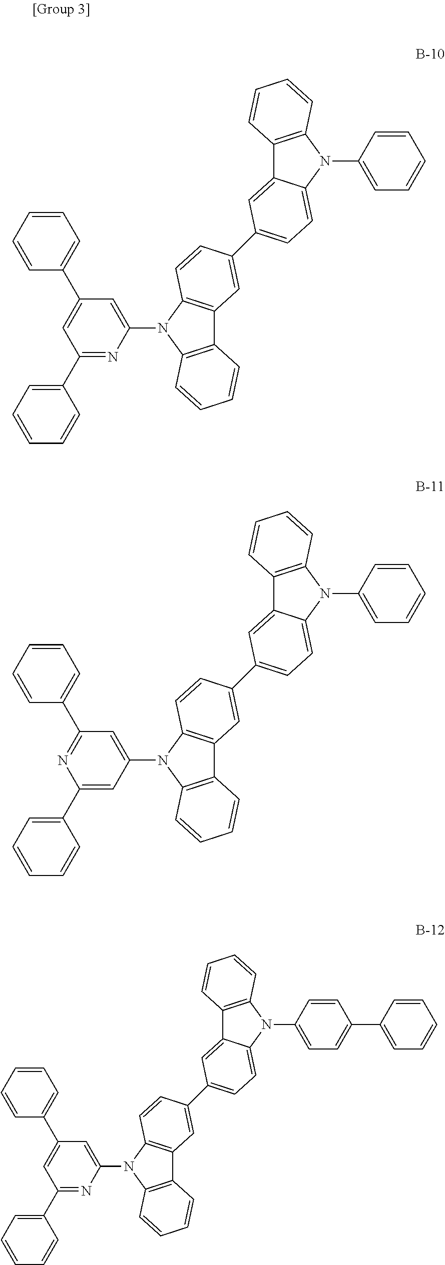 [SMILES: c1ccc(-c2cc(-c3ccccc3)nc(-n3c4ccccc4c4cc(-c5ccc6c(c5)c5ccccc5n6-c5ccccc5)ccc43)c2)cc1 ; c1ccc(-c2cc(-n3c4ccccc4c4cc(-c5ccc6c(c5)c5ccccc5n6-c5ccccc5)ccc43)cc(-c3ccccc3)n2)cc1 ; c1ccc(-c2ccc(-n3c4ccccc4c4cc(-c5ccc6c(c5)c5ccccc5n6-c5cc(-c6ccccc6)cc(-c6ccccc6)n5)ccc43)cc2)cc1]

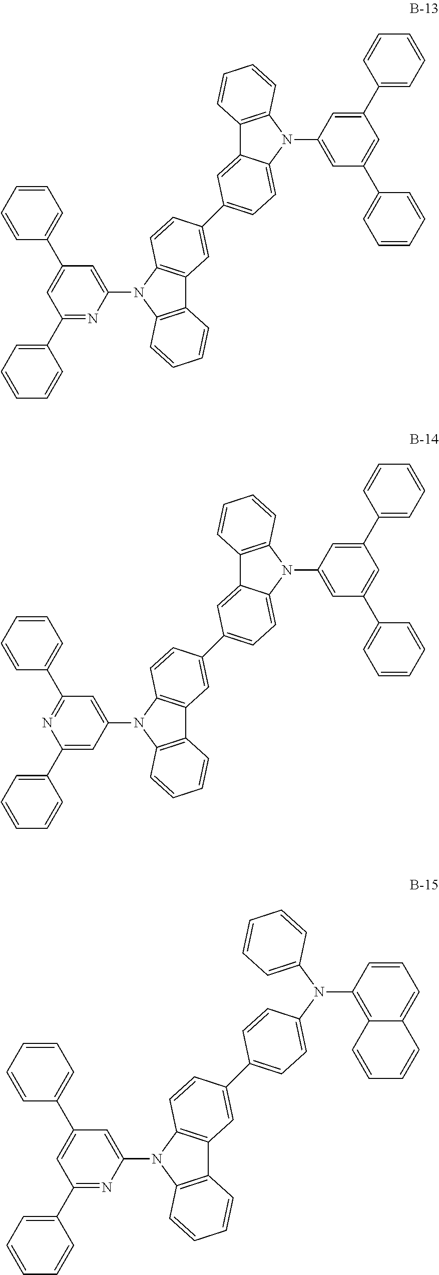 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)cc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-c3ccccc3)nc(-n3c4ccccc4c4cc(-c5ccc(N(c6ccccc6)c6cccc7ccccc67)cc5)ccc43)c2)cc1]

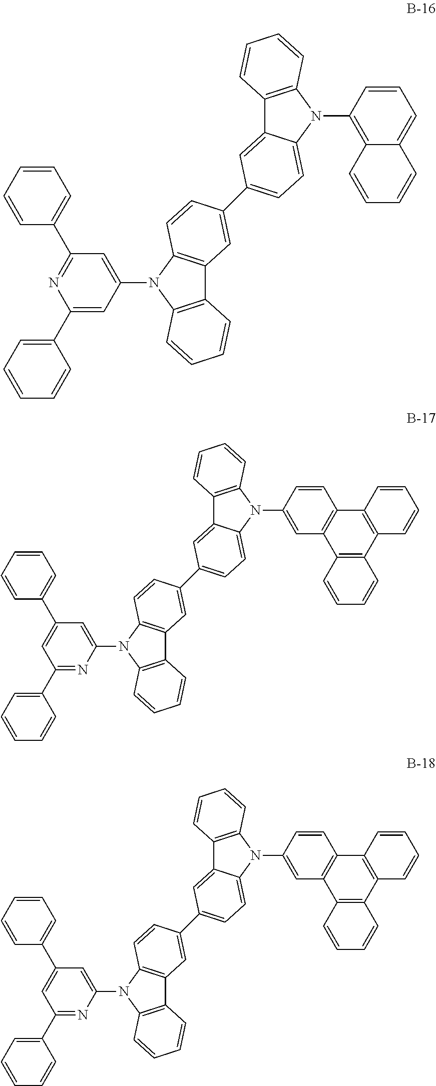 [SMILES: c1ccc(-c2cc(-n3c4ccccc4c4cc(-c5ccc6c(c5)c5ccccc5n6-c5cccc6ccccc56)ccc43)cc(-c3ccccc3)n2)cc1 ; c1ccc(-c2cc(-c3ccccc3)nc(-n3c4ccccc4c4cc(-c5ccc6c(c5)c5ccccc5n6-c5ccc6c7ccccc7c7ccccc7c6c5)ccc43)c2)cc1 ; c1ccc(-c2cc(-c3ccccc3)nc(-n3c4ccccc4c4cc(-c5ccc6c(c5)c5ccccc5n6-c5ccc6c7ccccc7c7ccccc7c6c5)ccc43)c2)cc1]

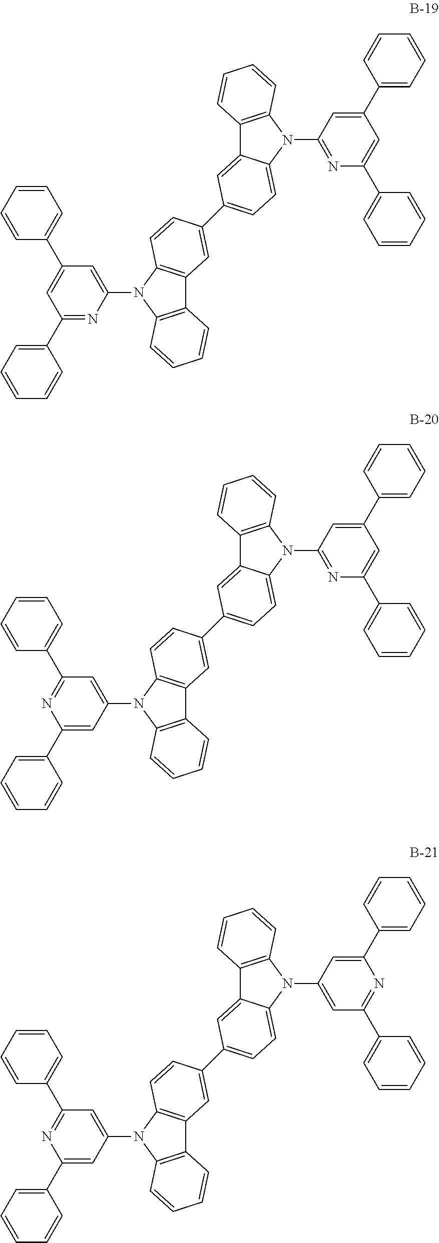 [SMILES: c1ccc(-c2cc(-c3ccccc3)nc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)nc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-n3c4ccccc4c4cc(-c5ccc6c(c5)c5ccccc5n6-c5cc(-c6ccccc6)nc(-c6ccccc6)c5)ccc43)cc(-c3ccccc3)n2)cc1]

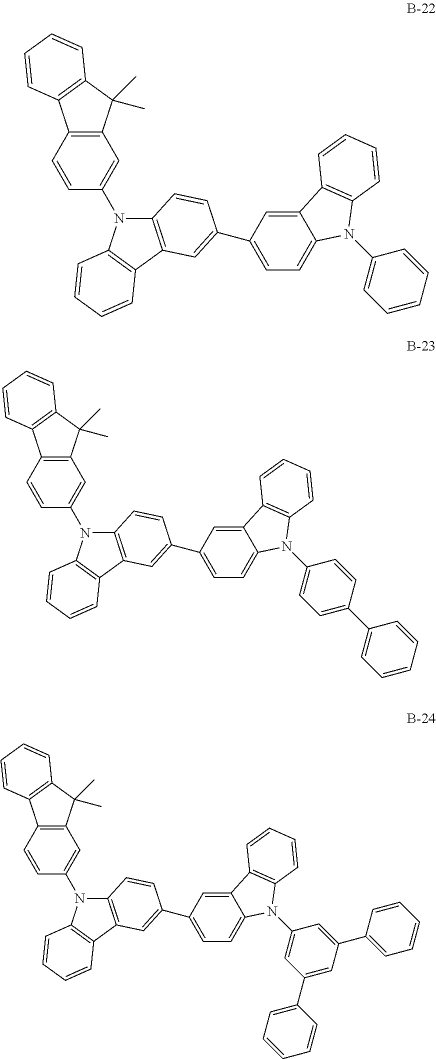 [SMILES: CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21]

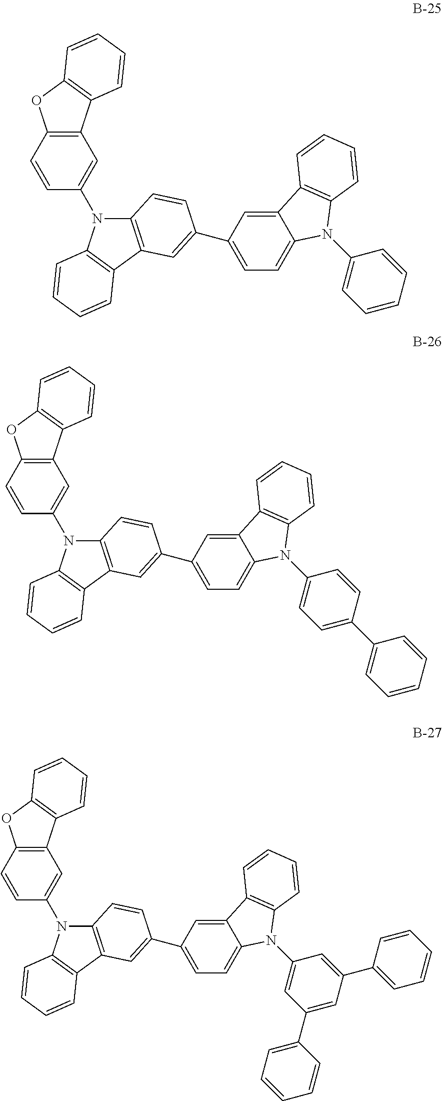 [SMILES: c1ccc(-n2c3ccccc3c3cc(-c4ccc5c(c4)c4ccccc4n5-c4ccc5oc6ccccc6c5c4)ccc32)cc1 ; c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6oc7ccccc7c6c5)ccc43)c2)cc1]

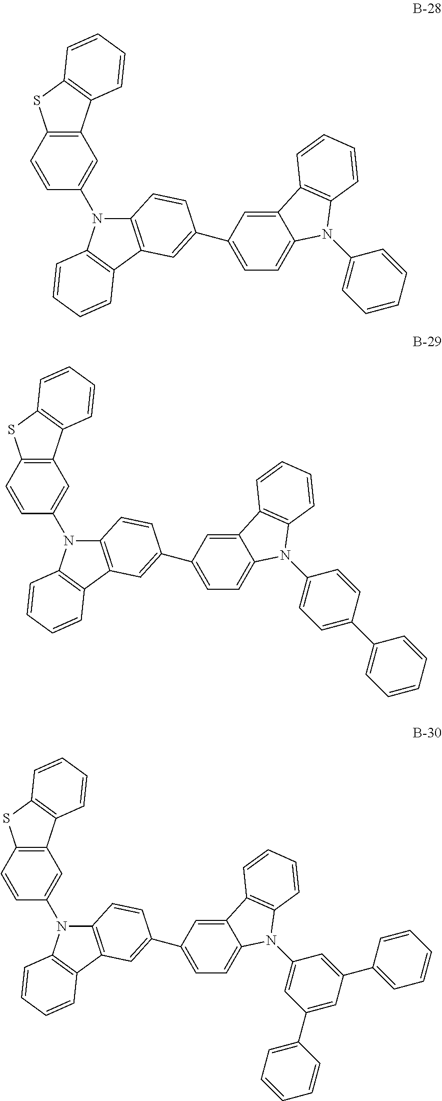 [SMILES: c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1]

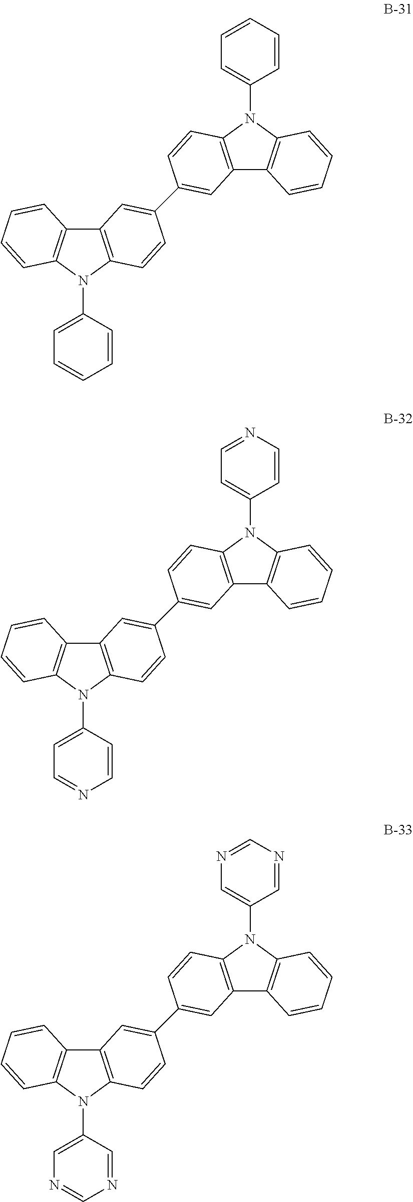 [SMILES: c1ccc(-n2c3ccccc3c3cc(-c4ccc5c(c4)c4ccccc4n5-c4ccccc4)ccc32)cc1 ; c1ccc2c(c1)c1cc(-c3ccc4c(c3)c3ccccc3n4-c3ccncc3)ccc1n2-c1ccncc1 ; c1ccc2c(c1)c1cc(-c3ccc4c(c3)c3ccccc3n4-c3cncnc3)ccc1n2-c1cncnc1]

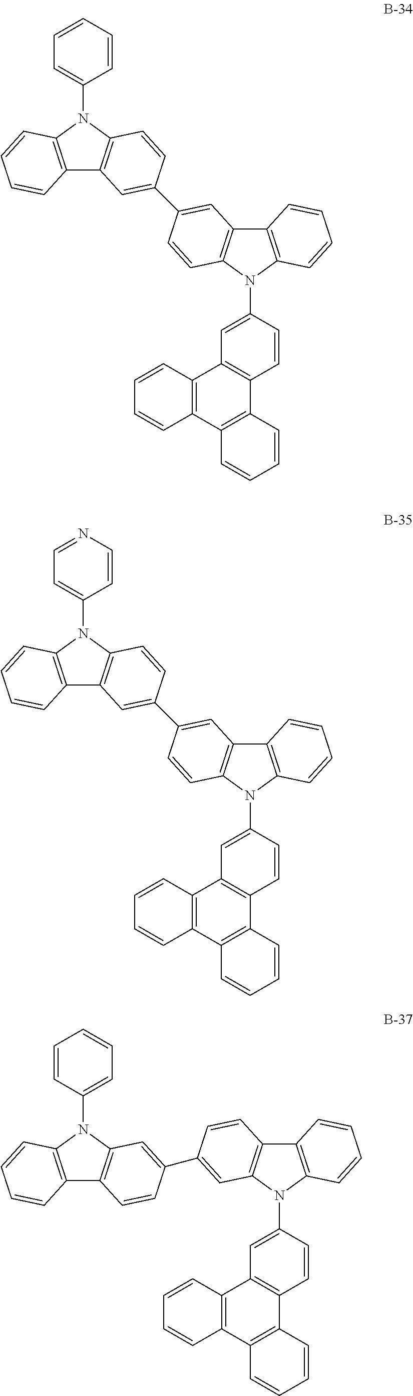 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc2c(c1)c1ccccc1c1cc(-n3c4ccccc4c4cc(-c5ccc6c(c5)c5ccccc5n6-c5ccncc5)ccc43)ccc21 ; c1ccc(-n2c3ccccc3c3ccc(-c4ccc5c6ccccc6n(-c6ccc7c8ccccc8c8ccccc8c7c6)c5c4)cc32)cc1]

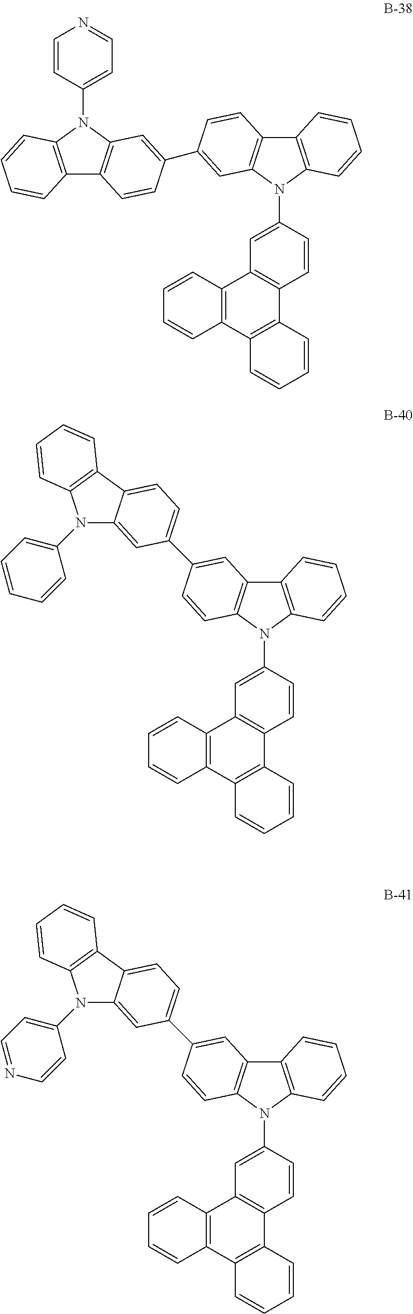 [SMILES: c1ccc2c(c1)c1ccccc1c1cc(-n3c4ccccc4c4ccc(-c5ccc6c7ccccc7n(-c7ccncc7)c6c5)cc43)ccc21 ; c1ccc(-n2c3ccccc3c3ccc(-c4ccc5c(c4)c4ccccc4n5-c4ccc5c6ccccc6c6ccccc6c5c4)cc32)cc1 ; c1ccc2c(c1)c1ccccc1c1cc(-n3c4ccccc4c4cc(-c5ccc6c7ccccc7n(-c7ccncc7)c6c5)ccc43)ccc21]

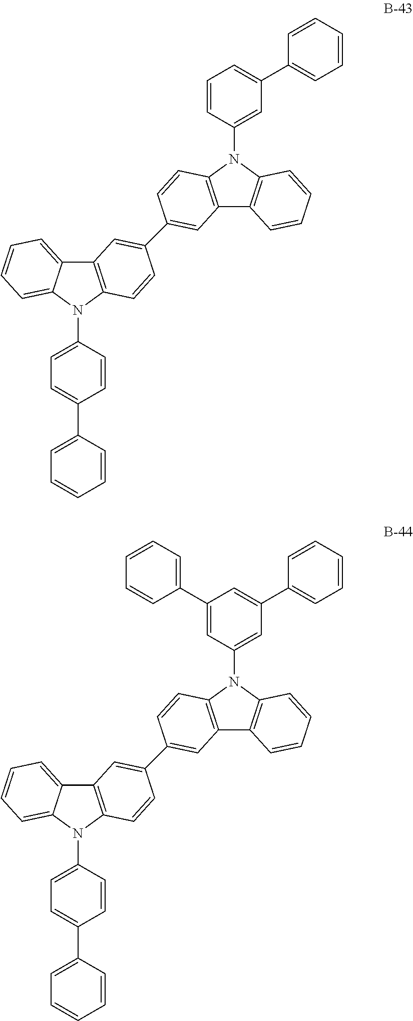 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

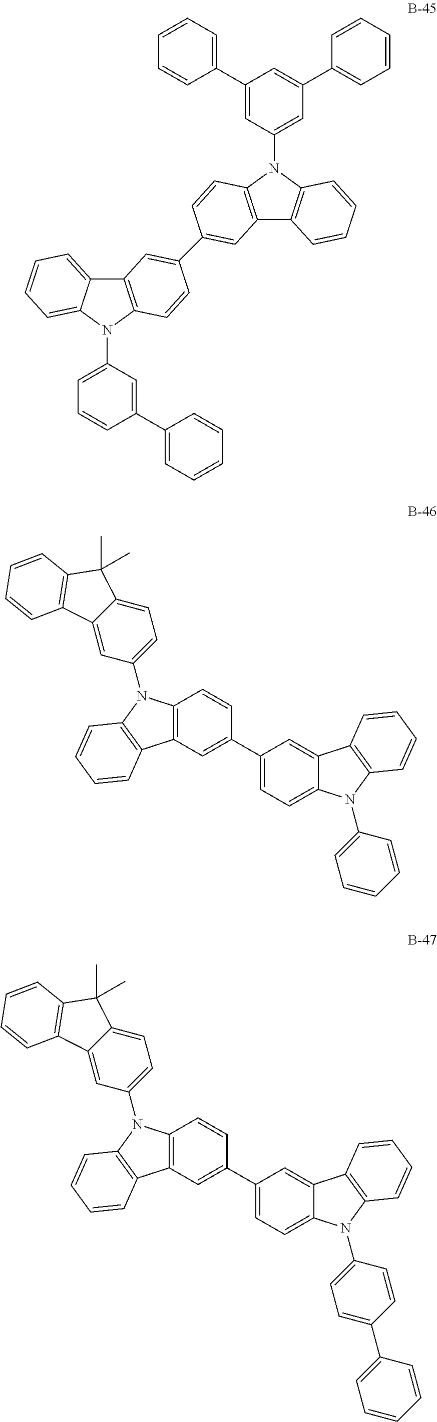 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; CC1(C)c2ccccc2-c2cc(-n3c4ccccc4c4cc(-c5ccc6c(c5)c5ccccc5n6-c5ccccc5)ccc43)ccc21 ; CC1(C)c2ccccc2-c2cc(-n3c4ccccc4c4cc(-c5ccc6c(c5)c5ccccc5n6-c5ccc(-c6ccccc6)cc5)ccc43)ccc21]

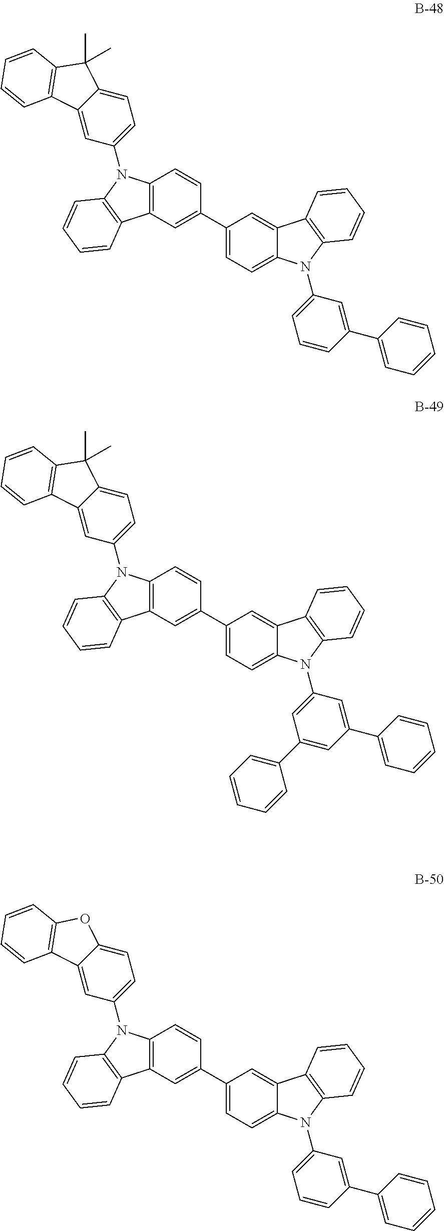 [SMILES: CC1(C)c2ccccc2-c2cc(-n3c4ccccc4c4cc(-c5ccc6c(c5)c5ccccc5n6-c5cccc(-c6ccccc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccc6)c5)ccc43)ccc21 ; c1ccc(-c2cccc(-n3c4ccccc4c4cc(-c5ccc6c(c5)c5ccccc5n6-c5ccc6oc7ccccc7c6c5)ccc43)c2)cc1]

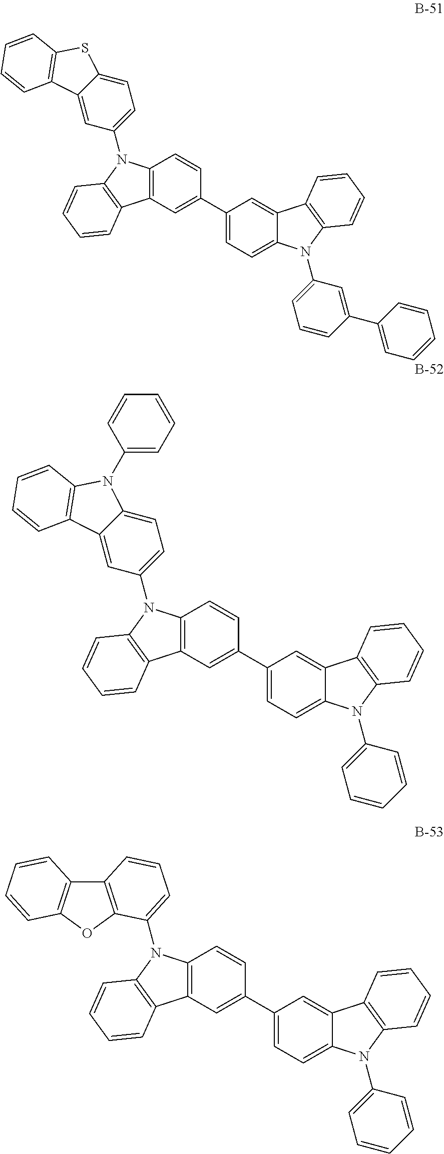 [SMILES: c1ccc(-c2cccc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5c4oc4ccccc45)ccc32)cc1]

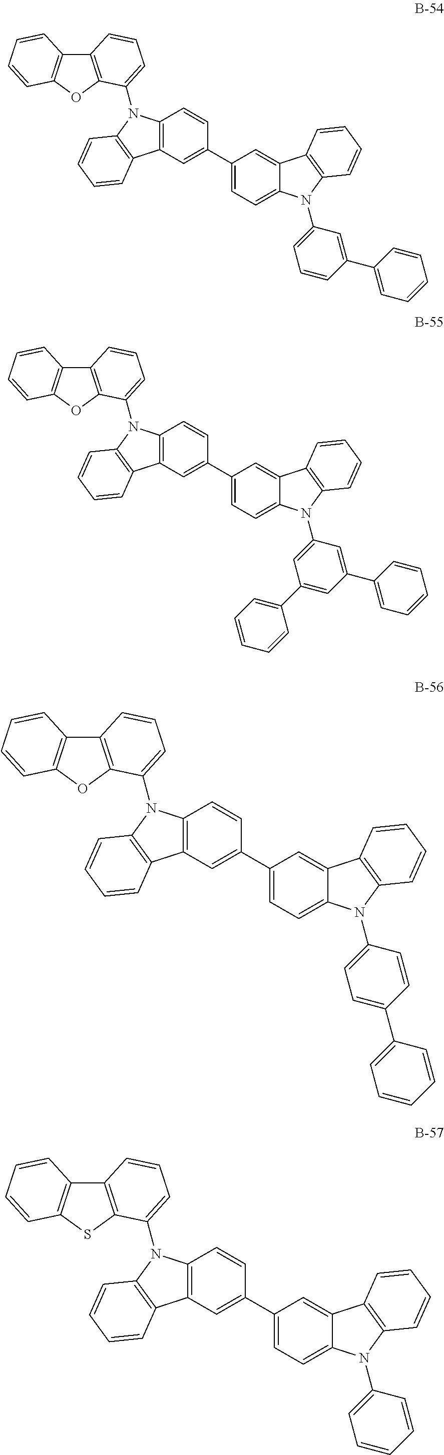 [SMILES: c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5oc5ccccc56)ccc43)c2)cc1 ; c1ccc(-c2ccc(-n3c4ccccc4c4cc(-c5ccc6c(c5)c5ccccc5n6-c5cccc6c5oc5ccccc56)ccc43)cc2)cc1 ; c1ccc(-n2c3ccccc3c3cc(-c4ccc5c(c4)c4ccccc4n5-c4cccc5c4sc4ccccc45)ccc32)cc1]

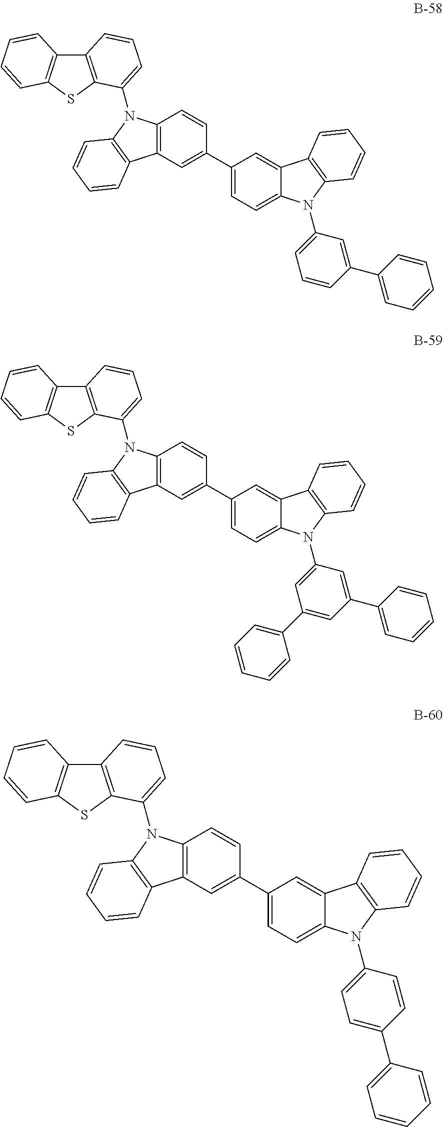 [SMILES: c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1 ; c1ccc(-c2ccc(-n3c4ccccc4c4cc(-c5ccc6c(c5)c5ccccc5n6-c5cccc6c5sc5ccccc56)ccc43)cc2)cc1]

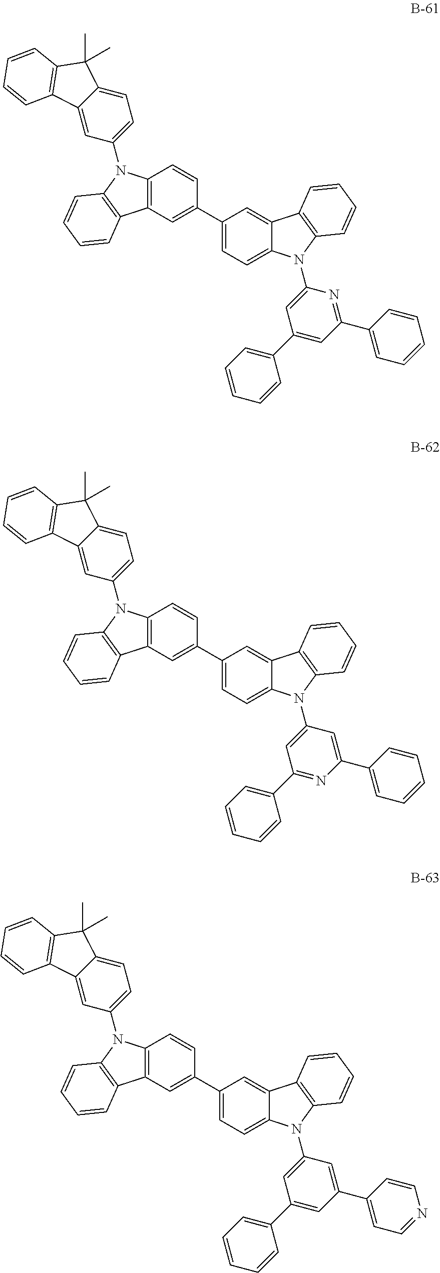 [SMILES: CC1(C)c2ccccc2-c2cc(-n3c4ccccc4c4cc(-c5ccc6c(c5)c5ccccc5n6-c5cc(-c6ccccc6)cc(-c6ccccc6)n5)ccc43)ccc21 ; CC1(C)c2ccccc2-c2cc(-n3c4ccccc4c4cc(-c5ccc6c(c5)c5ccccc5n6-c5cc(-c6ccccc6)nc(-c6ccccc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccncc6)c5)ccc43)ccc21]

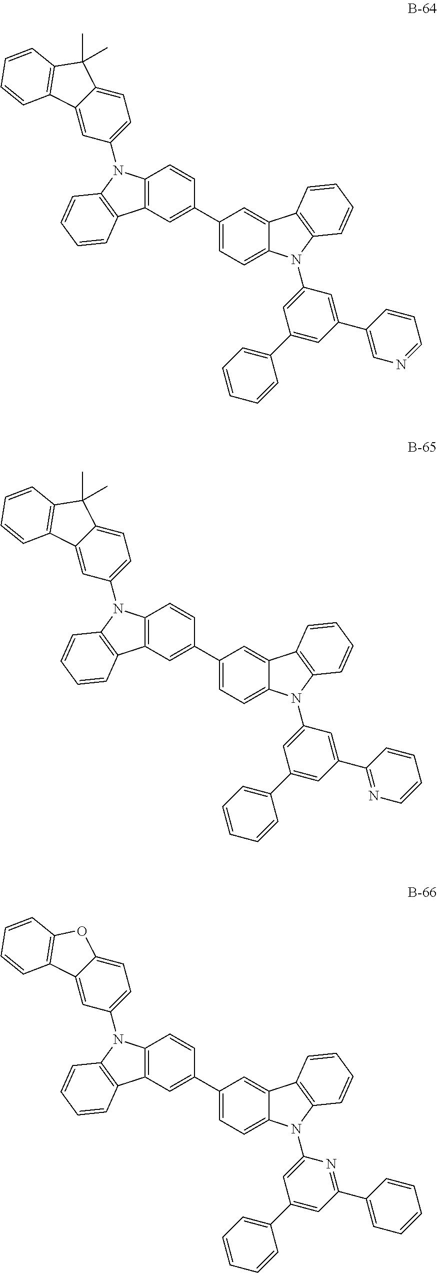 [SMILES: CC1(C)c2ccccc2-c2cc(-n3c4ccccc4c4cc(-c5ccc6c(c5)c5ccccc5n6-c5cc(-c6ccccc6)cc(-c6cccnc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccn6)c5)ccc43)ccc21 ; c1ccc(-c2cc(-c3ccccc3)nc(-n3c4ccccc4c4cc(-c5ccc6c(c5)c5ccccc5n6-c5ccc6oc7ccccc7c6c5)ccc43)c2)cc1]

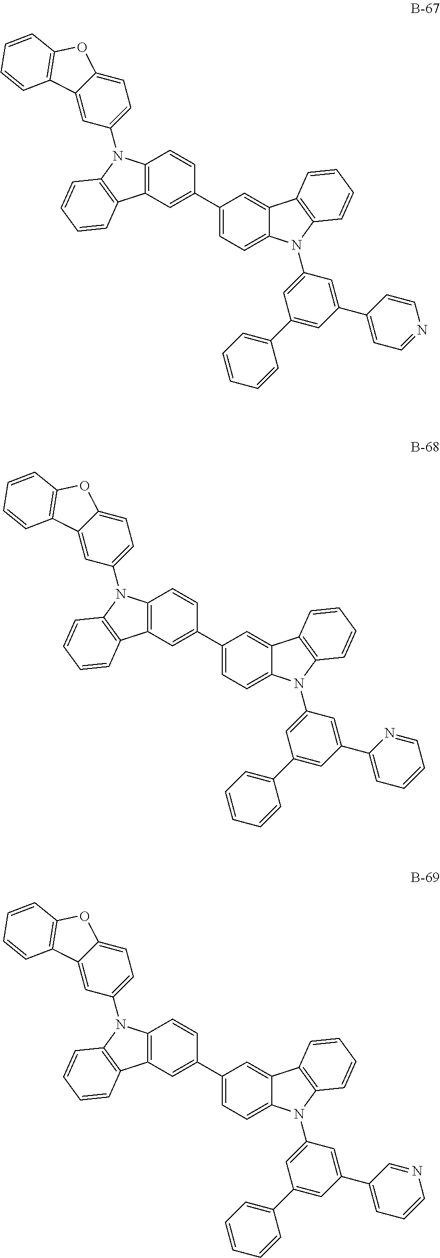 [SMILES: c1ccc(-c2cc(-c3ccncc3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3ccccn3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3cccnc3)cc(-n3c4ccccc4c4cc(-c5ccc6c(c5)c5ccccc5n6-c5ccc6oc7ccccc7c6c5)ccc43)c2)cc1]

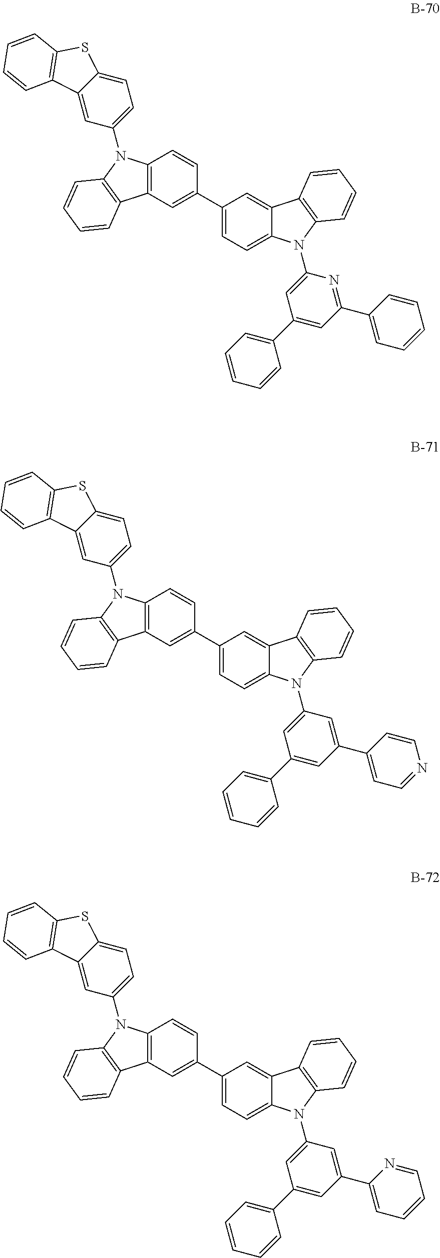 [SMILES: c1ccc(-c2cc(-c3ccccc3)nc(-n3c4ccccc4c4cc(-c5ccc6c(c5)c5ccccc5n6-c5ccc6sc7ccccc7c6c5)ccc43)c2)cc1 ; c1ccc(-c2cc(-c3ccncc3)cc(-n3c4ccccc4c4cc(-c5ccc6c(c5)c5ccccc5n6-c5ccc6sc7ccccc7c6c5)ccc43)c2)cc1 ; c1ccc(-c2cc(-c3ccccn3)cc(-n3c4ccccc4c4cc(-c5ccc6c(c5)c5ccccc5n6-c5ccc6sc7ccccc7c6c5)ccc43)c2)cc1]

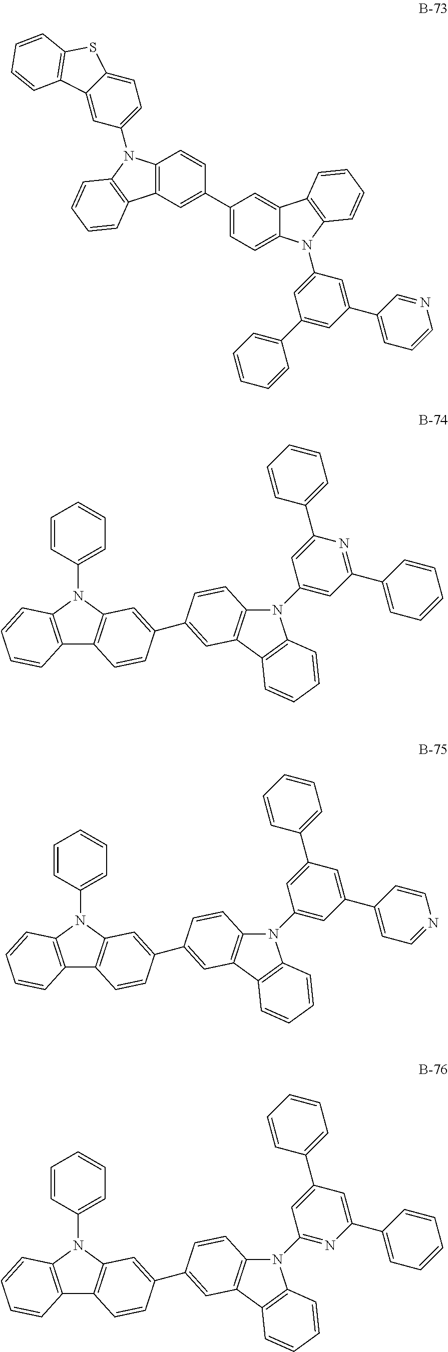 [SMILES: c1ccc(-c2cc(-c3cccnc3)cc(-n3c4ccccc4c4cc(-c5ccc6c(c5)c5ccccc5n6-c5ccc6sc7ccccc7c6c5)ccc43)c2)cc1 ; c1ccc(-c2cc(-n3c4ccccc4c4cc(-c5ccc6c7ccccc7n(-c7ccccc7)c6c5)ccc43)cc(-c3ccccc3)n2)cc1 ; c1ccc(-c2cc(-c3ccncc3)cc(-n3c4ccccc4c4cc(-c5ccc6c7ccccc7n(-c7ccccc7)c6c5)ccc43)c2)cc1 ; c1ccc(-c2cc(-c3ccccc3)nc(-n3c4ccccc4c4cc(-c5ccc6c7ccccc7n(-c7ccccc7)c6c5)ccc43)c2)cc1]

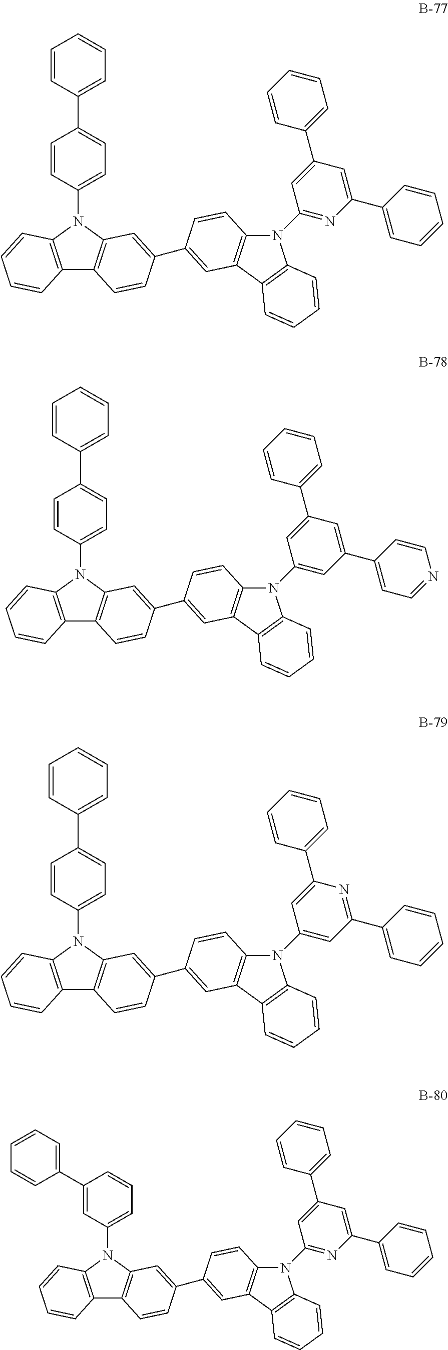 [SMILES: c1ccc(-c2ccc(-n3c4ccccc4c4ccc(-c5ccc6c(c5)c5ccccc5n6-c5cc(-c6ccccc6)cc(-c6ccccc6)n5)cc43)cc2)cc1 ; c1ccc(-c2ccc(-n3c4ccccc4c4ccc(-c5ccc6c(c5)c5ccccc5n6-c5cc(-c6ccccc6)cc(-c6ccncc6)c5)cc43)cc2)cc1 ; c1ccc(-c2ccc(-n3c4ccccc4c4ccc(-c5ccc6c(c5)c5ccccc5n6-c5cc(-c6ccccc6)nc(-c6ccccc6)c5)cc43)cc2)cc1 ; c1ccc(-c2cccc(-n3c4ccccc4c4ccc(-c5ccc6c(c5)c5ccccc5n6-c5cc(-c6ccccc6)cc(-c6ccccc6)n5)cc43)c2)cc1]

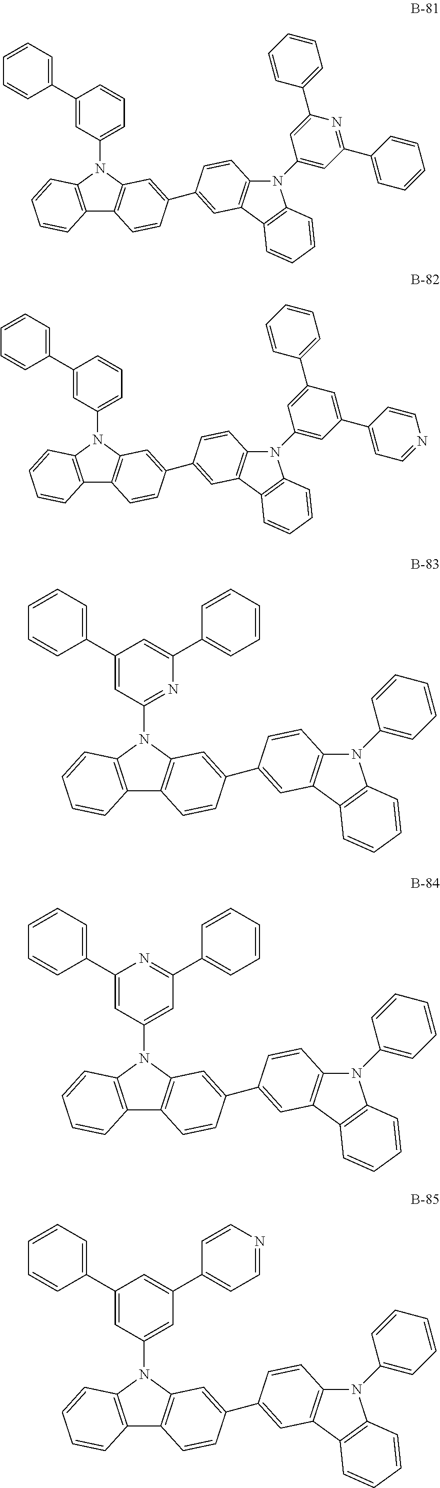

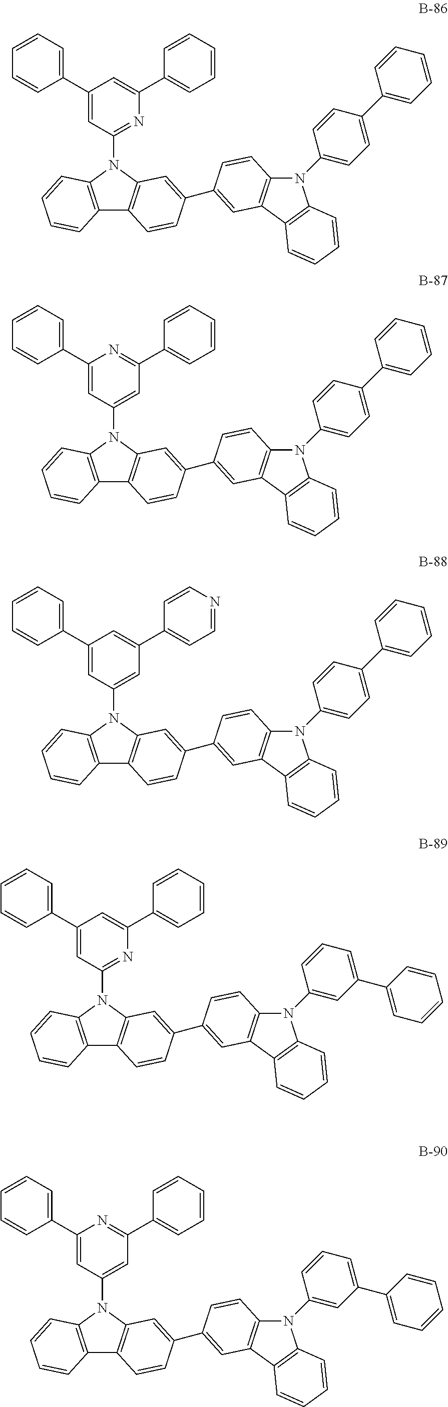 [SMILES: c1ccc(-c2ccc(-n3c4ccccc4c4cc(-c5ccc6c7ccccc7n(-c7cc(-c8ccccc8)cc(-c8ccccc8)n7)c6c5)ccc43)cc2)cc1 ; c1ccc(-c2ccc(-n3c4ccccc4c4cc(-c5ccc6c7ccccc7n(-c7cc(-c8ccccc8)nc(-c8ccccc8)c7)c6c5)ccc43)cc2)cc1 ; c1ccc(-c2ccc(-n3c4ccccc4c4cc(-c5ccc6c7ccccc7n(-c7cc(-c8ccccc8)cc(-c8ccncc8)c7)c6c5)ccc43)cc2)cc1 ; c1ccc(-c2cccc(-n3c4ccccc4c4cc(-c5ccc6c7ccccc7n(-c7cc(-c8ccccc8)cc(-c8ccccc8)n7)c6c5)ccc43)c2)cc1 ; c1ccc(-c2cccc(-n3c4ccccc4c4cc(-c5ccc6c7ccccc7n(-c7cc(-c8ccccc8)nc(-c8ccccc8)c7)c6c5)ccc43)c2)cc1]

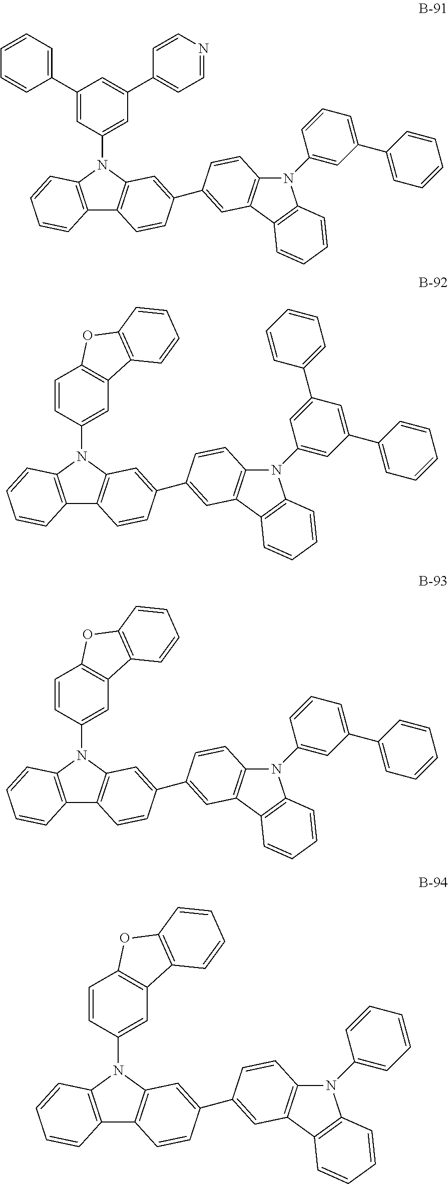 [SMILES: c1ccc(-c2cccc(-n3c4ccccc4c4cc(-c5ccc6c7ccccc7n(-c7cc(-c8ccccc8)cc(-c8ccncc8)c7)c6c5)ccc43)c2)cc1 ; c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1 ; c1ccc(-c2cccc(-n3c4ccccc4c4cc(-c5ccc6c7ccccc7n(-c7ccc8oc9ccccc9c8c7)c6c5)ccc43)c2)cc1 ; c1ccc(-n2c3ccccc3c3cc(-c4ccc5c6ccccc6n(-c6ccc7oc8ccccc8c7c6)c5c4)ccc32)cc1]

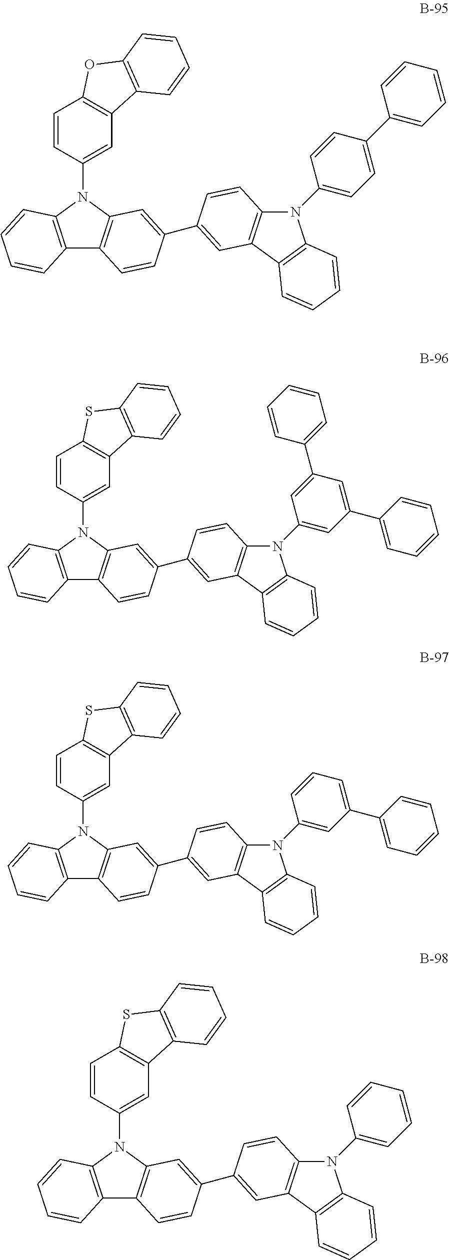 [SMILES: c1ccc(-c2ccc(-n3c4ccccc4c4cc(-c5ccc6c7ccccc7n(-c7ccc8oc9ccccc9c8c7)c6c5)ccc43)cc2)cc1 ; c1ccc(-c2cc(-c3ccccc3)cc(-n3c4ccccc4c4cc(-c5ccc6c7ccccc7n(-c7ccc8sc9ccccc9c8c7)c6c5)ccc43)c2)cc1 ; c1ccc(-c2cccc(-n3c4ccccc4c4cc(-c5ccc6c7ccccc7n(-c7ccc8sc9ccccc9c8c7)c6c5)ccc43)c2)cc1 ; c1ccc(-n2c3ccccc3c3cc(-c4ccc5c6ccccc6n(-c6ccc7sc8ccccc8c7c6)c5c4)ccc32)cc1]

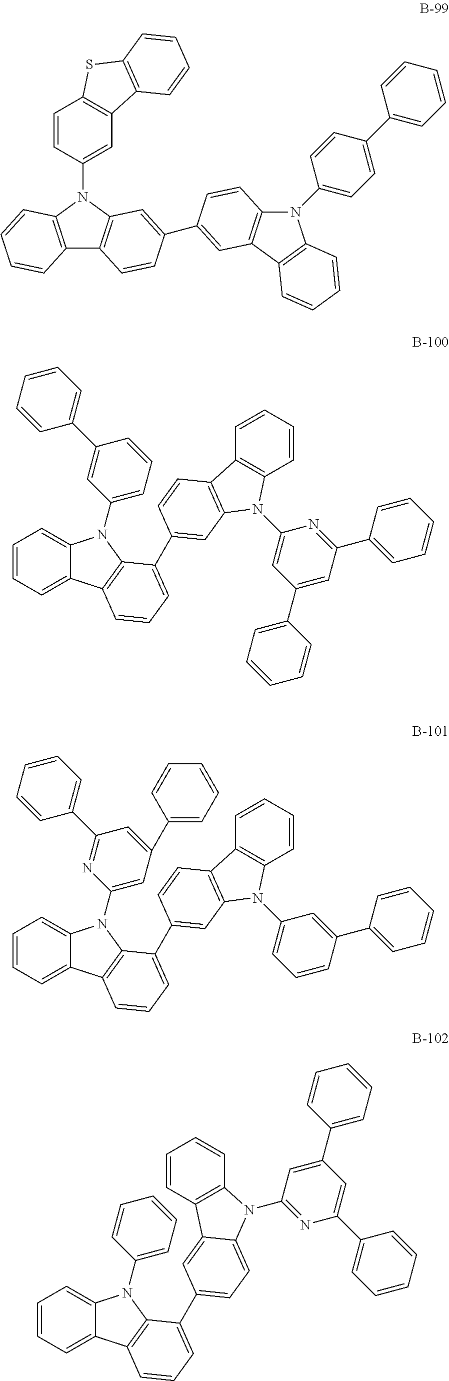 [SMILES: c1ccc(-c2ccc(-n3c4ccccc4c4cc(-c5ccc6c7ccccc7n(-c7ccc8sc9ccccc9c8c7)c6c5)ccc43)cc2)cc1 ; c1ccc(-c2cccc(-n3c4ccccc4c4cccc(-c5ccc6c7ccccc7n(-c7cc(-c8ccccc8)cc(-c8ccccc8)n7)c6c5)c43)c2)cc1 ; c1ccc(-c2cccc(-n3c4ccccc4c4ccc(-c5cccc6c7ccccc7n(-c7cc(-c8ccccc8)cc(-c8ccccc8)n7)c56)cc43)c2)cc1 ; c1ccc(-c2cc(-c3ccccc3)nc(-n3c4ccccc4c4cc(-c5cccc6c7ccccc7n(-c7ccccc7)c56)ccc43)c2)cc1]

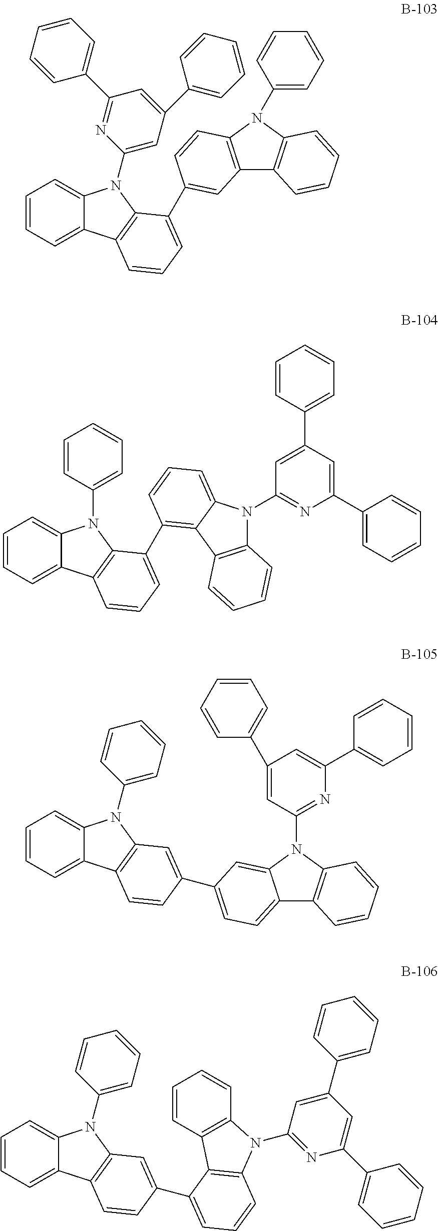 [SMILES: c1ccc(-c2cc(-c3ccccc3)nc(-n3c4ccccc4c4cccc(-c5ccc6c(c5)c5ccccc5n6-c5ccccc5)c43)c2)cc1 ; c1ccc(-c2cc(-c3ccccc3)nc(-n3c4ccccc4c4c(-c5cccc6c7ccccc7n(-c7ccccc7)c56)cccc43)c2)cc1 ; c1ccc(-c2cc(-c3ccccc3)nc(-n3c4ccccc4c4ccc(-c5ccc6c7ccccc7n(-c7ccccc7)c6c5)cc43)c2)cc1 ; c1ccc(-c2cc(-c3ccccc3)nc(-n3c4ccccc4c4c(-c5ccc6c7ccccc7n(-c7ccccc7)c6c5)cccc43)c2)cc1]

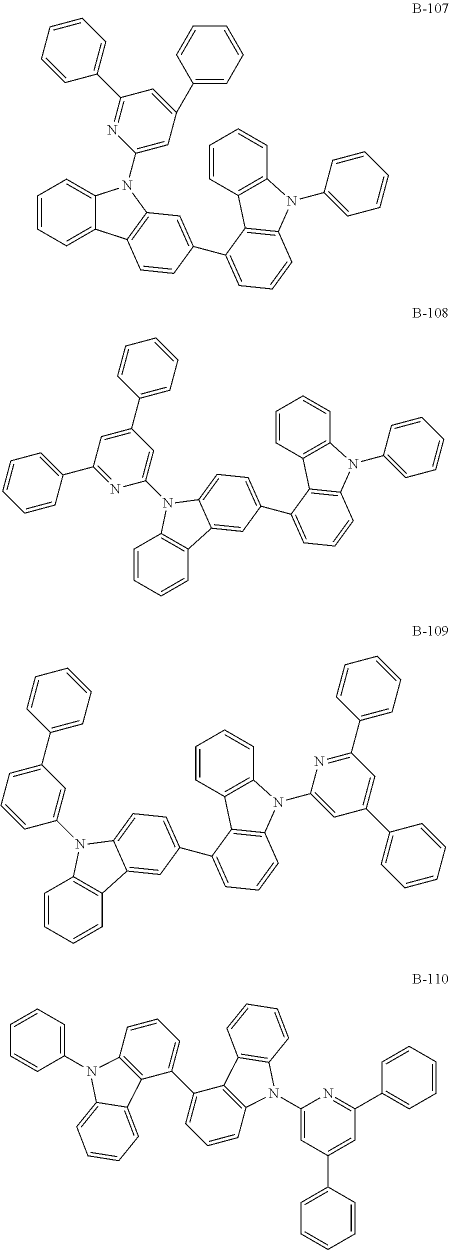 [SMILES: c1ccc(-c2cc(-c3ccccc3)nc(-n3c4ccccc4c4ccc(-c5cccc6c5c5ccccc5n6-c5ccccc5)cc43)c2)cc1 ; c1ccc(-c2cc(-c3ccccc3)nc(-n3c4ccccc4c4cc(-c5cccc6c5c5ccccc5n6-c5ccccc5)ccc43)c2)cc1 ; c1ccc(-c2cccc(-n3c4ccccc4c4cc(-c5cccc6c5c5ccccc5n6-c5cc(-c6ccccc6)cc(-c6ccccc6)n5)ccc43)c2)cc1 ; c1ccc(-c2cc(-c3ccccc3)nc(-n3c4ccccc4c4c(-c5cccc6c5c5ccccc5n6-c5ccccc5)cccc43)c2)cc1]

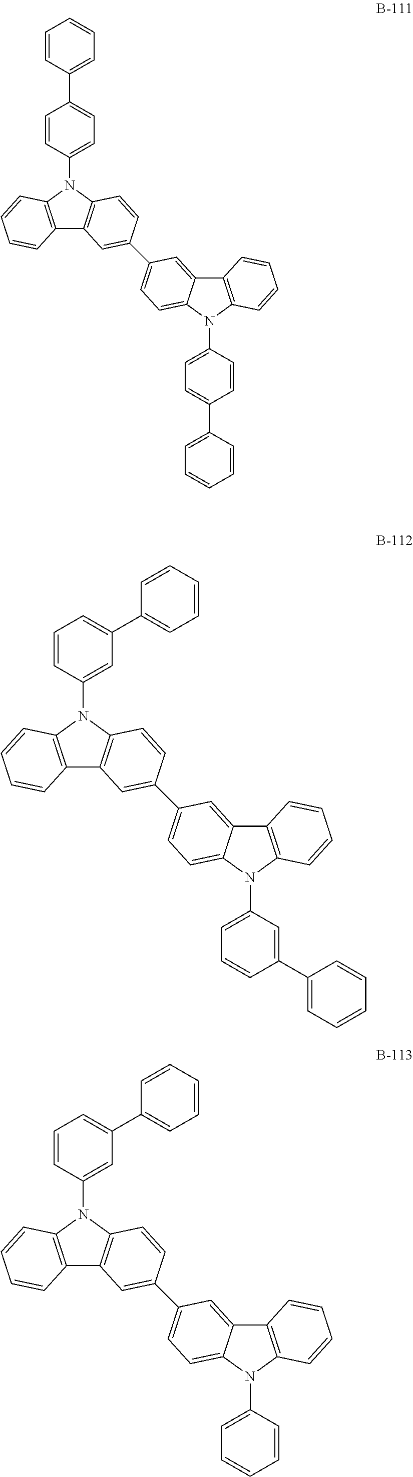 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccccc5)ccc43)c2)cc1]

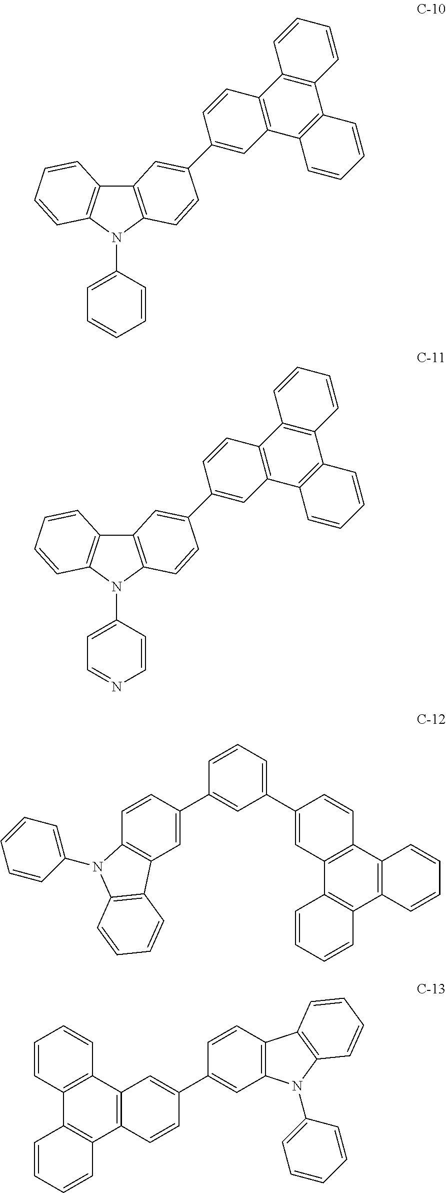

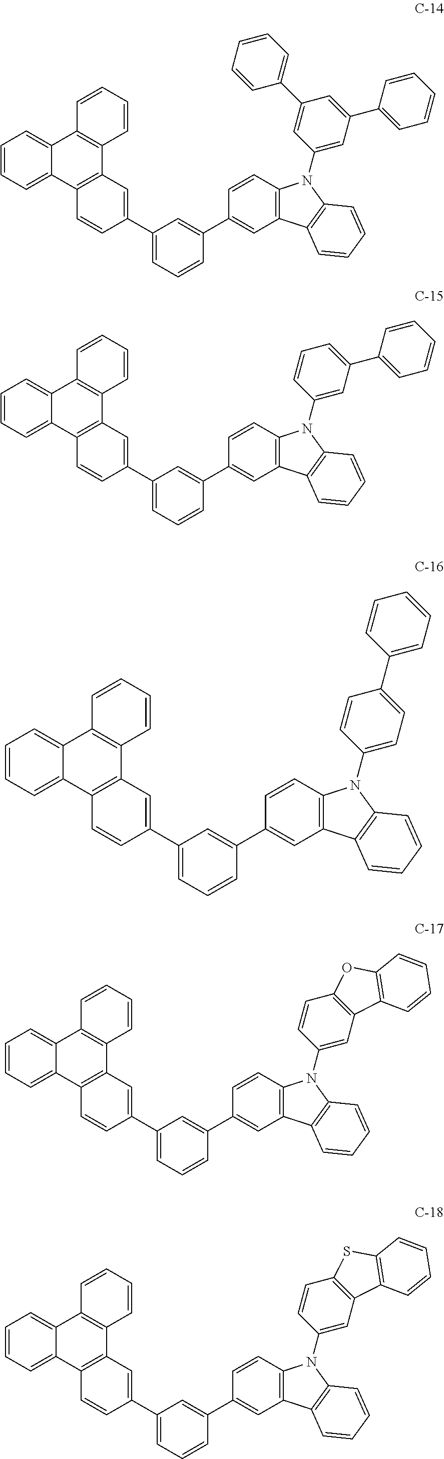 [SMILES: c1ccc(-c2cc(-c3ccccc3)cc(-n3c4ccccc4c4cc(-c5cccc(-c6ccc7c8ccccc8c8ccccc8c7c6)c5)ccc43)c2)cc1 ; c1ccc(-c2cccc(-n3c4ccccc4c4cc(-c5cccc(-c6ccc7c8ccccc8c8ccccc8c7c6)c5)ccc43)c2)cc1 ; c1ccc(-c2ccc(-n3c4ccccc4c4cc(-c5cccc(-c6ccc7c8ccccc8c8ccccc8c7c6)c5)ccc43)cc2)cc1 ; c1cc(-c2ccc3c4ccccc4c4ccccc4c3c2)cc(-c2ccc3c(c2)c2ccccc2n3-c2ccc3oc4ccccc4c3c2)c1 ; c1cc(-c2ccc3c4ccccc4c4ccccc4c3c2)cc(-c2ccc3c(c2)c2ccccc2n3-c2ccc3sc4ccccc4c3c2)c1]

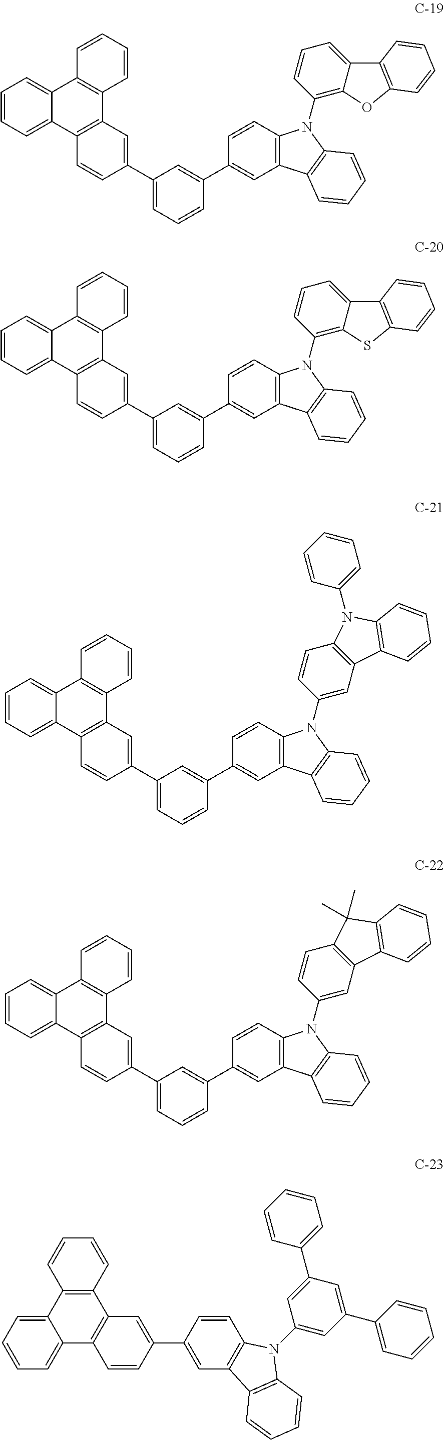 [SMILES: c1cc(-c2ccc3c4ccccc4c4ccccc4c3c2)cc(-c2ccc3c(c2)c2ccccc2n3-c2cccc3c2oc2ccccc23)c1 ; c1cc(-c2ccc3c4ccccc4c4ccccc4c3c2)cc(-c2ccc3c(c2)c2ccccc2n3-c2cccc3c2sc2ccccc23)c1 ; c1ccc(-n2c3ccccc3c3cc(-n4c5ccccc5c5cc(-c6cccc(-c7ccc8c9ccccc9c9ccccc9c8c7)c6)ccc54)ccc32)cc1 ; CC1(C)c2ccccc2-c2cc(-n3c4ccccc4c4cc(-c5cccc(-c6ccc7c8ccccc8c8ccccc8c7c6)c5)ccc43)ccc21 ; c1ccc(-c2cc(-c3ccccc3)cc(-n3c4ccccc4c4cc(-c5ccc6c7ccccc7c7ccccc7c6c5)ccc43)c2)cc1]

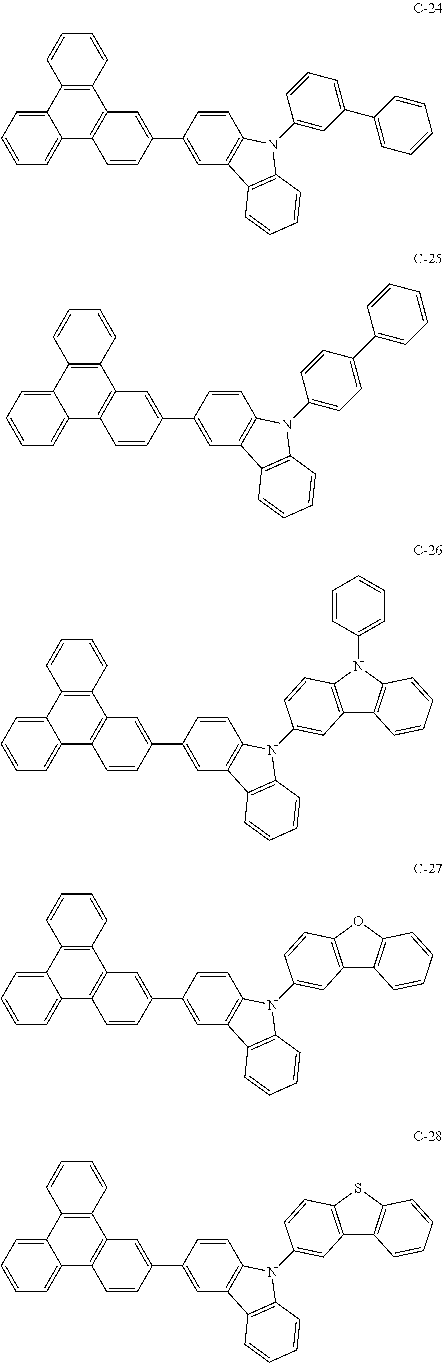 [SMILES: c1ccc(-c2cccc(-n3c4ccccc4c4cc(-c5ccc6c7ccccc7c7ccccc7c6c5)ccc43)c2)cc1 ; c1ccc(-c2ccc(-n3c4ccccc4c4cc(-c5ccc6c7ccccc7c7ccccc7c6c5)ccc43)cc2)cc1 ; c1ccc(-n2c3ccccc3c3cc(-n4c5ccccc5c5cc(-c6ccc7c8ccccc8c8ccccc8c7c6)ccc54)ccc32)cc1 ; c1ccc2c(c1)oc1ccc(-n3c4ccccc4c4cc(-c5ccc6c7ccccc7c7ccccc7c6c5)ccc43)cc12 ; c1ccc2c(c1)sc1ccc(-n3c4ccccc4c4cc(-c5ccc6c7ccccc7c7ccccc7c6c5)ccc43)cc12]

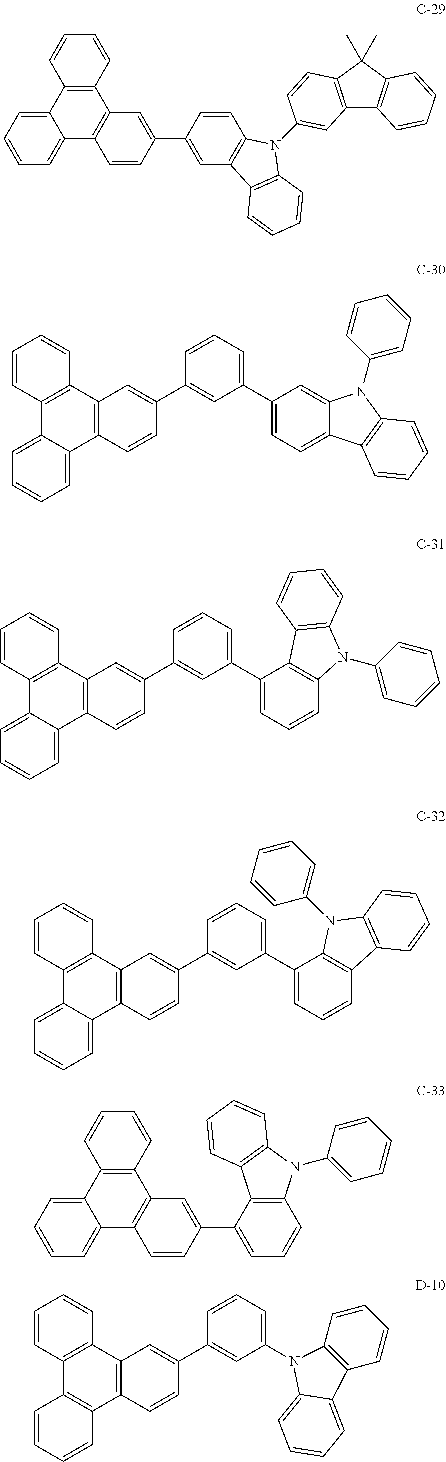

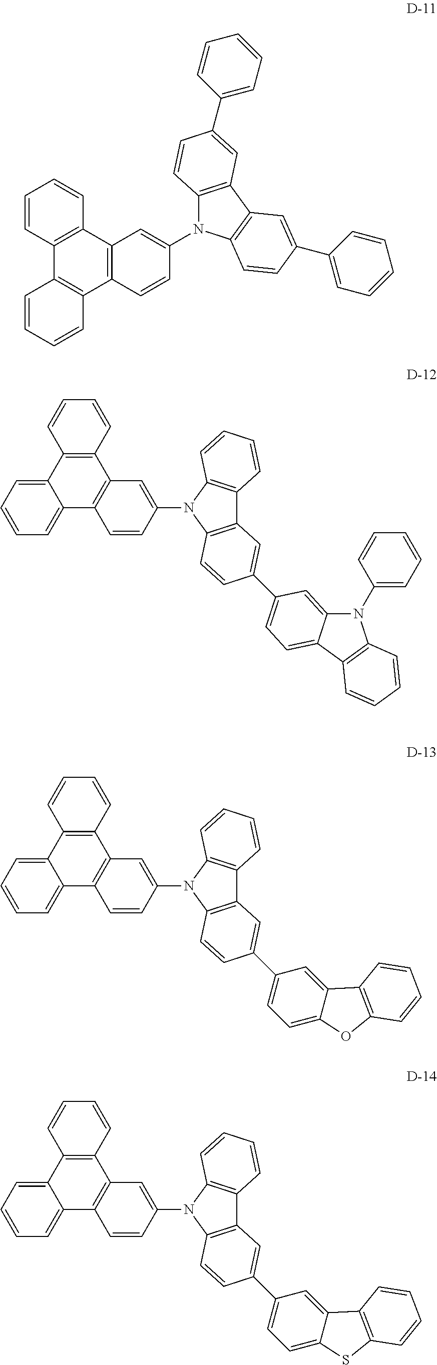 [SMILES: c1ccc(-c2ccc3c(c2)c2cc(-c4ccccc4)ccc2n3-c2ccc3c4ccccc4c4ccccc4c3c2)cc1 ; c1ccc(-n2c3ccccc3c3ccc(-c4ccc5c(c4)c4ccccc4n5-c4ccc5c6ccccc6c6ccccc6c5c4)cc32)cc1 ; c1ccc2c(c1)oc1ccc(-c3ccc4c(c3)c3ccccc3n4-c3ccc4c5ccccc5c5ccccc5c4c3)cc12 ; c1ccc2c(c1)sc1ccc(-c3ccc4c(c3)c3ccccc3n4-c3ccc4c5ccccc5c5ccccc5c4c3)cc12]

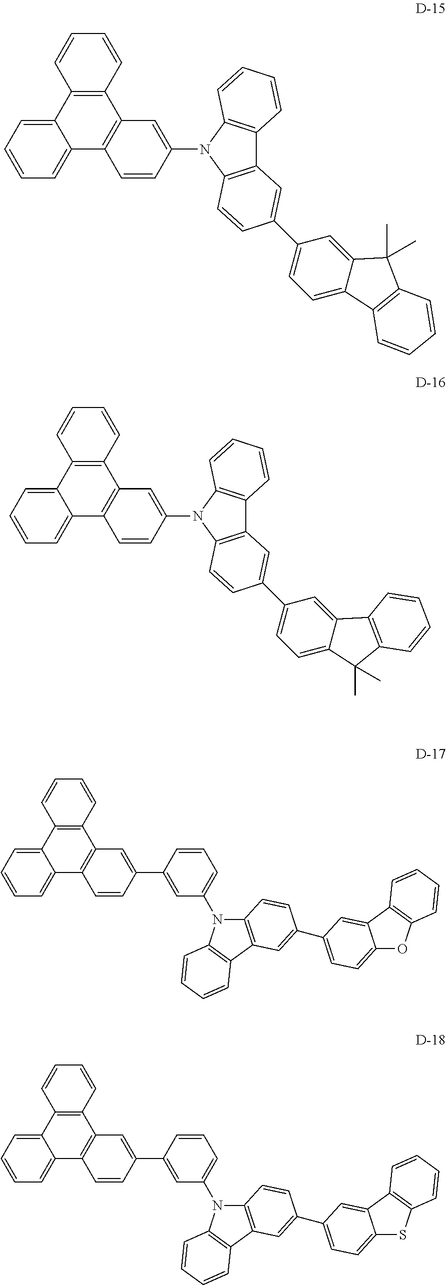 [SMILES: CC1(C)c2ccccc2-c2ccc(-c3ccc4c(c3)c3ccccc3n4-c3ccc4c5ccccc5c5ccccc5c4c3)cc21 ; CC1(C)c2ccccc2-c2cc(-c3ccc4c(c3)c3ccccc3n4-c3ccc4c5ccccc5c5ccccc5c4c3)ccc21 ; c1cc(-c2ccc3c4ccccc4c4ccccc4c3c2)cc(-n2c3ccccc3c3cc(-c4ccc5oc6ccccc6c5c4)ccc32)c1 ; c1cc(-c2ccc3c4ccccc4c4ccccc4c3c2)cc(-n2c3ccccc3c3cc(-c4ccc5sc6ccccc6c5c4)ccc32)c1]

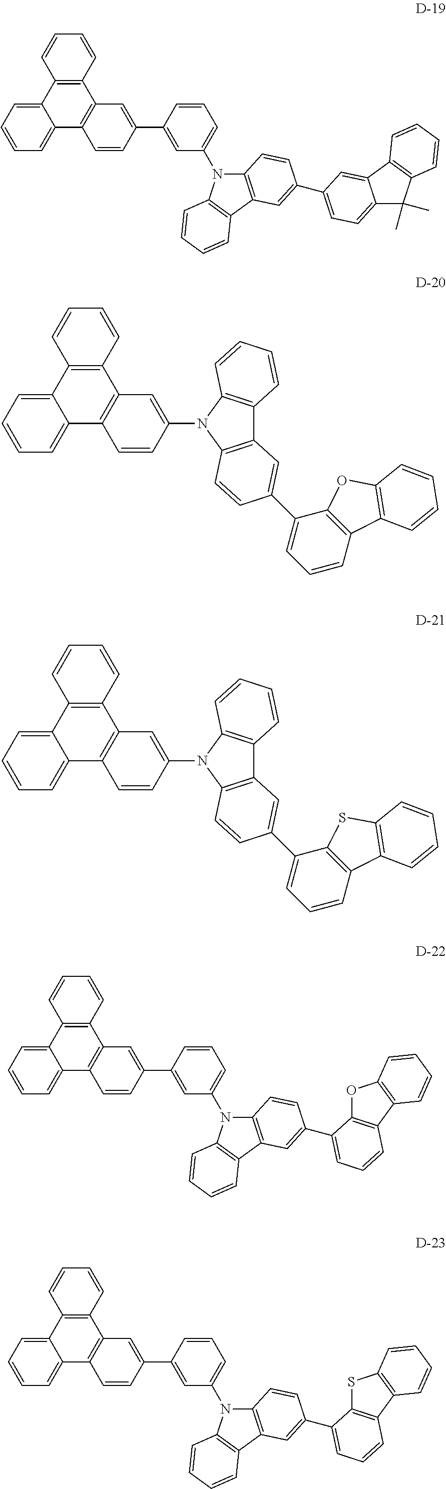 [SMILES: CC1(C)c2ccccc2-c2cc(-c3ccc4c(c3)c3ccccc3n4-c3cccc(-c4ccc5c6ccccc6c6ccccc6c5c4)c3)ccc21 ; c1ccc2c(c1)oc1c(-c3ccc4c(c3)c3ccccc3n4-c3ccc4c5ccccc5c5ccccc5c4c3)cccc12 ; c1ccc2c(c1)sc1c(-c3ccc4c(c3)c3ccccc3n4-c3ccc4c5ccccc5c5ccccc5c4c3)cccc12 ; c1cc(-c2ccc3c4ccccc4c4ccccc4c3c2)cc(-n2c3ccccc3c3cc(-c4cccc5c4oc4ccccc45)ccc32)c1 ; c1cc(-c2ccc3c4ccccc4c4ccccc4c3c2)cc(-n2c3ccccc3c3cc(-c4cccc5c4sc4ccccc45)ccc32)c1]

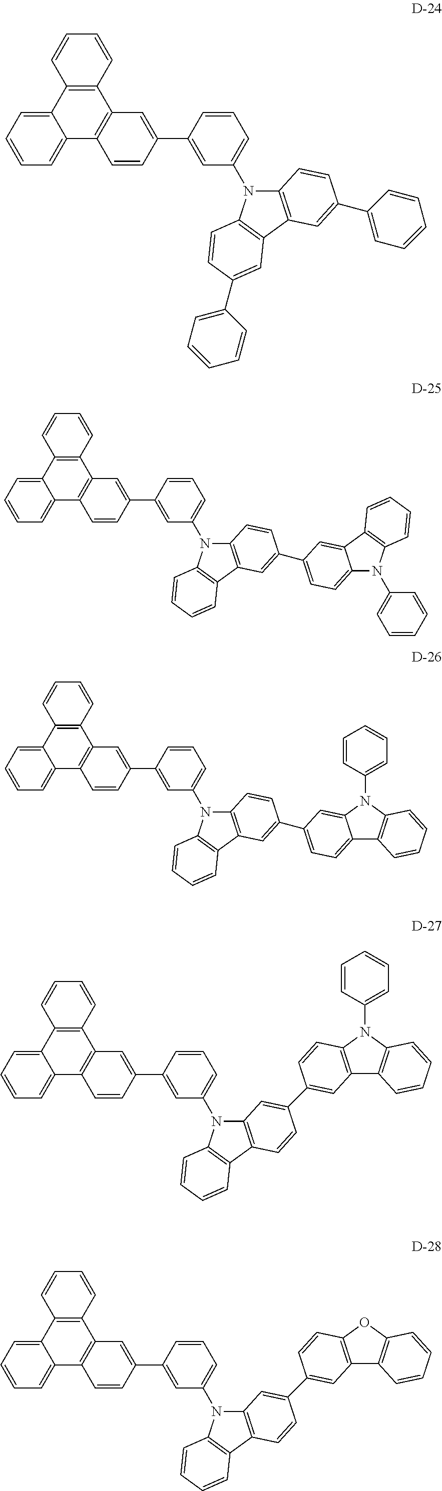 [SMILES: c1ccc(-c2ccc3c(c2)c2cc(-c4ccccc4)ccc2n3-c2cccc(-c3ccc4c5ccccc5c5ccccc5c4c3)c2)cc1 ; c1ccc(-n2c3ccccc3c3cc(-c4ccc5c(c4)c4ccccc4n5-c4cccc(-c5ccc6c7ccccc7c7ccccc7c6c5)c4)ccc32)cc1 ; c1ccc(-n2c3ccccc3c3ccc(-c4ccc5c(c4)c4ccccc4n5-c4cccc(-c5ccc6c7ccccc7c7ccccc7c6c5)c4)cc32)cc1 ; c1ccc(-n2c3ccccc3c3cc(-c4ccc5c6ccccc6n(-c6cccc(-c7ccc8c9ccccc9c9ccccc9c8c7)c6)c5c4)ccc32)cc1 ; c1cc(-c2ccc3c4ccccc4c4ccccc4c3c2)cc(-n2c3ccccc3c3ccc(-c4ccc5oc6ccccc6c5c4)cc32)c1]

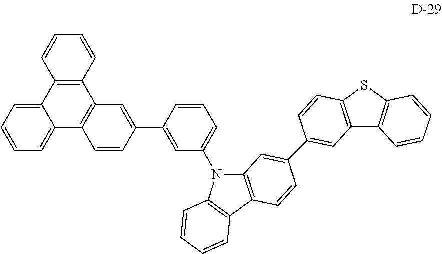 [SMILES: c1cc(-c2ccc3c4ccccc4c4ccccc4c3c2)cc(-n2c3ccccc3c3ccc(-c4ccc5sc6ccccc6c5c4)cc32)c1]

In an implementation, the second organic compound may be, e.g., a compound consisting of a combination of a moiety represented by the following Chemical Formula 5 and a moiety represented by the following Chemical Formula 6.

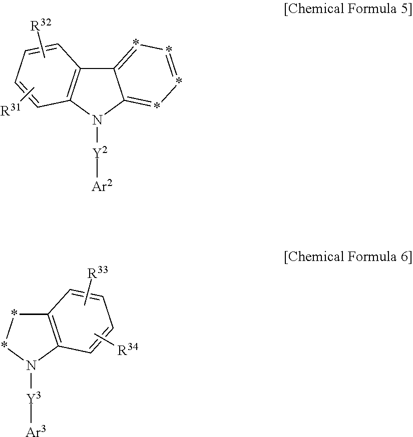

In Chemical Formulae 5 and 6,

Y² and Y³ may each independently be or include, e.g., a single bond, a substituted or unsubstituted C1 to C20 alkylene group, a substituted or unsubstituted C2 to C20 alkenylene group, a substituted or unsubstituted C6 to C30 arylene group, a substituted or unsubstituted C2 to C30 heterocyclic group, or a combination thereof.

Ar² and Ar³ may each independently be or include, e.g., a substituted or unsubstituted C6 to C30 aryl group, a substituted or unsubstituted C2 to C30 heterocyclic group, or a combination thereof.

R³¹ to R³⁴ may each independently be or include, e.g., hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof.

In an implementation, two adjacent *s of Chemical Formula 5 may be combined with the two *s of Chemical Formula 6 to form a fused ring. The *s of Chemical Formula 5 that does not do a fused ring may each independently be CR^{a}. R^{a} may be or include, e.g., hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof.

In an implementation, the organic compound consisting of a combination of the moiety represented by the Chemical Formula 5 and the moiety represented by the Chemical Formula 6 may be one of compounds of the following Group 4.

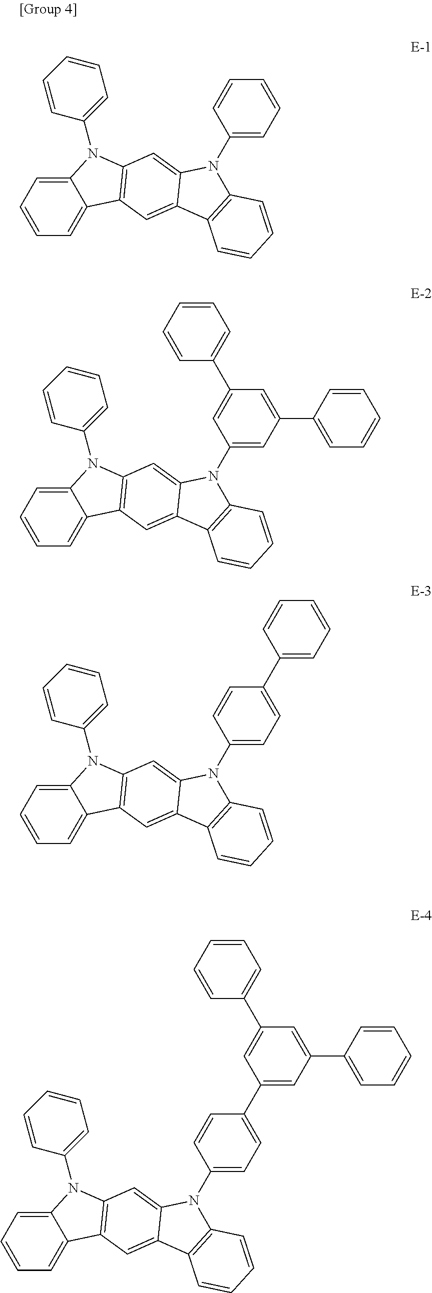

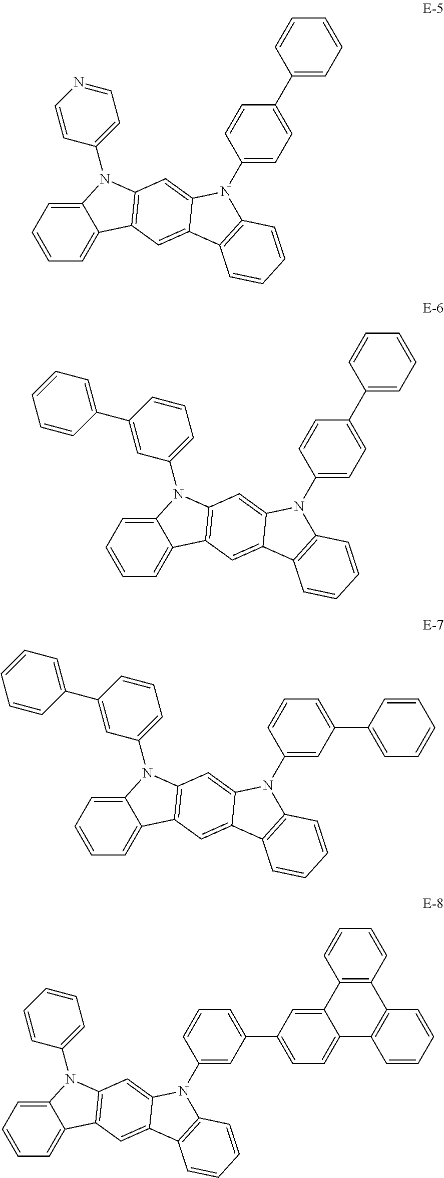 [SMILES: c1ccc(-c2ccc(-n3c4ccccc4c4cc5c6ccccc6n(-c6ccncc6)c5cc43)cc2)cc1 ; c1ccc(-c2ccc(-n3c4ccccc4c4cc5c6ccccc6n(-c6cccc(-c7ccccc7)c6)c5cc43)cc2)cc1 ; c1ccc(-c2cccc(-n3c4ccccc4c4cc5c6ccccc6n(-c6cccc(-c7ccccc7)c6)c5cc43)c2)cc1 ; c1ccc(-n2c3ccccc3c3cc4c5ccccc5n(-c5cccc(-c6ccc7c8ccccc8c8ccccc8c7c6)c5)c4cc32)cc1]

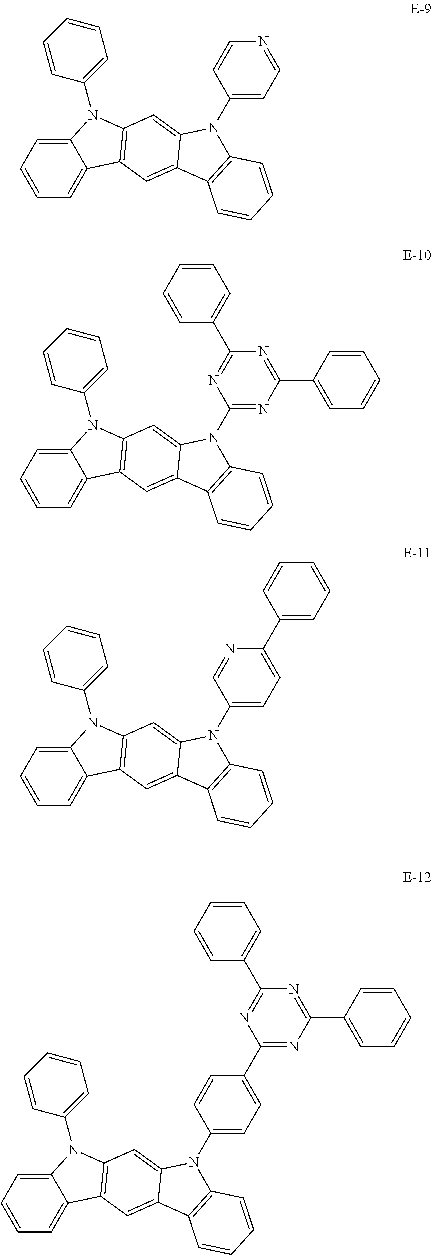

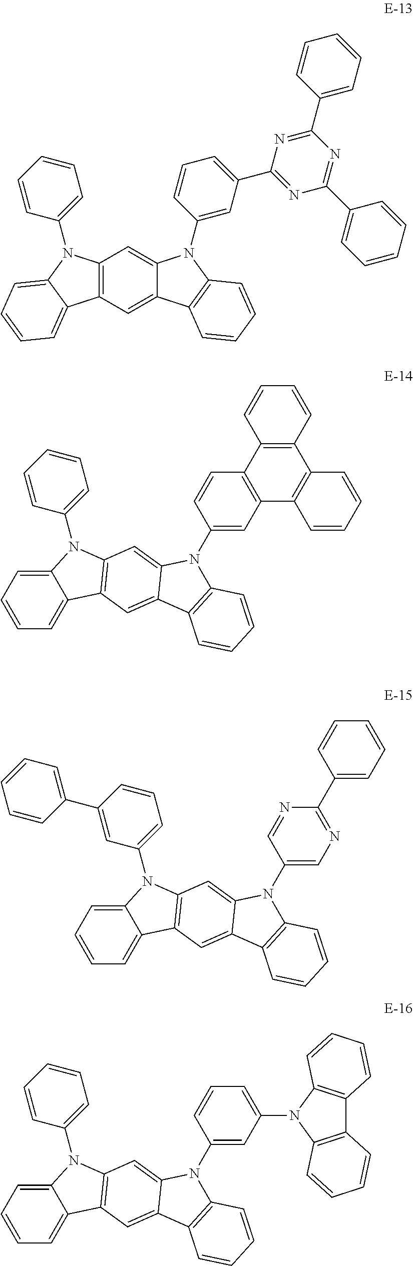 [SMILES: c1ccc(-c2nc(-c3ccccc3)nc(-c3cccc(-n4c5ccccc5c5cc6c7ccccc7n(-c7ccccc7)c6cc54)c3)n2)cc1 ; c1ccc(-n2c3ccccc3c3cc4c5ccccc5n(-c5ccc6c7ccccc7c7ccccc7c6c5)c4cc32)cc1 ; c1ccc(-c2cccc(-n3c4ccccc4c4cc5c6ccccc6n(-c6cnc(-c7ccccc7)nc6)c5cc43)c2)cc1 ; c1ccc(-n2c3ccccc3c3cc4c5ccccc5n(-c5cccc(-n6c7ccccc7c7ccccc76)c5)c4cc32)cc1]

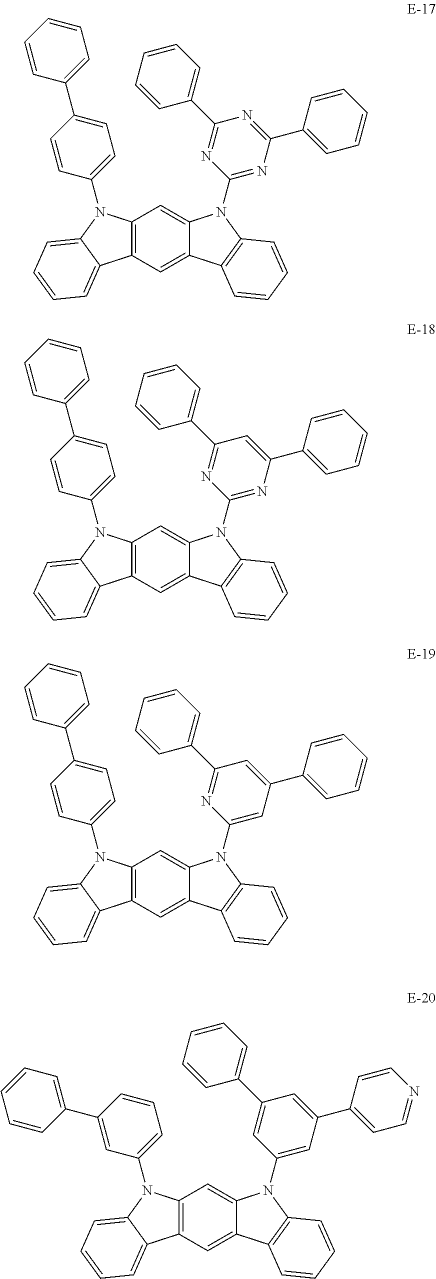

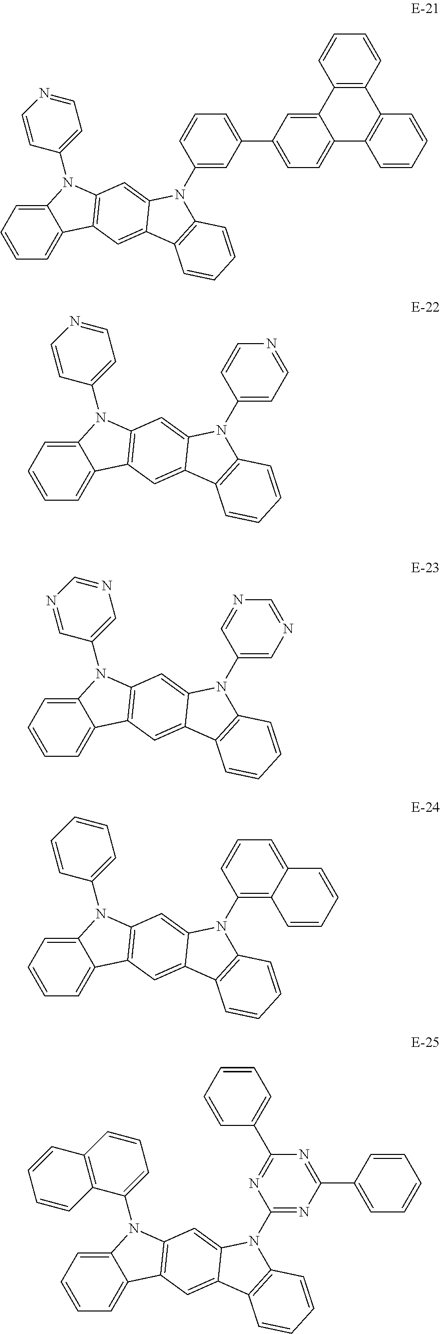 [SMILES: c1cc(-c2ccc3c4ccccc4c4ccccc4c3c2)cc(-n2c3ccccc3c3cc4c5ccccc5n(-c5ccncc5)c4cc32)c1 ; c1ccc2c(c1)c1cc3c4ccccc4n(-c4ccncc4)c3cc1n2-c1ccncc1 ; c1ccc2c(c1)c1cc3c4ccccc4n(-c4cncnc4)c3cc1n2-c1cncnc1 ; c1ccc(-n2c3ccccc3c3cc4c5ccccc5n(-c5cccc6ccccc56)c4cc32)cc1 ; c1ccc(-c2nc(-c3ccccc3)nc(-n3c4ccccc4c4cc5c6ccccc6n(-c6cccc7ccccc67)c5cc43)n2)cc1]

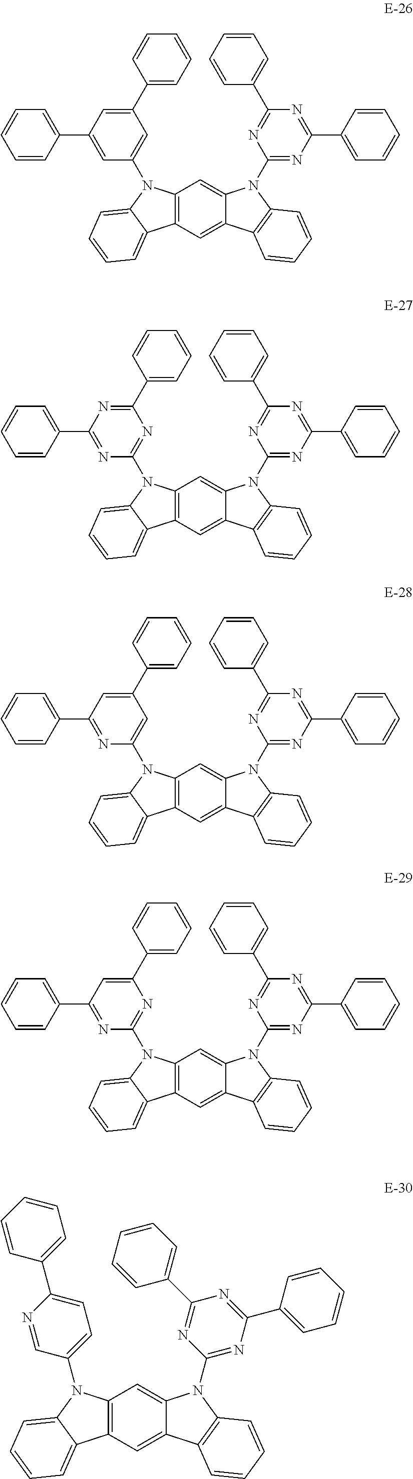 [SMILES: c1ccc(-c2cc(-c3ccccc3)cc(-n3c4ccccc4c4cc5c6ccccc6n(-c6nc(-c7ccccc7)nc(-c7ccccc7)n6)c5cc43)c2)cc1 ; c1ccc(-c2nc(-c3ccccc3)nc(-n3c4ccccc4c4cc5c6ccccc6n(-c6nc(-c7ccccc7)nc(-c7ccccc7)n6)c5cc43)n2)cc1 ; c1ccc(-c2cc(-c3ccccc3)nc(-n3c4ccccc4c4cc5c6ccccc6n(-c6nc(-c7ccccc7)nc(-c7ccccc7)n6)c5cc43)c2)cc1 ; c1ccc(-c2cc(-c3ccccc3)nc(-n3c4ccccc4c4cc5c6ccccc6n(-c6nc(-c7ccccc7)nc(-c7ccccc7)n6)c5cc43)n2)cc1 ; c1ccc(-c2ccc(-n3c4ccccc4c4cc5c6ccccc6n(-c6nc(-c7ccccc7)nc(-c7ccccc7)n6)c5cc43)cn2)cc1]

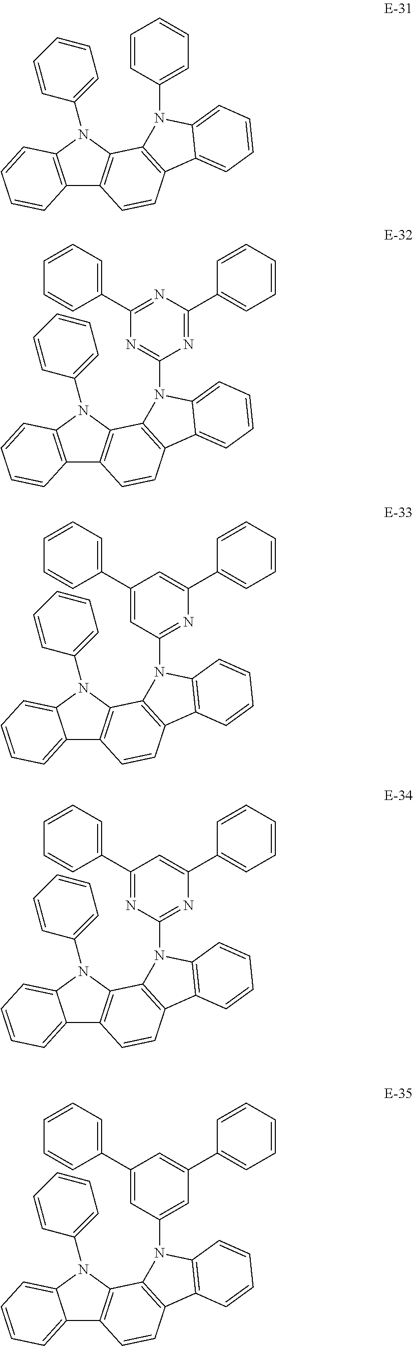

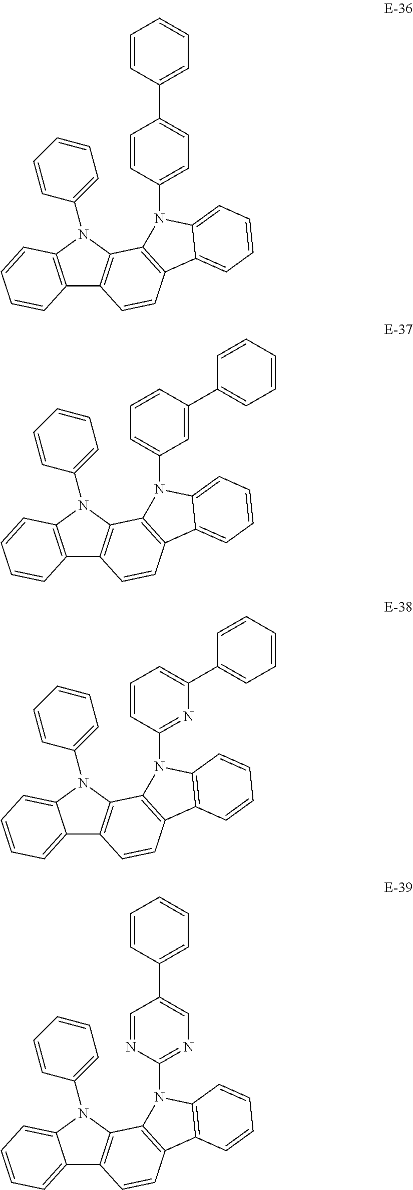

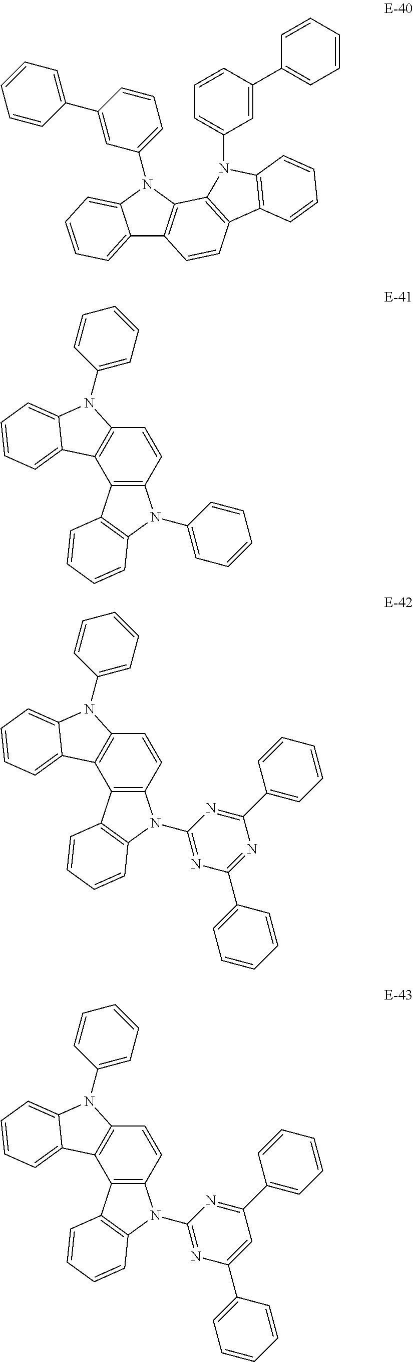

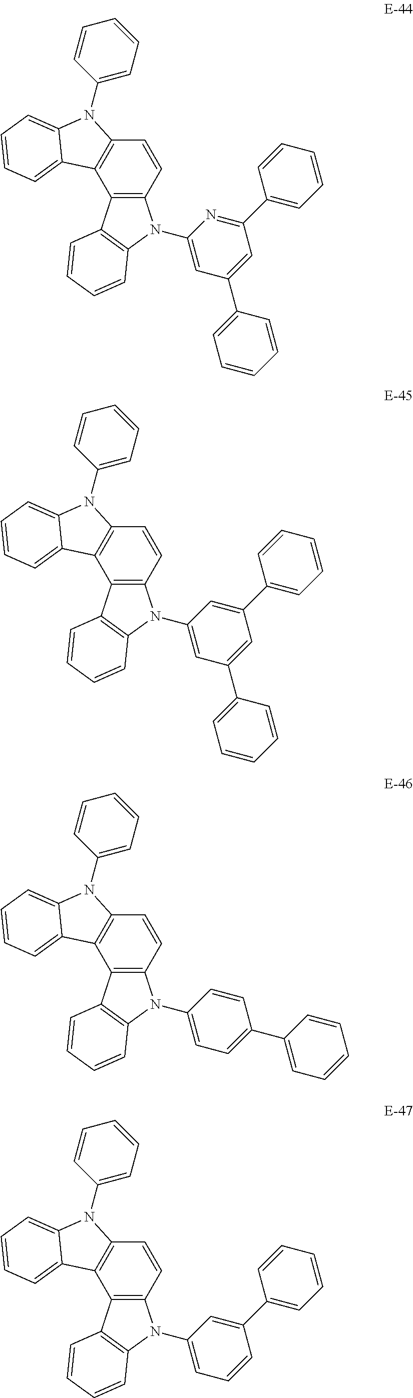 [SMILES: c1ccc(-c2cc(-c3ccccc3)nc(-n3c4ccccc4c4c5c6ccccc6n(-c6ccccc6)c5ccc43)c2)cc1 ; c1ccc(-c2cc(-c3ccccc3)cc(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1]

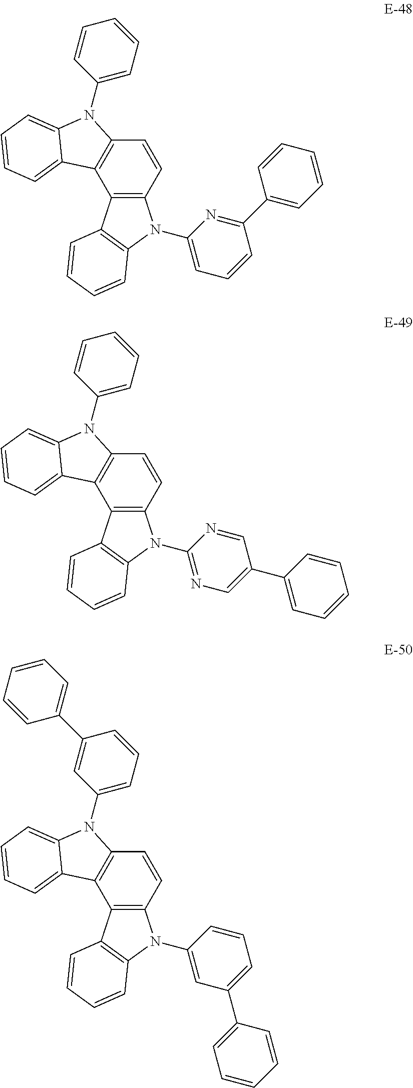

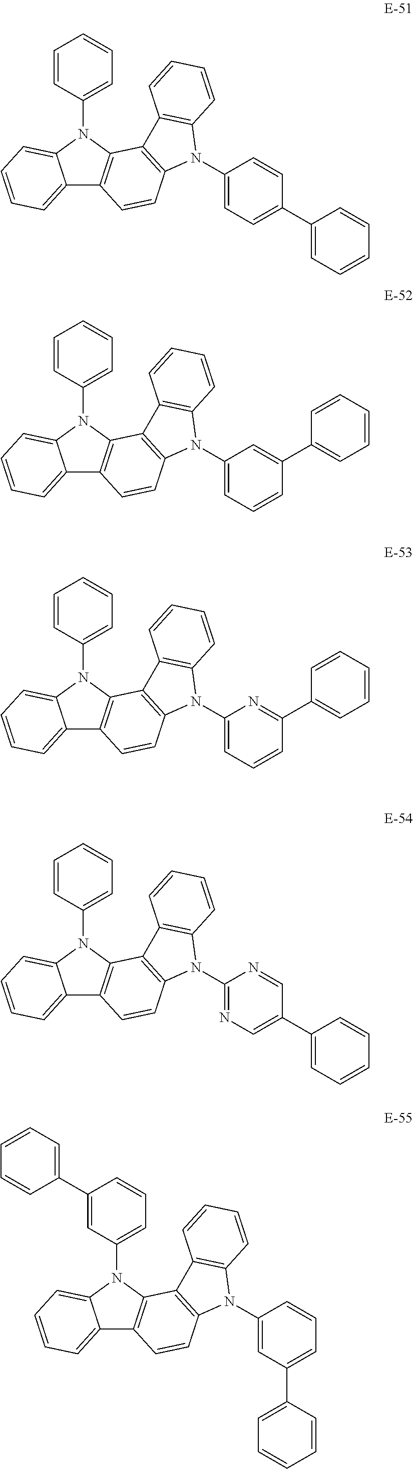 [SMILES: c1ccc(-c2ccc(-n3c4ccccc4c4c3ccc3c5ccccc5n(-c5ccccc5)c34)cc2)cc1 ; c1ccc(-c2cccc(-n3c4ccccc4c4c3ccc3c5ccccc5n(-c5ccccc5)c34)c2)cc1 ; c1ccc(-c2cccc(-n3c4ccccc4c4c3ccc3c5ccccc5n(-c5ccccc5)c34)n2)cc1 ; c1ccc(-c2cnc(-n3c4ccccc4c4c3ccc3c5ccccc5n(-c5ccccc5)c34)nc2)cc1 ; c1ccc(-c2cccc(-n3c4ccccc4c4c3ccc3c5ccccc5n(-c5cccc(-c6ccccc6)c5)c34)c2)cc1]

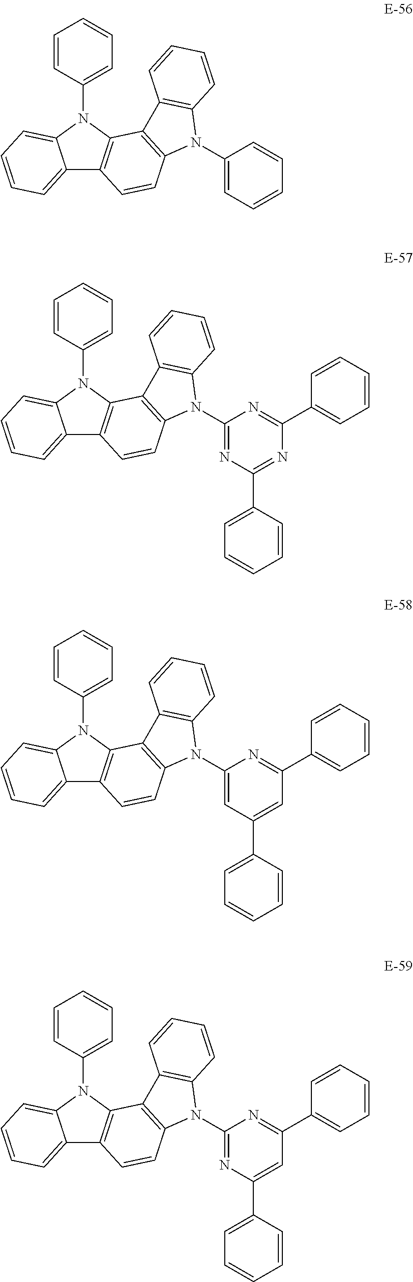 [SMILES: c1ccc(-n2c3ccccc3c3c2ccc2c4ccccc4n(-c4ccccc4)c23)cc1 ; c1ccc(-c2nc(-c3ccccc3)nc(-n3c4ccccc4c4c3ccc3c5ccccc5n(-c5ccccc5)c34)n2)cc1 ; c1ccc(-c2cc(-c3ccccc3)nc(-n3c4ccccc4c4c3ccc3c5ccccc5n(-c5ccccc5)c34)c2)cc1 ; c1ccc(-c2cc(-c3ccccc3)nc(-n3c4ccccc4c4c3ccc3c5ccccc5n(-c5ccccc5)c34)n2)cc1]

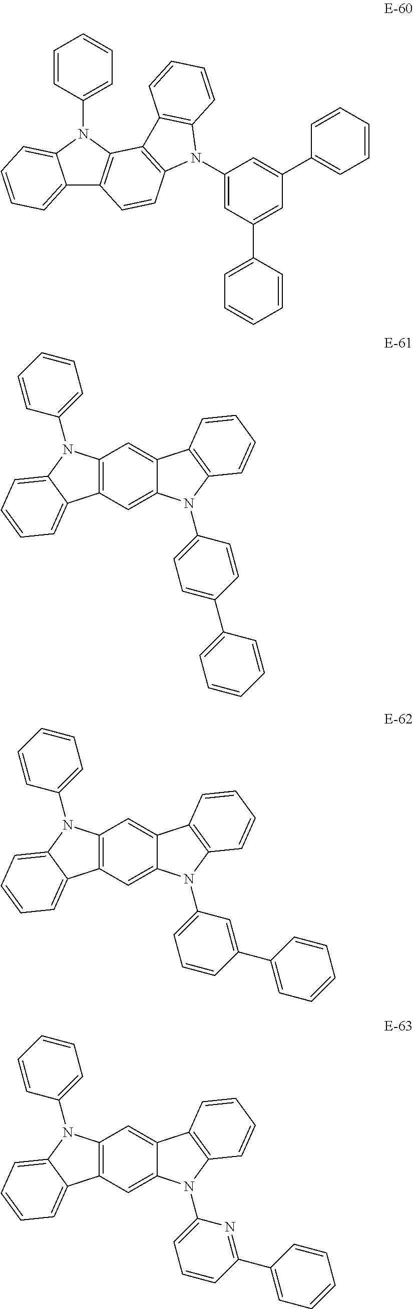 [SMILES: c1ccc(-c2cc(-c3ccccc3)cc(-n3c4ccccc4c4c3ccc3c5ccccc5n(-c5ccccc5)c34)c2)cc1 ; c1ccc(-c2ccc(-n3c4ccccc4c4cc5c(cc43)c3ccccc3n5-c3ccccc3)cc2)cc1 ; c1ccc(-c2cccc(-n3c4ccccc4c4cc5c(cc43)c3ccccc3n5-c3ccccc3)c2)cc1 ; c1ccc(-c2cccc(-n3c4ccccc4c4cc5c(cc43)c3ccccc3n5-c3ccccc3)n2)cc1]

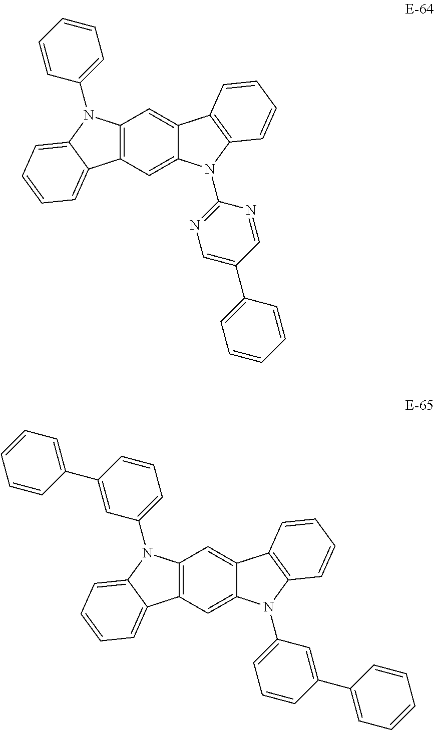 [SMILES: c1ccc(-c2cnc(-n3c4ccccc4c4cc5c(cc43)c3ccccc3n5-c3ccccc3)nc2)cc1 ; c1ccc(-c2cccc(-n3c4ccccc4c4cc5c(cc43)c3ccccc3n5-c3cccc(-c4ccccc4)c3)c2)cc1]

The second organic compound may include at least one of the compound represented by Chemical Formula 4 or the compound consisting of a combination of the moiety represented by Chemical Formula 5 and the moiety represented by Chemical Formula 6.

The composition may include the first organic compound and the second organic compound in a weight ratio of about 1:10 to about 10:1, preferably about 1:5 to about 5:1, most preferred about 1:4 to about 4:1.

The composition may be applied to or used in an organic layer of an organic optoelectric device. For example, the first organic compound and the second organic compound may play a role of a host. The first organic compound may have bipolar characteristics in which electron characteristics are relatively strong, the second organic compound may have bipolar characteristics in which hole characteristic is relatively strong, and the first and second organic compounds may be used together to increase charge mobility and stability and thus, to remarkably improve luminous efficiency and life-span characteristics.

In an implementation, the composition may further include another organic compound, in addition to the first organic compound and second organic compound.

In an implementation, the composition may further include a dopant. The dopant may be a red, green, or blue dopant, e.g., a phosphorescent dopant.

The dopant may be mixed with the host in a small amount to cause light emission, and may include a material such as a metal complex that emits light by multiple excitation into a triplet or more. The dopant may include, e.g., an inorganic, organic, or organic/inorganic compound, and one or more kinds thereof may be used.

Examples of the phosphorescent dopant may include an organic metal compound including Ir, Pt, Os, Ti, Zr, Hf, Eu, Tb, Tm, Fe, Co, Ni, Ru, Rh, Pd, or a combination thereof. The phosphorescent dopant may include, e.g., a compound represented by the following Chemical Formula Z.

L₂MX [Chemical Formula Z]

In Chemical Formula Z, M may be a metal, and L and X may each independently be a ligand to form a complex compound with M.

In an implementation, M may be, e.g., Ir, Pt, Os, Ti, Zr, Hf, Eu, Tb, Tm, Fe, Co, Ni, Ru, Rh, Pd or a combination thereof, and L and X may be, e.g., a bidentate ligand.

The composition may form a film using a dry film-forming method such as chemical vapor deposition.

Hereinafter, an organic optoelectric device to which the organic compound or the composition is applied is described.

The organic optoelectric device may include a device that converts electrical energy into photoenergy and/or vice versa, e.g., an organic photoelectric device, an organic light emitting diode, an organic solar cell, or an organic photo-conductor drum.

The organic optoelectric device may include an anode and a cathode facing each other, and at least one organic layer between the anode and the cathode. The organic layer may include the organic compound or composition according to an embodiment.

Herein, an organic light emitting diode (as one example of an organic optoelectric device) is described referring to drawings.

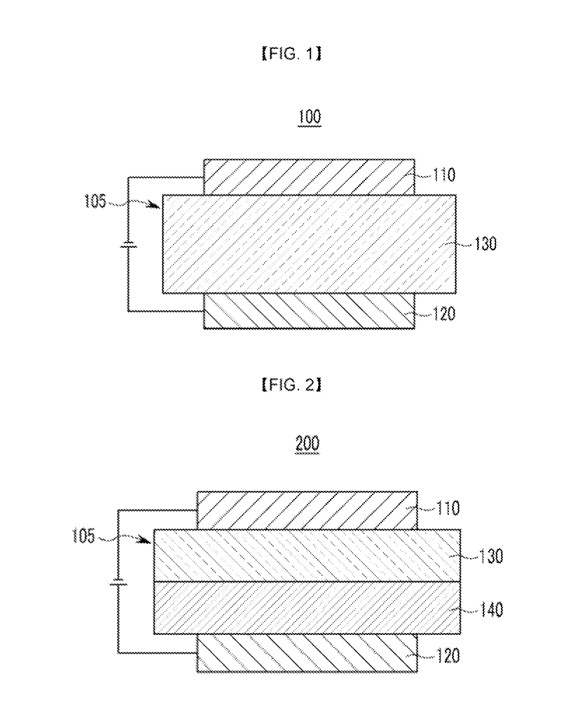

FIGS. 1 and 2 illustrate cross-sectional views of organic light emitting diodes according to the embodiments.

Referring to FIG. 1, an organic optoelectric device 100 according to one embodiment may include an anode 120 and a cathode 110 facing each other and an organic layer 105 between the anode 120 and cathode 110.

The anode 120 may be made of a conductor having a large work function to facilitate hole injection, and may include, e.g., a metal, metal oxide, and/or a conductive polymer. The anode 120 may include, e.g., a metal or an alloy thereof such as nickel, platinum, vanadium, chromium, copper, zinc, gold, or the like; metal oxide such as zinc oxide, indium oxide, indium tin oxide (ITO), indium zinc oxide (IZO), or the like; a combination of metal and oxide such as ZnO and Al or SnO₂ and Sb; a conductive polymer such as poly(3-methylthiophene), poly(3,4-(ethylene-1,2-dioxy)thiophene) (PEDT), polypyrrole, and polyaniline.

The cathode 110 may be made of a conductor having a small work function to facilitate electron injection, and may include, e.g., a metal, metal oxide and/or a conductive polymer. The cathode 110 may include, e.g., a metal or an alloy thereof such as magnesium, calcium, sodium, potassium, titanium, indium, yttrium, lithium, gadolinium, aluminum silver, tin, lead, cesium, barium, or the like; or a multi-layer structure material such as LiF/Al, LiO₂/Al, LiF/Ca, LiF/Al and BaF₂/Ca.

The organic layer 105 may include an emission layer 130 including the organic compound or the composition.

The emission layer 130 may include, e.g., the above organic compound at alone, a mixture of at least two kinds of the above organic compound, or the composition.

Referring to FIG. 2, an organic light emitting diode 200 may further include a hole auxiliary layer 140 as well as the emission layer 130. The hole auxiliary layer 140 may help further increase hole injection and/or hole mobility between the anode 120 and emission layer 130 and may help block electrons. The hole auxiliary layer 140 may include, e.g., a hole transport layer (HTL), a hole injection layer (HIL), and/or an electron blocking layer, and may include at least one layer.

In an implementation, the organic light emitting diode may further include, e.g., an electron transport layer (ETL), an electron injection layer (EIL), a hole injection layer (HIL), or the like, in the organic layer 105 in FIG. 1 or FIG. 2.

The organic light emitting diodes 100 and 200 may be manufactured by forming an anode or a cathode on a substrate, forming an organic layer (e.g., in accordance with a dry coating method such as evaporation, sputtering, plasma plating, and ion plating); and forming a cathode or an anode thereon.

The organic light emitting diode may be applied to an organic light emitting diode (OLED) display.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### Representative Synthesis Method

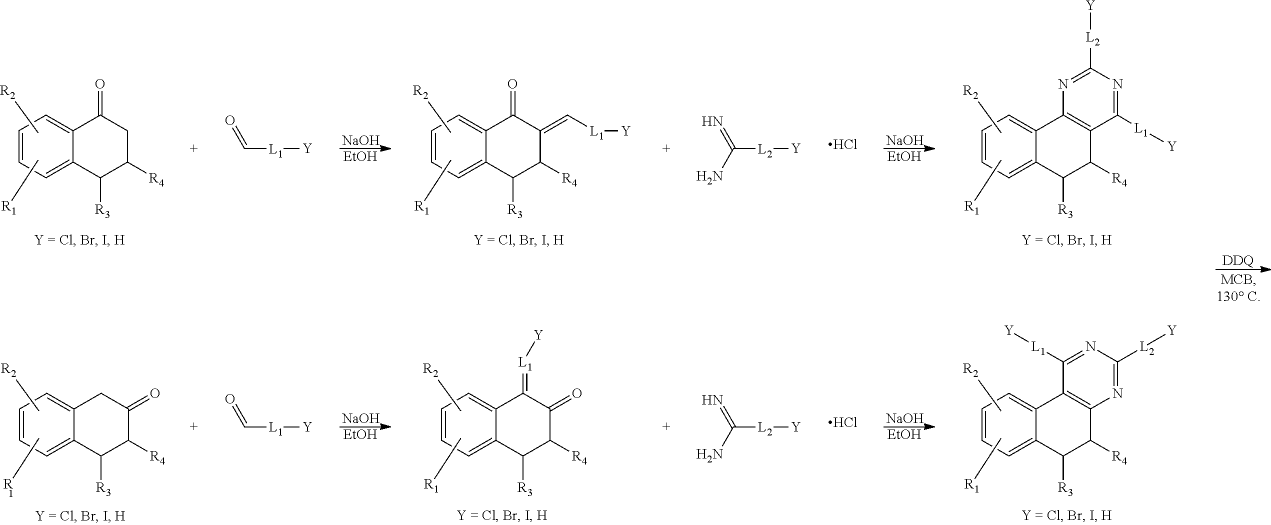

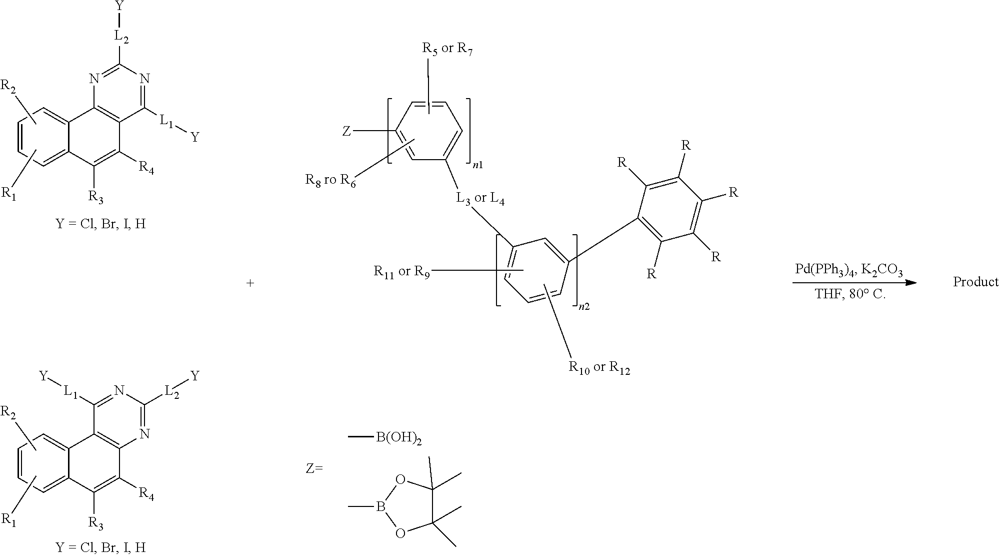

### Synthesis of Intermediate

### Synthesis Example 1

### Synthesis of Intermediate I-1

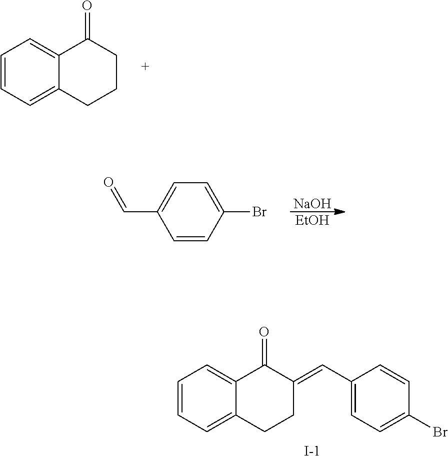

100 g (684 mmol) of α-tetralone was dissolved in 1 L of ethanol under a nitrogen atmpshere, 127 g (684 mmol) of 4-bromobenzaldehyde and 41.0 g (1026 mmol) of sodium hydroxide were added thereto, and the mixture was agitated at ambient temperature for 2 hours. When the reaction was terminated, the reaction solution was filtered and then washed with a small amount of ethanol. In this way, 179 g of an intermediate I-1 (a yield: 83%) was obtained.

HRMS (70 eV, EI+): m/z calcd for C₁₇H₁₃BrO: 312.0150. found: 312.

Elemental Analysis: C, 65%; H, 4%

### Synthesis Example 2

### Synthesis of Intermediate I-2

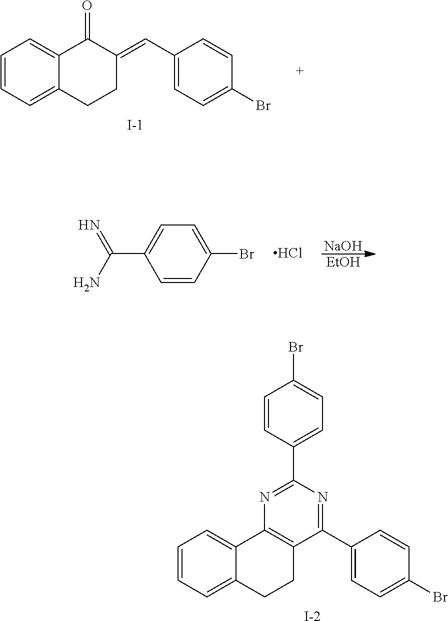

170 g (543 mmol) of the intermediate I-1 was dissolved in 1.5 L of ethanol under a nitrogen atmosphere, 128 g (543 mmol) of 4-bromobenzimidamide hydrochloride and 65.2 g (1,629 mmol) of sodium hydroxide were added thereto, and the mixture was agitated at ambient temperature for 17 hours. When the reaction was terminated, the reaction solution was filtered and then washed with a small amount of ethanol. In this way, 120 g of an intermediate I-2 (a yield: 45%) was obtained.

HRMS (70 eV, EI+): m/z calcd for C₂₄H₁₆Br₂N₂: 489.9680. found: 490.

Elemental Analysis: C, 59%; H, 3%

### Synthesis Example 3

### Synthesis of Intermediate I-3

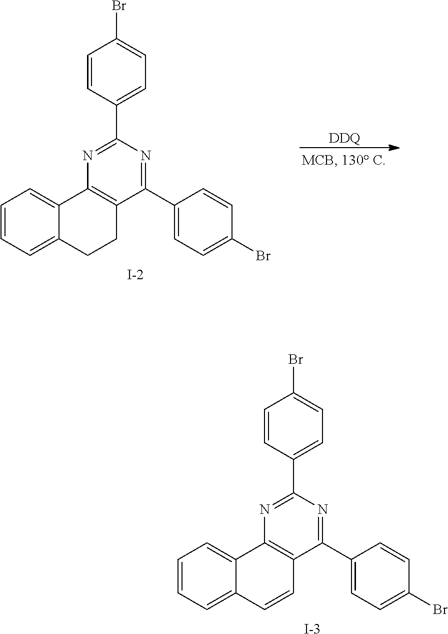

110 g (223 mmol) of the intermediate I-2 was dissolved in 1 L of monochlorobenzene (MCB) under a nitrogen atmosphere, 101 g (446 mmol) of 2,3-dichloro-5,6-dicyano-1,4-benzoquinone (DDQ) was added thereto, and the mixture was heated and refluxed at 130° C. for 15 hours. When the reaction was terminated, water was added to the reaction solution, and the mixture was extracted with dichloromethane (DCM), treated with anhydrous MgSO₄ to remove moisture and then, filtered and concentrated under a reduced pressure. The obtained residue was separated and purified through flash column chromatography, obtaining 76.5 g of an intermediate I-3 (a yield: 70%).

HRMS (70 eV, EI+): m/z calcd for C₂₄H₁₄Br₂N₂: 487.9524. found: 488.

Elemental Analysis: C, 59%; H, 3%

### Synthesis Example 4

### Synthesis of Intermediate I-4

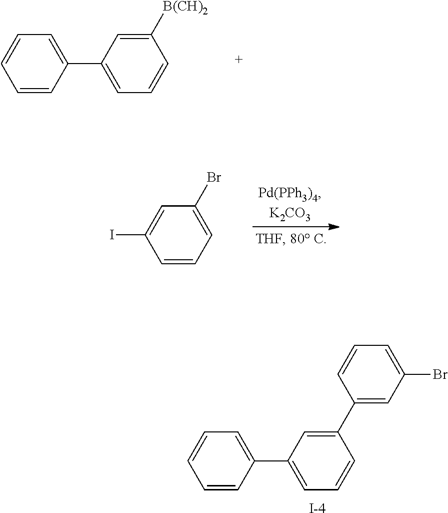

100 g (505 mmol) of biphenyl-3-ylboronic acid was dissolved in 1.4 L of tetrahydrofuran (THF) under a nitrogen atmosphere, 171 g (606 mmol) of 1-bromo-3-iodobenzene and 5.84 g (5.05 mmol) of tetrakis(triphenylphosphine)palladium were added thereto, and the mixture was agitated. 174 g (1,263 mmol) of potassium carbonate saturated in water was added thereto, and the resulting mixture was heated and refluxed at 80° C. for 8 hours. When the reaction was terminated, water was added to the reaction solution, and the mixture was extracted with dichloromethane (DCM), treated with anhydrous MgSO₄ to remove moisture and then, filtered and concentrated under a reduced pressure. The obtained residue was separated and purified through flash column chromatography, obtaining 141 g of an intermediate I-4 (a yield: 90%).

HRMS (70 eV, EI+): m/z calcd for C₁₈H₁₃Br: 308.0201. found: 308.

Elemental Analysis: C, 70%; H, 4%

### Synthesis Example 5

### Synthesis of Intermediate I-5

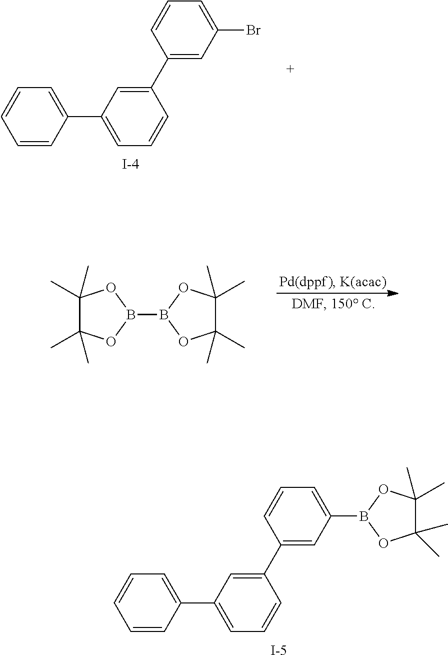

130 g (420 mmol) of the intermediate I-4 was dissolved in 1.3 L of dimethylforamide (DMF) under a nitrogen atmosphere, 128 g (505 mmol) of bis(pinacolato)diboron, 3.43 g (4.2 mmol) of (1,1′-bis(diphenylphosphine)ferrocene)dichloropalladium (II) and 124 g (1,260 mmol) of potassium acetate were added thereto, and the mixture was heated and refluxed at 150° C. for 6 hours. When the reaction was terminated, water was added to the reaction solution, and the mixture was filtered and dried in a vacuum oven. Then, the obtained residue was separated and purified through flash column chromatography, obtaining 106 g of an intermediate I-5 (a yield: 71%).

HRMS (70 eV, EI+): m/z calcd for C₂₄H₂₅BO₂: 356.1948. found: 356.

Elemental Analysis: C, 81%; H, 7%

### Synthesis Example 6

### Synthesis of Intermediate I-6

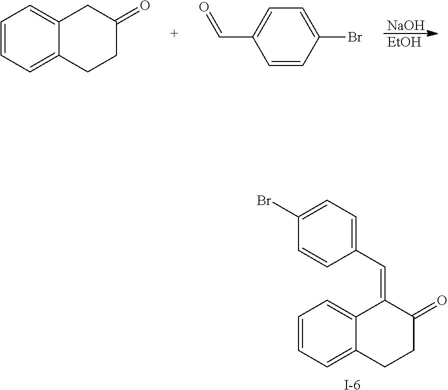

100 g (684 mmol) of β-tetralone was dissolved in 1 L of ethanol under a nitrogen atmosphere, 127 g (684 mmol) of 4-bromobenzaldehyde and 41.0 g (1026 mmol) of sodium hydroxide were added thereto, and the mixture was agitated at ambient temperature for 2 hours. When the reaction was terminated, the reaction solution was filtered and washed with a small amount of ethanol. In this way, 161 g of an intermediate I-6 (a yield: 75%) was obtained.

HRMS (70 eV, EI+): m/z calcd for C₁₇H₁₃BrO: 312.0150. found: 312.

Elemental Analysis: C, 65%; H, 4%

### Synthesis Example 7

### Synthesis of Intermediate I-7

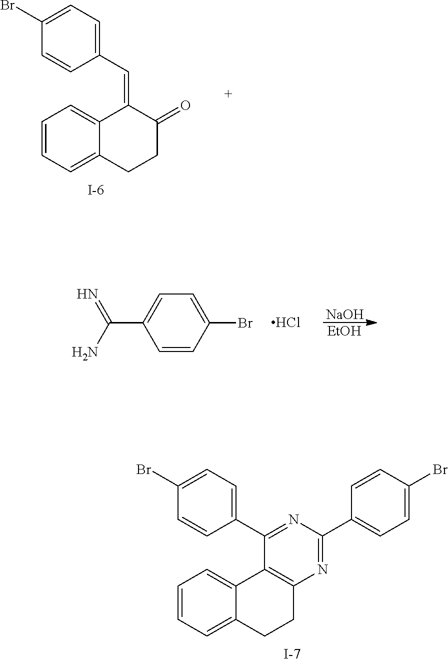

150 g (479 mmol) of the intermediate I-6 was dissolved in 1.5 L of ethanol under a nitrogen atmosphere, 95.3 g (479 mmol) of 4-bromobenzimidamide hydrochloride and 65.2 g (1,437 mmol) of sodium hydroxide were added thereto, and the mixture was agitated at ambient temperature for 15 hours. When the reaction was terminated, the reaction solution was filtered and washed with a small amount of ethanol. In this way, 91.9 g of an intermediate I-7 (a yield: 39%) was obtained.

HRMS (70 eV, EI+): m/z calcd for C₂₄H₁₆Br₂N₂: 489.9680. found: 490.

Elemental Analysis: C, 59%; H, 3%

### Synthesis Example 8

### Synthesis of Intermediate I-8

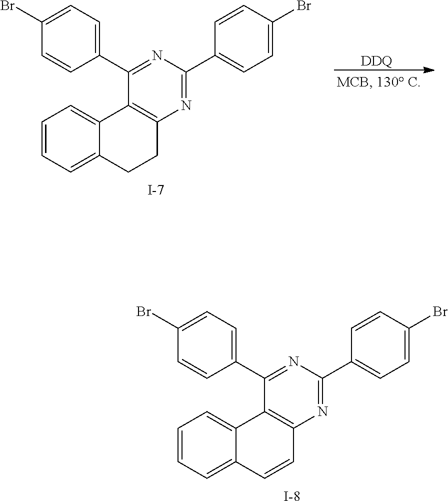

85 g (173 mmol) of the intermediate I-7 was dissolved in 0.8 L of monochlorobenzene

(MCB) under a nitrogen atmosphere, 78.4 g (345 mmol) of 2,3-dichloro-5,6-dicyano-1,4-benzoquinone (DDQ) was added thereto, and the mixture was heated and refluxed at 130° C. for 15 hours. When the reaction was terminated, water was added to the reaction solution, and the mixture was extracted with dichloromethane (DCM), treated with anhydrous MgSO₄ to remove moisture, and then, filtered and concentrated under a reduced pressure. Then, the obtained residue was separated and purified through flash column chromatography, obtaining 57.7 g of an intermediate I-8 (a yield: 68%).

HRMS (70 eV, EI+): m/z calcd for C₂₄H₁₄Br₂N₂: 487.9524. found: 488.

Elemental Analysis: C, 59%; H, 3%

### Synthesis Example 9

### Synthesis of Intermediate I-9

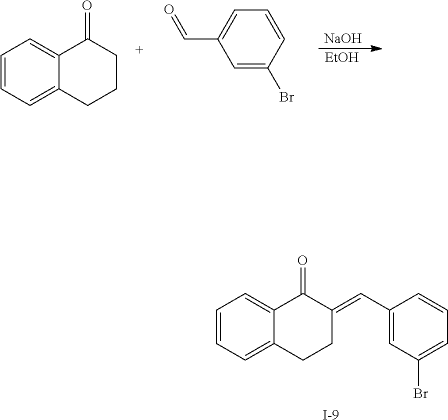

100 g (684 mmol) of α-tetralone was dissolved in 1 L of ethanol under a nitrogen atmosphere, 127 g (684 mmol) of 3-bromobenzaldehyde and 41.0 g (1026 mmol) of sodium hydroxide were added thereto, and the mixture was agitated at ambient temperature for 2 hours. When the reaction was terminated, the reaction solution was filtered and then, washed with a small amount of ethanol. In this way, 171 g of an intermediate I-9 (a yield: 80%) was obtained.

HRMS (70 eV, EI+): m/z calcd for C₁₇H₁₃BrO: 312.0150. found: 312.

Elemental Analysis: C, 65%; H, 4%

### Synthesis Example 10

### Synthesis of Intermediate I-10

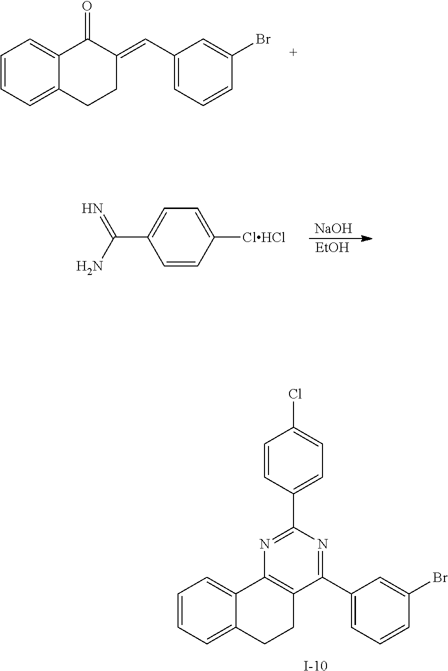

165 g (527 mmol) of the intermediate I-9 was dissolved in 1.5 L of ethanol under a nitrogen atmosphere, 101 g (527 mmol) of 4-chlorobenzimidamide hydrochloride and 63.2 g (1,581 mmol) of sodium hydroxide were added thereto, and the mixture was agitated at ambient temperature for 15 hours. When the reaction was terminated, the reaction solution was filtered and then, washed with a small amount of ethanol. In this way, 99.1 g of an intermediate I-10 (a yield: 42%) was obtained.

HRMS (70 eV, EI+): m/z calcd for C₂₄H₁₆BrClN₂: 446.0185. found: 446.

Elemental Analysis: C, 64%; H, 4%

### Synthesis Example 11

### Synthesis of Intermediate I-11

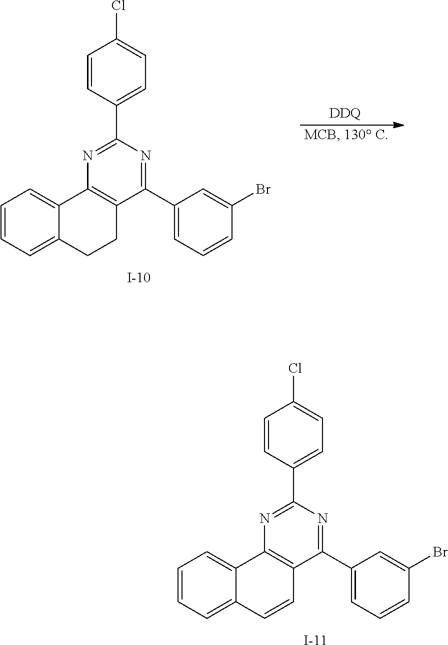

90 g (201 mmol) of the intermediate I-10 was dissolved in 1 L of monochlorobenzene (MCB) under a nitrogen atmosphere, 91.3 g (402 mmol) of 2,3-dichloro-5,6-dicyano-1,4-benzoquinone (DDQ) was added thereto, and the mixture was refluxed and heated at 130° C. for 15 hours. When the reaction was terminated, water was added to the reaction solution, and the mixture was extracted with dichloromethane (DCM), treated with anhydrous MgSO₄ to remove moisture and then, filtered and concentrated under a reduced pressure. Then, the obtained residue was separated and purified through flash column chromatography, obtaining 58.2 g of an intermediate I-11 (a yield: 65%).

HRMS (70 eV, EI+): m/z calcd for C₂₄H₁₄BrClN₂: 444.0029. found: 444.

Elemental Analysis: C, 65%; H, 3%

### Synthesis Example 12

### Synthesis of Intermediate I-12

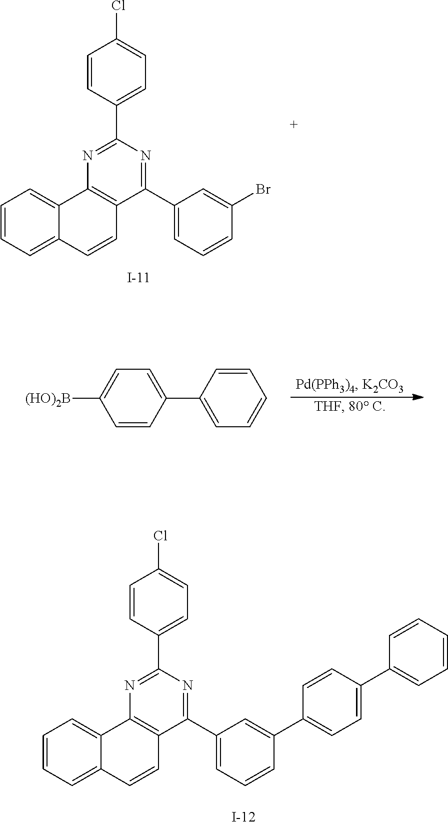

50 g (112 mmol) of the intermediate I-11 was dissolved in 0.4 L of tetrahydrofuran (THF) under a nitrogen atmosphere, 24.4 g (123 mmol) of biphenyl-4-ylboronic acid and 1.29 g (1.12 mmol) of tetrakis(triphenylphosphine)palladium were added thereto, and the mixture was agitated. 38.7 g (280 mmol) of potassium carbonate saturated in water was added thereto, and the mixture was heated and refluxed at 80° C. for 18 hours. When the reaction was terminated, water was added to the reaction solution, and the mixture was extracted with dichloromethane (DCM) and treated with anhydrous MgSO₄ to remove moisture and then, filtered and concentrated under a reduced pressure. Then, the obtained residue was separated and purified through flash column chromatography, obtaining 48.2 g of an intermediate I-12 (a yield: 83%).

HRMS (70 eV, EI+): m/z calcd for C₃₆H₂₃C1N₂: 518.1550. found: 518.

Elemental Analysis: C, 83%; H, 4%

### Synthesis Example 13

### Synthesis of Intermediate I-13

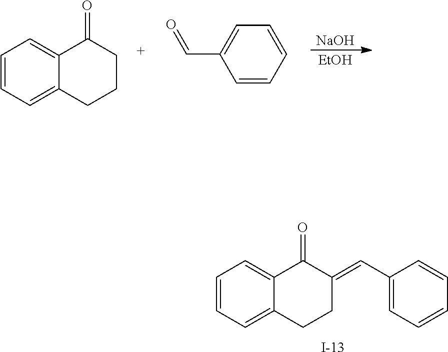

100 g (684 mmol) of α-tetralone was dissolved in 1 L of ethanol under a nitrogen atmosphere, 72.6 g (684 mmol) of benzaldehyde and 41.0 g (1026 mmol) of sodium hydroxide were added thereto, and the mixture was agitated at ambient temperature for 2 hours. When the reaction was terminated, the reaction solution was filtered and washed with a small amount of ethanol. In this way, 139 g of an intermediate I-13 (a yield: 87%) was obtained.

HRMS (70 eV, EI+): m/z calcd for C₁₇H₁₄O: 234.1045. found: 234.

Elemental Analysis: C, 87%; H, 6%

### Synthesis Example 14

### Synthesis of Intermediate I-14

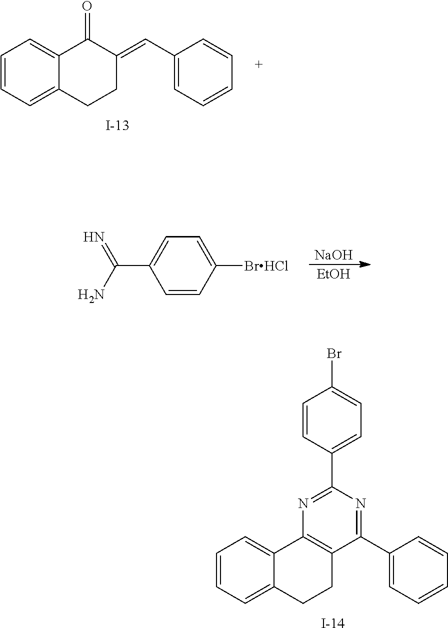

130 g (555 mmol) of the intermediate I-13 was dissolved in 1.5 L of ethanol under a nitrogen atmosphere, 131 g (555 mmol) of 4-bromobenzimidamide hydrochloride and 66.6 g (1,665 mmol) of sodium hydroxide were added thereto, and the mixture was agitated at ambient temperature for 15 hours. When the reaction was terminated, the reaction solution was filtered and washed with a small amount of ethanol. In this way, 115 g of an intermediate I-14 (a yield: 50%) was obtained.

HRMS (70 eV, EI+): m/z calcd for C₂₄H₁₇BrN₂: 412.0575. found: 412.

Elemental Analysis: C, 70%; H, 4%

### Synthesis Example 15

### Synthesis of Intermediate I-15

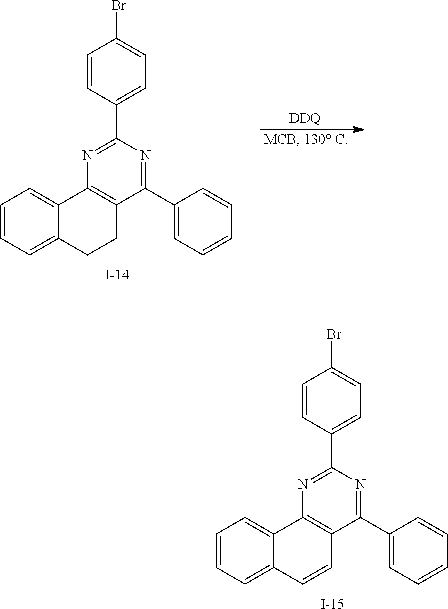

110 g (266 mmol) of the intermediate I-14 was dissolved in 1.2 L of monochlorobenzene (MCB) under a nitrogen atmosphere, 121 g (532 mmol) of 2,3-dichloro-5,6-dicyano-1,4-benzoquinone (DDQ) was added thereto, and the mixture was heated and refluxed at 130° C. for 15 hours. When the reaction was terminated, water was added to the reaction solution, and the mixture was extracted with dichloromethane (DCM) and treated with anhydrous MgSO₄ to remove moisture and then, filtered and concentrated under a reduced pressure. Then, the obtained residue was separated and purified through flash column chromatography, obtaining 72.2 g (66%) of an intermediate I-15.

HRMS (70 eV, EI+): m/z calcd for C₂₄H₁₅BrN₂: 410.0419. found: 410.

Elemental Analysis: C, 70%; H, 4%

### Synthesis Example 16

### Synthesis of Intermediate I-16

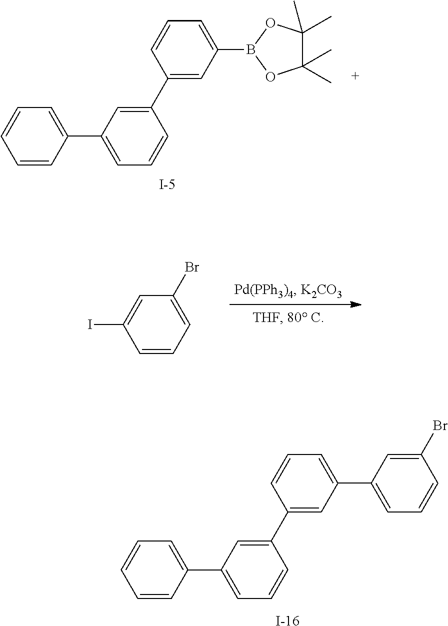

100 g (281 mmol) of the intermediate I-5 was dissolved in 1 L of tetrahydrofuran (THF) under a nitrogen atmosphere, 95.3 g (337 mmol) of 1-bromo-3-iodobenzene and 3.25 g (2.81 mmol) of tetrakis(triphenylphosphine)palladium were added thereto, and the mixture was agitated. 97.1 g (703 mmol) of potassium carbonate saturated in water was added thereto, and the resulting mixture was heated and refluxed at 80° C. for 11 hours. When the reaction was terminated, water was added to the reaction solution, and the mixture was extracted with dichloromethane (DCM), treated with anhydrous MgSO₄ to remove moisture and then, filtered and concentrated under a reduced pressure. Then, the obtained residue was separated and purified through flash column chromatography, obtaining 92.0 g of an intermediate I-16 (a yield: 85%).

HRMS (70 eV, EI+): m/z calcd for C₂₄H₁₇Br: 384.0514. found: 384.

Elemental Analysis: C, 75%; H, 4%

### Synthesis Example 17

### Synthesis of Intermediate I-17

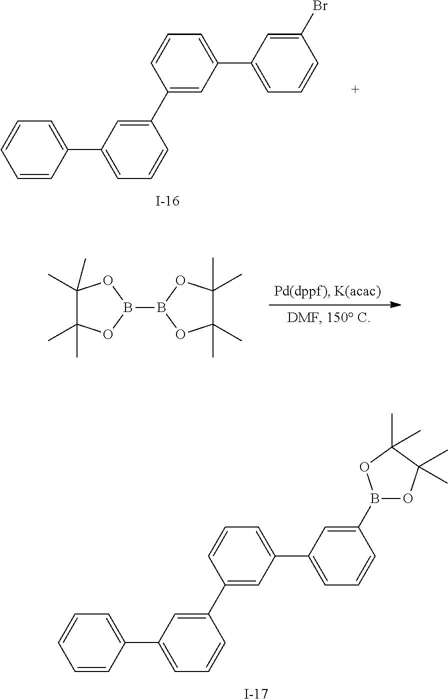

85 g (221 mmol) of the intermediate I-16 was dissolved in 0.8 L of dimethylforamide (DMF) under a nitrogen atmosphere, 67.2 g (265 mmol) of bis(pinacolato)diboron, 1.80 g (2.21 mmol) of (1,1′-bis(diphenylphosphine)ferrocene)dichloropalladium (II) and 65.1 g (663 mmol) of potassium acetate were added thereto, and the mixture was heated and refluxed at 150° C. for 5 hours. When the reaction was terminated, water was added to the reaction solution, and the mixture was filtered and then, dried in a vacuum oven. Then, the obtained residue was separated and purified through flash column chromatography, obtaining 74.5 g of an intermediate I-17 (a yield: 78%).

HRMS (70 eV, EI+): m/z calcd for C₃₀H₂₉BO₂: 432.2261. found: 432.

Elemental Analysis: C, 83%; H, 7%

### Synthesis Example 18

### Synthesis of Intermediate I-18

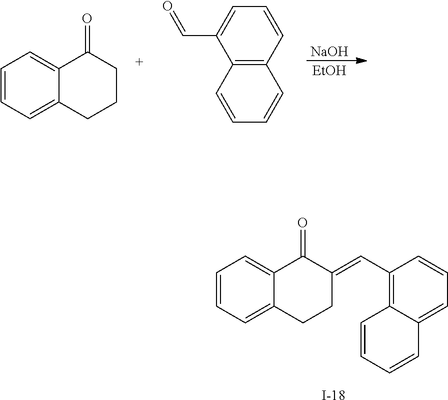

100 g (684 mmol) of α-tetralone was dissolved in 1 L of ethanol under a nitrogen atmosphere, 107 g (684 mmol) of 1-naphthaldehyde and 41.0 g (1026 mmol) of sodium hydroxide were added thereto, and the mixture was agitated at ambient temperature for 2 hours. When the reaction was terminated, the reaction solution was filtered and washed with a small amount of ethanol. In this way, 173 g of an intermediate I-18 (a yield: 89%) was obtained.

HRMS (70 eV, EI+): m/z calcd for C₂₁H₆O: 284.1201. found: 284.

Elemental Analysis: C, 89%; H, 6%

### Synthesis Example 19

### Synthesis of Intermediate I-19

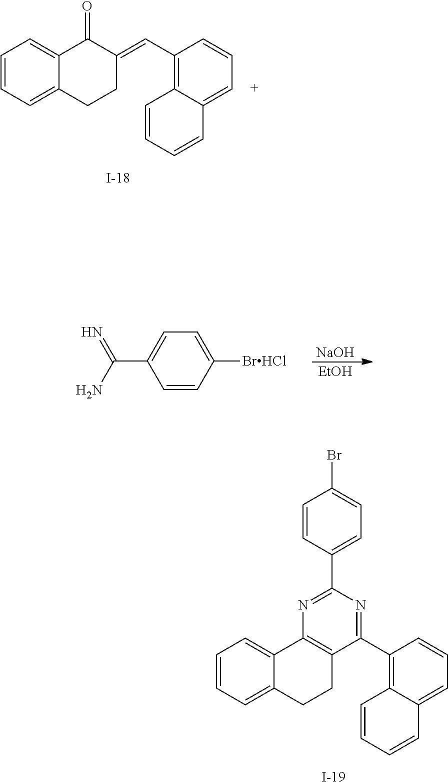

170 g (598 mmol) of the intermediate I-18 was dissolved in 1.5 L of ethanol under a nitrogen atmosphere, 141 g (598 mmol) of 4-bromobenzimidamide hydrochloride and 71.8 g (1,794 mmol) of sodium hydroxide were added thereto, and the mixture was agitated at ambient temperature for 15 hours. When the reaction was terminated, the reaction solution was filtered and then, washed with a small amount of ethanol. In this way, 114 g of an intermediate I-19 (a yield: 41%) was obtained.

HRMS (70 eV, EI+): m/z calcd for C₂₈H₁₉BrN₂: 462.0732. found: 462.

Elemental Analysis: C, 73%; H, 4%

### Synthesis Example 20

### Synthesis of Intermediate I-20

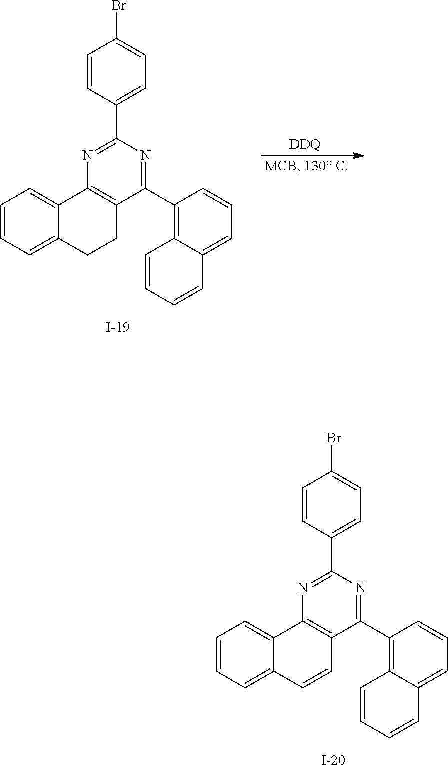

105 g (227 mmol) of the intermediate I-19 was dissolved in 1 L of monochlorobenzene (MCB) under a nitrogen atmosphere, 103 g (453 mmol) of 2,3-dichloro-5,6-dicyano-1,4-benzoquinone (DDQ) was added thereto, and the mixture was heated and refluxed at 130° C. for 15 hours. When the reaction was terminated, water was added to the reaction solution, and the mixture was extracted with dichloromethane (DCM) and treated with anhydrous MgSO₄ to remove moisture and then, filtered and concentrated under a reduced pressure. Then, the obtained residue was separated and purified through flash column chromatography, obtaining 68.1 g of an intermediate I-20 (a yield: 65%).

HRMS (70 eV, EI+): m/z calcd for C₂₈H₁₇BrN₂: 460.0575. found: 460.

Elemental Analysis: C, 73%; H, 4%

### Synthesis of Final Compound

### Synthesis Example 21

### Synthesis of Compound 1

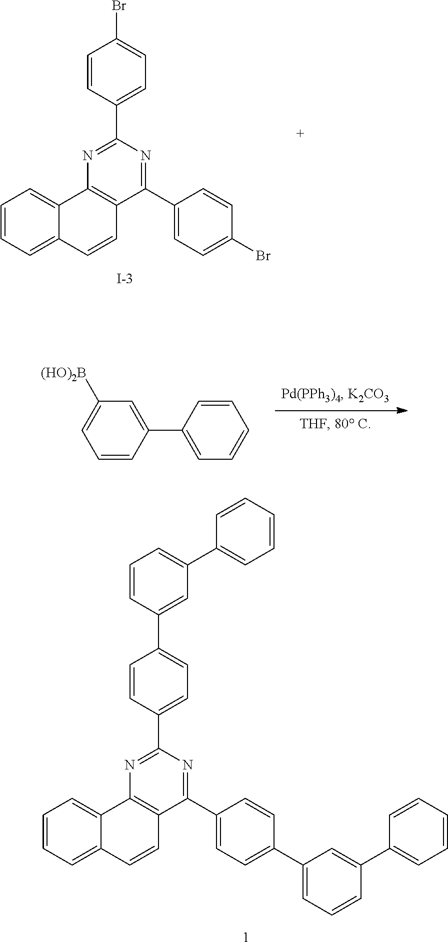

20 g (40.8 mmol) of the intermediate I-3 was dissolved in 0.2 L of tetrahydrofuran (THF) under a nitrogen atmosphere, 16.2 g (81.6 mmol) of biphenyl-3-ylboronic acid and 0.94 g (0.82 mmol) of tetrakis(triphenylphosphine)palladium were added thereto, and the mixture was agitated. 28.2 g (204 mmol) of potassium carbonate saturated in water was added thereto, and the mixture was heated and refluxed at 80° C. for 12 hours. When the reaction was terminated, water was added to the reaction solution, and the mixture was extracted with dichloromethane (DCM) and treated with anhydrous MgSO₄ to remove moisture and then, filtered and concentrated under a reduced pressure. Then, the obtained residue was separated and purified through flash column chromatography, obtaining 24.9 g of a compound 1 (a yield: 96%).

HRMS (70 eV, EI+): m/z calcd for C₄₈H₃₂N₂: 636.2565. found: 636.

Elemental Analysis: C, 91%; H, 5%

### Synthesis Example 22

### Synthesis of Compound 2

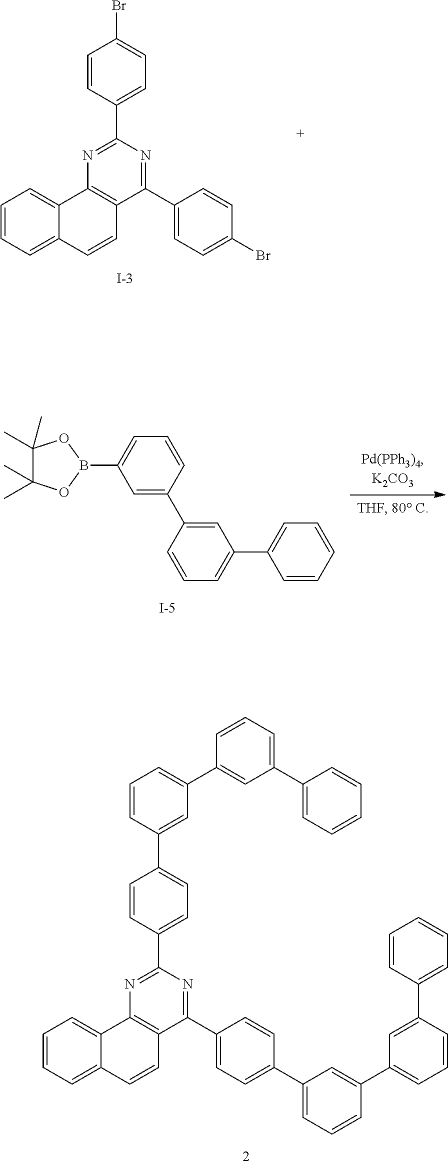

20 g (40.8 mmol) of the intermediate I-3 was dissolved in 0.2 L of tetrahydrofuran (THF) under a nitrogen atmosphere, 29.1 g (81.6 mmol) of the intermediate I-5 and 0.94 g (0.82 mmol) of tetrakis(triphenylphosphine)palladium were added thereto, and the mixture was agitated. 28.2 g (204 mmol) of potassium carbonate saturated in water was added thereto, and the mixture was heated and refluxed at 80° C. for 15 hours. When the reaction was terminated, water was added to the reaction solution, and the mixture was extracted with dichloromethane (DCM) and treated with anhydrous MgSO₄ to remove moisture and then, filtered and concentrated under a reduced pressure. Then, the obtained residue was separated and purified through flash column chromatography, obtaining 29.6 g of a compound 2 (a yield: 92%).

HRMS (70 eV, EI+): m/z calcd for C₆₀H₄₀N₂: 788.3191. found: 788.

Elemental Analysis: C, 91%; H, 5%

### Synthesis Example 23

### Synthesis of Compound 5

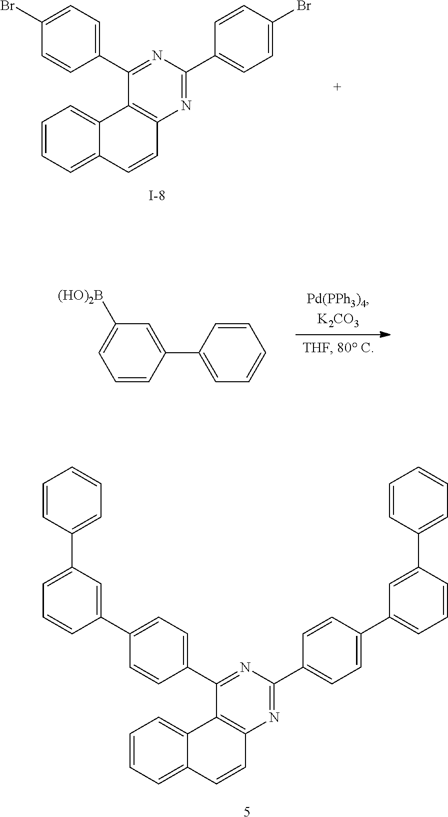

20 g (40.8 mmol) of the intermediate I-8 was dissolved in 0.2 L of tetrahydrofuran (THF) under a nitrogen atmosphere, 16.2 g (81.6 mmol) of biphenyl-3-yl boronic acid and 0.94 g (0.82 mmol) of tetrakis(triphenylphosphine)palladium were added thereto, and the mixture was agitated. 28.2 g (204 mmol) of potassium carbonate saturated in water was added thereto, and the mixture was heated and refluxed at 80° C. for 13 hours. When the reaction was terminated, water was added to the reaction solution, and the mixture was extracted with dichloromethane (DCM) and treated with anhydrous MgSO₄ and then, filtered and concentrated under a reduced pressure. Then, the obtained residue was separated and purified through flash column chromatography, obtaining 24.7 g of a compound 5 (a yield: 95%).

HRMS (70 eV, EI+): m/z calcd for C₄₈H₃₂N₂: 636.2565. found: 636.

Elemental Analysis: C, 91%; H, 5%

### Synthesis Example 24

### Synthesis of Compound 6

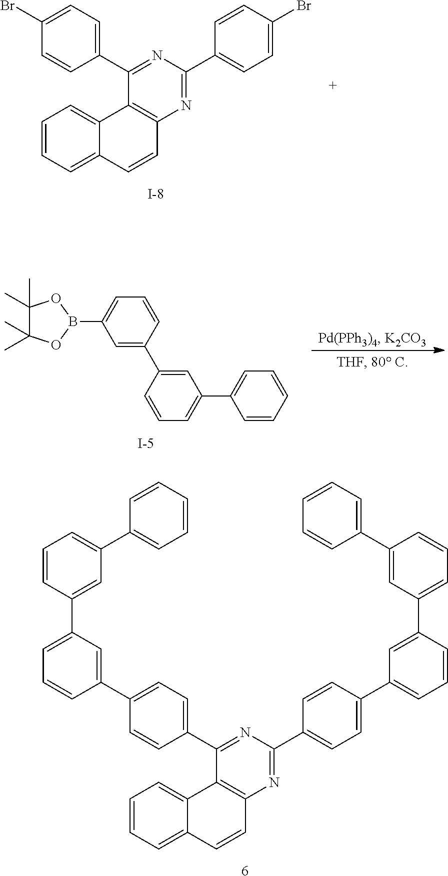

20 g (40.8 mmol) of the intermediate I-8 was dissolved in 0.2 L of tetrahydrofuran (THF) under a nitrogen atmosphere, 29.1 g (81.6 mmol) of the intermediate I-5 and 0.94 g (0.82 mmol) of tetrakis(triphenylphosphine)palladium were added thereto, and the mixture was agitated. 28.2 g (204 mmol) of potassium carbonate saturated in water was heated and refluxed at 80° C. for 15 hours. When the reaction was terminated, water was added to the reaction solution, and the mixture was extracted with dichloromethane (DCM) and treated with anhydrous MgSO₄ to remove moisture and then, filtered and concentrated under a reduced pressure. Then, the obtained residue was separated and purified through flash column chromatography, obtaining 29.0 g of a compound 6 (a yield: 90%).

HRMS (70 eV, EI+): m/z calcd for C₆₀H₄₀N₂: 788.3191. found: 788.

Elemental Analysis: C, 91%; H, 5%

### Synthesis Example 25

### Synthesis of Compound 19

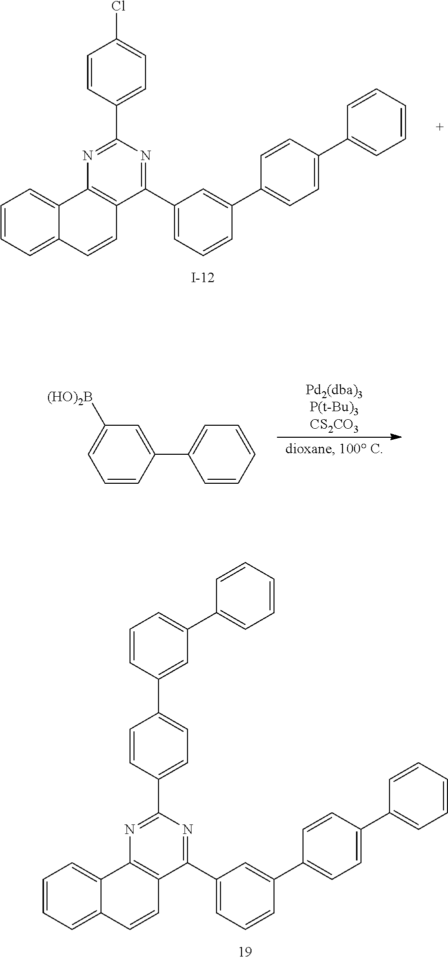

20 g (38.5 mmol) of the intermediate I-12 was dissolved in 0.15 L of dioxane under a nitrogen atmosphere, 7.63 g (38.5 mmol) of biphenyl-3-ylboronic acid, 0.36 g (0.39 mmol) of tris(diphenylideneacetone)dipalladium (0), 0.39 g (1.95 mmol) of tris-tert-butylphosphine and 31.4 g (96.3 mmol) of cesium carbonate were sequentially added thereto, and the mixture was heated and refluxed at 100° C. for 18 hours. When the reaction was terminated, water was added to the reaction solution, and the mixture was extracted with dichloromethane (DCM) and treated with anhydrous MgSO₄ and then, filtered and concentrated under a reduced pressure. Then, the obtained residue was separated and purified through flash column chromatography, obtaining 22.1 g of a compound 19 (a yield: 90%).

HRMS (70 eV, EI+): m/z calcd for C₄₈H₃₂N₂: 636.2565. found: 636.

Elemental Analysis: C, 91%; H, 5%

### Synthesis Example 26

### Synthesis of Compound 66

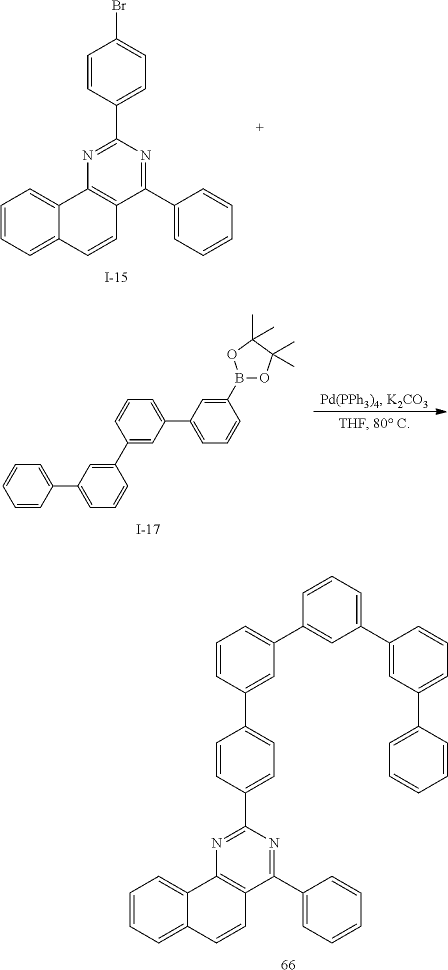

20 g (48.6 mmol) of the intermediate I-15 was dissolved in 0.2 L of tetrahydrofuran (THF) under a nitrogen atmosphere, 21.0 g (48.6 mmol) of the intermediate I-17 and 0.57 g (0.49 mmol) of tetrakis(triphenylphosphine)palladium were added thereto, and the mixture was agitated. 16.8 g (122 mmol) of potassium carbonate saturated in water was added thereto, and the mixture was heated and refluxed at 80° C. for 18 hours. When the reaction was terminated, water was added to the reaction solution, and the mixture was extracted with dichloromethane (DCM) and treated with anhydrous MgSO₄ to remove moisture and then, filtered and concentrated under a reduced pressure. Then, the obtained residue was separated and purified through flash column chromatography, obtaining 27.2 g of a compound 66 (a yield: 88%).

HRMS (70 eV, EI+): m/z calcd for C₄₈H₃₂N₂: 636.2565. found: 636.

Elemental Analysis: C, 91%; H, 5%

### Synthesis Example 27

### Synthesis of Compound 114

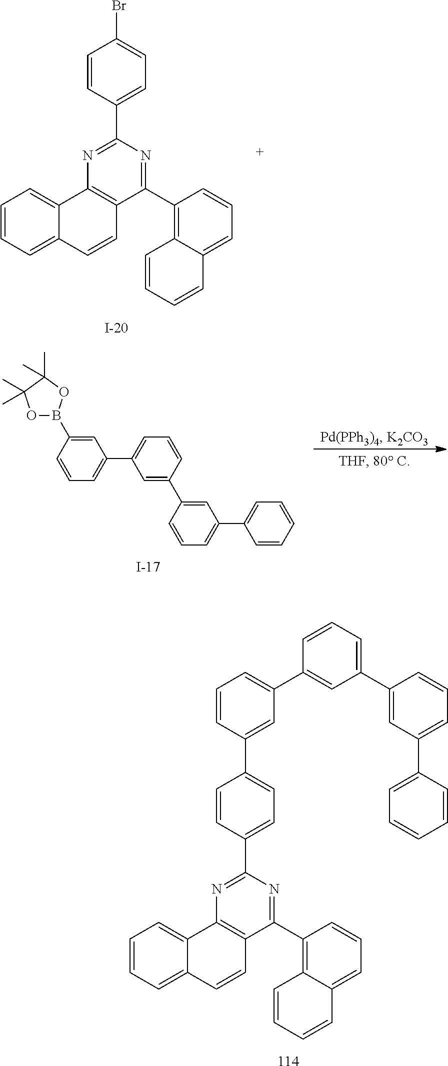

20 g (43.4 mmol) of the intermediate I-20 was dissolved in 0.2 L of tetrahydrofuran (THF) under a nitrogen atmosphere, 18.7 g (43.4 mmol) of the intermediate I-17 and 0.50 g (0.43 mmol) of tetrakis(triphenylphosphine)palladium were added thereto, and the mixture was agitated. 15.0 g (109 mmol) of potassium carbonate saturated in water was added thereto, and the mixture was heated and refluxed at 80° C. for 16 hours. When the reaction was terminated, water was added to the reaction solution, and the mixture was extracted with dichloromethane (DCM) and treated with anhydrous MgSO₄ to remove moisture and then, filtered and concentrated under a reduced pressure. Then, the obtained residue was separated and purified through flash column chromatography, obtaining 23.8 g of a compound 114 (a yield: 80%).

HRMS (70 eV, EI+): m/z calcd for C₅₂H₃₄N₂: 686.2722. found: 686.

Elemental Analysis: C, 91%; H, 5%

### Manufacture of Organic Light Emitting Diode

### Example 1

Compound 1 obtained in Synthesis Example 21 was used as a host, and acetylacetonatobis(2-phenylquinolinato)iridium (Ir(pq)₂acac) was used as a dopant to manufacture an organic light emitting diode.

A 1,500 Å-thick ITO layer was used as an anode, and a 1,000 Å-thick aluminum (Al) layer was used as a cathode. Specifically, an organic light emitting diode was manufactured by manufacturing the anode by cutting an ITO glass substrate having 15 Ω/cm² of sheet resistance into a size of 50 mm×50 mm×0.7 mm and cleaning with an ultrasonic wave in acetone, isopropyl alcohol, and pure water respectively for 15 minutes and with UV ozone for 30 minutes.

On an upper side of a anode, a 600 Å-thick hole injection layer (HIL) was formed by vacuum-depositing 4,4′-bis[N-[4-{N,N-bis(3-methylphenyl)amino}-phenyl]-N-phenylamino]biphenyl [DNTPD] under a vacuum degree of 650×10⁻⁷ Pa at a deposition rate ranging from 0.1 to 0.3 nm/s. Subsequently, a 300 Å-thick hole transport layer (HTL) was formed by vacuum-depositing HT-1 under the same vacuum deposition condition. Then, a 300 Å-thick emission layer was formed by vacuum-depositing Compound 1 of Synthesis Example 21 under the same vacuum deposition condition, and a phosphorescent dopant, acetylacetonatobis (2-phenylquinolinato)iridium (Ir(pq)₂acac) was simultaneously deposited. Herein, 7 wt % of the phosphorescent dopant was deposited by adjusting a deposition rate of the phosphorescent dopant, based on 100 wt % (e.g., of the total weight) of the emission layer.

On the emission layer, a 50 Å-thick hole blocking layer was formed by depositing bis(2-methyl-8-quinolinolate)-4-(phenylphenolato)aluminium (BAlq) under the same vacuum deposition condition. Subsequently, a 250 Å-thick electron transport layer (ETL) was formed by depositing tris(8-hydroxyquinolinato)aluminium (Alq3) under the same vacuum deposition condition. LiF and Al were sequentially deposited to form a cathode on the electron transport layer (ETL), manufacturing an organic light emitting diode.

The organic light emitting diode had a structure of ITO/DNTPD 60 nm/HT-1 30 nm/EML (Compound 1 (93 wt %)+Ir(pq)2acac (7 wt %), 30 nm)/Balq (5 nm)/Alq3 25 nm/LiF (1 nm)/Al (100 nm).

### Example 2

An organic light emitting diode was manufactured according to the same method as Example 1 except for using Compound 2 according to Synthesis Example 22 instead of Compound 1 according to Synthesis Example 21.

### Example 3

An organic light emitting diode was manufactured according to the same method as Example 1 except for using Compound 5 according to Synthesis Example 23 instead of Compound 1 according to Synthesis Example 21.

### Example 4

An organic light emitting diode was manufactured according to the same method as Example 1 except for using Compound 6 according to Synthesis Example 24 instead of Compound 1 according to Synthesis Example 21.

### Example 5

An organic light emitting diode was manufactured according to the same method as Example 1 except for using Compound 19 according to Synthesis Example 25 instead of Compound 1 according to Synthesis Example 21.

### Example 6

An organic light emitting diode was manufactured according to the same method as Example 1 except for using Compound 66 according to Synthesis Example 26 instead of Compound 1 according to Synthesis Example 21.

### Example 7

An organic light emitting diode was manufactured according to the same method as Example 1 except for using Compound 114 according to Synthesis Example 27 instead of Compound 1 according to Synthesis Example 21.

### Comparative Example 1

An organic light emitting diode was manufactured according to the same method as Example 1 except for using 4,4′-di (9H-carbazol-9-yl)biphenyl (CBP) instead of Compound 1 according to Synthesis Example 21.

The structures of DNTPD, BAlq, HT-1, CBP, Ir(pq)₂acac and Alq3 used to manufacture the organic light emitting diode are as follows.

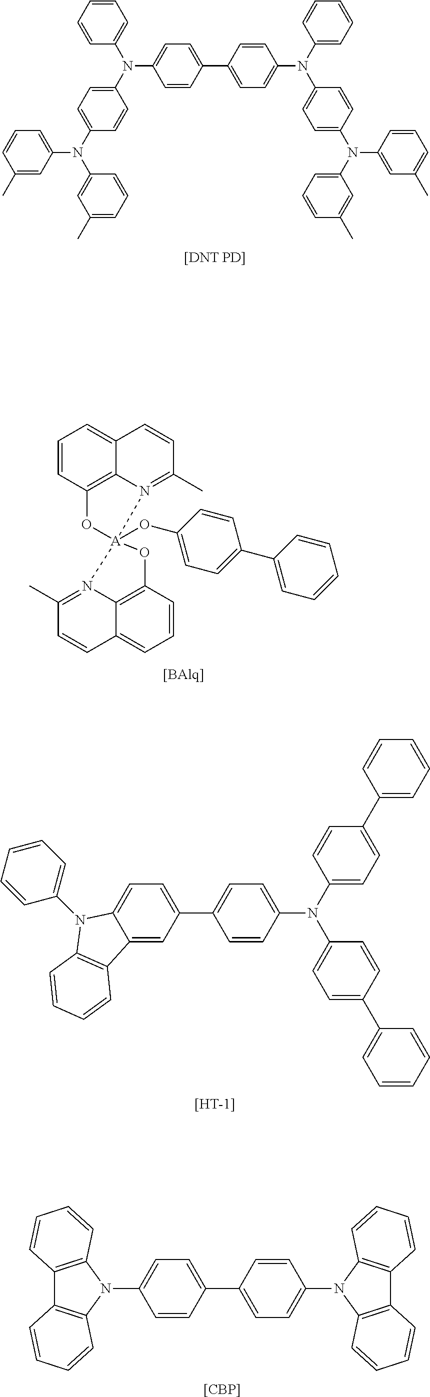

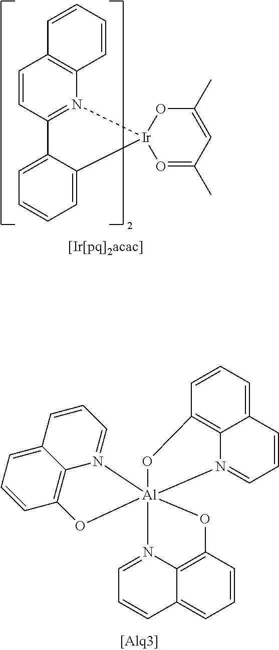

Evaluation

Current density and luminance changes depending on a voltage and luminous efficiency of each organic light emitting diode according to Examples 1 to 7 and Comparative Example 1 were measured.

The measurements were specifically performed according to the following methods, and the results were provided in the following Table 1.

(1) Measurement of Current Density Change Depending on Voltage Change

Current values flowing in the unit device of the manufactured organic light emitting diodes were measured for, while increasing the voltage from 0 V to 10 V using a current-voltage meter (Keithley 2400), and the measured current values were divided by an area to provide the results.

(2) Measurement of Luminance Change Depending on Voltage Change

Luminance of the manufactured organic light emitting diodes was measured for luminance, while increasing the voltage from 0 V to 10 V using a luminance meter (Minolta Cs-1000 A).

(3) Measurement of Luminous Efficiency

Current efficiency (cd/A) at the same current density (10 mA/cm²) were calculated by using the luminance, current density, and voltages (V) from the items (1) and (2).

(4) Measurement of Life-Span

Life-span of the manufactured organic light emitting diodes was obtained by emitting light with 3,000 cd/m² in the initial luminance (cd/m²), measuring luminance decrease as time goes and measuring time taken until the luminance decreased by 90% relative to the initial luminance.

TABLE 1

| | | Driving | Color | Effi- | 90% life- |
|---|---|---|---|---|---|
| | | voltage | (EL | ciency | span (h) at |
| Nos. | Compound | (V) | color) | (cd/A) | 3000 cd/m² |
| Example 1 | Compound 1 | 7.0 | Red | 48.4 | 75 |
| Example 2 | Compound 2 | 6.9 | Red | 46.5 | 100 |
| Example 3 | Compound 5 | 7.1 | Red | 47.2 | 72 |
| Example 4 | Compound 6 | 7.0 | Red | 47.0 | 97 |
| Example 5 | Compound 19 | 6.9 | Red | 49.1 | 76 |
| Example 6 | Compound 66 | 7.0 | Red | 47.9 | 60 |
| Example 7 | Compound 114 | 7.1 | Red | 45.5 | 81 |
| Comparative | CBP | 7.4 | Red | 37.2 | 50 |
| Example 1 | | | | | |

Referring to Table 1, the organic light emitting diodes according to Examples 1 to 7 showed remarkably improved luminous efficiency and life-span characteristics, compared with the organic light emitting diode according to Comparative Example 1.

### Synthesis Examples of Second Host Compound

### Synthesis Example 1 of Second Host Compound

### Synthesis of Intermediate I-30

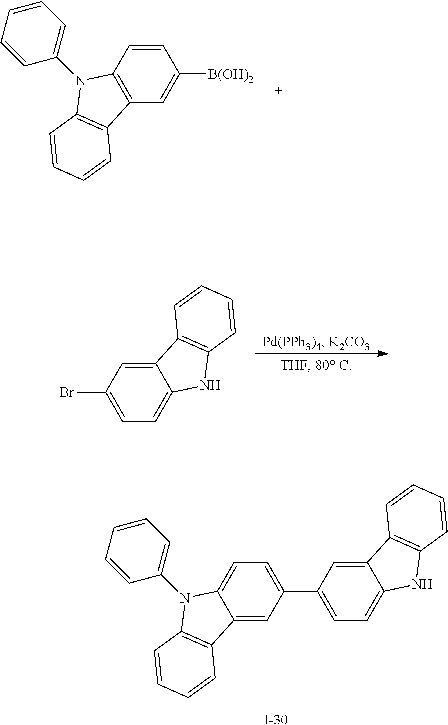

100 g (348 mmol) of 9-phenyl-9H-carbazol-3-ylboronic acid was dissolved in 0.93 L of tetrahydrofuran (THF) under a nitrogen atmosphere, 85.6 g (348 mmol) of 3-bromo-9H-carbazole and 4.02 g (3.48 mmol) of tetrakis(triphenylphosphine)palladium were added thereto, and the mixture was agitated. Then, 120 g (870 mmol) of potassium carbonate was added thereto, and the mixture was heated and refluxed at 80° C. for 10 hours. When the reaction was terminated, water was added to the reaction solution, and the mixture was extracted with dichloromethane (DCM) and treated with anhydrous MgSO₄ to remove moisture and then, filtered and concentrated under a reduced pressure. Then, the obtained residue was separated and purified through flash column chromatography, obtaining 85.3 g of a compound I-30 (a yield: 60%).

HRMS (70 eV, EI+): m/z calcd for C₃₀H₂₀N₂: 408.1626. found: 408.

Elemental Analysis: C, 88%; H, 5%

### Synthesis Example 2 of Second Host Compound

### Synthesis of Intermediate I-31

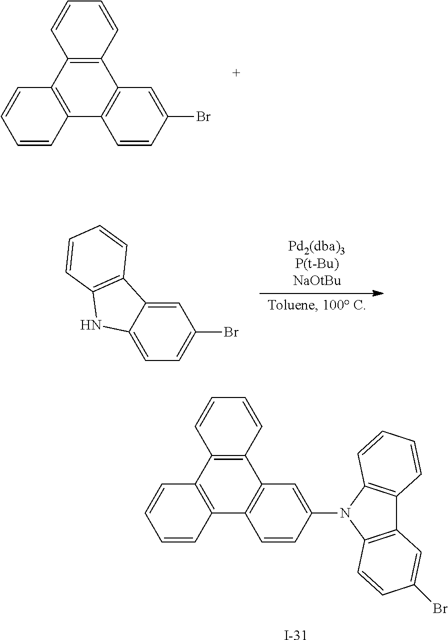

100 g (326 mmol) of 2-bromotriphenylene was dissolved in 0.18 L of toluene under a nitrogen atmosphere, 80.1 g (326 mmol) of 3-bromo-9H-carbazole, 2.99 g (3.26 mmol) of tris(diphenylideneacetone)dipalladium (0), 2.64 g (13.0 mmol) of tris-tert-butylphosphine and 37.6 g (391 mmol) of sodium tert-butoxide were sequentially added thereto, and the mixture was heated and refluxed at 100° C. for 15 hours. When the reaction was terminated, water was added to the reaction solution, and the mixture was extracted with dichloromethane (DCM) and treated with anhydrous MgSO₄ to remove moisture and then, filtered and concentrated under a reduced pressure. Then, the obtained residue was separated and purified through flash column chromatography, obtaining 109 g of a compound I-31 (a yield: 71%).

HRMS (70 eV, EI+): m/z calcd for C₃₀H₁₈BrN: 471.0623. found: 471.

Elemental Analysis: C, 76%; H, 4%

### Synthesis Example 3 of Second Host Compound

### Synthesis of Intermediate I-32

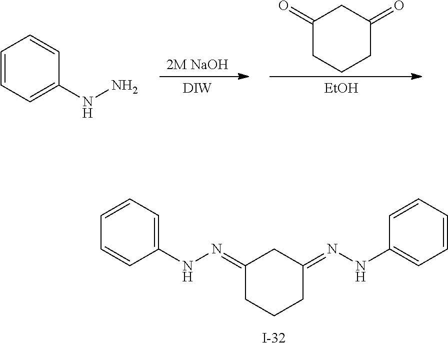

100 g (925 mmol) of phenylhydrazine hydrochloride was dissolved in 0.5 L of distilled water under a nitrogen atmosphere, and a 2M NaOH aqueous solution was added thereto. A solid produced therein was filtered, obtaining phenylhydrazine. Then, the obtained phenylhydrazine was slowly added to the solution that 51.8 g (462 mmol) of cyclohexane-1,3-dione was dissolved in 1.0 L of ethanol, and the mixture was reacted for 20 minutes. When the reaction was terminated, ice water was added thereto. Then, a solid produced therein was filtered while washed with ethanol. The filtered product was dried under a reduced pressure, obtaining 108 g of a compound I-32 (a yield: 40%).

HRMS (70 eV, EI+): m/z calcd for C₁₈H₂₀N₄: 292.1688. found: 292.

Elemental Analysis: C, 74%; H, 7%

### Synthesis Example 4 of Second Host Compound

### Synthesis of Intermediate I-33

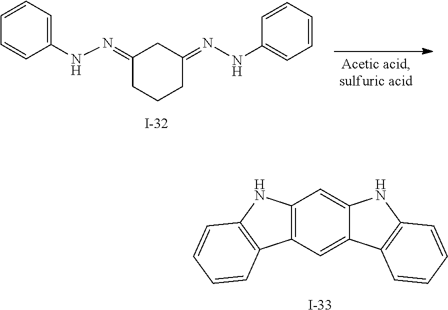

100 g (342 mmol) of the compound I-32 was slowly added to 0.5 L of a mixed solution of acetic acid and sulfuric acid in a volume ratio of 1:4 under a nitrogen atmosphere at 0° C. The mixture was agitated for 5 minutes and then, rapidly heated up to 50° C. and slowly heated up to 110° C. Twenty minutes later, the resultant was cooled down to ambient temperature and agitated for 12 hours. 0.5 L of ethanol was added thereto, and a solid produced one hour later was filtered under a reduced pressure and neutralized. The solid was dried under a reduced pressure, obtaining 43.8 g of the compound I-33 (a yield: 50%).

HRMS (70 eV, EI+): m/z calcd for C₁₈H₁₂N₂: 256.1000. found: 256.

Elemental Analysis: C, 84%; H, 5%

### Synthesis Example 5 of Second Host Compound

### Synthesis of Compound C-10

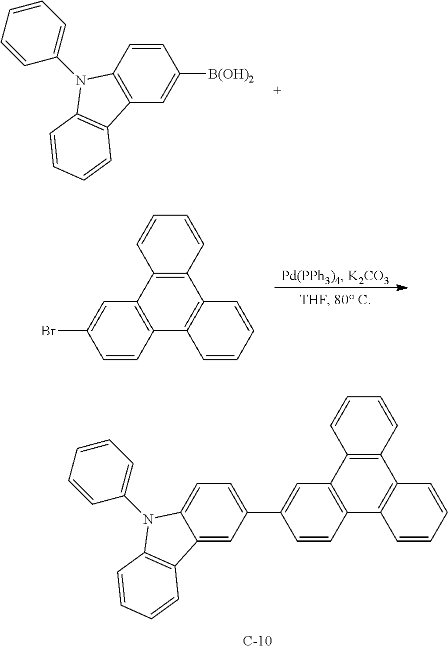

20 g (69.7 mmol) of 9-phenyl-9H-carbazol-3-ylboronic acid was dissolved in 0.21 L of tetrahydrofuran (THF) under a nitrogen atmosphere, 21.4 g (69.7 mmol) of 2-bromotriphenylene and 0.81 g (0.70 mmol) of tetrakis(triphenylphosphine)palladium were added thereto, and the mixture was agitated. Then, 24.1 g (174 mmol) of potassium carbonate saturated in water was added thereto, and the mixture was heated and refluxed at 80° C. for 8 hours. When the reaction was terminated, water was added to the reaction solution, and the mixture was extracted with dichloromethane (DCM) and treated with anhydrous MgSO₄ to remove moisture and then, filtered and concentrated under a reduced pressure. Then, the obtained residue was separated and purified through flash column chromatography, obtaining 29.5 g of a compound C-10 (a yield: 90%).

HRMS (70 eV, EI+): m/z calcd for C₃₆H₂₃N: 469.1830. found: 469.

Elemental Analysis: C, 92%; H, 5%

### Synthesis Example 6 of Second Host Compound

### Synthesis of Compound B-10

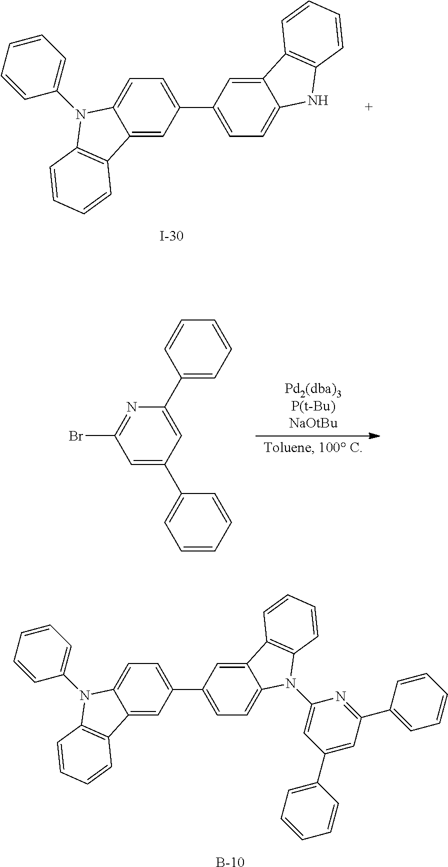

20 g (49.0 mmol) of the intermediate I-30 was dissolved in 0.18 L of toluene under a nitrogen atmosphere, 15.1 g (49.0 mmol) of 2-bromo-4,6-diphenylpyridine made by Amadis Chemical, 0.45 g (0.49 mmol) of tris(diphenylideneacetone)dipalladium (0), 0.40 g (1.96 mmol) of tris-tert-butylphosphine and 5.65 g (58.8 mmol) of sodium tert-butoxide were sequentially added thereto, and the mixture was heated and refluxed at 100° C. for 18 hours. When the reaction was terminated, water was added to the reaction solution, and the mixture was extracted with dichloromethane (DCM) and treated with anhydrous MgSO₄ to remove moisture and then, filtered and concentrated under a reduced pressure. Then, the obtained residue was separated and purified through flash column chromatography, obtaining 31.3 g (77%) of a compound B-10.

HRMS (70 eV, EI+): m/z calcd for C₄₇H₃₁N₃: 637.2518. found: 637.

Elemental Analysis: C, 89%; H, 5%

### Synthesis Example 7 of Second Host Compound

### Synthesis of Compound B-31

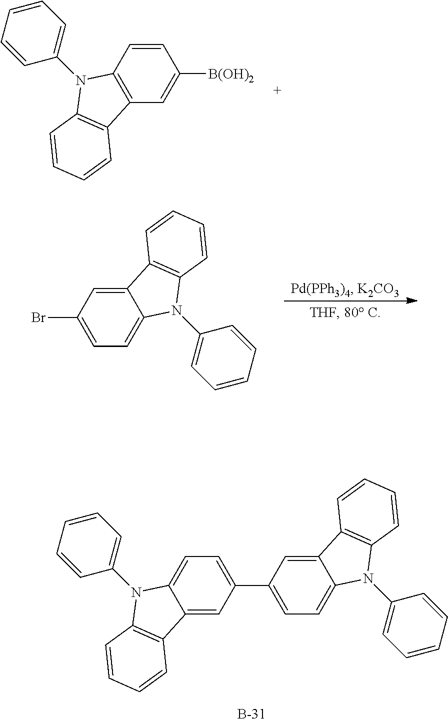

20 g (69.7 mmol) of 9-phenyl-9H-carbazol-3-ylboronic acid was dissolved in 0.21 L of tetrahydrofuran (THF) under a nitrogen atmosphere, 22.5 g (69.7 mmol) of 3-bromo-9-phenyl-9H-carbazole and 0.81 g (0.70 mmol) of tetrakis(triphenylphosphine)palladium were added thereto, and the mixture was agitated. Then, 24.1 g (174 mmol) of potassium carbonate saturated in water was heated and refluxed at 80° C. for 12 hours. When the reaction was terminated, water was added to the reaction solution, and the mixture was extracted with dichloromethane (DCM) and treated with anhydrous MgSO₄ to remove moisture and then, filtered and concentrated under a reduced pressure. Then, the obtained residue was separated and purified through flash column chromatography, obtaining 31.1 g of a compound B-31 (a yield: 92%).

HRMS (70 eV, EI+): m/z calcd for C₃₆H₂₄N₂: 484.1939. found: 484.

Elemental Analysis: C, 89%; 5%

### Synthesis Example 8 of Second Host Compound

### Synthesis of Compound B-34

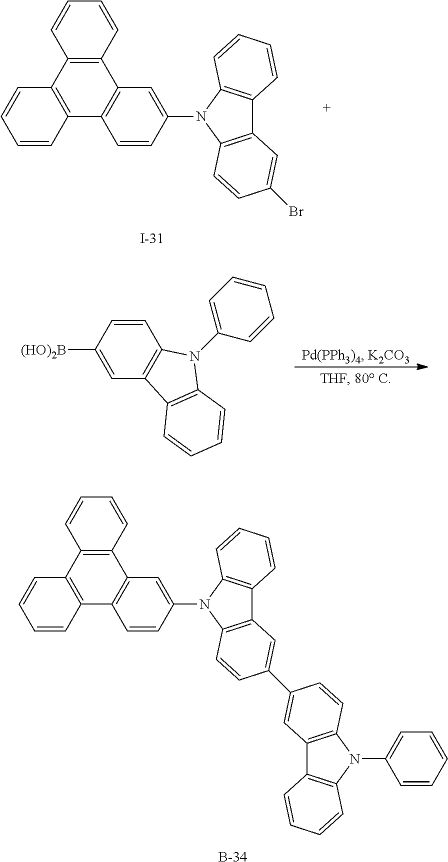

20 g (42.3 mmol) of the intermediate I-31 was dissolved in 0.16 L of tetrahydrofuran (THF) under a nitrogen atmosphere, 12.1 g (42.3 mmol) of 9-phenyl-9H-carbazol-3-ylboronic acid and 0.49 g (0.42 mmol) of tetrakis(triphenylphosphine)palladium were added thereto, and the mixture was agitated. Then, 14.7 g (106 mmol) of potassium carbonate saturated in water was added thereto, and the mixture was heated and refluxed at 80° C. for 16 hours. When the reaction was terminated, water was added to the reaction solution, and the mixture was extracted with dichloromethane (DCM) and treated with anhydrous MgSO₄ to remove moisture and then, filtered and concentrated under a reduced pressure. Then, the obtained residue was separated and purified through flash column chromatography, obtaining 22.8 g (85%) of a compound B-34.

HRMS (70 eV, EI+): m/z calcd for C₄₈H₃₀N₂: 634.2409. found: 634.

Elemental Analysis: C, 91%; H, 5%

### Synthesis Example 9 of Second Host Compound

### Synthesis of Compound B-43

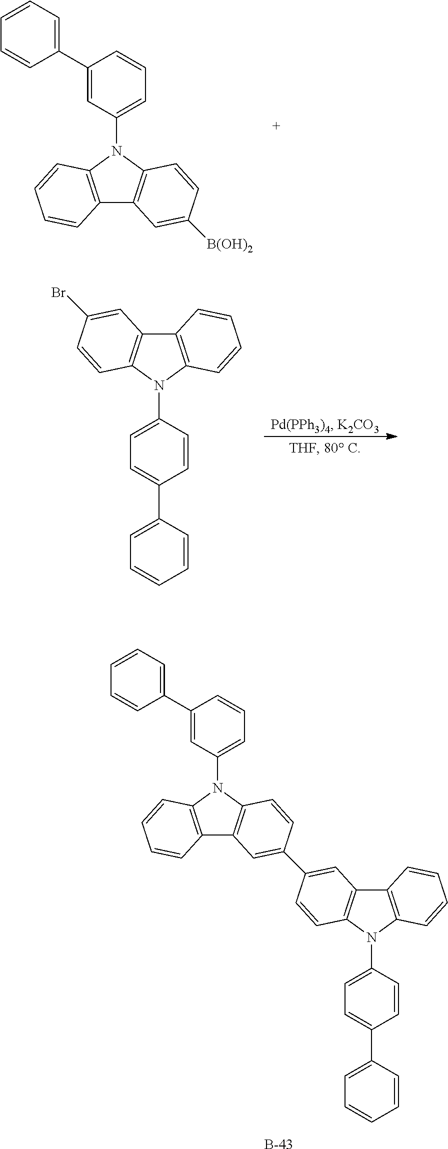

20 g (55.1 mmol) of 9-(biphenyl-3-yl)-9H-carbazol-3-ylboronic acid made by Amadis Chemical was dissolved in 0.21 L of tetrahydrofuran (THF) under a nitrogen atmosphere, 21.9 g (55.1 mmol) of 9-(biphenyl-4-yl)-3-bromo-9H-carbazole made by Amadis Chemical and 0.64 g (0.55 mmol) of tetrakis(triphenylphosphine)palladium were added thereto, and the mixture was agitated. Then, 19.0 g (138 mmol) of potassium carbonate saturated in water was added thereto, and the mixture was heated and refluxed at 80° C. for 18 hours. When the reaction was terminated, water was added to the reaction solution, and the mixture was extracted with dichloromethane (DCM) and treated with anhydrous MgSO₄ to remove moisture and then, filtered and concentrated under a reduced pressure. Then, the obtained residue was separated and purified through flash column chromatography, obtaining 33.7 g of a compound B-43 (a yield: 96%).

HRMS (70 eV, EI+): m/z calcd for C₄₈H₃₂N₂: 636.2565. found: 636.

Elemental Analysis: C, 91%; H, 5%

### Synthesis Example 10 of Second Host Compound

### Synthesis of Compound E-1

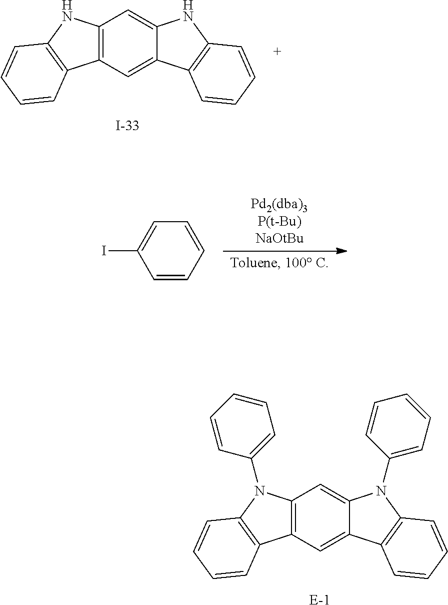

20 g (78.0 mmol) of the intermediate I-33 was dissolved in 0.26 L of toluene under a nitrogen atmosphere, 31.8 g (156 mmol) of iodobenzene, 0.71 g (0.78 mmol) of tris(diphenylideneacetone)dipalladium (0), 0.63 g (3.12 mmol) of tris-tert-butylphosphine and 8.99 g (93.6 mmol) of sodium tert-butoxide were sequentially added thereto, and the mixture was heated and refluxed at 100° C. for 10 hours. When the reaction was terminated, water was added to the reaction solution, and the mixture was extracted with dichloromethane (DCM) and treated with anhydrous MgSO₄ to remove moisture and then, filtered and concentrated under a reduced pressure. Then, the obtained residue was separated and purified through flash column chromatography, obtaining 25.5 g of a compound E-1 (a yield: 80%).

HRMS (70 eV, EI+): m/z calcd for C₃₀H₂₀N₂: 408.1626. found: 408.

Elemental Analysis: C, 88%; 14, 5%

### Manufacture of Organic Light Emitting Diode Using Second Host

### Example 8

A 1,500 Å-thick ITO layer was used as an anode, and a 1,000 Å-thick aluminum (Al) layer was used as a cathode. Specifically, an organic light emitting diode was manufactured by manufacturing the anode by cutting an ITO glass substrate having 15 Ω/cm² of sheet resistance into a size of 50 mm×50 mm×0.7 mm and cleaning with an ultrasonic wave in acetone, isopropyl alcohol, and pure water respectively for 15 minutes and with UV ozone for 30 minutes.

On the upper side of a anode, a 600 Å-thick hole injection layer (HIL) was formed by vacuum-depositing 4,4′-bis[N-[4-{N,N-bis(3-methylphenyl)amino}-phenyl]-N-phenylamino]biphenyl [DNTPD] under a vacuum degree of 650×10⁻⁷ Pa at a deposition rate ranging from 0.1 to 0.3 nm/s. Subsequently, a 300 Å-thick hole transport layer (HTL) was formed by vacuum-depositing HT-1 under the same vacuum deposition condition. Then, a 300 Å-thick emission layer was formed by vacuum-depositing Compound 1 of Synthesis Example 21 and a second host, Compound C-10 obtained in Synthesis Example 5 under the same vacuum deposition condition. Compound 1 and Compound C-10 were used in a weight ratio of 4:1. When depositing the hosts, a phosphorescent dopant, acetylacetonatobis(2-phenylquinolinato)iridium (Ir(pq)₂acac) was simultaneously deposited. Herein, 7 wt % of the phosphorescent dopant was deposited by adjusting a deposition rate of the phosphorescent dopant, based on 100 wt % (e.g., of the total weight) of the emission layer.

On the emission layer, a 50 Å-thick hole blocking layer was formed by depositing bis(2-methyl-8-quinolinolate)-4-(phenylphenolato)aluminium (BAlq) under the same vacuum deposition condition. Subsequently, a 250 Å-thick electron transport layer (ETL) was formed by depositing tris(8-hydroxyquinolinato)aluminium (Alq3) under the same vacuum deposition condition. LiF and Al were sequentially deposited to form a cathode on the electron transport layer (ETL), manufacturing an organic light emitting diode.

The organic light emitting diode had a structure of ITO/DNTPD (60 nm)/HT-1 (30 nm)/EML (Compounds 1:C-10=4:1, 93 wt %)+Ir(pq)₂acac (7 wt %), 30 nm)/Balq (5 nm)/Alq3 (25 nm)/LiF (1 nm)/Al (100 nm).

### Example 9

An organic light emitting diode was manufactured according to the same method as Example 8 except for using Compounds 1 and C-10 in a ratio of 1:1.

### Example 10

An organic light emitting diode was manufactured according to the same method as Example 8 except for using Compounds 1 and C-10 in a ratio of 1:4.

### Example 11

An organic light emitting diode was manufactured according to the same method as Example 8 except for using Compound B-10 instead of Compound C-10.

### Example 12

An organic light emitting diode was manufactured according to the same method as Example 11 except for using Compounds 1 and B-10 in a ratio of 1:1.

### Example 13

An organic light emitting diode was manufactured according to the same method as Example 11 except for using Compounds 1 and B-10 in a ratio of 1:4.

### Example 14

An organic light emitting diode was manufactured according to the same method as Example 8 except for using Compound B-31 instead of Compound C-10.

### Example 15

An organic light emitting diode was manufactured according to the same method as Example 14 except for using Compounds 1 and B-31 in a ratio of 1:1.

### Example 16

An organic light emitting diode was manufactured according to the same method as Example 8 except for using Compound B-34 instead of Compound C-10.

### Example 17

An organic light emitting diode was manufactured according to the same method as Example 16 except for using Compounds 1 and B-34 in a ratio of 1:1.

### Example 18

An organic light emitting diode was manufactured according to the same method as Example 8 except for using Compound B-43 instead of Compound C-10.

### Example 19

An organic light emitting diode was manufactured according to the same method as Example 8 except for using Compound E-1 instead of Compound C-10.

### Example 20

An organic light emitting diode was manufactured according to the same method as Example 8 except for using Compound 1 as a single host, instead of including two hosts of Compounds 1 and C-10.

### Comparative Example 2

An organic light emitting diode was manufactured according to the same method as Example 8 except for using CBP as a host instead of two hosts of Compounds 1 and C-10.

### Comparative Example 3

An organic light emitting diode was manufactured according to the same method as Example 8 except for using Compound C-10 as a single host instead of two hosts of Compounds 1 and C-10.

### Comparative Example 4

An organic light emitting diode was manufactured according to the same method as Example 8 except for using Compound B-10 as a single host instead of two hosts of Compounds 1 and C-10.

### Comparative Example 5

An organic light emitting diode was manufactured according to the same method as Example 8 except for using Compound B-31 as a single host instead of two hosts of Compounds 1 and C-10.

### Comparative Example 6

An organic light emitting diode was manufactured according to the same method as Example 8 except for using Compound B-34 as a single host instead of two hosts of Compounds 1 and C-10.

### Comparative Example 7

An organic light emitting diode was manufactured according to the same method as Example 8 except for using Compound B-43 as a single host instead of two hosts of Compounds 1 and C-10.

### Comparative Example 8

An organic light emitting diode was manufactured according to the same method as Example 8 except for using Compound E-1 as a single host instead of two hosts of Compounds 1 and C-10.

Evaluation

Current density and luminance changes depending on a voltage and luminous efficiency of each organic light emitting diode according to Examples 8 to 20 and Comparative Examples 2 to 8 were measured.

The measurements were specifically performed according the following methods, and the results were provided in the following Table 2.

(1) Measurement of Current Density Change Depending on Voltage Change

Current values flowing in the unit device of the manufactured organic light emitting diodes were measured for, while increasing the voltage from 0 V to 10 V using a current-voltage meter (Keithley 2400), and the measured current values were divided by an area to provide the results.

(2) Measurement of Luminance Change Depending on Voltage Change

Luminance of the manufactured organic light emitting diodes was measured for luminance, while increasing the voltage from 0 V to 10 V using a luminance meter (Minolta Cs-1000 A).

(3) Measurement of Luminous Efficiency

Current efficiency (cd/A) at the same current density (10 mA/cm²) were calculated by using the luminance, current density, and voltages (V) from the items (1) and (2).

(4) Measurement of Life-Span

Life-span of the organic light emitting diodes was obtained by emitting light with 3,000 cd/m² in the initial luminance (cd/m²) and measuring time taken until luminance decreased by 50% relative to the initial luminance as time goes.

TABLE 2

| | | | First | Effi- | 50% life- |
|---|---|---|---|---|---|
| | First | Second | host:sec- | ciency | span (h) at |
| | host | host | ond host | (cd/A) | 3000 cd/m² |
| Example 8 | Compound 1 | C-10 | 4:1 | 49.5 | 480 |
| Example 9 | Compound 1 | C-10 | 1:1 | 50.0 | 510 |
| Example 10 | Compound 1 | C-10 | 1:4 | 43.6 | 470 |
| Example 11 | Compound 1 | B-10 | 4:1 | 48.4 | 280 |
| Example 12 | Compound 1 | B-10 | 1:1 | 48.1 | 330 |
| Example 13 | Compound 1 | B-10 | 1:4 | 45.6 | 250 |
| Example 14 | Compound 1 | B-31 | 4:1 | 48.2 | 285 |
| Example 15 | Compound 1 | B-31 | 1:1 | 49.2 | 310 |
| Example 16 | Compound 1 | B-34 | 4:1 | 47.5 | 465 |
| Example 17 | Compound 1 | B-34 | 1:1 | 48.7 | 500 |
| Example 18 | Compound 1 | B-43 | 4:1 | 49.0 | 300 |
| Example 19 | Compound 1 | E-1 | 4:1 | 49.1 | 320 |
| Example 20 | Compound 1 | — | — | 48.4 | 250 |

| | | | | |
|---|---|---|---|---|
| Comparative | CBP | — | 37.2 | 220 |
| Example 2 | | | | |
| Comparative | C-10 | — | 10 | 30 |
| Example 3 | | | | |
| Comparative | B-10 | — | 10 | 30 |
| Example 4 | | | | |
| Comparative | B-31 | — | 5 | 0 |
| Example 5 | | | | |
| Comparative | B-34 | — | 20.1 | 10 |
| Example 6 | | | | |
| Comparative | B-43 | — | 10 | 30 |
| Example 7 | | | | |
| Comparative | E-1 | — | 5 | 50 |
| Example 8 | | | | |

Referring to Table 2, the organic light emitting diodes according to Examples 8 to 20 showed remarkably improved luminous efficiency and life-span characteristics, compared with the organic light emitting diodes according to Comparative Examples 2 to 8.

By way of summation and review, performance of an organic light emitting diode may be affected by characteristics of the organic layer, e.g., may be mainly affected by characteristics of an organic material of the organic layer. For example, an organic material capable of increasing hole and electron mobility and simultaneously increasing electrochemical stability may be used so that the organic light emitting diode may be applied to a large-size flat panel display.

The embodiments may provide an organic compound that is capable of realizing an organic optoelectric device having high efficiency and long life-span.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

### DESCRIPTION OF SYMBOLS

100, 200: organic light emitting diode
105: organic layer
110: cathode
120: anode
130: emission layer
140: hole auxiliary layer

What is claimed is:

1. An organic compound represented by the following Chemical Formula 1: 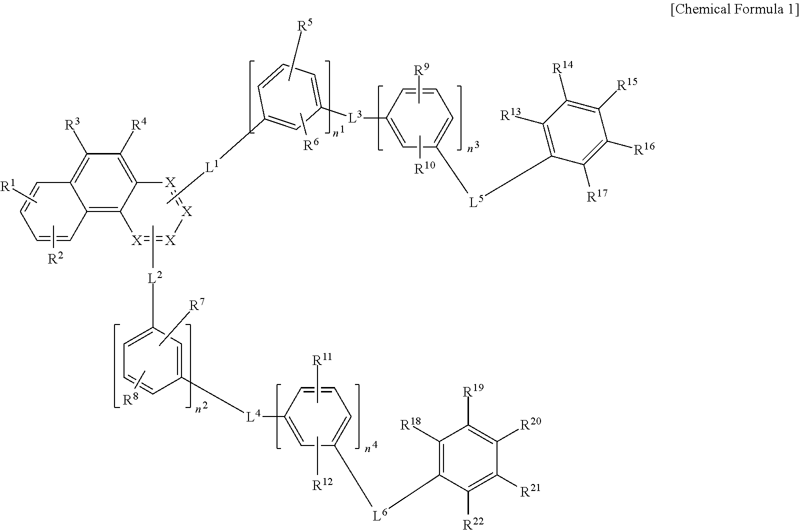
wherein, in Chemical Formula 1,
two of X are nitrogen and two of X are carbon,
R¹ to R⁴ are each independently hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof,
R⁵ to R¹² are each independently hydrogen, deuterium, a substituted or unsubstituted C1 to C10 alkyl group, a substituted or unsubstituted C6 to C12 aryl group, or a combination thereof,
R¹³ to R²² are each independently hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof, or two adjacent ones thereof are linked to each other to form a fused ring,
L¹ to L⁶ are each independently a single bond, a substituted or unsubstituted phenylene group, a substituted or unsubstituted biphenylene group, a substituted or unsubstituted terphenylene group, or a substituted or unsubstituted quaterphenylene group,
n¹ to n⁴ are each independently an integer of 0 to 5, and
a sum of n¹ to n⁴ is an integer of 2 or more.

2. The organic compound as claimed in claim 1, wherein the compound represented by Chemical Formula 1 is represented by one of the following Chemical Formula 2 or Chemical Formula 3: 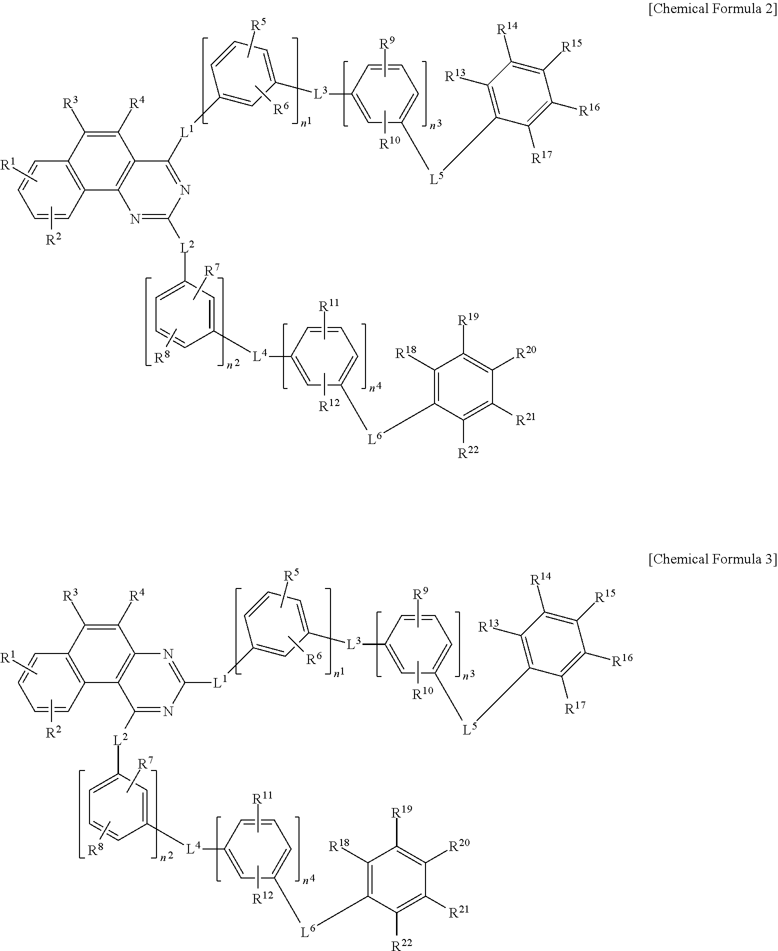
wherein, in Chemical Formulae 2 and 3,
R¹ to R⁴ are each independently hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof,
R⁵ to R¹² are each independently hydrogen, deuterium, a substituted or unsubstituted C1 to C10 alkyl group, a substituted or unsubstituted C6 to C12 aryl group, or a combination thereof,
R¹³ to R²² are each independently hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof, or two adjacent ones thereof are linked to each other to form a fused ring,
L¹ to L⁶ are each independently a single bond, a substituted or unsubstituted phenylene group, a substituted or unsubstituted biphenylene group, a substituted or unsubstituted terphenylene group, or a substituted or unsubstituted quaterphenylene group,
n¹ to n⁴ are each independently an integer of 0 to 5, and
a sum of n¹ to n⁴ is an integer of 2 or more.

3. The organic compound as claimed in claim 2, wherein the compound represented by Chemical Formula 1 is represented by one of the following Chemical Formula 2A or Chemical Formula 3A: 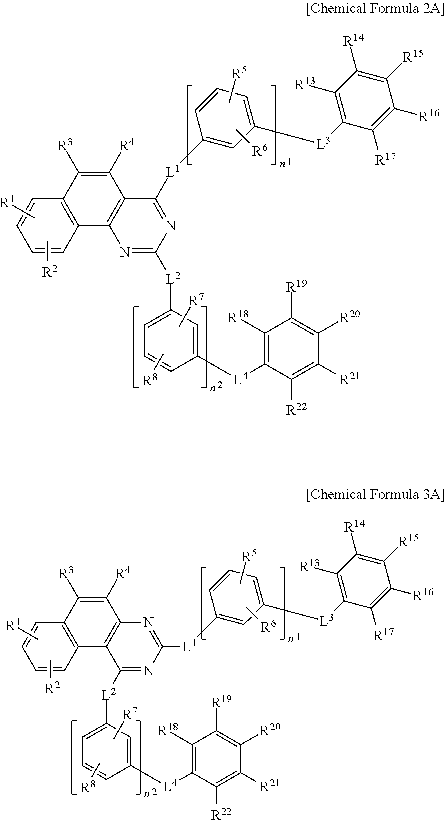
wherein, in Chemical Formulae 2A and 3A,
R¹ to R⁴ are each independently hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof,
R⁵ to R⁸ are each independently hydrogen, deuterium, a substituted or unsubstituted C1 to C10 alkyl group, a substituted or unsubstituted C6 to C12 aryl group, or a combination thereof,
R¹³ to R²² are each independently hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof, or two adjacent ones thereof are linked to each other to form a fused ring,
L¹ to L⁴ are each independently a single bond, a substituted or unsubstituted phenylene group, a substituted or unsubstituted biphenylene group, a substituted or unsubstituted terphenylene group, or a substituted or unsubstituted quaterphenylene group,
n¹ and n² are each independently an integer of 0 to 5, and
a sum of n¹ and n² is an integer of 2 or more.

4. The organic compound as claimed in claim 1, wherein n¹ and n² of Chemical Formula 1 are each independently an integer of 1 to 5.

5. The organic compound as claimed in claim 1, wherein R³ and R⁴ of Chemical Formula 1 are hydrogen.

6. The organic compound as claimed in claim 1, wherein L¹ to L⁶ of Chemical Formula 1 are each independently a single bond or a group of the following Group 1: 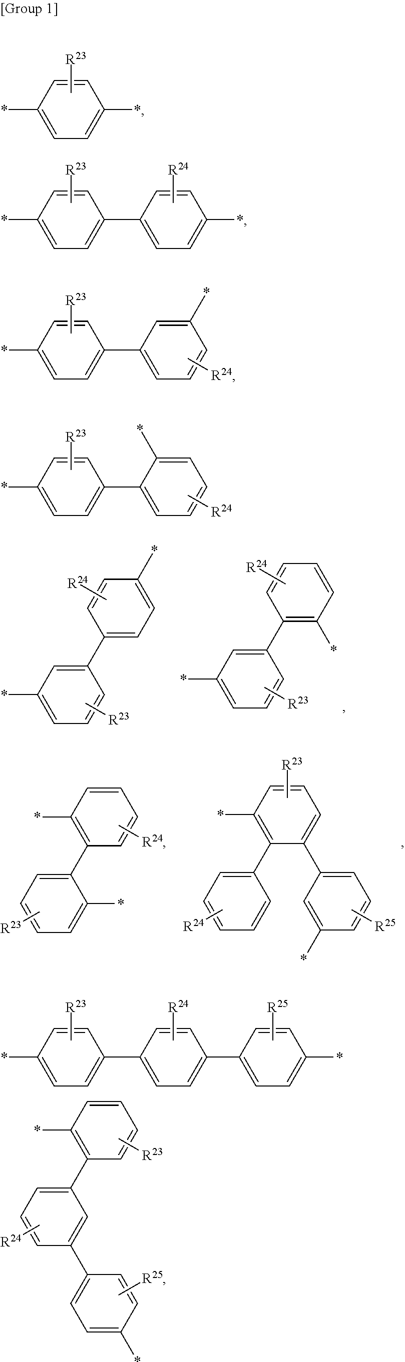 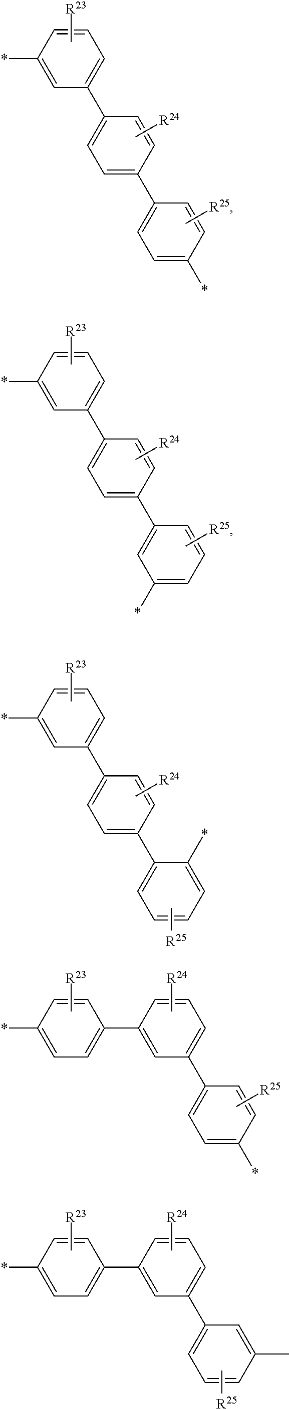 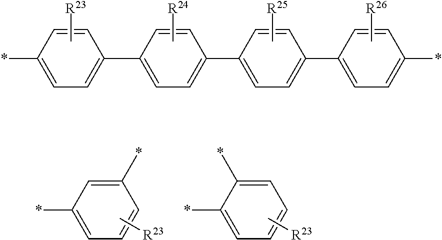
wherein, in Group 1,
R²³ to R²⁶ are each independently hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof, and
* is a linking point to a neighboring atom.

7. The organic compound as claimed in claim 6, wherein R²³ to R²⁶ are hydrogen.

8. A composition for an organic optoelectric device, the composition comprising:
the organic compound as claimed in claim 1, and
a second organic compound that includes a carbazole moiety.

9. The composition for an organic optoelectric device as claimed in claim 8, wherein the second organic compound includes at least one of a compound represented by the following Chemical Formula 4 or a compound consisting of a combination of a moiety represented by the following Chemical Formula 5 and a moiety represented by the following Chemical Formula 6: 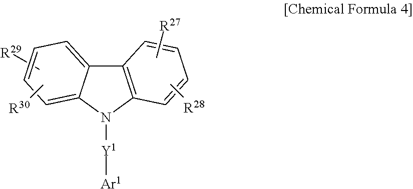
wherein, in Chemical Formula 4,
Y¹ is a single bond, a substituted or unsubstituted C1 to C20 alkylene group, a substituted or unsubstituted C2 to C20 alkenylene group, a substituted or unsubstituted C6 to C30 arylene group, a substituted or unsubstituted C2 to C30 heterocyclic group, or a combination thereof,
Ar¹ is a substituted or unsubstituted C6 to C30 aryl group, a substituted or unsubstituted C2 to C30 heterocyclic group, or a combination thereof,
R²⁷ to R³⁰ are each independently hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof, and
at least one of R²⁷ to R³⁰ and Ar¹ includes a substituted or unsubstituted triphenylene group or a substituted or unsubstituted carbazole group, 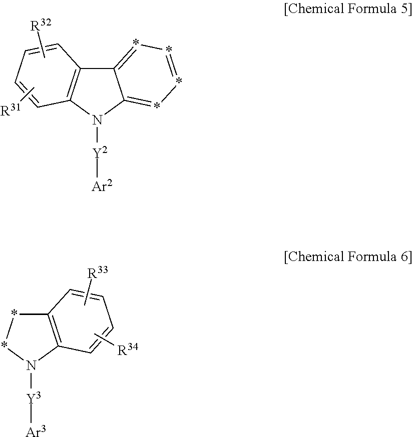
wherein, in Chemical Formulae 5 and 6,
Y² and Y³ are each independently a single bond, a substituted or unsubstituted C1 to C20 alkylene group, a substituted or unsubstituted C2 to C20 alkenylene group, a substituted or unsubstituted C6 to C30 arylene group, a substituted or unsubstituted C2 to C30 heterocyclic group, or a combination thereof,
Ar² and Ar^{a} are each independently substituted or unsubstituted C6 to C30 aryl group, a substituted or unsubstituted C2 to C30 heterocyclic group, or a combination thereof,
R³¹ to R³⁴ are each independently hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof,
adjacent two *s of Chemical Formula 5 are combined with two *s of Chemical Formula 6 to form a fused ring, and *s of Chemical Formula 5 that do not form a fused ring are each independently CR^{a}, and
R^{a} is hydrogen, deuterium, a substituted or unsubstituted C1 to C10 alkyl group, a substituted or unsubstituted C6 to C12 aryl group, a substituted or unsubstituted C3 to C12 heterocyclic group, or a combination thereof.

10. The composition for an organic optoelectric device as claimed in claim 9, wherein the compound represented by Chemical Formula 4 is represented by one of the following Chemical Formulae 4-I to 4-III: 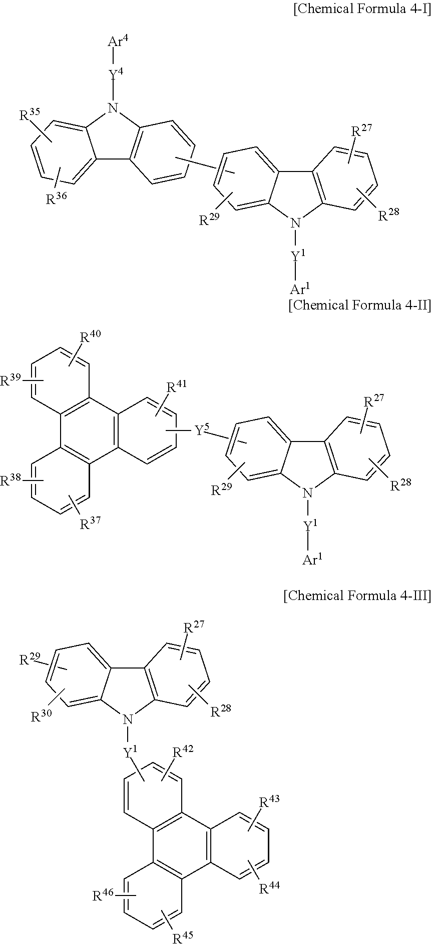
wherein, in Chemical Formulae 4-I to 4-III,
Y¹, Y⁴, and Y⁵ are each independently a single bond, a substituted or unsubstituted C1 to C20 alkylene group, a substituted or unsubstituted C2 to C20 alkenylene group, a substituted or unsubstituted C6 to C30 arylene group, a substituted or unsubstituted C2 to C30 heterocyclic group, or a combination thereof,
Ar¹ and Ar⁴ are each independently a substituted or unsubstituted C6 to C30 aryl group, a substituted or unsubstituted C2 to C30 heterocyclic group, or a combination thereof, and
R²⁷ to R³⁰ and R³⁵ to R⁴⁶ are each independently hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof.

11. The composition for an organic optoelectric device as claimed in claim 8, wherein the first organic compound and the second organic compound are included in the composition in a weight ratio of about 1:10 to about 10:1.

12. The composition for an organic optoelectric device as claimed in claim 8, further comprising a phosphorescent dopant.

13. An organic optoelectric device, comprising:
an anode and a cathode facing each other, and
at least one organic layer between the anode and the cathode,
wherein the organic layer includes the organic compound as claimed in claim 1.

14. The organic optoelectric device as claimed in claim 13, wherein:
the organic layer includes an emission layer, and
the emission layer includes the organic compound.

15. The organic optoelectric device as claimed in claim 14, wherein the organic compound is a host in the emission layer.

16. A display device comprising the organic optoelectric device as claimed in claim 13.

17. An organic optoelectric device, comprising:
an anode and a cathode facing each other, and
at least one organic layer between the anode and the cathode,
wherein the organic layer includes the composition as claimed in claim 8.

18. The organic optoelectric device as claimed in claim 17, wherein:
the organic layer includes an emission layer, and
the emission layer includes the composition.

19. The organic optoelectric device as claimed in claim 18, wherein the composition is a host in the emission layer.

20. A display device comprising the organic optoelectric device as claimed in claim 17.
